(19) United States
(12) Patent Application Publication
(10) Pub. No.: US 2010/0267806 A1
(43) Pub. Date: Oct. 21, 2010

(54) LIPID FORMULATED COMPOSITIONS AND METHODS FOR INHIBITING EXPRESSION OF Eg5 AND VEGF GENES

(76) Inventors: David Bumcrot, Belmont, MA (US); Akin Akinc, Needham, MA (US); Dinah Wen-Yee Sah, Boston, MA (US); Tatiana Novobrantseva, Cambridge, MA (US)

Correspondence Address: ALNYLAM/FENWICK, SILICON VALLEY CENTER, 801 CALIFORNIA STREET, MOUNTAIN VIEW, CA, 94041, US

(21) Appl. No.: 12/723,471

(22) Filed: Mar. 12, 2010

Related U.S. Application Data
(60) Provisional application No. 61/159,788, filed on Mar. 12, 2009.
(60) Provisional application No. 61/231,579, filed on Aug. 5, 2009.
(60) Provisional application No. 61/285,947, filed on Dec. 11, 2009.

(51) Int. Cl. A61K 31/713; A61P 35/00; C12N 5/02

(52) U.S. Cl. 514/44 A; 435/375

(57) ABSTRACT

This invention relates to compositions containing double-stranded ribonucleic acid (dsRNA) in a lipid formulation, and methods of using the compositions to inhibit the expression of the Human kinesin family member 11 (Eg5) and Vascular Endothelial Growth Factor (VEGF), and methods of using the compositions to treat pathological processes mediated by Eg5 and VEGF expression, such as cancer.

### CROSS REFERENCE TO RELATED APPLICATIONS

This application claims the benefit of U.S. Provisional Application Ser. No. 61/159,788, filed Mar. 12, 2009; U.S. Provisional Application Ser. No. 61/231,579, filed Aug. 5, 2009, and U.S. Provisional Application Ser. No. 61/285,947, filed Dec. 11, 2009, all of which are incorporated herein by reference, in their entirety, for all purposes.

### FIELD OF THE INVENTION

This invention relates to lipid formulated compositions containing double-stranded ribonucleic acid(dsRNA), and their use in mediating RNA interference to inhibit the expression of a combination of genes, e.g., the Eg5 and Vascular Endothelial Growth Factor (VEGF) genes. The dsRNA are formulated in lipid formulation and can include a lipoprotein, e.g., apolipoprotein E. Also included in the invention is the use of the compositions to treat pathological processes mediated by Eg5 and VEGF expression, such as cancer.

### REFERENCE TO A SEQUENCE LISTING

This application includes a Sequence Listing submitted electronically as a text file named 16564US_sequencelisting.txt, created on Month, ______, 2010, with a size of ______ bytes. The sequence listing is incorporated by reference.

### BACKGROUND OF THE INVENTION

The maintenance of cell populations within an organism is governed by the cellular processes of cell division and programmed cell death. Within normal cells, the cellular events associated with the initiation and completion of each process is highly regulated. In proliferative disease such as cancer, one or both of these processes may be perturbed. For example, a cancer cell may have lost its regulation (checkpoint control) of the cell division cycle through either the overexpression of a positive regulator or the loss of a negative regulator, perhaps by mutation.

Alternatively, a cancer cell may have lost the ability to undergo programmed cell death through the overexpression of a negative regulator. Hence, there is a need to develop new chemotherapeutic drugs that will restore the processes of checkpoint control and programmed cell death to cancerous cells.

One approach to the treatment of human cancers is to target a protein that is essential for cell cycle progression. In order for the cell cycle to proceed from one phase to the next, certain prerequisite events must be completed. There are checkpoints within the cell cycle that enforce the proper order of events and phases. One such checkpoint is the spindle checkpoint that occurs during the metaphase stage of mitosis. Small molecules that target proteins with essential functions in mitosis may initiate the spindle checkpoint to arrest cells in mitosis. Of the small molecules that arrest cells in mitosis, those which display anti-tumor activity in the clinic also induce apoptosis, the morphological changes associated with programmed cell death. An effective chemotherapeutic for the treatment of cancer may thus be one which induces checkpoint control and programmed cell death. Unfortunately, there are few compounds available for controlling these processes within the cell. Most compounds known to cause mitotic arrest and apoptosis act as tubulin binding agents. These compounds alter the dynamic instability of microtubules and indirectly alter the function/structure of the mitotic spindle thereby causing mitotic arrest. Because most of these compounds specifically target the tubulin protein which is a component of all microtubules, they may also affect one or more of the numerous normal cellular processes in which microtubules have a role. Hence, there is also a need for agents that more specifically target proteins associated with proliferating cells.

Eg5 is one of several kinesin-like motor proteins that are localized to the mitotic spindle and known to be required for formation and/or function of the bipolar mitotic spindle. Recently, there was a report of a small molecule that disturbs bipolarity of the mitotic spindle (Mayer, T.

U. et al. 1999. Science 286(5441) 971-4, herein incorporated by reference). More specifically, the small molecule induced the formation of an aberrant mitotic spindle wherein a monoastral array of microtubules emanated from a central pair of centrosomes, with chromosomes attached to the distal ends of the microtubules. The small molecule was dubbed “monastrol” after the monoastral array. This monoastral array phenotype had been previously observed in mitotic cells that were immunodepleted of the Eg5 motor protein. This distinctive monoastral array phenotype facilitated identification of monastrol as a potential inhibitor of Eg5. Indeed, monastrol was further shown to inhibit the Eg5 motor-driven motility of microtubules in an in vitro assay. The Eg5 inhibitor monastrol had no apparent effect upon the related kinesin motor or upon the motor(s) responsible for golgi apparatus movement within the cell. Cells that display the monoastral array phenotype either through immunodepletion of Eg5 or monastrol inhibition of Eg5 arrest in M-phase of the cell cycle. However, the mitotic arrest induced by either immunodepletion or inhibition of Eg5 is transient (Kapoor, T. M., 2000. J Cell Biol 150(5) 975-80). Both the monoastral array phenotype and the cell cycle arrest in mitosis induced by monastrol are reversible. Cells recover to form a normal bipolar mitotic spindle, to complete mitosis and to proceed through the cell cycle and normal cell proliferation. These data suggest that an inhibitor of Eg5 which induced a transient mitotic arrest may not be effective for the treatment of cancer cell proliferation. Nonetheless, the discovery that monastrol causes mitotic arrest is intriguing and hence there is a need to further study and identify compounds which can be used to modulate the Eg5 motor protein in a manner that would be effective in the treatment of human cancers. There is also a need to explore the use of these compounds in combination with other antineoplastic agents.

VEGF (vascular endothelial growth factor, also known as vascular permeability factor, VPF) is a multifunctional cytokine that stimulates angiogenesis, epithelial cell proliferation, and endothelial cell survival. VEGF can be produced by a wide variety of tissues, and its overexpression or aberrant expression can result in a variety disorders, including cancers and retinal disorders, such as age-related macular degeneration and other angiogenic disorders.

Recently, double-stranded RNA molecules (dsRNA) have been shown to block gene expression in a highly conserved regulatory mechanism known as RNA interference (RNAi). WO 99/32619 (Fire et al.) discloses the use of a dsRNA of at least 25 nucleotides in length to inhibit the expression of genes in C. elegans. dsRNA has also been shown to degrade target RNA in other organisms, including plants (see, e.g., WO 99/53050, Waterhouse et al.; and WO 99/61631, Heifetz et al.), Drosophila (see, e.g., Yang, D., et al., Curr. Biol. (2000) 10:1191-1200), and mammals (see WO 00/44895, Limmer; and DE 101 00 586.5, Kreutzer et al.). This natural mechanism has now become the focus for the development of a new class of pharmaceutical agents for treating disorders that are caused by the aberrant or unwanted regulation of a gene.

### SUMMARY OF THE INVENTION

The invention provides compositions and methods for inhibiting the expression of human Eg5/KSP and VEGF genes in a cell using lipid formulated compositions containing dsRNA.

Compositions of the invention include a nucleic acid lipid particle having a first double-stranded ribonucleic acid (dsRNA) for inhibiting the expression of a human kinesin family member 11 (Eg5/KSP) gene in a cell and a second dsRNA for inhibiting expression of a human VEGF in a cell. The nucleic acid lipid particle has a lipid formulation having 45-65 mol % of a cationic lipid, 5 mol % to about 10 mol %, of a non-cationic lipid, 25-40 mol % of a sterol, and 0.5-5 mol % of a PEG or PEG-modified lipid. The first dsRNA targeting Eg5/KSP includes a first sense strand and a first antisense strand, and the first sense strand having a first sequence and the first antisense strand has a second sequence complementary to at least 15 contiguous nucleotides of SEQ ID NO:1311 (5′-UCGAGAAUCUAAACUAACU-3′), wherein the first sequence is complementary to the second sequence and wherein the first dsRNA is between 15 and 30 base pairs in length. The second dsRNA includes a second sense strand and a second antisense strand, the second sense strand having a third sequence and the second antisense strand having a fourth sequence complementary to at least 15 contiguous nucleotides of SEQ ID NO:1538 (5′-GCACAUAGGAGAGAUGAGCUU-3′), wherein the third sequence is complementary to the fourth sequence and wherein the second dsRNA is between 15 and 30 base pairs in length.

In one embodiment, the cationic lipid of the composition has formula A, wherein formula A is

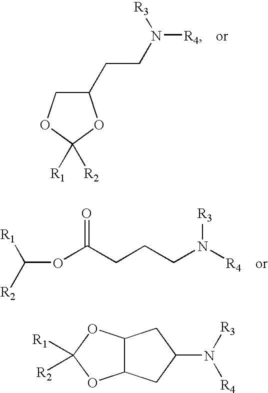

where R1 and R2 are independently alkyl, alkenyl or alkynyl, each can be optionally substituted, and R3 and R4 are independently lower alkyl or R3 and R4 can be taken together to form an optionally substituted heterocyclic ring.

In other embodiments, the cationic lipid is XTC (2,2-Dilinoleyl-4-dimethylaminoethyl-[1,3]-dioxolane). In a related embodiment, the cationic lipid is XTC, the non-cationic lipid is DSPC, the sterol is cholesterol and the PEG lipid has PEG-DMG. In a yet related embodiment, the cationic lipid is XTC and the formulation is selected from the group consisting of:

| | |
|---|---|
| LNP05 | XTC/DSPC/Cholesterol/PEG-DMG |
| | 57.5/7.5/31.5/3.5 |
| | lipid:siRNA ~6:1 |
| LNP06 | XTC/DSPC/Cholesterol/PEG-DMG |
| | 57.5/7.5/31.5/3.5 |
| | lipid:siRNA ~11:1 |
| LNP07 | XTC/DSPC/Cholesterol/PEG-DMG |
| | 60/7.5/31/1.5, |
| | lipid:siRNA ~6:1 |
| LNP08 | XTC/DSPC/Cholesterol/PEG-DMG |
| | 60/7.5/31/1.5, |
| | lipid:siRNA ~11:1 |
| LNP09 | XTC/DSPC/Cholesterol/PEG-DMG |
| | 50/10/38.5/1.5 |
| | lipid:siRNA ~10:1 |
| LNP13 | XTC/DSPC/Cholesterol/PEG-DMG |
| | 50/10/38.5/1.5 |
| | lipid:siRNA ~33:1 |
| LNP22 | XTC/DSPC/Cholesterol/PEG-DSG |
| | 50/10/38.5/1.5 |
| | lipid:siRNA ~10 |

In another embodiment, the cationic lipid of the composition is ALNY-100 ((3aR,5s,6aS)-N,N-dimethyl-2,2-di((9Z,12Z)-octadeca-9,12-dienyl)tetrahydro-3aH-cyclopenta[d][1,3]dioxol-5-amine)). In other embodiments, the cationic lipid is ALNY-100 and the formulation includes:

| | |
|---|---|
| LNP10 | ALNY-100/DSPC/Cholesterol/PEG-DMG |
| | 50/10/38.5/1.5 |
| | lipid:siRNA ~10:1 |

In other embodiments, the cationic lipid is MC3 (((6Z,9Z,28Z,31Z)-heptatriaconta-6,9,28,31-tetraen-19-yl 4-(dimethylamino)butanoate). In a related embodiment, the cationic lipid 9s MC3 and the lipid formulation is selected from the group consisting of:

| | |
|---|---|
| LNP11 | MC3/DSPC/Cholesterol/PEG-DMG |
| | 50/10/38.5/1.5 |
| | lipid:siRNA ~10:1 |
| LNP14 | MC3/DSPC/Cholesterol/PEG-DMG |
| | 40/15/40/5 |
| | lipid:siRNA ~11 |
| LNP15 | MC3/DSPC/Cholesterol/PEG-DSG/GalNAc-PEG- |
| | DSG |
| | 50/10/35/4.5/0.5 |
| | lipid:siRNA ~11 |
| LNP16 | MC3/DSPC/Cholesterol/PEG-DMG |
| | 50/10/38.5/1.5 |
| | lipid:siRNA ~7 |
| LNP17 | MC3/DSPC/Cholesterol/PEG-DSG |
| | 50/10/38.5/1.5 |
| | lipid:siRNA ~10 |
| LNP18 | MC3/DSPC/Cholesterol/PEG-DMG |
| | 50/10/38.5/1.5 |
| | lipid:siRNA ~12 |
| LNP19 | MC3/DSPC/Cholesterol/PEG-DMG |
| | 50/10/35/5 |
| | lipid:siRNA ~8 |
| LNP20 | MC3/DSPC/Cholesterol/PEG-DPG |
| | 50/10/38.5/1.5 |
| | lipid:siRNA ~10 |

In another embodiment, the first dsRNA includes a sense strand consisting of SEQ ID NO:1534 (5′-UCGAGAAUCUAAACUAACUTT-3′) and an antisense strand consisting of SEQ ID NO:1535 (5′-AGUUAGUUUAGAUUCCUGATT-3′) and the second dsRNA includes a sense strand consisting of SEQ ID NO:1536 (5′-GCACAUAGGAGAGAUGAGCUU-3′), and an antisense strand consisting of SEQ ID NO:1537 (5′-AAGCUCAUCUCUCCUAUGUGCUG-3′). In yet another embodiment, each strand is modified as follows to include a 2′-O-methyl ribonucleotide as indicated by a lower case letter “c” or “u” and a phosphorothioate as indicated by a lower case letter “s”: the first dsRNA includes a sense strand consisting of SEQ ID NO:1240 (5′-ucGAGAAucuAAAcuAAcuTsT-3′) and an antisense strand consisting of SEQ ID NO:1241 (5′-AGUuAGUUuAGAUUCUCGATsT); the second dsRNA includes a sense strand consisting of SEQ ID NO:1242 (5′-GcAcAuAGGAGAGAuGAGCUsU-3′) and an antisense strand consisting of SEQ ID NO:1243 (5′-AAGCUcAUCUCUCCuAuGuGCusG-3′).

In other embodiments, the first and second dsRNA includes at least one modified nucleotide. In some embodiments, the modified nucleotide is chosen from the group of: a 2′-O-methyl modified nucleotide, a nucleotide having a 5′-phosphorothioate group, and a terminal nucleotide linked to a cholesteryl derivative or dodecanoic acid bisdecylamide group. In another embodiment, the modified nucleotide is chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base having nucleotide. In yet another embodiment, the first and second dsRNA each comprise at least one 2′-O-methyl modified ribonucleotide and at least one nucleotide having a 5′-phosphorothioate group.

In some embodiments, each dsRNA is 19-23 bases in length. In another embodiment, each strand of each dsRNA is 21-23 bases in length. In yet another embodiment, each strand of the first dsRNA is 21 bases in length, the sense strand of the second dsRNA is 21 bases in length and the antisense strand of the second dsRNA is 23 bases in length. In other embodiments, the first and second dsRNA are present in an equimolar ratio. In one embodiment, the composition further has Sorafenib. In another embodiment, the composition further has a lipoprotein. In another embodiment, the composition further has apolipoprotein E (ApoE).

In another embodiment, the composition, upon contact with a cell expressing Eg5, inhibits expression of Eg5 by at least 40%. In yet another embodiment, the composition, upon contact with a cell expressing VEGF, inhibits expression of VEGF by at least 40%. In other embodiments, the administration of the composition to a cell decreases expression of Eg5 and VEGF in the cell. In a related embodiment, the composition is administered in a nM concentration. In a yet related embodiment, the administration of the composition to a cell increases monoaster formation in the cell.

In other embodiments, the administration of the composition to a mammal results in at least one effect selected from the group consisting of prevention of tumor growth, reduction in tumor growth, or prolonged survival in the mammal. In some embodiments, the effect is measured using at least one assay selected from the group consisting of determination of body weight, determination of organ weight, visual inspection, mRNA analysis, serum AFP analysis and survival monitoring.

The invention also provides methods for inhibiting the expression of Eg5/KSP and VEGF in a cell. The methods includes the steps ofadministering the composition of the invention to a cell. The invention also provides methods for preventing tumor growth, reducing tumor growth, or prolonging survival in a mammal in need of treatment for cancer. The methods include the step of administering the composition of the inventionto the mammal. In one embodiment, the mammal has liver cancer. In another embodiment, the mammal is a human with liver cancer. In some embodiments, a dose containing between 0.25 mg/kg and 4 mg/kg dsRNA is administered to the mammal. In other embodiments, the dsRNA is administered to a human at about 0.01, 0.1, 0.5, 1.0, 2.5, or 5.0 mg/kg.

In yet another embodiment, the invention provides methods for reducing tumor growth in a mammal in need of treatment for cancer. The methods include administering the composition of the invention to the mammal, the method reducing tumor growth by at least 20%. In another embodiment, the method reduces KSP expression by at least 60%.

### BRIEF DESCRIPTION OF THE FIGURES

FIG. 1 is a graph showing liver weights as a percentage of body weight following administration of SNALP-siRNAs in a Hep3B mouse model.

FIG. 2A is a graph showing the effect of PBS on body weight in a Hep3B mouse model. FIG. 2B is a graph showing the effect of a SNALP-siRNA (VEGF/KSP) on body weight in a Hep3B mouse model.

FIG. 2C is a graph showing the effect of a SNALP-siRNA (KSP/Luciferase) on body weight in a Hep3B mouse model.

FIG. 2D is a graph showing the effect of SNALP-siRNA (VEGF/Luciferase) on body weight in a Hep3B mouse model.

FIG. 3 is a graph showing the effects of SNALP-siRNAs on body weight in a Hep3B mouse model.

FIG. 4 is a graph showing the body weight in untreated control animals.

FIG. 5 is a graph showing the effects of control luciferase-SNALP siRNAs on body weight in a Hep3B mouse model.

FIG. 6 is a graph showing the effects of VSP-SNALP siRNAs on body weight in a Hep3B mouse model.

FIG. 7A is a graph showing the effects of SNALP-siRNAs on human GAPDH levels normalized to mouse GAPDH levels in a Hep3B mouse model.

FIG. 7B is a graph showing the effects of SNALP-siRNAs on serum AFP levels as measured by serum ELISA in a Hep3B mouse model.

FIG. 8 is a graph showing the effects of SNALP-siRNAs on human GAPDH levels normalized to mouse GAPDH levels in a Hep3B mouse model.

FIG. 9 is a graph showing the effects of SNALP-siRNAs on human KSP levels normalized to human GAPDH levels in a Hep3B mouse model.

FIG. 10 is a graph showing the effects of SNALP-siRNAs on human VEGF levels normalized to human GAPDH levels in a Hep3B mouse model.

FIG. 11A is a graph showing the effects of SNALP-siRNAs on mouse VEGF levels normalized to human GAPDH levels in a Hep3B mouse model.

FIG. 11B is a set of graphs showing the effects of SNALP-siRNAs on human GAPDH levels and serum AFP levels in a Hep3B mouse model.

FIG. 12A is a graph showing the effect of PBS, Luciferase, and ALN-VSP on tumor KSP measured by percentage of relative hKSP mRNA in a Hep3B mouse model.

FIG. 12B is a graph showing the effect of PBS, Luciferase, and SNALP-VSP on tumor VEGF measured by percentage of relative hVEGF mRNA in a Hep3B mouse model.

FIG. 12C is a graph showing the effect of PBS, Luciferase, and SNALP-VSP on GAPDH levels measured by percentage of relative hGAPDH mRNA in a Hep3B mouse model.

FIG. 13A is a graph showing the effect of SNALP si-RNAs on survival in mice with hepatic tumors. Treatment was started at 18 days after tumor cell seeding.

FIG. 13B is a graph showing the effect of SNALP-siRNAs on survival in mice with hepatic tumors. Treatment was started at 26 days after tumor cell seeding.

FIG. 14 is a graph showing the effects of SNALP-siRNAs on serum alpha fetoprotein (AFP) levels.

FIG. 15A is an image of H&E stained sections in tumor bearing animals (three weeks after Hep3B cell implantation) that were administered 2 mg/kg SNALP-VSP. Twenty four hours later, tumor bearing liver lobes were processed for histological analysis. Arrows indicate mono asters.

FIG. 15B is an image of H&E stained sections in tumor bearing animals (three weeks after Hep3B cell implantation) that were administered 2 mg/kg SNALP-Luc. Twenty four hours later, tumor bearing liver lobes were processed for histological analysis.

FIG. 16 is a graph illustrating the effects on survival of administration SNALP formulated siRNA and Sorafenib.

FIG. 17 is a flow chart of the in-line mixing method.

FIG. 18 are graphs illustrating the effects on KSP and VEGF expression in intrahepatic Hep3B tumors in mice following treatment with LNP-08 formulated VSP.

FIG. 19 illustrates the chemical structures of PEG-DSG and PEG-C-DSA.

FIG. 20 illustrates the structures of cationic lipids ALNY-100, MC3, and XTC.

FIG. 21 are graphs illustrating the effects on KSP and VEGF expression in intrahepatic Hep3B tumors in mice treated with SNALP-1955 (Luc), ALN-VSP02, and SNALP-T-VSP LNP11 and LNP-12 formulated VSP.

FIG. 22 is a set of graphs comparing the effects on KSP and VEGF expression in intrahepatic Hep3B tumors in mice treated with LNP08-Luc, ALN-VSP02, and LNP-08 and LNP08-C18 formulated VSP.

### DETAILED DESCRIPTION OF THE INVENTION

The invention provides compositions and methods for inhibiting the expression of the Eg5 gene and VEGF gene in a cell or mammal using the dsRNAs. The dsRNAs are packaged in a lipid nucleic acid particle. The invention also provides compositions and methods for treating pathological conditions and diseases, such as liver cancer, in a mammal caused by the expression of the Eg5 gene and VEGF genes. The dsRNA directs the sequence-specific degradation of mRNA through a process known as RNA interference (RNAi).

The following detailed description discloses how to make and use the compositions containing dsRNAs to inhibit the expression of the Eg5 gene and VEGF genes, respectively, as well as compositions and methods for treating diseases and disorders caused by the expression of these genes, such as cancer. The pharmaceutical compositions featured in the invention include a dsRNA having an antisense strand comprising a region of complementarity which is less than 30 nucleotides in length, generally 19-24 nucleotides in length, and is substantially complementary to at least part of an RNA transcript of the Eg5 gene, together with a pharmaceutically acceptable carrier. The compositions featured in the invention also include a dsRNA having an antisense strand having a region of complementarity which is less than 30 nucleotides in length, generally 19-24 nucleotides in length, and is substantially complementary to at least part of an RNA transcript of the VEGF gene.

Accordingly, certain aspects of the invention provide pharmaceutical compositions containing the Eg5 and VEGF dsRNAs and a pharmaceutically acceptable carrier, methods of using the compositions to inhibit expression of the Eg5 gene and the VEGF gene respectively, and methods of using the pharmaceutical compositions to treat diseases caused by expression of the Eg5 and VEGF genes.

I. Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

“G,” “C,” “A” and “U” each generally stand for a nucleotide that contains guanine, cytosine, adenine, and uracil as a base, respectively. “T” and “dT” are used interchangeably herein and refer to a deoxyribonucleotide wherein the nucleobase is thymine, e.g., deoxyribothymine. However, it will be understood that the term “ribonucleotide” or “nucleotide” can also refer to a modified nucleotide, as further detailed below, or a surrogate replacement moiety. The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine. In another example, adenine and cytosine anywhere in the oligonucleotide can be replaced with guanine and uracil, respectively to form G-U Wobble base pairing with the target mRNA. Sequences comprising such replacement moieties are embodiments of the invention.

As used herein, “Eg5” refers to the human kinesin family member 11, which is also known as KIF11, Eg5, HKSP, KSP, KNSL1 or TRIPS. Eg5 sequence can be found as NCBI GeneID:3832, HGNC ID: HGNC:6388 and RefSeq ID number:NM_{—}004523. The terms “Eg5” and “KSP” and “Eg5/KSP” are used interchangeably

As used herein, “VEGF,” also known as vascular permeability factor, is an angiogenic growth factor. VEGF is a homodimeric 45 kDa glycoprotein that exists in at least three different isoforms. VEGF isoforms are expressed in endothelial cells. The VEGF gene contains 8 exons that express a 189-amino acid protein isoform. A 165-amino acid isoform lacks the residues encoded by exon 6, whereas a 121-amino acid isoform lacks the residues encoded by exons 6 and 7. VEGF145 is an isoform predicted to contain 145 amino acids and to lack exon 7. VEGF can act on endothelial cells by binding to an endothelial tyrosine kinase receptor, such as Flt-1 (VEGFR-1) or KDR/flk-1 (VEGFR-2). VEGFR-2 is expressed in endothelial cells and is involved in endothelial cell differentiation and vasculogenesis. A third receptor, VEGFR-3, has been implicated in lymphogenesis.

The various isoforms have different biologic activities and clinical implications. For example, VEGF145 induces angiogenesis and like VEGF189 (but unlike VEGF165), VEGF145 binds efficiently to the extracellular matrix by a mechanism that is not dependent on extracellular matrix-associated heparin sulfates. VEGF displays activity as an endothelial cell mitogen and chemoattractant in vitro and induces vascular permeability and angiogenesis in vivo. VEGF is secreted by a wide variety of cancer cell types and promotes the growth of tumors by inducing the development of tumor-associated vasculature. Inhibition of VEGF function has been shown to limit both the growth of primary experimental tumors as well as the incidence of metastases in immunocompromised mice. Various dsRNAs directed to VEGF are described in co-pending U.S. Ser. Nos. 11/078,073 and 11/340,080, which are hereby incorporated by reference in their entirety.

As used herein, “target sequence” refers to a contiguous portion of the nucleotide sequence of an mRNA molecule formed during the transcription of the Eg5/KSP and/or VEGF gene, including mRNA that is a product of RNA processing of a primary transcription product.

As used herein, the term “strand comprising a sequence” refers to an oligonucleotide comprising a chain of nucleotides that is described by the sequence referred to using the standard nucleotide nomenclature.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

The term “complementary” includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be referred to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide which is “substantially complementary to at least part of a messenger RNA (mRNA) refers to a polynucleotide which is substantially complementary to a contiguous portion of the mRNA of interest (e.g., encoding Eg5/KSP and/or VEGF) including a 5′ untranslated region (UTR), an open reading frame (ORF), or a 3′ UTR. For example, a polynucleotide is complementary to at least a part of a Eg5 mRNA if the sequence is substantially complementary to a non-interrupted portion of a mRNA encoding Eg5.

The term “double-stranded RNA” or “dsRNA”, as used herein, refers to a duplex structure comprising two anti-parallel and substantially complementary, as defined above, nucleic acid strands. In general, the majority of nucleotides of each strand are ribonucleotides, but as described in detail herein, each or both strands can also include at least one non-ribonucleotide, e.g., a deoxyribonucleotide and/or a modified nucleotide. In addition, as used in this specification, “dsRNA” may include chemical modifications to ribonucleotides, including substantial modifications at multiple nucleotides and including all types of modifications disclosed herein or known in the art. Any such modifications, as used in an siRNA type molecule, are encompassed by “dsRNA” for the purposes of this specification and claims.

The two strands forming the duplex structure may be different portions of one larger RNA molecule, or they may be separate RNA molecules. Where the two strands are part of one larger molecule, and therefore are connected by an uninterrupted chain of nucleotides between the 3′ end of one strand and the 5′ end of the respective other strand forming the duplex structure, the connecting RNA chain is referred to as a “hairpin loop”. Where the two strands are connected covalently by means other than an uninterrupted chain of nucleotides between the 3′ end of one strand and the 5′ end of the respective other strand forming the duplex structure, the connecting structure is referred to as a “linker.” The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs. In general, the majority of nucleotides of each strand are ribonucleotides, but as described in detail herein, each or both strands can also include at least one non-ribonucleotide, e.g., a deoxyribonucleotide and/or a modified nucleotide. In addition, as used in this specification, “dsRNA” may include chemical modifications to ribonucleotides, including substantial modifications at multiple nucleotides and including all types of modifications disclosed herein or known in the art. Any such modifications, as used in an siRNA type molecule, are encompassed by “dsRNA” for the purposes of this specification and claims.

As used herein, a “nucleotide overhang” refers to the unpaired nucleotide or nucleotides that protrude from the duplex structure of a dsRNA when a 3′ end of one strand of the dsRNA extends beyond the 5′ end of the other strand, or vice versa. “Blunt” or “blunt end” means that there are no unpaired nucleotides at that end of the dsRNA, i.e., no nucleotide overhang. A “blunt ended” dsRNA is a dsRNA that is double-stranded over its entire length, i.e., no nucleotide overhang at either end of the molecule. In some embodiments the dsRNA can have a nucleotide overhang at one end of the duplex and a blunt end at the other end.

The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches may be in the internal or terminal regions of the molecule. Generally, the most tolerated mismatches are in the terminal regions, e.g., within 6, 5, 4, 3, or 2 nucleotides of the 5′ and/or 3′ terminus.

The term “sense strand,” as used herein, refers to the strand of a dsRNA that includes a region that is substantially complementary to a region of the antisense strand.

“Introducing into a cell,” when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro. A dsRNA may also be “introduced into a cell”, wherein the cell is part of a living organism. In such instance, introduction into the cell will include the delivery to the organism. For example, for in vivo delivery, dsRNA can be injected into a tissue site or administered systemically. In vitro introduction into a cell includes methods known in the art such as electroporation and lipofection.

The terms “silence” and “inhibit the expression of “down-regulate the expression of,” “suppress the expression of and the like, in as far as they refer to the Eg5 and/or VEGF gene, herein refer to the at least partial suppression of the expression of the Eg5 gene, as manifested by a reduction of the amount of Eg5 mRNA and/or VEGF mRNA which may be isolated from a first cell or group of cells in which the Eg5 and/or VEGF gene is transcribed and which has or have been treated such that the expression of the Eg5 and/or VEGF gene is inhibited, as compared to a second cell or group of cells substantially identical to the first cell or group of cells but which has or have not been so treated (control cells). The degree of inhibition is usually expressed in terms of

$$\frac{( mRNA in control cells ) - ( mRNA in treated cells )}{( mRNA in control cells )} \cdot 100 \%$$

Alternatively, the degree of inhibition may be given in terms of a reduction of a parameter that is functionally linked to Eg5 and/or VEGF gene expression, e.g. the amount of protein encoded by the Eg5 and/or VEGF gene which is produced by a cell, or the number of cells displaying a certain phenotype, e.g. apoptosis. In principle, target gene silencing can be determined in any cell expressing the target, either constitutively or by genomic engineering, and by any appropriate assay. However, when a reference is needed in order to determine whether a given dsRNA inhibits the expression of the Eg5 gene by a certain degree and therefore is encompassed by the instant invention, the assay provided in the Examples below shall serve as such reference.

For example, in certain instances, expression of the Eg5 gene (or VEGF gene) is suppressed by at least about 5%, 10%, 15%, 20%, 25%, 30%, 35%, 40%, 45%, or 50% by administration of the double-stranded oligonucleotide of the invention. In some embodiments, the Eg5 and/or VEGF gene is suppressed by at least about 60%, 70%, or 80% by administration of the double-stranded oligonucleotide of the invention. In other embodiments, the Eg5 and/or VEGF gene is suppressed by at least about 85%, 90%, or 95% by administration of the double-stranded oligonucleotide of the invention. The Tables and Example below provides values for inhibition of expression using various Eg5 and/or VEGF dsRNA molecules at various concentrations.

As used herein in the context of Eg5 expression (or VEGF expression), the terms “treat,” “treatment,” and the like, refer to relief from or alleviation of pathological processes mediated by Eg5 and/or VEGF expression. In the context of the present invention, insofar as it relates to any of the other conditions recited herein below (other than pathological processes mediated by Eg5 and/or VEGF expression), the terms “treat,” “treatment,” and the like mean to relieve or alleviate at least one symptom associated with such condition, or to slow or reverse the progression of such condition, such as the slowing and progression of hepatic carcinoma.

As used herein, the phrases “therapeutically effective amount” and “prophylactically effective amount” refer to an amount that provides a therapeutic benefit in the treatment, prevention, or management of pathological processes mediated by Eg5 and/or VEGF expression or an overt symptom of pathological processes mediated by Eg5 and/or VEGF expression. The specific amount that is therapeutically effective can be readily determined by ordinary medical practitioner, and may vary depending on factors known in the art, such as, e.g., the type of pathological processes mediated by Eg5 and/or VEGF expression, the patient's history and age, the stage of pathological processes mediated by Eg5 and/or VEGF expression, and the administration of other anti-pathological processes mediated by Eg5 and/or VEGF expression agents.

As used herein, a “pharmaceutical composition” comprises a pharmacologically effective amount of a dsRNA and a pharmaceutically acceptable carrier. As used herein, “pharmacologically effective amount,” “therapeutically effective amount” or simply “effective amount” refers to that amount of an RNA effective to produce the intended pharmacological, therapeutic or preventive result. For example, if a given clinical treatment is considered effective when there is at least a 25% reduction in a measurable parameter associated with a disease or disorder, a therapeutically effective amount of a drug for the treatment of that disease or disorder is the amount necessary to effect at least a 25% reduction in that parameter.

The term “pharmaceutically acceptable carrier” refers to a carrier for administration of a therapeutic agent. As described in more detail below, such carriers include, but are not limited to, saline, buffered saline, dextrose, water, glycerol, ethanol, and combinations thereof. The term specifically excludes cell culture medium. For drugs administered orally, pharmaceutically acceptable carriers include, but are not limited to, pharmaceutically acceptable excipients, such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

As used herein, a “transformed cell” is a cell into which a vector has been introduced from which a dsRNA molecule may be expressed.

II. Double-Stranded Ribonucleic Acid (dsRNA)

As described in more detail herein, the invention provides double-stranded ribonucleic acid (dsRNA) molecules for inhibiting the expression of the Eg5 and/or VEGF gene in a cell or mammal, wherein the dsRNA comprises an antisense strand comprising a region of complementarity which is complementary to at least a part of an mRNA formed in the expression of the Eg5 and/or VEGF gene, and wherein the region of complementarity is less than 30 nucleotides in length, generally 19-24 nucleotides in length, and wherein said dsRNA, upon contact with a cell expressing said Eg5 and/or VEGF gene, inhibits the expression of said Eg5 and/or VEGF gene. The dsRNA of the invention can further include one or more single-stranded nucleotide overhangs.

The dsRNA can be synthesized by standard methods known in the art as further discussed below, e.g., by use of an automated DNA synthesizer, such as are commercially available from, for example, Biosearch, Applied Biosystems, Inc. The dsRNA comprises two strands that are sufficiently complementary to hybridize to form a duplex structure. One strand of the dsRNA (the antisense strand) comprises a region of complementarity that is substantially complementary, and generally fully complementary, to a target sequence, derived from the sequence of an mRNA formed during the expression of the Eg5 and/or VEGF gene, the other strand (the sense strand) comprises a region which is complementary to the antisense strand, such that the two strands hybridize and form a duplex structure when combined under suitable conditions. Generally, the duplex structure is between 15 and 30, or between 25 and 30, or between 18 and 25, or between 19 and 24, or between 19 and 21, or 19, 20, or 21 base pairs in length. In one embodiment the duplex is 19 base pairs in length. In another embodiment the duplex is 21 base pairs in length. When two different siRNAs are used in combination, the duplex lengths can be identical or can differ.

Each strand of the dsRNA of invention is generally between 15 and 30, or between 18 and 25, or 18, 19, 20, 21, 22, 23, or 24 nucleotides in length. In other embodiments, each is strand is 25-30 base pairs in length. Each strand of the duplex can be the same length or of different lengths. When two different siRNAs are used in combination, the lengths of each strand of each siRNA can be identical or can differ. For example, a composition can include a dsRNA targeted to Eg5 with a sense strand of 21 nucleotides and an antisense strand of 21 nucleotides, and a second dsRNA targeted to VEGF with a sense strand of 21 nucleotides and an antisense strand of 23 nucleotides.

The dsRNA of the invention can include one or more single-stranded overhang(s) of one or more nucleotides. In one embodiment, at least one end of the dsRNA has a single-stranded nucleotide overhang of 1 to 4, generally 1 or 2 nucleotides. In another embodiment, the antisense strand of the dsRNA has 1-10 nucleotides overhangs each at the 3′ end and the 5′ end over the sense strand. In further embodiments, the sense strand of the dsRNA has 1-10 nucleotides overhangs each at the 3′ end and the 5′ end over the antisense strand.

A dsRNA having at least one nucleotide overhang can have unexpectedly superior inhibitory properties than the blunt-ended counterpart. In some embodiments the presence of only one nucleotide overhang strengthens the interference activity of the dsRNA, without affecting its overall stability. A dsRNA having only one overhang has proven particularly stable and effective in vivo, as well as in a variety of cells, cell culture mediums, blood, and serum. Generally, the single-stranded overhang is located at the 3′ terminal end of the antisense strand or, alternatively, at the 3′ terminal end of the sense strand. The dsRNA can also have a blunt end, generally located at the 5′ end of the antisense strand. Such dsRNAs can have improved stability and inhibitory activity, thus allowing administration at low dosages, i.e., less than 5 mg/kg body weight of the recipient per day. Generally, the antisense strand of the dsRNA has a nucleotide overhang at the 3′ end, and the 5′ end is blunt. In another embodiment, one or more of the nucleotides in the overhang is replaced with a nucleoside thiophosphate.

As described in more detail herein, the composition of the invention includes a first dsRNA targeting Eg5 and a second dsRNA targeting VEGF. The first and second dsRNA can have the same overhang architecture, e.g., number of nucleotide overhangs on each strand, or each dsRNA can have a different architecture. In one embodiment, the first dsRNA targeting Eg5 includes a 2 nucleotide overhang at the 3′ end of each strand and the second dsRNA targeting VEGF includes a 2 nucleotide overhang on the 3′ end of the antisense strand and a blunt end at the 5′ end of the antisense strand (e.g., the 3′ end of the sense strand).

In one embodiment, the Eg5 gene targeted by the dsRNA of the invention is the human Eg5 gene. In one embodiment, the antisense strand of the dsRNA targeting Eg5 comprises at least 15 contiguous nucleotides of one of the antisense sequences of Tables 1-3. In specific embodiments, the first sequence of the dsRNA is selected from one of the sense strands of Tables 1-3, and the second sequence is selected from the group consisting of the antisense sequences of Tables 1-3. Alternative antisense agents that target elsewhere in the target sequence provided in Tables 1-3 can readily be determined using the target sequence and the flanking Eg5 sequence. In some embodiments, the dsRNA targeted to Eg5 will comprise at least two nucleotide sequence selected from the groups of sequences provided in Tables 1-3. One of the two sequences is complementary to the other of the two sequences, with one of the sequences being substantially complementary to a sequence of an mRNA generated in the expression of the Eg5 gene. As such, the dsRNA will comprises two oligonucleotides, wherein one oligonucleotide is described as the sense strand in Tables 1-3, and the second oligonucleotide is described as the antisense strand in Tables 1-3.

In embodiments using a second dsRNA targeting VEGF, such agents are exemplified in the Examples, Tables 4a and 4b, and in co-pending U.S. Ser. Nos. 11/078,073 and 11/340,080, herein incorporated by reference. In one embodiment the dsRNA targeting VEGF has an antisense strand complementary to at least 15 contiguous nucleotides of the VEGF target sequences described in Table 4a. In other embodiments, the dsRNA targeting VEGF comprises one of the antisense sequences of Table 4b, or one of the sense sequences of Table 4b, or comprises one of the duplexes (sense and antisense strands) of Table 4b.

The skilled person is well aware that dsRNAs comprising a duplex structure of between 20 and 23, but specifically 21, base pairs have been hailed as particularly effective in inducing RNA interference (Elbashir et al., EMBO 2001, 20:6877-6888). However, others have found that shorter or longer dsRNAs can be effective as well. In the embodiments described above, by virtue of the nature of the oligonucleotide sequences provided in Tables 1-3, the dsRNAs of the invention can comprise at least one strand of a length of minimally 21 nt. It can be reasonably expected that shorter dsRNAs comprising one of the sequences of Tables 1-3 minus only a few nucleotides on one or both ends may be similarly effective as compared to the dsRNAs described above. Hence, dsRNAs comprising a partial sequence of at least 15, 16, 17, 18, 19, 20, or more contiguous nucleotides from one of the sequences of Tables 1-3, and differing in their ability to inhibit the expression of the Eg5 gene in a FACS assay as described herein below by not more than 5, 10, 15, 20, 25, or 30% inhibition from a dsRNA comprising the full sequence, are contemplated by the invention. Further dsRNAs that cleave within the target sequence provided in Tables 1-3 can readily be made using the Eg5 sequence and the target sequence provided. Additional dsRNA targeting VEGF can be designed in a similar matter using the sequences disclosed in Tables 4a and 4b, the Examples and co-pending U.S. Ser. Nos. 11/078,073 and 11/340,080, herein incorporated by reference.

In addition, the RNAi agents provided in Tables 1-3 identify a site in the Eg5 mRNA that is susceptible to RNAi based cleavage. As such the present invention further includes RNAi agents, e.g., dsRNA, that target within the sequence targeted by one of the agents of the present invention. As used herein a second RNAi agent is said to target within the sequence of a first RNAi agent if the second RNAi agent cleaves the message anywhere within the mRNA that is complementary to the antisense strand of the first RNAi agent. Such a second agent will generally consist of at least 15 contiguous nucleotides from one of the sequences provided in Tables 1-3 coupled to additional nucleotide sequences taken from the region contiguous to the selected sequence in the Eg5 gene. For example, the last 15 nucleotides of SEQ ID NO:1 combined with the next 6 nucleotides from the target Eg5 gene produces a single strand agent of 21 nucleotides that is based on one of the sequences provided in Tables 1-3. Additional RNAi agents, e.g., dsRNA, targeting VEGF can be designed in a similar matter using the sequences disclosed in Tables 4a and 4b, the Examples and co-pending U.S. Ser. Nos. 11/078,073 and 11/340,080, herein incorporated by reference.

The dsRNA of the invention can contain one or more mismatches to the target sequence. In a preferred embodiment, the dsRNA of the invention contains no more than 3 mismatches. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity. For example, for a 23 nucleotide dsRNA strand which is complementary to a region of the Eg5 gene, the dsRNA generally does not contain any mismatch within the central 13 nucleotides. The methods described within the invention can be used to determine whether a dsRNA containing a mismatch to a target sequence is effective in inhibiting the expression of the Eg5 gene. Consideration of the efficacy of dsRNAs with mismatches in inhibiting expression of the Eg5 gene is important, especially if the particular region of complementarity in the Eg5 gene is known to have polymorphic sequence variation within the population.

Modifications

In yet another embodiment, the dsRNA is chemically modified to enhance stability. The nucleic acids of the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Specific examples of preferred dsRNA compounds useful in this invention include dsRNAs containing modified backbones or no natural internucleoside linkages. As defined in this specification, dsRNAs having modified backbones include those that retain a phosphorus atom in the backbone and those that do not have a phosphorus atom in the backbone. For the purposes of this specification, and as sometimes referenced in the art, modified dsRNAs that do not have a phosphorus atom in their internucleoside backbone can also be considered to be oligonucleosides.

Preferred modified dsRNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; and 5,625,050, each of which is herein incorporated by reference

Preferred modified dsRNA backbones that do not include a phosphorus atom therein have backbones that are formed by short chain alkyl or cycloalkyl internucleoside linkages, mixed heteroatoms and alkyl or cycloalkyl internucleoside linkages, or ore or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other preferred dsRNA mimetics, both the sugar and the internucleoside linkage, i.e., the backbone, of the nucleotide units are replaced with novel groups. The base units are maintained for hybridization with an appropriate nucleic acid target compound. One such oligomeric compound, a dsRNA mimetic that has been shown to have excellent hybridization properties, is referred to as a peptide nucleic acid (PNA). In PNA compounds, the sugar backbone of a dsRNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al., Science, 1991, 254, 1497-1500.

Most preferred embodiments of the invention are dsRNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂N(CH₃)—N(CH₃)—CH₂— and —N(CH₃)—CH₂—CH₂' [wherein the native phosphodiester backbone is represented as —O—P—O—CH₂—] of the above-referenced U.S. Pat. No. 5,489,677, and the amide backbones of the above-referenced U.S. Pat. No. 5,602,240. Also preferred are dsRNAs having morpholino backbone structures of the above-referenced U.S. Pat. No. 5,034,506.

Modified dsRNAs may also contain one or more substituted sugar moieties. Preferred dsRNAs comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. Other preferred dsRNAs comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an dsRNA, or a group for improving the pharmacodynamic properties of an dsRNA, and other substituents having similar properties. A preferred modification includes 2′-methoxyethoxy (2′-O—CH₂CH₂OCH₃, also known as 2′-O-(2-methoxyethyl) or 2′-MOE) (Martin et al., Hely. Chim. Acta, 1995, 78, 486-504) i.e., an alkoxy-alkoxy group. A further preferred modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other preferred modifications include 2′-methoxy (2′-OCH₃), 2′-aminopropoxy (2′-OCH₂CH₂CH₂NH₂) and 2′-fluoro (2′-F). Similar modifications may also be made at other positions on the dsRNA, particularly the 3′ position of the sugar on the 3′ terminal nucleotide or in 2′-5′ linked dsRNAs and the 5′ position of 5′ terminal nucleotide. dsRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference in its entirety.

dsRNAs may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosine's, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. L, ed. John Wiley & Sons, 1990, those disclosed by Englisch et al., Angewandte Chemie, International Edition, 1991, 30, 613, and those disclosed by Sanghvi, Y S., Chapter 15, DsRNA Research and Applications, pages 289-302, Crooke, S. T. and Lebleu, B., Ed., CRC Press, 1993. Certain of these nucleobases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2 degrees Celcius. (Sanghvi, Y. S., Crooke, S. T. and Lebleu, B., Eds., DsRNA Research and Applications, CRC Press, Boca Raton, 1993, pp. 276-278) and are presently preferred base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, each of which is herein incorporated by reference, and U.S. Pat. No. 5,750,692, also herein incorporated by reference.

Conjugates

Another modification of the dsRNAs of the invention involves chemically linking to the dsRNA one or more moieties or conjugates which enhance the activity, cellular distribution or cellular uptake of the dsRNA. Such moieties include but are not limited to lipid moieties such as a cholesterol moiety (Letsinger et al., Proc. Natl. Acid. Sci. USA, 199, 86, 6553-6556), cholic acid (Manoharan et al., Biorg. Med. Chem. Let., 1994 4 1053-1060), a thioether, e.g., beryl-S-tritylthiol (Manoharan et al., Ann. N.Y. Acad. Sci., 1992, 660, 306-309; Manoharan et al., Biorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyloxycholesterol moiety (Crooke et al., J. Pharmacol. Exp. Ther., 1996, 277, 923-937).

Representative U.S. patents that teach the preparation of such dsRNA conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within a dsRNA. The present invention also includes dsRNA compounds which are chimeric compounds. “Chimeric” dsRNA compounds or “chimeras,” in the context of this invention, are dsRNA compounds, particularly dsRNAs, which contain two or more chemically distinct regions, each made up of at least one monomer unit, i.e., a nucleotide in the case of an dsRNA compound. These dsRNAs typically contain at least one region wherein the dsRNA is modified so as to confer upon the dsRNA increased resistance to nuclease degradation, increased cellular uptake, and/or increased binding affinity for the target nucleic acid. An additional region of the dsRNA may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of dsRNA inhibition of gene expression. Consequently, comparable results can often be obtained with shorter dsRNAs when chimeric dsRNAs are used, compared to phosphorothioate deoxy dsRNAs hybridizing to the same target region. Cleavage of the RNA target can be routinely detected by gel electrophoresis and, if necessary, associated nucleic acid hybridization techniques known in the art.

In certain instances, the dsRNA may be modified by a non-ligand group. A number of non-ligand molecules have been conjugated to dsRNAs in order to enhance the activity, cellular distribution or cellular uptake of the dsRNA, and procedures for performing such conjugations are available in the scientific literature. Such non-ligand moieties have included lipid moieties, such as cholesterol (Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such dsRNA conjugates have been listed above. Typical conjugation protocols involve the synthesis of dsRNAs bearing an aminolinker at one or more positions of the sequence. The amino group is then reacted with the molecule being conjugated using appropriate coupling or activating reagents. The conjugation reaction may be performed either with the dsRNA still bound to the solid support or following cleavage of the dsRNA in solution phase. Purification of the dsRNA conjugate by HPLC typically affords the pure conjugate.

In some cases, a ligand can be multifunctional and/or a dsRNA can be conjugated to more than one ligand. For example, the dsRNA can be conjugated to one ligand for improved uptake and to a second ligand for improved release.

Vector Encoded siRNA Agents

In another aspect of the invention, Eg5 and VEGF specific dsRNA molecules that are expressed from transcription units inserted into DNA or RNA vectors (see, e.g., Couture, A, et al., TIG. (1996), 12:5-10; Skillern, A., et al., International PCT Publication No. WO 00/22113, Conrad, International PCT Publication No. WO 00/22114, and Conrad, US Pat. No. 6,054,299). These transgenes can be introduced as a linear construct, a circular plasmid, or a viral vector, which can be incorporated and inherited as a transgene integrated into the host genome. The transgene can also be constructed to permit it to be inherited as an extrachromosomal plasmid (Gassmann, et al., Proc. Natl. Acad. Sci. USA (1995) 92:1292).

The individual strands of a dsRNA can be transcribed by promoters on two separate expression vectors and co-transfected into a target cell. Alternatively each individual strand of the dsRNA can be transcribed by promoters both of which are located on the same expression plasmid. In a preferred embodiment, a dsRNA is expressed as an inverted repeat joined by a linker polynucleotide sequence such that the dsRNA has a stem and loop structure.

The recombinant dsRNA expression vectors are generally DNA plasmids or viral vectors. dsRNA expressing viral vectors can be constructed based on, but not limited to, adeno-associated virus (for a review, see Muzyczka, et al., Curr. Topics Micro. Immunol. (1992) 158:97-129));

adenovirus (see, for example, Berkner, et al., BioTechniques (1998) 6:616), Rosenfeld et al. (1991, Science 252:431-434), and Rosenfeld et al. (1992), Cell 68:143-155)); or alphavirus as well as others known in the art. Retroviruses have been used to introduce a variety of genes into many different cell types, including epithelial cells, in vitro and/or in vivo (see, e.g., Eglitis, et al., Science (1985) 230:1395-1398; Danos and Mulligan, Proc. Natl. Acad. Sci. USA (1998) 85:6460-6464; Wilson et al., 1988, Proc. Natl. Acad. Sci. USA 85:3014-3018; Armentano et al., 1990, Proc. Natl. Acad. Sci. USA 87:61416145; Huber et al., 1991, Proc. Natl. Acad. Sci. USA 88:8039-8043; Ferry et al., 1991, Proc. Natl. Acad. Sci. USA 88:8377-8381; Chowdhury et al., 1991, Science 254:1802-1805; van Beusechem. et al., 1992, Proc. Natl. Acad. Sci. USA 89:7640-19 ; Kay et al., 1992, Human Gene Therapy 3:641-647; Dai et al., 1992, Proc.

Natl. Acad. Sci. USA 89:10892-10895; Hwu et al., 1993, J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573). Recombinant retroviral vectors capable of transducing and expressing genes inserted into the genome of a cell can be produced by transfecting the recombinant retroviral genome into suitable packaging cell lines such as PA317 and Psi-CRIP (Comette et al., 1991, Human Gene Therapy 2:5-10; Cone et al., 1984, Proc. Natl. Acad. Sci. USA 81:6349). Recombinant adenoviral vectors can be used to infect a wide variety of cells and tissues in susceptible hosts (e.g., rat, hamster, dog, and chimpanzee) (Hsu et al., 1992, J. Infectious Disease, 166:769), and also have the advantage of not requiring mitotically active cells for infection.

Any viral vector capable of accepting the coding sequences for the dsRNA molecule(s) to be expressed can be used, for example vectors derived from adenovirus (AV); adeno-associated virus (AAV); retroviruses (e.g., lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate.

For example, lentiviral vectors of the invention can be pseudotyped with surface proteins from vesicular stomatitis virus (VSV), rabies, Ebola, Mokola, and the like. AAV vectors of the invention can be made to target different cells by engineering the vectors to express different capsid protein serotypes. For example, an AAV vector expressing a serotype 2 capsid on a serotype 2 genome is called AAV 2/2. This serotype 2 capsid gene in the AAV 2/2 vector can be replaced by a serotype 5 capsid gene to produce an AAV 2/5 vector. Techniques for constructing AAV vectors which express different capsid protein serotypes are within the skill in the art; see, e.g., Rabinowitz J E et al. (2002), J Virol 76:791-801, the entire disclosure of which is herein incorporated by reference.

Selection of recombinant viral vectors suitable for use in the invention, methods for inserting nucleic acid sequences for expressing the dsRNA into the vector, and methods of delivering the viral vector to the cells of interest are within the skill in the art. See, for example, Dornburg R (1995), Gene Therap. 2: 301-310; Eglitis M A (1988), Biotechniques 6: 608-614; Miller A D (1990), Hum Gene Therap. 1: 5-14; Anderson W F (1998), Nature 392: 25-30; and Rubinson D A et al., Nat. Genet. 33: 401-406, the entire disclosures of which are herein incorporated by reference.

Preferred viral vectors are those derived from AV and AAV. In a particularly preferred embodiment, the dsRNA of the invention is expressed as two separate, complementary single-stranded RNA molecules from a recombinant AAV vector having, for example, either the U6 or H1 RNA promoters, or the cytomegalovirus (CMV) promoter.

A suitable AV vector for expressing the dsRNA of the invention, a method for constructing the recombinant AV vector, and a method for delivering the vector into target cells, are described in Xia H et al. (2002), Nat. Biotech. 20: 1006-1010.

Suitable AAV vectors for expressing the dsRNA of the invention, methods for constructing the recombinant AV vector, and methods for delivering the vectors into target cells are described in Samulski R et al. (1987), J. Virol. 61: 3096-3101; Fisher K J et al. (1996), J. Virol, 70: 520-532; Samulski R et al. (1989), J. Virol. 63: 3822-3826; U.S. Pat. No. 5,252,479; U.S. Pat. No. 5,139,941; International Patent Application No. WO 94/13788; and International Patent Application No. WO 93/24641, the entire disclosures of which are herein incorporated by reference.

The promoter driving dsRNA expression in either a DNA plasmid or viral vector of the invention may be a eukaryotic RNA polymerase I (e.g. ribosomal RNA promoter), RNA polymerase II (e.g. CMV early promoter or actin promoter or Ul snRNA promoter) or generally RNA polymerase III promoter (e.g. U6 snRNA or 7SK RNA promoter) or a prokaryotic promoter, for example the T7 promoter, provided the expression plasmid also encodes T7 RNA polymerase required for transcription from a T7 promoter. The promoter can also direct transgene expression to the pancreas (see, e.g., the insulin regulatory sequence for pancreas (Bucchini et al., 1986, Proc. Natl. Acad. Sci. USA 83:2511-2515)).

In addition, expression of the transgene can be precisely regulated, for example, by using an inducible regulatory sequence and expression systems such as a regulatory sequence that is sensitive to certain physiological regulators, e.g., circulating glucose levels, or hormones (Docherty et al., 1994, FASEB J. 8:20-24). Such inducible expression systems, suitable for the control of transgene expression in cells or in mammals include regulation by ecdysone, by estrogen, progesterone, tetracycline, chemical inducers of dimerization, and isopropyl-beta-D1-thiogalactopyranoside (EPTG). A person skilled in the art would be able to choose the appropriate regulatory/promoter sequence based on the intended use of the dsRNA transgene.

Generally, recombinant vectors capable of expressing dsRNA molecules are delivered as described below, and persist in target cells. Alternatively, viral vectors can be used that provide for transient expression of dsRNA molecules. Such vectors can be repeatedly administered as necessary. Once expressed, the dsRNAs bind to target RNA and modulate its function or expression. Delivery of dsRNA expressing vectors can be systemic, such as by intravenous or intramuscular administration, by administration to target cells ex-planted from the patient followed by reintroduction into the patient, or by any other means that allows for introduction into a desired target cell.

dsRNA expression DNA plasmids are typically transfected into target cells as a complex with cationic lipid carriers (e.g. Oligofectamine) or non-cationic lipid-based carriers (e.g. Transit-TKO™). Multiple lipid transfections for dsRNA-mediated knockdowns targeting different regions of a single EG5 gene (or VEGF gene) or multiple Eg5 genes (or VEGF genes) over a period of a week or more are also contemplated by the invention. Successful introduction of the vectors of the invention into host cells can be monitored using various known methods. For example, transient transfection can be signaled with a reporter, such as a fluorescent marker, such as Green Fluorescent Protein (GFP). Stable transfection of ex vivo cells can be ensured using markers that provide the transfected cell with resistance to specific environmental factors (e.g., antibiotics and drugs), such as hygromycin B resistance.

The Eg5 specific dsRNA molecules and VEGF specific dsRNA molecules can also be inserted into vectors and used as gene therapy vectors for human patients. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

Pharmaceutical Compositions Containing dsRNA

In one embodiment, the invention provides pharmaceutical compositions containing a dsRNA, as described herein, and a pharmaceutically acceptable carrier and methods of administering the same. The pharmaceutical composition containing the dsRNA is useful for treating a disease or disorder associated with the expression or activity of a Eg5/KSP and/or VEGF gene, such as pathological processes mediated by Eg5/KSP and/or VEGF expression, e.g., liver cancer. Such pharmaceutical compositions are formulated based on the mode of delivery.

Dosage

The pharmaceutical compositions featured herein are administered in dosages sufficient to inhibit expression of EG5/KSP and/or VEGF genes. In general, a suitable dose of dsRNA will be in the range of 0.01 to 200.0 milligrams (mg) per kilogram (kg) body weight of the recipient per day, generally in the range of 1 to 50 mg per kilogram body weight per day. For example, the dsRNA can be administered at 0.01 mg/kg, 0.05 mg/kg, 0.5 mg/kg, 1 mg/kg, 1.5 mg/kg, 2 mg/kg, 3 mg/kg, 5.0 mg/kg, 10 mg/kg, 20 mg/kg, 30 mg/kg, 40 mg/kg, or 50 mg/kg per single dose.

The pharmaceutical composition can be administered once daily, or the dsRNA may be administered as two, three, or more sub-doses at appropriate intervals throughout the day. The effect of a single dose on EG5/KSP and/or VEGF levels is long lasting, such that subsequent doses are administered at not more than 7 day intervals, or at not more than 1, 2, 3, or 4 week intervals.

In some embodiments the dsRNA is administered using continuous infusion or delivery through a controlled release formulation. In that case, the dsRNA contained in each sub-dose must be correspondingly smaller in order to achieve the total daily dosage. The dosage unit can also be compounded for delivery over several days, e.g., using a conventional sustained release formulation which provides sustained release of the dsRNA over a several day period. Sustained release formulations are well known in the art and are particularly useful for delivery of agents at a particular site, such as could be used with the agents of the present invention. In this embodiment, the dosage unit contains a corresponding multiple of the daily dose.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual dsRNAs encompassed by the invention can be made using conventional methodologies or on the basis of in vivo testing using an appropriate animal model, as described elsewhere herein.

Advances in mouse genetics have generated a number of mouse models for the study of various human diseases, such as pathological processes mediated by EG5/KSP AND/OR VEGF expression. Such models are used for in vivo testing of dsRNA, as well as for determining a therapeutically effective dose. A suitable mouse model is, for example, a mouse containing a plasmid expressing human EG5/KSP AND/OR VEGF. Another suitable mouse model is a transgenic mouse carrying a transgene that expresses human EG5/KSP AND/OR VEGF.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred.

The data obtained from cell culture assays and animal studies can be used in formulating a range of dosage for use in humans. The dosage of compositions featured in the invention lies generally within a range of circulating concentrations that include the ED50 with little or no toxicity. The dosage may vary within this range depending upon the dosage form employed and the route of administration utilized. For any compound used in the methods featured in the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately to determine useful doses in humans. Levels in plasma may be measured, for example, by high performance liquid chromatography.

In addition to their administration, as discussed above, the dsRNAs featured in the invention can be administered in combination with other known agents effective in treatment of pathological processes mediated by target gene expression. In any event, the administering physician can adjust the amount and timing of dsRNA administration on the basis of results observed using standard measures of efficacy known in the art or described herein.

Administration

The pharmaceutical compositions of the present invention may be administered in a number of ways depending upon whether local or systemic treatment is desired and upon the area to be treated. Administration may be topical, pulmonary, e.g., by inhalation or insufflation of powders or aerosols, including by nebulizer; intratracheal, intranasal, epidermal and transdermal, and subdermal, oral or parenteral, e.g., subcutaneous.

Typically, when treating a mammal with hyperlipidemia, the dsRNA molecules are administered systemically via parental means. Parenteral administration includes intravenous, intra-arterial, subcutaneous, intraperitoneal or intramuscular injection or infusion; or intracranial, e.g., intraparenchymal, intrathecal or intraventricular, administration. For example, dsRNAs, conjugated or unconjugated or formulated with or without liposomes, can be administered intravenously to a patient. For such, a dsRNA molecule can be formulated into compositions such as sterile and non-sterile aqueous solutions, non-aqueous solutions in common solvents such as alcohols, or solutions in liquid or solid oil bases. Such solutions also can contain buffers, diluents, and other suitable additives. For parenteral, intrathecal, or intraventricular administration, a dsRNA molecule can be formulated into compositions such as sterile aqueous solutions, which also can contain buffers, diluents, and other suitable additives (e.g., penetration enhancers, carrier compounds, and other pharmaceutically acceptable carriers). Formulations are described in more detail herein.

The dsRNA can be delivered in a manner to target a particular tissue, such as the liver (e.g., the hepatocytes of the liver).

Formulations

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids. In one aspect are formulations that target the liver when treating hepatic disorders such as hyperlipidemia.

In addition, dsRNA that target the EG5/KSP and/or VEGF gene can be formulated into compositions containing the dsRNA admixed, encapsulated, conjugated, or otherwise associated with other molecules, molecular structures, or mixtures of nucleic acids. For example, a composition containing one or more dsRNA agents that target the Eg5/KSP and/or VEGF gene can contain other therapeutic agents, such as other cancer therapeutics or one or more dsRNA compounds that target non-EG5/KSP AND/OR VEGF genes.

Oral, Parenteral, Topical, and Biologic Formulations

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. In some embodiments, oral formulations are those in which dsRNAs featured in the invention are administered in conjunction with one or more penetration enhancers surfactants and chelators. Suitable surfactants include fatty acids and/or esters or salts thereof, bile acids and/or salts thereof. Suitable bile acids/salts include chenodeoxycholic acid (CDCA) and ursodeoxychenodeoxycholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether. dsRNAs featured in the invention may be delivered orally, in granular form including sprayed dried particles, or complexed to form micro or nanoparticles. dsRNA complexing agents include poly-amino acids; polyimines; polyacrylates; polyalkylacrylates, polyoxethanes, polyalkylcyanoacrylates; cationized gelatins, albumins, starches, acrylates, polyethyleneglycols (PEG) and starches; polyalkylcyanoacrylates; DEAE-derivatized polyimines, pollulans, celluloses and starches. Suitable complexing agents include chitosan, N-trimethylchitosan, poly-L-lysine, polyhistidine, polyornithine, polyspermines, protamine, polyvinylpyridine, polythiodiethylaminomethylethylene P(TDAE), polyaminostyrene (e.g., p-amino), poly(methylcyanoacrylate), poly(ethylcyanoacrylate), poly(butylcyanoacrylate), poly(isobutylcyanoacrylate), poly(isohexylcynaoacrylate), DEAE-methacrylate, DEAE-hexylacrylate, DEAE-acrylamide, DEAE-albumin and DEAE-dextran, polymethylacrylate, polyhexylacrylate, poly(D,L-lactic acid), poly(DL-lactic-co-glycolic acid (PLGA), alginate, and polyethyleneglycol (PEG). Oral formulations for dsRNAs and their preparation are described in detail in U.S. Pat. No. 6,887,906, U.S. Patent Publication. No. 20030027780, and U.S. Pat. No. 6,747,014, each of which is incorporated herein by reference.

Compositions and formulations for parenteral, intraparenchymal (into the brain), intrathecal, intraventricular or intrahepatic administration may include sterile aqueous solutions which may also contain buffers, diluents and other suitable additives such as, but not limited to, penetration enhancers, carrier compounds and other pharmaceutically acceptable carriers or excipients.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Suitable topical formulations include those in which the dsRNAs featured in the invention are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). dsRNAs featured in the invention may be encapsulated within liposomes or may form complexes thereto, in particular to cationic liposomes. Alternatively, dsRNAs may be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C₁₋₁₀ alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference. In addition, dsRNA molecules can be administered to a mammal as biologic or abiologic means as described in, for example, U.S. Pat. No. 6,271,359. Abiologic delivery can be accomplished by a variety of methods including, without limitation, (1) loading liposomes with a dsRNA acid molecule provided herein and (2) complexing a dsRNA molecule with lipids or liposomes to form nucleic acid-lipid or nucleic acid-liposome complexes. The liposome can be composed of cationic and neutral lipids commonly used to transfect cells in vitro. Cationic lipids can complex (e.g., charge-associate) with negatively charged nucleic acids to form liposomes. Examples of cationic liposomes include, without limitation, lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin™ (Invitrogen/Life Technologies, Carlsbad, Calif.) and Effectene™ (Qiagen, Valencia, Calif.). In addition, systemic delivery methods can be optimized using commercially available cationic lipids such as DDAB or DOTAP, each of which can be mixed with a neutral lipid such as DOPE or cholesterol. In some cases, liposomes such as those described by Templeton et al. (Nature Biotechnology, 15: 647-652 (1997)) can be used. In other embodiments, polycations such as polyethyleneimine can be used to achieve delivery in vivo and ex vivo (Boletta et al., J. Am Soc. Nephrol. 7: 1728 (1996)). Additional information regarding the use of liposomes to deliver nucleic acids can be found in U.S. Pat. No. 6,271,359, PCT Publication WO 96/40964 and Morrissey, D. et al. 2005. Nat Biotechnol. 23(8):1002-7.

Biologic delivery can be accomplished by a variety of methods including, without limitation, the use of viral vectors. For example, viral vectors (e.g., adenovirus and herpes virus vectors) can be used to deliver dsRNA molecules to liver cells. Standard molecular biology techniques can be used to introduce one or more of the dsRNAs provided herein into one of the many different viral vectors previously developed to deliver nucleic acid to cells. These resulting viral vectors can be used to deliver the one or more dsRNAs to cells by, for example, infection.

Liposomal Formulations

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include: liposomes obtained from natural phospholipids are biocompatible and biodegradable; liposomes can incorporate a wide range of water and lipid soluble drugs; and liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, p. 245). Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones and high-molecular weight DNAs have been administered to the skin. The majority of applications resulted in the targeting of the upper epidermis

Liposomes fall into two broad classes. Cationic liposomes are positively charged liposomes which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985).

Liposomes which are pH-sensitive or negatively-charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g., as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/po-lyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al., S.T.P. Pharma. Sci., 1994, 4, 6, 466).

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside G_{M1}, galactocerebroside sulfate and phosphatidylinositol to improve blood half-lives of liposomes. These findings were expounded upon by Gabizon et al. (Proc. Natl. Acad. Sci. U.S.A., 1988, 85, 6949). U.S. Pat. No. 4,837,028 and WO 88/04924, both to Allen et al., disclose liposomes comprising (1) sphingomyelin and (2) the ganglioside G_{M1} or a galactocerebroside sulfate ester. U.S. Pat. No. 5,543,152 (Webb et al.) discloses liposomes comprising sphingomyelin. Liposomes comprising 1,2-sn-dimyristoylphosphat- idylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C_{1215G}, that contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene.

Transfersomes are yet another type of liposomes and are highly deformable lipid aggregates which are attractive candidates for drug delivery vehicles. Transfersomes may be described as lipid droplets which are so highly deformable that they are easily able to penetrate through pores which are smaller than the droplet. Transfersomes are adaptable to the environment in which they are used, e.g., they are self-optimizing (adaptive to the shape of pores in the skin), self-repairing, frequently reach their targets without fragmenting, and often self-loading. To make transfersomes, it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines and phosphatides.

The use of surfactants in drug products, formulations and in emulsions has been reviewed (Rieger, in Pharmaceutical Dosage Forms, Marcel Dekker, Inc., New York, N.Y., 1988, p. 285).

Nucleic Acid Lipid Particles

In one embodiment, a dsRNA featured in the invention is fully encapsulated in the lipid formulation, e.g., to form a nucleic acid-lipid particle. Nucleic acid-lipid particles typically contain a cationic lipid, a non-cationic lipid, a sterol, and a lipid that prevents aggregation of the particle (e.g., a PEG-lipid conjugate). Nucleic acid-lipid particles are extremely useful for systemic applications, as they exhibit extended circulation lifetimes following intravenous (i.v.) injection and accumulate at distal sites (e.g., sites physically separated from the administration site). In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; and PCT Publication No. WO 96/40964.

Nucleic acid-lipid particles can further include one or more additional lipids and/or other components such as cholesterol. Other lipids may be included in the liposome compositions for a variety of purposes, such as to prevent lipid oxidation or to attach ligands onto the liposome surface. Any of a number of lipids may be present, including amphipathic, neutral, cationic, and anionic lipids. Such lipids can be used alone or in combination. Specific examples of additional lipid components that may be present are described herein.

Additional components that may be present in a nucleic acid-lipid particle include bilayer stabilizing components such as polyamide oligomers (see, e.g., U.S. Pat. No. 6,320,017), peptides, proteins, detergents, lipid-derivatives, such as PEG coupled to phosphatidylethanolamine and PEG conjugated to ceramides (see, U.S. Pat. No. 5,885,613).

A nucleic acid-lipid particle can include one or more of a second amino lipid or cationic lipid, a neutral lipid, a sterol, and a lipid selected to reduce aggregation of lipid particles during formation, which may result from steric stabilization of particles which prevents charge-induced aggregation during formation.

Nucleic acid-lipid particles include, e.g., a SPLP, pSPLP, and SNALP. The term“SNALP” refers to a stable nucleic acid-lipid particle, including SPLP. The term “SPLP” refers to a nucleic acid-lipid particle comprising plasmid DNA encapsulated within a lipid vesicle. SPLPs include “pSPLP,” which include an encapsulated condensing agent-nucleic acid complex as set forth in PCT Publication No. WO 00/03683.

The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic

In one embodiment, the lipid to drug ratio (mass/mass ratio) (e.g., lipid to dsRNA ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1, or about 6:1, 7:1, 8:1, 9:1, 10:1, 11:1, 12:1, or 33:1.

Cationic Lipids

The nucleic acid-lipid particles of the invention typically include a cationic lipid. The cationic lipid may be, for example, N,N-dioleyl-N,N-dimethylammonium chloride (DODAC), N,N-distearyl-N,N-dimethylammonium bromide (DDAB), N-(I-(2,3-dioleoyloxy)propyl)-N,N,N--trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyl)tetrahydro-3aH-cyclopenta[d][1,3]dioxol-5-amine (ALNY-100), (6Z,9Z,28Z,31Z)-heptatriaconta-6,9,28,31-tetraen-19-yl 4-(dimethylamino)butanoate (MC3), or a mixture thereof.

Other cationic lipids, which carry a net positive charge at about physiological pH, in addition to those specifically described above, may also be included in lipid particles of the invention. Such cationic lipids include, but are not limited to, N,N-dioleyl-N,N-dimethylammonium chloride (“DODAC”); N-(2,3-dioleyloxy)propyl-N,N-N-triethylammonium chloride (“DOTMA”); N,N-distearyl-N,N-dimethylammonium bromide (“DDAB”); N-(2,3-dioleoyloxy)propyl)-N,N,N-trimethylammonium chloride (“DOTAP”); 1,2-Dioleyloxy-3-trimethylaminopropane chloride salt (“DOTAP.Cl”); 3β-(N-(N′,N′-dimethylaminoethane)-carbamoyl)cholesterol (“DC-Chol”), N-(1-(2,3-dioleyloxy)propyl)-N-2-(sperminecarboxamido)ethyl)-N,N-dimethylammonium trifluoracetate (“DOSPA”), dioctadecylamidoglycyl carboxyspermine (“DOGS”), 1,2-dileoyl-sn-3-phosphoethanolamine (“DOPE”), 1,2-dioleoyl-3-dimethylammonium propane (“DODAP”), N,N-dimethyl-2,3-dioleyloxy)propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic lipids can be used, such as, e.g., LIPOFECTIN (including DOTMA and DOPE, available from GIBCO/BRL), and LIPOFECTAMINE (comprising DOSPA and DOPE, available from GIBCO/BRL). In particular embodiments, a cationic lipid is an amino lipid.

As used herein, the term “amino lipid” is meant to include those lipids having one or two fatty acid or fatty alkyl chains and an amino head group (including an alkylamino or dialkylamino group) that may be protonated to form a cationic lipid at physiological pH.

Other amino lipids would include those having alternative fatty acid groups and other dialkylamino groups, including those in which the alkyl substituents are different (e.g., N-ethyl-N-methylamino-, N-propyl-N-ethylamino- and the like). For those embodiments in which R¹¹ and R¹² are both long chain alkyl or acyl groups, they can be the same or different. In general, amino lipids having less saturated acyl chains are more easily sized, particularly when the complexes must be sized below about 0.3 microns, for purposes of filter sterilization. Amino lipids containing unsaturated fatty acids with carbon chain lengths in the range of C₁₄ to C₂₂ are preferred. Other scaffolds can also be used to separate the amino group and the fatty acid or fatty alkyl portion of the amino lipid. Suitable scaffolds are known to those of skill in the art.

In certain embodiments, amino or cationic lipids of the invention have at least one protonatable or deprotonatable group, such that the lipid is positively charged at a pH at or below physiological pH (e.g. pH 7.4), and neutral at a second pH, preferably at or above physiological pH. It will, of course, be understood that the addition or removal of protons as a function of pH is an equilibrium process, and that the reference to a charged or a neutral lipid refers to the nature of the predominant species and does not require that all of the lipid be present in the charged or neutral form. Lipids that have more than one protonatable or deprotonatable group, or which are zwiterrionic, are not excluded from use in the invention.

In certain embodiments, protonatable lipids according to the invention have a pKa of the protonatable group in the range of about 4 to about 11. Most preferred is pKa of about 4 to about 7, because these lipids will be cationic at a lower pH formulation stage, while particles will be largely (though not completely) surface neutralized at physiological pH around pH 7.4. One of the benefits of this pKa is that at least some nucleic acid associated with the outside surface of the particle will lose its electrostatic interaction at physiological pH and be removed by simple dialysis; thus greatly reducing the particle's susceptibility to clearance.

One example of a cationic lipid is 1,2-Dilinolenyloxy-N,N-dimethylaminopropane (DLinDMA). Synthesis and preparation of nucleic acid-lipid particles including DlinDMA is described in International application number PCT/CA2009/00496, filed Apr. 15, 2009.

In one embodiment, the cationic lipid XTC (2,2-Dilinoleyl-4-dimethylaminoethyl-[1,3]-dioxolane) is used to prepare nucleic acid-lipid particles . Synthesis of XTC is described in U.S. provisional patent application No. 61/107,998 filed on Oct. 23, 2008, which is herein incorporated by reference.

In another embodiment, the cationic lipid MC3 ((6Z,9Z,28Z,31Z)-heptatriaconta-6,9,28,31-tetraen-19-yl 4-(dimethylamino)butanoate), (e.g., DLin-M-C3-DMA) is used to prepare nucleic acid-lipid particles . Synthesis of MC3 and MC3 comprising formulations are described, e.g., in U.S. Provisional Ser. No. 61/244,834, filed Sep. 22, 2009, and U.S. Provisional Ser. No. 61/185,800, filed Jun. 10, 2009, which are hereby incorporated by reference.

In another embodiment, the cationic lipid ALNY-100 ((3aR,5s,6aS)-N,N-dimethyl-2,2-di((9Z,12Z)-octadeca-9,12-dienyl)tetrahydro-3aH-cyclopenta[d][1,3]dioxol-5-amine) is used to prepare nucleic acid-lipid particles . Synthesis of ALNY-100 is described in International patent application number PCT/US09/63933 filed on Nov. 10, 2009, which is herein incorporated by reference.

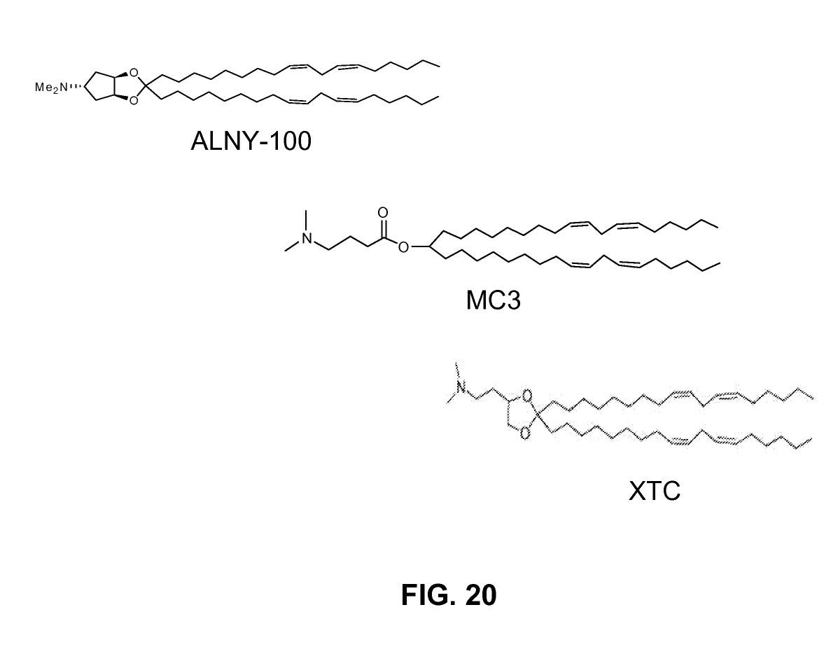

FIG. 20 illustrates the structures of ALNY-100, MC3, and XTC. The cationic lipid may comprise from about 20 mol % to about 70 mol % or about 45-65 mol % or about 40 mol % of the total lipid present in the particle.

Non-Cationic Lipids

The nucleic acid-lipid particles of the invention can include a non-cationic lipid. The non-cationic lipid may be an anionic lipid or a neutral lipid. Examples include but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl- phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1 -trans PE, 1 -stearoyl-2-oleoyl-phosphatidyethanolamine (SOPE), cholesterol, or a mixture thereof.

Anionic lipids suitable for use in lipid particles of the invention include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Neutral lipids, when present in the lipid particle, can be any of a number of lipid species which exist either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., liposome size and stability of the liposomes in the bloodstream. Preferably, the neutral lipid component is a lipid having two acyl groups, (i.e., diacylphosphatidylcholine and diacylphosphatidylethanolamine). Lipids having a variety of acyl chain groups of varying chain length and degree of saturation are available or may be isolated or synthesized by well-known techniques. In one group of embodiments, lipids containing saturated fatty acids with carbon chain lengths in the range of C₁₄ to C₂₂ are preferred. In another group of embodiments, lipids with mono- or di-unsaturated fatty acids with carbon chain lengths in the range of C₁₄ to C₂₂ are used. Additionally, lipids having mixtures of saturated and unsaturated fatty acid chains can be used. Preferably, the neutral lipids used in the invention are DOPE, DSPC, POPC, or any related phosphatidylcholine. The neutral lipids useful in the invention may also be composed of sphingomyelin, dihydrosphingomyeline, or phospholipids with other head groups, such as serine and inositol.

In one embodiment the non-cationic lipid is distearoylphosphatidylcholine (DSPC). In another embodiment the non-cationic lipid is dipalmitoylphosphatidylcholine (DPPC). The non-cationic lipid may be from about 5 mol % to about 90 mol %, about 5 mol % to about 10 mol %, about 10 mol %, or about 58 mol % if cholesterol is included, of the total lipid present in the particle.

Conjugated Lipids

Conjugated lipids can be used in nucleic acid-lipid particle to prevent aggregation, including polyethylene glycol (PEG)-modified lipids, monosialoganglioside Gml, and polyamide oligomers (“PAO”) such as (described in U.S. Pat. No. 6,320,017). Other compounds with uncharged, hydrophilic, steric-barrier moieties, which prevent aggregation during formulation, like PEG, Gml or ATTA, can also be coupled to lipids for use as in the methods and compositions of the invention. ATTA-lipids are described, e.g., in U.S. Pat. No. 6,320,017, and PEG-lipid conjugates are described, e.g., in U.S. Pat. Nos. 5,820,873, 5,534,499 and 5,885,613. Typically, the concentration of the lipid component selected to reduce aggregation is about 1 to 15% (by mole percent of lipids).

Specific examples of PEG-modified lipids (or lipid-polyoxyethylene conjugates) that are useful in the invention can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in co-pending U.S. Ser. No. 08/486,214, incorporated herein by reference, PEG-modified dialkylamines and PEG-modified 1,2-diacyloxypropan-3-amines. Particularly preferred are PEG-modified diacylglycerols and dialkylglycerols.

In embodiments where a sterically-large moiety such as PEG or ATTA are conjugated to a lipid anchor, the selection of the lipid anchor depends on what type of association the conjugate is to have with the lipid particle. It is well known that mePEG (mw2000)-diastearoylphosphatidylethanolamine (PEG-DSPE) will remain associated with a liposome until the particle is cleared from the circulation, possibly a matter of days. Other conjugates, such as PEG-CerC20 have similar staying capacity. PEG-CerC14, however, rapidly exchanges out of the formulation upon exposure to serum, with a T_{1/2} less than 60 mins. in some assays. As illustrated in U.S. patent application Ser. No. 08/486,214, at least three characteristics influence the rate of exchange: length of acyl chain, saturation of acyl chain, and size of the steric-barrier head group. Compounds having suitable variations of these features may be useful for the invention. For some therapeutic applications, it may be preferable for the PEG-modified lipid to be rapidly lost from the nucleic acid-lipid particle in vivo and hence the PEG-modified lipid will possess relatively short lipid anchors. In other therapeutic applications, it may be preferable for the nucleic acid-lipid particle to exhibit a longer plasma circulation lifetime and hence the PEG-modified lipid will possess relatively longer lipid anchors. Exemplary lipid anchors include those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons.

It should be noted that aggregation preventing compounds do not necessarily require lipid conjugation to function properly. Free PEG or free ATTA in solution may be sufficient to prevent aggregation. If the particles are stable after formulation, the PEG or ATTA can be dialyzed away before administration to a subject.

The conjugated lipid that inhibits aggregation of particles may be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate may be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). Additional conjugated lipids include polyethylene glycol-didimyristoyl glycerol (C14-PEG or PEG-C14, where PEG has an average molecular weight of 2000 Da) (PEG-DMG); (R)-2,3-bis(octadecyloxy)propyl1-(methoxy poly(ethylene glycol)2000)propylcarbamate) (PEG-DSG); PEG-carbamoyl-1,2-dimyristyloxypropylamine, in which PEG has an average molecular weight of 2000 Da (PEG-cDMA); N-Acetylgalactosamine-(R)-2,3-bis(octadecyloxy)propyl1-(methoxy poly(ethylene glycol)2000)propylcarbamate)) (GalNAc-PEG-DSG); and polyethylene glycol-dipalmitoylglycerol (PEG-DPG).

In one embodiment the conjugated lipid is PEG-DMG. In another embodiment the conjugated lipid is PEG-cDMA. In still another embodiment the conjugated lipid is PEG-DPG. Alternatively the conjugated lipid is GalNAc-PEG-DSG.

The conjugated lipid that prevents aggregation of particles may be from 0 mol % to about 20 mol % or about 0.5 to about 5.0 mol % or about 2 mol % of the total lipid present in the particle.

The sterol component of the lipid mixture, when present, can be any of those sterols conventionally used in the field of liposome, lipid vesicle or lipid particle preparation. A preferred sterol is cholesterol.

In some embodiments, the nucleic acid-lipid particle further includes a sterol, e.g., a cholesterol at, e.g., about 10 mol % to about 60 mol % or about 25 to about 40 mol % or about 48 mol % of the total lipid present in the particle.

Lipoproteins

In one embodiment, the formulations of the invention further comprise an apolipoprotein. As used herein, the term “apolipoprotein” or “lipoprotein” refers to apolipoproteins known to those of skill in the art and variants and fragments thereof and to apolipoprotein agonists, analogues or fragments thereof described below.

Suitable apolipoproteins include, but are not limited to, ApoA-I, ApoA-II, ApoA-IV, ApoA-V and ApoE, and active polymorphic forms, isoforms, variants and mutants as well as fragments or truncated forms thereof. In certain embodiments, the apolipoprotein is a thiol containing apolipoprotein. “Thiol containing apolipoprotein” refers to an apolipoprotein, variant, fragment or isoform that contains at least one cysteine residue. The most common thiol containing apolipoproteins are ApoA-I Milano (ApoA-I_{M}) and ApoA-I Paris (ApoA-I_{P}) which contain one cysteine residue (Jia et al., 2002, Biochem. Biophys. Res. Comm. 297: 206-13; Bielicki and Oda, 2002, Biochemistry 41: 2089-96). ApoA-II, ApoE2 and ApoE3 are also thiol containing apolipoproteins. Isolated ApoE and/or active fragments and polypeptide analogues thereof, including recombinantly produced forms thereof, are described in U.S. Pat. Nos. 5,672,685; 5,525,472; 5,473,039; 5,182,364; 5,177,189; 5,168,045; 5,116,739; the disclosures of which are herein incorporated by reference. ApoE3 is disclosed in Weisgraber, et al., “Human E apoprotein heterogeneity: cysteine-arginine interchanges in the amino acid sequence of the apo-E isoforms,” J. Biol. Chem. (1981) 256: 9077-9083; and Rall, et al., “Structural basis for receptor binding heterogeneity of apolipoprotein E from type III hyperlipoproteinemic subjects,” Proc. Nat. Acad. Sci. (1982) 79: 4696-4700. (See also GenBank accession number K00396.)

In certain embodiments, the apolipoprotein can be in its mature form, in its preproapolipoprotein form or in its proapolipoprotein form. Homo- and heterodimers (where feasible) of pro- and mature ApoA-I (Duverger et al., 1996, Arterioscler. Thromb. Vasc. Biol. 16(12):1424-29), ApoA-I Milano (Klon et al., 2000, Biophys. J. 79:(3)1679-87; Franceschini et al., 1985, J. Biol. Chem. 260: 1632-35), ApoA-I Paris (Daum et al., 1999, J. Mol. Med. 77:614-22), ApoA-II (Shelness et al., 1985, J. Biol. Chem. 260(14):8637-46; Shelness et al., 1984, J. Biol. Chem. 259(15):9929-35), ApoA-IV (Duverger et al., 1991, Euro. J. Biochem. 201(2):373-83), and ApoE (McLean et al., 1983, J. Biol. Chem. 258(14):8993-9000) can also be utilized within the scope of the invention.

In certain embodiments, the apolipoprotein can be a fragment, variant or isoform of the apolipoprotein. The term “fragment” refers to any apolipoprotein having an amino acid sequence shorter than that of a native apolipoprotein and which fragment retains the activity of native apolipoprotein, including lipid binding properties. By “variant” is meant substitutions or alterations in the amino acid sequences of the apolipoprotein, which substitutions or alterations, e.g., additions and deletions of amino acid residues, do not abolish the activity of native apolipoprotein, including lipid binding properties. Thus, a variant can comprise a protein or peptide having a substantially identical amino acid sequence to a native apolipoprotein provided herein in which one or more amino acid residues have been conservatively substituted with chemically similar amino acids. Examples of conservative substitutions include the substitution of at least one hydrophobic residue such as isoleucine, valine, leucine or methionine for another. Likewise, the present invention contemplates, for example, the substitution of at least one hydrophilic residue such as, for example, between arginine and lysine, between glutamine and asparagine, and between glycine and serine (see U.S. Pat. Nos. 6,004,925, 6,037,323 and 6,046,166). The term “isoform” refers to a protein having the same, greater or partial function and similar, identical or partial sequence, and may or may not be the product of the same gene and usually tissue specific (see Weisgraber 1990, J. Lipid Res. 31(8):1503-11; Hixson and Powers 1991, J. Lipid Res. 32(9):1529-35; Lackner et al., 1985, J. Biol. Chem. 260(2):703-6; Hoeg et al., 1986, J. Biol. Chem. 261(9):3911-4; Gordon et al., 1984, J. Biol. Chem. 259(1):468-74; Powell et al., 1987, Cell 50(6):831-40; Aviram et al., 1998, Arterioscler. Thromb. Vase. Biol. 18(10):1617-24; Aviram et al., 1998, J. Clin. Invest. 101(8):1581-90; Billecke et al., 2000, Drug Metab. Dispos. 28(11):1335-42; Draganov et al., 2000, J. Biol. Chem. 275(43):33435-42; Steinmetz and Utermann 1985, J. Biol. Chem. 260(4):2258-64; Widler et al., 1980, J. Biol. Chem. 255(21):10464-71; Dyer et al., 1995, J. Lipid Res. 36(1):80-8; Sacre et al., 2003, FEBS Lett. 540(1-3):181-7; Weers, et al., 2003, Biophys. Chem. 100(1-3):481-92; Gong et al., 2002, J. Biol. Chem. 277(33):29919-26; Ohta et al., 1984, J. Biol. Chem. 259(23):14888-93 and U.S. Pat. No. 6,372,886).

In certain embodiments, the methods and compositions of the present invention include the use of a chimeric construction of an apolipoprotein. For example, a chimeric construction of an apolipoprotein can be comprised of an apolipoprotein domain with high lipid binding capacity associated with an apolipoprotein domain containing ischemia reperfusion protective properties. A chimeric construction of an apolipoprotein can be a construction that includes separate regions within an apolipoprotein (i.e., homologous construction) or a chimeric construction can be a construction that includes separate regions between different apolipoproteins (i.e., heterologous constructions). Compositions comprising a chimeric construction can also include segments that are apolipoprotein variants or segments designed to have a specific character (e.g., lipid binding, receptor binding, enzymatic, enzyme activating, antioxidant or reduction-oxidation property) (see Weisgraber 1990, J. Lipid Res. 31(8):1503-1 1; Hixson and Powers 1991, J. Lipid Res. 32(9):1529-35; Lackner et al., 1985, J. Biol. Chem. 260(2):703-6; Hoeg et al., 1986, J. Biol. Chem. 261(9):3911-4; Gordon et al., 1984, J. Biol. Chem. 259(1):468-74; Powell et al., 1987, Cell 50(6):831-40; Aviram et al., 1998, Arterioscler. Thromb. Vasc. Biol. 18(10):1617-24; Aviram et al., 1998, J. Clin. Invest. 101(8):1581-90; Billecke et al., 2000, Drug Metab. Dispos. 28(11):1335-42; Draganov et al., 2000, J. Biol. Chem. 275(43):33435-42; Steinmetz and Utermann 1985, J. Biol. Chem. 260(4):2258-64; Widler et al., 1980, J. Biol. Chem. 255(21):10464-71; Dyer et al., 1995, J. Lipid Res. 36(1):80-8; Sorenson et al., 1999, Arterioscler. Thromb. Vasc. Biol. 19(9):2214-25; Palgunachari 1996, Arterioscler. Throb. Vasc. Biol. 16(2):328-38: Thurberg et al., J. Biol. Chem. 271(11):6062-70; Dyer 1991, J. Biol. Chem. 266(23):150009-15; Hill 1998, J. Biol. Chem. 273(47):30979-84).

Apolipoproteins utilized in the invention also include recombinant, synthetic, semi-synthetic or purified apolipoproteins. Methods for obtaining apolipoproteins or equivalents thereof, utilized by the invention are well-known in the art. For example, apolipoproteins can be separated from plasma or natural products by, for example, density gradient centrifugation or immunoaffinity chromatography, or produced synthetically, semi-synthetically or using recombinant DNA techniques known to those of the art (see, e.g., Mulugeta et al., 1998, J. Chromatogr. 798(1-2): 83-90; Chung et al., 1980, J. Lipid Res. 21(3):284-91; Cheung et al., 1987, J. Lipid Res. 28(8):913-29; Persson, et al., 1998, J. Chromatogr. 711:97-109; U.S. Pat. Nos. 5,059,528, 5,834,596, 5,876,968 and 5,721,114; and PCT Publications WO 86/04920 and WO 87/02062).

Apolipoproteins utilized in the invention further include apolipoprotein agonists such as peptides and peptide analogues that mimic the activity of ApoA-I, ApoA-I Milano (ApoA-I_{M}), ApoA-I Paris (ApoA-I_{P}), ApoA-II, ApoA-IV, and ApoE. For example, the apolipoprotein can be any of those described in U.S. Pat. Nos. 6,004,925, 6,037,323, 6,046,166, and 5,840,688, the contents of which are incorporated herein by reference in their entireties.

Apolipoprotein agonist peptides or peptide analogues can be synthesized or manufactured using any technique for peptide synthesis known in the art including, e.g., the techniques described in U.S. Pat. Nos. 6,004,925, 6,037,323 and 6,046,166. For example, the peptides may be prepared using the solid-phase synthetic technique initially described by Merrifield (1963, J. Am. Chem. Soc. 85:2149-2154). Other peptide synthesis techniques may be found in Bodanszky et al., Peptide Synthesis, John Wiley & Sons, 2d Ed., (1976) and other references readily available to those skilled in the art. A summary of polypeptide synthesis techniques can be found in Stuart and Young, Solid Phase Peptide. Synthesis, Pierce Chemical Company, Rockford, Ill., (1984). Peptides may also be synthesized by solution methods as described in The Proteins, Vol. II, 3d Ed., Neurath et al., Eds., p. 105-237, Academic Press, New York, N.Y. (1976). Appropriate protective groups for use in different peptide syntheses are described in the above-mentioned texts as well as in McOmie, Protective Groups in Organic Chemistry, Plenum Press, New York, N.Y. (1973). The peptides of the present invention might also be prepared by chemical or enzymatic cleavage from larger portions of, for example, apolipoprotein A-I.

In certain embodiments, the apolipoprotein can be a mixture of apolipoproteins. In one embodiment, the apolipoprotein can be a homogeneous mixture, that is, a single type of apolipoprotein. In another embodiment, the apolipoprotein can be a heterogeneous mixture of apolipoproteins, that is, a mixture of two or more different apolipoproteins. Embodiments of heterogenous mixtures of apolipoproteins can comprise, for example, a mixture of an apolipoprotein from an animal source and an apolipoprotein from a semi-synthetic source. In certain embodiments, a heterogenous mixture can comprise, for example, a mixture of ApoA-I and ApoA-I Milano. In certain embodiments, a heterogeneous mixture can comprise, for example, a mixture of ApoA-I Milano and ApoA-I Paris. Suitable mixtures for use in the methods and compositions of the invention will be apparent to one of skill in the art.

If the apolipoprotein is obtained from natural sources, it can be obtained from a plant or animal source. If the apolipoprotein is obtained from an animal source, the apolipoprotein can be from any species. In certain embodiments, the apolipoprotien can be obtained from an animal source. In certain embodiments, the apolipoprotein can be obtained from a human source. In preferred embodiments of the invention, the apolipoprotein is derived from the same species as the individual to which the apolipoprotein is administered.

Other Components

In numerous embodiments, amphipathic lipids are included in lipid particles of the invention. “Amphipathic lipids” refer to any suitable material, wherein the hydrophobic portion of the lipid material orients into a hydrophobic phase, while the hydrophilic portion orients toward the aqueous phase. Such compounds include, but are not limited to, phospholipids, aminolipids, and sphingolipids. Representative phospholipids include sphingomyelin, phosphatidylcholine, phosphatidylethanolamine, phosphatidylserine, phosphatidylinositol, phosphatidic acid, palmitoyloleoyl phosphatdylcholine, lysophosphatidylcholine, lysophosphatidylethanolamine, dipalmitoylphosphatidylcholine, dioleoylphosphatidylcholine, distearoylphosphatidylcholine, or dilinoleylphosphatidylcholine. Other phosphorus-lacking compounds, such as sphingolipids, glycosphingolipid families, diacylglycerols, and β-acyloxyacids, can also be used. Additionally, such amphipathic lipids can be readily mixed with other lipids, such as triglycerides and sterols.

Also suitable for inclusion in the lipid particles of the invention are programmable fusion lipids. Such lipid particles have little tendency to fuse with cell membranes and deliver their payload until a given signal event occurs. This allows the lipid particle to distribute more evenly after injection into an organism or disease site before it starts fusing with cells. The signal event can be, for example, a change in pH, temperature, ionic environment, or time. In the latter case, a fusion delaying or “cloaking” component, such as an ATTA-lipid conjugate or a PEG-lipid conjugate, can simply exchange out of the lipid particle membrane over time. Exemplary lipid anchors include those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons.

A lipid particle conjugated to a nucleic acid agent can also include a targeting moiety, e.g., a targeting moiety that is specific to a cell type or tissue. Targeting of lipid particles using a variety of targeting moieties, such as ligands, cell surface receptors, glycoproteins, vitamins (e.g., riboflavin) and monoclonal antibodies, has been previously described (see, e.g., U.S. Pat. Nos. 4,957,773 and 4,603,044). The targeting moieties can include the entire protein or fragments thereof. Targeting mechanisms generally require that the targeting agents be positioned on the surface of the lipid particle in such a manner that the targeting moiety is available for interaction with the target, for example, a cell surface receptor. A variety of different targeting agents and methods are known and available in the art, including those described, e.g., in Sapra, P. and Allen, T M, Prog. Lipid Res. 42(5):439-62 (2003); and Abra, R M et al., J. Liposome Res. 12:1-3, (2002).

The use of lipid particles, i.e., liposomes, with a surface coating of hydrophilic polymer chains, such as polyethylene glycol (PEG) chains, for targeting has been proposed (Allen, et al., Biochimica et Biophysica Acta 1237: 99-108 (1995); DeFrees, et al., Journal of the American Chemistry Society 118: 6101-6104 (1996); Blume, et al., Biochimica et Biophysica Acta 1149: 180-184 (1993); Klibanov, et al., Journal of Liposome Research 2: 321-334 (1992); U.S. Pat. No. 5,013556; Zalipsky, Bioconjugate Chemistry 4: 296-299 (1993); Zalipsky, FEBS Letters 353: 71-74 (1994); Zalipsky, in Stealth Liposomes Chapter 9 (Lasic and Martin, Eds) CRC Press, Boca Raton Fla. (1995). In one approach, a ligand, such as an antibody, for targeting the lipid particle is linked to the polar head group of lipids forming the lipid particle. In another approach, the targeting ligand is attached to the distal ends of the PEG chains forming the hydrophilic polymer coating (Klibanov, et al., Journal of Liposome Research 2: 321-334 (1992); Kirpotin et al., FEBS Letters 388: 115-118 (1996)).

Standard methods for coupling the target agents can be used. For example, phosphatidylethanolamine, which can be activated for attachment of target agents, or derivatized lipophilic compounds, such as lipid-derivatized bleomycin, can be used. Antibody-targeted liposomes can be constructed using, for instance, liposomes that incorporate protein A (see, Renneisen, et al., J. Bio. Chem., 265:16337-16342 (1990) and Leonetti, et al., Proc. Natl. Acad. Sci. (USA), 87:2448-2451 (1990). Other examples of antibody conjugation are disclosed in U.S. Pat. No. 6,027,726, the teachings of which are incorporated herein by reference. Examples of targeting moieties can also include other proteins, specific to cellular components, including antigens associated with neoplasms or tumors. Proteins used as targeting moieties can be attached to the liposomes via covalent bonds (see, Heath, Covalent Attachment of Proteins to Liposomes, 149 Methods in Enzymology 111-119 (Academic Press, Inc. 1987)). Other targeting methods include the biotin-avidin system.

Production of Nucleic Acid-Lipid Particles

In one embodiment, the nucleic acid-lipid particle formulations of the invention are produced via an extrusion method or an in-line mixing method.

The extrusion method (also refer to as preformed method or batch process) is a method where the empty liposomes (i.e. no nucleic acid) are prepared first, followed by the addition of nucleic acid to the empty liposome. Extrusion of liposome compositions through a small-pore polycarbonate membrane or an asymmetric ceramic membrane results in a relatively well-defined size distribution. Typically, the suspension is cycled through the membrane one or more times until the desired liposome complex size distribution is achieved. The liposomes may be extruded through successively smaller-pore membranes, to achieve a gradual reduction in liposome size. In some instances, the lipid-nucleic acid compositions which are formed can be used without any sizing. These methods are disclosed in the U.S. Pat. No. 5,008,050; U.S. Pat. No. 4,927,637; U.S. Pat. No. 4,737,323; Biochim Biophys Acta. 1979 Oct. 19;557(1):9-23; Biochim Biophys Acta. 1980 Oct. 2;601(3):559-7; Biochim Biophys Acta. 1986 Jun. 13;858(1):161-8; and Biochim. Biophys. Acta 1985 812, 55-65, which are hereby incorporated by reference in their entirety.

The in-line mixing method is a method wherein both the lipids and the nucleic acid are added in parallel into a mixing chamber. The mixing chamber can be a simple T-connector or any other mixing chamber that is known to one skill in the art. These methods are disclosed in U.S. Pat. No. 6,534,018 and U.S. Pat. No. 6,855,277; US publication 2007/0042031 and Pharmaceuticals Research, Vol. 22, No. 3, Mar. 2005, p. 362-372, which are hereby incorporated by reference in their entirety.

It is further understood that the formulations of the invention can be prepared by any methods known to one of ordinary skill in the art.

Characterization of Nucleic Acid-Lipid Particles

Formulations prepared by either the standard or extrusion-free method can be characterized in similar manners. For example, formulations are typically characterized by visual inspection. They should be whitish translucent solutions free from aggregates or sediment. Particle size and particle size distribution of lipid-nanoparticles can be measured by light scattering using, for example, a Malvern Zetasizer Nano ZS (Malvern, USA). Particles should be about 20-300 nm, such as 40-100 nm in size. The particle size distribution should be unimodal. The total siRNA concentration in the formulation, as well as the entrapped fraction, is estimated using a dye exclusion assay. A sample of the formulated siRNA can be incubated with an RNA-binding dye, such as Ribogreen (Molecular Probes) in the presence or absence of a formulation disrupting surfactant, e.g., 0.5% Triton-X100. The total siRNA in the formulation can be determined by the signal from the sample containing the surfactant, relative to a standard curve. The entrapped fraction is determined by subtracting the “free” siRNA content (as measured by the signal in the absence of surfactant) from the total siRNA content. Percent entrapped siRNA is typically >85%. In one embodiment, the formulations of the invention are entrapped by at least 75%, at least 80% or at least 90%.

For nucleic acid-lipid particle formulations, the particle size is at least 30 nm, at least 40 nm, at least 50 nm, at least 60 nm, at least 70 nm, at least 80 nm, at least 90 nm, at least 100 nm, at least 110 nm, and at least 120 nm. The suitable range is typically about at least 50 nm to about at least 110 nm, about at least 60 nm to about at least 100 nm, or about at least 80 nm to about at least 90 nm.

Formulations of Nucleic Acid-Lipid Particles

LNP01

One example of synthesis of a nucleic acid-lipid particle is as follows. Nucleic acid-lipid particles are synthesized using the lipidoid ND98.4HCl (MW 1487) (Formula 1), Cholesterol (Sigma-Aldrich), and PEG-Ceramide C16 (Avanti Polar Lipids) ,. This nucleic acid-lipid particle is sometimes referred to as a LNP01 particles. Stock solutions of each in ethanol can be prepared as follows: ND98, 133 mg/ml; Cholesterol, 25 mg/ml, PEG-Ceramide C16, 100 mg/ml. The ND98, Cholesterol, and PEG-Ceramide C16 stock solutions can then be combined in a, e.g., 42:48:10 molar ratio. The combined lipid solution can be mixed with aqueous siRNA (e.g., in sodium acetate pH 5) such that the final ethanol concentration is about 35-45% and the final sodium acetate concentration is about 100-300 mM. Lipid-siRNA nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

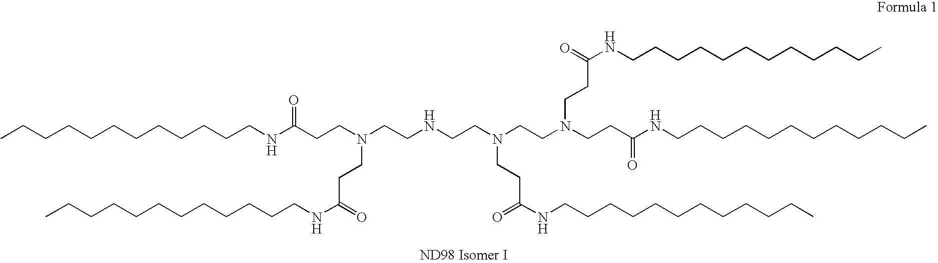

LNP01 formulations are described, e.g., in International Application Publication No. WO 2008/042973, which is hereby incorporated by reference.

Additional exemplary nucleic acid-lipid particle formulations are described in the following table. It is to be understood that the name of the nucleic acid-lipid particle in the table is not meant to be limiting. For example, as used herein, the term SNALP refers to a formulations that includes the cationic lipid DLinDMA.

| | cationic lipid/non-cationic lipid/cholesterol/ |
|---|---|
| | PEG-lipid conjugate mol % ratio |
| Name | Lipid:siRNA ratio |
| SNALP | DLinDMA/DPPC/Cholesterol/PEG-cDMA |
| | (57.1/7.1/34.4/1.4) |
| | lipid:siRNA ~7:1 |
| LNP-S-X | XTC/DPPC/Cholesterol/PEG-cDMA |
| | 57.1/7.1/34.4/1.4 |
| | lipid:siRNA ~7:1 |
| LNP05 | XTC/DSPC/Cholesterol/PEG-DMG |
| | 57.5/7.5/31.5/3.5 |
| | lipid:siRNA ~6:1 |
| LNP06 | XTC/DSPC/Cholesterol/PEG-DMG |
| | 57.5/7.5/31.5/3.5 |
| | lipid:siRNA ~11:1 |
| LNP07 | XTC/DSPC/Cholesterol/PEG-DMG |
| | 60/7.5/31/1.5, |
| | lipid: siRNA ~6:1 |
| LNP08 | XTC/DSPC/Cholesterol/PEG-DMG |
| | 60/7.5/31/1.5, |
| | lipid: siRNA ~11:1 |
| LNP09 | XTC/DSPC/Cholesterol/PEG-DMG |
| | 50/10/38.5/1.5 |
| | lipid:siRNA ~10:1 |
| LNP10 | ALNY-100/DSPC/Cholesterol/PEG-DMG |
| | 50/10/38.5/1.5 |
| | lipid:siRNA ~10:1 |
| LNP11 | MC3/DSPC/Cholesterol/PEG-DMG |
| | 50/10/38.5/1.5 |
| | lipid:siRNA ~10:1 |
| LNP13 | XTC/DSPC/Cholesterol/PEG-DMG |
| | 50/10/38.5/1.5 |
| | lipid:siRNA ~33:1 |
| LNP14 | MC3/DSPC/Cholesterol/PEG-DMG |
| | 40/15/40/5 |
| | lipid:siRNA ~11:1 |
| LNP15 | MC3/DSPC/Cholesterol/PEG-DSG/ |
| | GalNAc-PEG-DSG 50/10/35/4.5/0.5 |
| | lipid:siRNA ~11:1 |
| LNP16 | MC3/DSPC/Cholesterol/PEG-DMG |
| | 50/10/38.5/1.5 |
| | lipid:siRNA ~7:1 |
| LNP17 | MC3/DSPC/Cholesterol/PEG-DSG |
| | 50/10/38.5/1.5 |
| | lipid:siRNA ~10:1 |
| LNP18 | MC3/DSPC/Cholesterol/PEG-DMG |
| | 50/10/38.5/1.5 |
| | lipid:siRNA ~12:1 |
| LNP19 | MC3/DSPC/Cholesterol/PEG-DMG |
| | 50/10/35/5 |
| | lipid:siRNA ~8:1 |
| LNP20 | MC3/DSPC/Cholesterol/PEG-DPG |
| | 50/10/38.5/1.5 |
| | lipid:siRNA ~10:1 |
| LNP22 | XTC/DSPC/Cholesterol/PEG-DSG |
| | 50/10/38.5/1.5 |
| | lipid: siRNA ~10:1 |

XTC comprising formulations are described, e.g., in U.S. Provisional Ser. No. 61/239,686, filed Sep. 3, 2009, which is hereby incorporated by reference.

MC3 comprising formulations are described, e.g., in U.S. Provisional Ser. No. 61/244,834, filed Sep. 22, 2009, and U.S. Provisional Ser. No. 61/185,800, filed Jun. 10, 2009, which are hereby incorporated by reference.

ALNY-100 comprising formulations are described, e.g., International patent application number PCT/US09/63933, filed on Nov. 10, 2009, which is hereby incorporated by reference.

Additional representative formulations delineated in Tables 25 and 26. Lipid refers to a cationic lipid.

TABLE 25
Composition of exemplary nucleic acid-lipid particle (mole %)
prepared via extrusion methods.

| Lipid | | | | |
|---|---|---|---|---|
| (mol %) | DSPC (mol %) | Chol (mol %) | PEG (mol %) | Lipid/siRNA |
| 20 | 30 | 40 | 10 | 2.13 |
| 20 | 30 | 40 | 10 | 2.35 |
| 20 | 30 | 40 | 10 | 2.37 |
| 20 | 30 | 40 | 10 | 3.23 |
| 20 | 30 | 40 | 10 | 3.91 |
| 30 | 20 | 40 | 10 | 2.89 |
| 30 | 20 | 40 | 10 | 3.34 |
| 30 | 20 | 40 | 10 | 3.34 |
| 30 | 20 | 40 | 10 | 4.10 |
| 30 | 20 | 40 | 10 | 5.64 |
| 40 | 10 | 40 | 10 | 3.02 |
| 40 | 10 | 40 | 10 | 3.35 |
| 40 | 10 | 40 | 10 | 3.74 |
| 40 | 10 | 40 | 10 | 5.80 |
| 40 | 10 | 40 | 10 | 8.00 |
| 45 | 5 | 40 | 10 | 3.27 |
| 45 | 5 | 40 | 10 | 3.30 |
| 45 | 5 | 40 | 10 | 4.45 |
| 45 | 5 | 40 | 10 | 7.00 |
| 45 | 5 | 40 | 10 | 9.80 |
| 50 | 0 | 40 | 10 | 27.03 |
| 20 | 35 | 40 | 5 | 3.00 |
| 20 | 35 | 40 | 5 | 3.32 |
| 20 | 35 | 40 | 5 | 3.05 |
| 20 | 35 | 40 | 5 | 3.67 |
| 20 | 35 | 40 | 5 | 4.71 |
| 30 | 25 | 40 | 5 | 2.47 |
| 30 | 25 | 40 | 5 | 2.98 |
| 30 | 25 | 40 | 5 | 3.29 |
| 30 | 25 | 40 | 5 | 4.99 |
| 30 | 25 | 40 | 5 | 7.15 |
| 40 | 15 | 40 | 5 | 2.79 |
| 40 | 15 | 40 | 5 | 3.29 |
| 40 | 15 | 40 | 5 | 4.33 |
| 40 | 15 | 40 | 5 | 7.05 |
| 40 | 15 | 40 | 5 | 9.63 |
| 45 | 10 | 40 | 5 | 2.44 |
| 45 | 10 | 40 | 5 | 3.21 |
| 45 | 10 | 40 | 5 | 4.29 |
| 45 | 10 | 40 | 5 | 6.50 |
| 45 | 10 | 40 | 5 | 8.67 |
| 20 | 35 | 40 | 5 | 4.10 |
| 20 | 35 | 40 | 5 | 4.83 |
| 30 | 25 | 40 | 5 | 3.86 |
| 30 | 25 | 40 | 5 | 5.38 |
| 30 | 25 | 40 | 5 | 7.07 |
| 40 | 15 | 40 | 5 | 3.85 |
| 40 | 15 | 40 | 5 | 4.88 |
| 40 | 15 | 40 | 5 | 7.22 |
| 40 | 15 | 40 | 5 | 9.75 |
| 45 | 10 | 40 | 5 | 2.83 |
| 45 | 10 | 40 | 5 | 3.85 |
| 45 | 10 | 40 | 5 | 4.88 |
| 45 | 10 | 40 | 5 | 7.05 |
| 45 | 10 | 40 | 5 | 9.29 |
| 45 | 20 | 30 | 5 | 4.01 |
| 45 | 20 | 30 | 5 | 3.70 |
| 50 | 15 | 30 | 5 | 4.75 |
| 50 | 15 | 30 | 5 | 3.80 |
| 55 | 10 | 30 | 5 | 3.85 |
| 55 | 10 | 30 | 5 | 4.13 |
| 60 | 5 | 30 | 5 | 5.09 |
| 60 | 5 | 30 | 5 | 4.67 |
| 65 | 0 | 30 | 5 | 4.75 |
| 65 | 0 | 30 | 5 | 6.06 |
| 56.5 | 10 | 30 | 3.5 | 3.70 |
| 56.5 | 10 | 30 | 3.5 | 3.56 |
| 57.5 | 10 | 30 | 2.5 | 3.48 |
| 57.5 | 10 | 30 | 2.5 | 3.20 |
| 58.5 | 10 | 30 | 1.5 | 3.24 |
| 58.5 | 10 | 30 | 1.5 | 3.13 |
| 59.5 | 10 | 30 | 0.5 | 3.24 |
| 59.5 | 10 | 30 | 0.5 | 3.03 |
| 45 | 10 | 40 | 5 | 7.57 |
| 45 | 10 | 40 | 5 | 7.24 |
| 45 | 10 | 40 | 5 | 7.48 |
| 45 | 10 | 40 | 5 | 7.84 |
| 65 | 0 | 30 | 5 | 4.01 |
| 60 | 5 | 30 | 5 | 3.70 |
| 55 | 10 | 30 | 5 | 3.65 |
| 50 | 10 | 35 | 5 | 3.43 |
| 50 | 15 | 30 | 5 | 3.80 |
| 45 | 15 | 35 | 5 | 3.70 |
| 45 | 20 | 30 | 5 | 3.75 |
| 45 | 25 | 25 | 5 | 3.85 |
| 55 | 10 | 32.5 | 2.5 | 3.61 |
| 60 | 10 | 27.5 | 2.5 | 3.65 |
| 60 | 10 | 25 | 5 | 4.07 |
| 55 | 5 | 38.5 | 1.5 | 3.75 |
| 60 | 10 | 28.5 | 1.5 | 3.43 |
| 55 | 10 | 33.5 | 1.5 | 3.48 |
| 60 | 5 | 33.5 | 1.5 | 3.43 |
| 55 | 5 | 37.5 | 2.5 | 3.75 |
| 60 | 5 | 32.5 | 2.5 | 4.52 |
| 60 | 5 | 32.5 | 2.5 | 3.52 |
| 45 | 15 (DMPC) | 35 | 5 | 3.20 |
| 45 | 15 (DPPC) | 35 | 5 | 3.43 |
| 45 | 15 (DOPC) | 35 | 5 | 4.52 |
| 45 | 15 (POPC) | 35 | 5 | 3.85 |
| 55 | 5 | 37.5 | 2.5 | 3.96 |
| 55 | 10 | 32.5 | 2.5 | 3.56 |
| 60 | 5 | 32.5 | 2.5 | 3.80 |
| 60 | 10 | 27.5 | 2.5 | 3.75 |
| 60 | 5 | 30 | 5 | 4.19 |
| 60 | 5 | 33.5 | 1.5 | 3.48 |
| 60 | 5 | 33.5 | 1.5 | 6.64 |
| 60 | 5 | 30 | 5 | 3.90 |
| 60 | 5 | 30 | 5 | 4.65 |
| 60 | 5 | 30 | 5 | 5.88 |
| 60 | 5 | 30 | 5 | 7.51 |
| 60 | 5 | 30 | 5 | 9.51 |
| 60 | 5 | 30 | 5 | 11.06 |
| 62.5 | 2.5 | 50 | 5 | 6.63 |
| 45 | 15 | 35 | 5 | 3.31 |
| 45 | 15 | 35 | 5 | 6.80 |
| 60 | 5 | 25 | 10 | 6.48 |
| 60 | 5 | 32.5 | 2.5 | 3.43 |
| 60 | 5 | 30 | 5 | 3.90 |
| 60 | 5 | 30 | 5 | 7.61 |
| 45 | 15 | 35 | 5 | 3.13 |
| 45 | 15 | 35 | 5 | 6.42 |
| 60 | 5 | 25 | 10 | 6.48 |
| 60 | 5 | 32.5 | 2.5 | 3.03 |
| 60 | 5 | 30 | 5 | 3.43 |
| 60 | 5 | 30 | 5 | 6.72 |
| 60 | 5 | 30 | 5 | 4.13 |
| 70 | 5 | 20 | 5 | 5.48 |
| 80 | 5 | 10 | 5 | 5.94 |
| 90 | 5 | 0 | 5 | 9.50 |
| 60 | 5 | 30 | 5 C12PEG | 3.85 |
| 60 | 5 | 30 | 5 | 3.70 |
| 60 | 5 | 30 | 5 C16PEG | 3.80 |
| 60 | 5 | 30 | 5 | 4.19 |
| 60 | 5 | 29 | 5 | 4.07 |
| 60 | 5 | 30 | 5 | 3.56 |
| 60 | 5 | 30 | 5 | 3.39 |
| 60 | 5 | 30 | 5 | 3.96 |
| 60 | 5 | 30 | 5 | 4.01 |
| 60 | 5 | 30 | 5 | 4.07 |
| 60 | 5 | 30 | 5 | 4.25 |
| 60 | 5 | 30 | 5 | 3.80 |
| 60 | 5 | 30 | 5 | 3.31 |
| 60 | 5 | 30 | 5 | 4.83 |
| 60 | 5 | 30 | 5 | 4.67 |
| 60 | 5 | 30 | 5 | 3.96 |
| 57.5 | 7.5 | 33.5 | 1.5 | 3.39 |
| 57.5 | 7.5 | 32.5 | 2.5 | 3.39 |
| 57.5 | 7.5 | 31.5 | 3.5 | 3.52 |
| 57.5 | 7.5 | 30 | 5 | 4.19 |
| 60 | 5 | 30 | 5 | 3.96 |
| 60 | 5 | 30 | 5 | 3.96 |
| 60 | 5 | 30 | 5 | 3.56 |
| 60 | 5 | 33.5 | 1.5 | 3.52 |
| 60 | 5 | 25 | 10 | 5.18 |
| 60 | 5 (DPPC) | 30 | 5 | 4.25 |
| 60 | 5 | 32.5 | 2.5 | 3.70 |
| 57.5 | 7.5 | 31.5 | 3.5 | 3.06 |
| 57.5 | 7.5 | 31.5 | 3.5 | 3.65 |
| 57.5 | 7.5 | 31.5 | 3.5 | 4.70 |
| 57.5 | 7.5 | 31.5 | 3.5 | 6.56 |

TABLE 26
Composition of exemplary nucleic acid-lipid particles prepared
via in-line mixing

| Lipid | DSPC | Chol | PEG | Lipid A/ |
|---|---|---|---|---|
| (mol %) | (mol %) | (mol %) | (mol %) | siRNA |
| 55 | 5 | 37.5 | 2.5 | 3.96 |
| 55 | 10 | 32.5 | 2.5 | 3.56 |
| 60 | 5 | 32.5 | 2.5 | 3.80 |
| 60 | 10 | 27.5 | 2.5 | 3.75 |
| 60 | 5 | 30 | 5 | 4.19 |
| 60 | 5 | 33.5 | 1.5 | 3.48 |
| 60 | 5 | 33.5 | 1.5 | 6.64 |
| 60 | 5 | 25 | 10 | 6.79 |
| 60 | 5 | 32.5 | 2.5 | 3.96 |
| 60 | 5 | 34 | 1 | 3.75 |
| 60 | 5 | 34.5 | 0.5 | 3.28 |
| 50 | 5 | 40 | 5 | 3.96 |
| 60 | 5 | 30 | 5 | 4.75 |
| 70 | 5 | 20 | 5 | 5.00 |
| 80 | 5 | 10 | 5 | 5.18 |
| 60 | 5 | 30 | 5 | 13.60 |
| 60 | 5 | 30 | 5 | 14.51 |
| 60 | 5 | 30 | 5 | 6.20 |
| 60 | 5 | 30 | 5 | 4.60 |
| 60 | 5 | 30 | 5 | 6.20 |
| 60 | 5 | 30 | 5 | 5.82 |
| 40 | 5 | 54 | 1 | 3.39 |
| 40 | 7.5 | 51.5 | 1 | 3.39 |
| 40 | 10 | 49 | 1 | 3.39 |
| 50 | 5 | 44 | 1 | 3.39 |
| 50 | 7.5 | 41.5 | 1 | 3.43 |
| 50 | 10 | 39 | 1 | 3.35 |
| 60 | 5 | 34 | 1 | 3.52 |
| 60 | 7.5 | 31.5 | 1 | 3.56 |
| 60 | 10 | 29 | 1 | 3.80 |
| 70 | 5 | 24 | 1 | 3.70 |
| 70 | 7.5 | 21.5 | 1 | 4.13 |
| 70 | 10 | 19 | 1 | 3.85 |
| 60 | 5 | 34 | 1 | 3.52 |
| 60 | 5 | 34 | 1 | 3.70 |
| 60 | 5 | 34 | 1 | 3.52 |
| 60 | 7.5 | 27.5 | 5 | 5.18 |
| 60 | 7.5 | 29 | 3.5 | 4.45 |
| 60 | 5 | 31.5 | 3.5 | 4.83 |
| 60 | 7.5 | 31 | 1.5 | 3.48 |
| 57.5 | 7.5 | 30 | 5 | 4.75 |
| 57.5 | 7.5 | 31.5 | 3.5 | 4.83 |
| 57.5 | 5 | 34 | 3.5 | 4.67 |
| 57.5 | 7.5 | 33.5 | 1.5 | 3.43 |
| 55 | 7.5 | 32.5 | 5 | 4.38 |
| 55 | 7.5 | 34 | 3.5 | 4.13 |
| 55 | 5 | 36.5 | 3.5 | 4.38 |
| 55 | 7.5 | 36 | 1.5 | 3.35 |

Synthesis of Cationic Lipids.

Any of the compounds, e.g., cationic lipids and the like, used in the nucleic acid-lipid particles of the invention may be prepared by known organic synthesis techniques, including the methods described in more detail in the Examples. All substituents are as defined below unless indicated otherwise.

“Alkyl” means a straight chain or branched, noncyclic or cyclic, saturated aliphatic hydrocarbon containing from 1 to 24 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tent-butyl, isopentyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like.

“Alkenyl” means an alkyl, as defined above, containing at least one double bond between adjacent carbon atoms. Alkenyls include both cis and trans isomers. Representative straight chain and branched alkenyls include ethylenyl, propylenyl, 1-butenyl, 2-butenyl, isobutylenyl, 1-pentenyl, 2-pentenyl, 3-methyl-l-butenyl, 2-methyl-2-butenyl, 2,3-dimethyl-2-butenyl, and the like.

“Alkynyl” means any alkyl or alkenyl, as defined above, which additionally contains at least one triple bond between adjacent carbons. Representative straight chain and branched alkynyls include acetylenyl, propynyl, 1-butynyl, 2-butynyl, 1-pentynyl, 2-pentynyl, 3-methyl-1 butynyl, and the like.

“Acyl” means any alkyl, alkenyl, or alkynyl wherein the carbon at the point of attachment is substituted with an oxo group, as defined below. For example, —C(═O)alkyl, —C(═O)alkenyl, and —C(═O)alkynyl are acyl groups.

“Heterocycle” means a 5- to 7-membered monocyclic, or 7- to 10-membered bicyclic, heterocyclic ring which is either saturated, unsaturated, or aromatic, and which contains from 1 or 2 heteroatoms independently selected from nitrogen, oxygen and sulfur, and wherein the nitrogen and sulfur heteroatoms may be optionally oxidized, and the nitrogen heteroatom may be optionally quatermized, including bicyclic rings in which any of the above heterocycles are fused to a benzene ring. The heterocycle may be attached via any heteroatom or carbon atom. Heterocycles include heteroaryls as defined below. Heterocycles include morpholinyl, pyrrolidinonyl, pyrrolidinyl, piperidinyl, piperizynyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

The terms “optionally substituted alkyl”, “optionally substituted alkenyl”, “optionally substituted alkynyl”, “optionally substituted acyl”, and “optionally substituted heterocycle” means that, when substituted, at least one hydrogen atom is replaced with a substituent. In the case of an oxo substituent (═O) two hydrogen atoms are replaced. In this regard, substituents include oxo, halogen, heterocycle, —CN, —OR^{x}, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x} and —SOₙNR^{x}R^{y}, wherein n is 0, 1 or 2, R^{x} and R^{y} are the same or different and independently hydrogen, alkyl or heterocycle, and each of said alkyl and heterocycle substituents may be further substituted with one or more of oxo, halogen, —OH, —CN, alkyl, —OR^{x}, heterocycle, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x} and —SOₙNR^{x}R^{y}.

“Halogen” means fluoro, chloro, bromo and iodo.

In some embodiments, the methods of the invention may require the use of protecting groups. Protecting group methodology is well known to those skilled in the art (see, for example, PROTECTIVE GROUPS IN ORGANIC SYNTHESIS, Green, T. W. et al., Wiley-Interscience, New York City, 1999). Briefly, protecting groups within the context of this invention are any group that reduces or eliminates unwanted reactivity of a functional group. A protecting group can be added to a functional group to mask its reactivity during certain reactions and then removed to reveal the original functional group. In some embodiments an “alcohol protecting group” is used. An “alcohol protecting group” is any group which decreases or eliminates unwanted reactivity of an alcohol functional group. Protecting groups can be added and removed using techniques well known in the art.

Synthesis of Formula A

In one embodiments, nucleic acid-lipid particles of the invention are formulated using a cationic lipid of formula A:

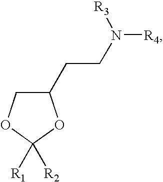

where R1 and R2 are independently alkyl, alkenyl or alkynyl, each can be optionally substituted, and R3 and R4 are independently lower alkyl or R3 and R4 can be taken together to form an optionally substituted heterocyclic ring. In some embodiments, the cationic lipid is XTC (2,2-Dilinoleyl-4-dimethylaminoethyl-[1,3]-dioxolane). In general, the lipid of formula A above may be made by the following Reaction Schemes 1 or 2, wherein all substituents are as defined above unless indicated otherwise.

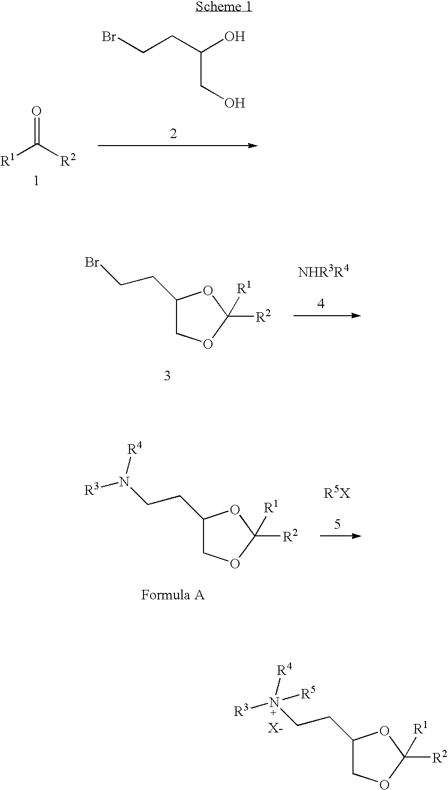

Lipid A, where R₁ and R₂ are independently alkyl, alkenyl or alkynyl, each can be optionally substituted, and R₃ and R₄ are independently lower alkyl or R₃ and R₄ can be taken together to form an optionally substituted heterocyclic ring, can be prepared according to Scheme 1. Ketone 1 and bromide 2 can be purchased or prepared according to methods known to those of ordinary skill in the art. Reaction of 1 and 2 yields ketal 3. Treatment of ketal 3 with amine 4 yields lipids of formula A. The lipids of formula A can be converted to the corresponding ammonium salt with an organic salt of formula 5, where X is anion counter ion selected from halogen, hydroxide, phosphate, sulfate, or the like.

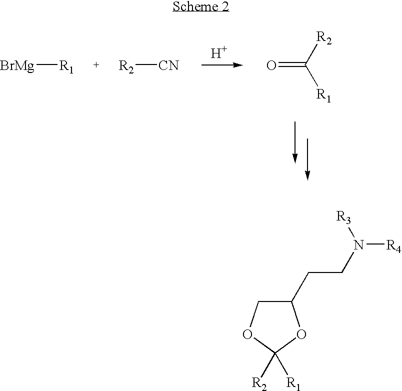

Alternatively, the ketone 1 starting material can be prepared according to Scheme 2. Grignard reagent 6 and cyanide 7 can be purchased or prepared according to methods known to those of ordinary skill in the art. Reaction of 6 and 7 yields ketone 1. Conversion of ketone 1 to the corresponding lipids of formula A is as described in Scheme 1.

Synthesis of MC3

Preparation of DLin-M-C3-DMA (i.e., (6Z,9Z,28Z,31Z)-heptatriaconta-6,9,28,31-tetraen-19-yl 4-(dimethylamino)butanoate) was as follows. A solution of (6Z,9Z,28Z,31Z)-heptatriaconta-6,9,28,31-tetraen-19-ol (0.53 g), 4-N,N-dimethylaminobutyric acid hydrochloride (0.51 g), 4-N,N-dimethylaminopyridine (0.61 g) and 1-ethyl-3-(3-dimethylaminopropyl)carbodiimide hydrochloride (0.53 g) in dichloromethane (5 mL) was stirred at room temperature overnight. The solution was washed with dilute hydrochloric acid followed by dilute aqueous sodium bicarbonate. The organic fractions were dried over anhydrous magnesium sulphate, filtered and the solvent removed on a rotovap. The residue was passed down a silica gel column (20 g) using a 1-5% methanol/dichloromethane elution gradient.

Fractions containing the purified product were combined and the solvent removed, yielding a colorless oil (0.54 g).

Synthesis of ALNY-100

Synthesis of ketal 519 [ALNY-100] was performed using the following scheme 3:

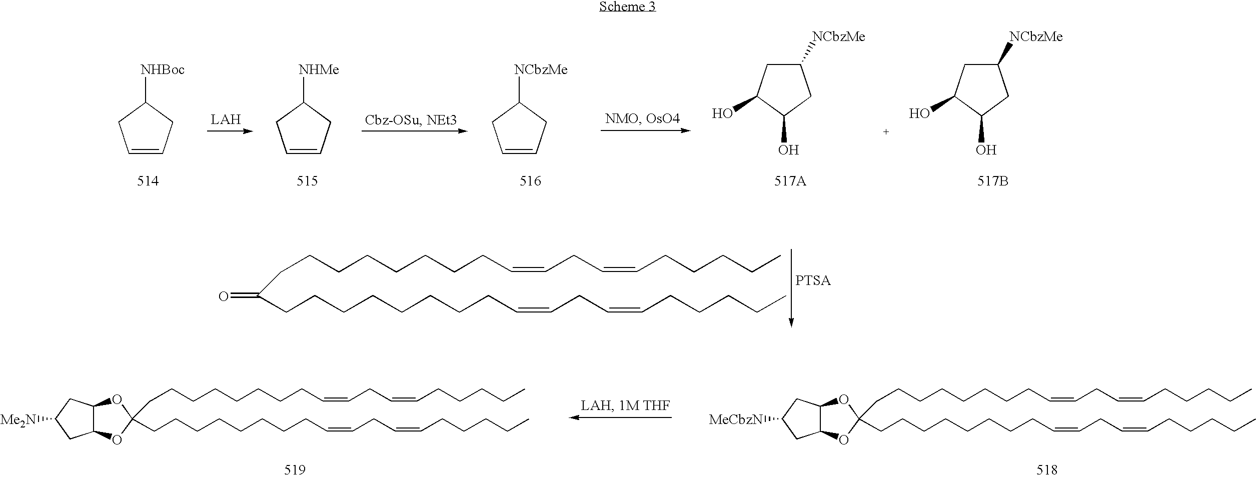

Synthesis of 515:

To a stirred suspension of LiAlH4 (3.74 g, 0.09852 mol) in 200 ml anhydrous THF in a two neck RBF (1 L), was added a solution of 514 (10 g, 0.04926 mol) in 70 mL of THF slowly at 0 0 C under nitrogen atmosphere. After complete addition, reaction mixture was warmed to room temperature and then heated to reflux for 4 h. Progress of the reaction was monitored by TLC. After completion of reaction (by TLC) the mixture was cooled to 0 0 C and quenched with careful addition of saturated Na2SO4 solution. Reaction mixture was stirred for 4 h at room temperature and filtered off. Residue was washed well with THF. The filtrate and washings were mixed and diluted with 400 mL dioxane and 26 mL conc. HCl and stirred for 20 minutes at room temperature. The volatilities were stripped off under vacuum to furnish the hydrochloride salt of 515 as a white solid. Yield: 7.12 g 1H-NMR (DMSO, 400 MHz): δ=9.34 (broad, 2H), 5.68 (s, 2H), 3.74 (m, 1H), 2.66-2.60 (m, 2H), 2.50-2.45 (m, 5H).

Synthesis of 516:

To a stirred solution of compound 515 in 100 mL dry DCM in a 250 mL two neck RBF, was added NEt3 (37.2 mL, 0.2669 mol) and cooled to 0 0 C under nitrogen atmosphere. After a slow addition of N-(benzyloxy-carbonyloxy)-succinimide (20 g, 0.08007 mol) in 50 mL dry DCM, reaction mixture was allowed to warm to room temperature. After completion of the reaction (2-3 h by TLC) mixture was washed successively with 1N HCl solution (1×100 mL) and saturated NaHCO3 solution (1×50 mL). The organic layer was then dried over anhyd. Na2SO4 and the solvent was evaporated to give crude material which was purified by silica gel column chromatography to get 516 as sticky mass. Yield: 11 g (89%). 1H-NMR (CDCl3, 400 MHz): δ=7.36-7.27(m, 5H), 5.69 (s, 2H), 5.12 (s, 2H), 4.96 (br., 1H) 2.74 (s, 3H), 2.60(m, 2H), 2.30-2.25(m, 2H). LC-MS [M+H]−232.3 (96.94%).

Synthesis of 517A and 517B:

The cyclopentene 516 (5 g, 0.02164 mol) was dissolved in a solution of 220 mL acetone and water (10:1) in a single neck 500 mL RBF and to it was added N-methyl morpholine-N-oxide (7.6 g, 0.06492 mol) followed by 4.2 mL of 7.6% solution of OsO4 (0.275 g, 0.00108 mol) in tert-butanol at room temperature. After completion of the reaction (˜3 h), the mixture was quenched with addition of solid Na2SO3 and resulting mixture was stirred for 1.5 h at room temperature. Reaction mixture was diluted with DCM (300 mL) and washed with water (2×100 mL) followed by saturated NaHCO3 (1×50 mL) solution, water (1×30 mL) and finally with brine (1×50 mL). Organic phase was dried over an Na2SO4 and solvent was removed in vacuum. Silica gel column chromatographic purification of the crude material was afforded a mixture of diastereomers, which were separated by prep HPLC. Yield: −6 g crude

517A—Peak-1 (white solid), 5.13 g (96%). 1H-NMR (DMSO, 400 MHz): δ=7.39-7.31(m, 5H), 5.04(s, 2H), 4.78-4.73 (m, 1H), 4.48-4.47(d, 2H), 3.94-3.93(m, 2H), 2.71(s, 3H), 1.72-1.67(m, 4H). LC-MS−[M+H]−266.3, [M+NH4+]−283.5 present, HPLC-97.86%. Stereochemistry confirmed by X-ray.

Synthesis of 518:

Using a procedure analogous to that described for the synthesis of compound 505, compound 518 (1.2 g, 41%) was obtained as a colorless oil. 1H-NMR (CDC13, 400MHz): δ=7.35-7.33(m, 4H), 7.30-7.27(m, 1H), 5.37-5.27(m, 8H), 5.12(s, 2H), 4.75(m,1H), 4.58-4.57(m,2H), 2.78-2.74(m,7H), 2.06-2.00(m,8H), 1.96-1.91(m, 2H), 1.62(m, 4H), 1.48(m, 2H), 1.37-1.25(br m, 36H), 0.87(m, 6H). HPLC-98.65%.

General Procedure for the Synthesis of Compound 519:

A solution of compound 518 (1 eq) in hexane (15 mL) was added in a drop-wise fashion to an ice-cold solution of LAH in THF (1 M, 2 eq). After complete addition, the mixture was heated at 40 C over 0.5 h then cooled again on an ice bath. The mixture was carefully hydrolyzed with saturated aqueous Na2SO4 then filtered through celite and reduced to an oil. Column chromatography provided the pure 519 (1.3 g, 68%) which was obtained as a colorless oil. 13C NMR=130.2, 130.1 (x2), 127.9 (x3), 112.3, 79.3, 64.4, 44.7, 38.3, 35.4, 31.5, 29.9 (x2), 29.7, 29.6 (x2), 29.5 (x3), 29.3 (x2), 27.2 (x3), 25.6, 24.5, 23.3, 226, 14.1; Electrospray MS (+ve): Molecular weight for C44H80NO2 (M+H)+Calc. 654.6, Found 654.6.

Therapeutic Agent-Lipid Particle Compositions and Formulations

The invention includes compositions comprising a lipid particle of the invention and an active agent, wherein the active agent is associated with the lipid particle. In particular embodiments, the active agent is a therapeutic agent. In particular embodiments, the active agent is encapsulated within an aqueous interior of the lipid particle. In other embodiments, the active agent is present within one or more lipid layers of the lipid particle. In other embodiments, the active agent is bound to the exterior or interior lipid surface of a lipid particle.

“Fully encapsulated” as used herein indicates that the nucleic acid in the particles is not significantly degraded after exposure to serum or a nuclease assay that would significantly degrade free DNA. In a fully encapsulated system, preferably less than 25% of particle nucleic acid is degraded in a treatment that would normally degrade 100% of free nucleic acid, more preferably less than 10% and most preferably less than 5% of the particle nucleic acid is degraded. Alternatively, full encapsulation may be determined by an Oligreen® assay. Oligreen® is an ultra-sensitive fluorescent nucleic acid stain for quantitating oligonucleotides and single-stranded DNA in solution (available from Invitrogen Corporation, Carlsbad, Calif.). Fully encapsulated also suggests that the particles are serum stable, that is, that they do not rapidly decompose into their component parts upon in vivo administration.

Active agents, as used herein, include any molecule or compound capable of exerting a desired effect on a cell, tissue, organ, or subject. Such effects may be biological, physiological, or cosmetic, for example. Active agents may be any type of molecule or compound, including e.g., nucleic acids, peptides and polypeptides, including, e.g., antibodies, such as, e.g., polyclonal antibodies, monoclonal antibodies, antibody fragments; humanized antibodies, recombinant antibodies, recombinant human antibodies, and PrimatizedTM antibodies, cytokines, growth factors, apoptotic factors, differentiation-inducing factors, cell surface receptors and their ligands; hormones; and small molecules, including small organic molecules or compounds.

In one embodiment, the active agent is a therapeutic agent, or a salt or derivative thereof. Therapeutic agent derivatives may be therapeutically active themselves or they may be prodrugs, which become active upon further modification. Thus, in one embodiment, a therapeutic agent derivative retains some or all of the therapeutic activity as compared to the unmodified agent, while in another embodiment, a therapeutic agent derivative lacks therapeutic activity.

In various embodiments, therapeutic agents include any therapeutically effective agent or drug, such as anti-inflammatory compounds, anti-depressants, stimulants, analgesics, antibiotics, birth control medication, antipyretics, vasodilators, anti-angiogenics, cytovascular agents, signal transduction inhibitors, cardiovascular drugs, e.g., anti-arrhythmic agents, vasoconstrictors, hormones, and steroids.

In certain embodiments, the therapeutic agent is an oncology drug, which may also be referred to as an anti-tumor drug, an anti-cancer drug, a tumor drug, an antineoplastic agent, or the like. Examples of oncology drugs that may be used according to the invention include, but are not limited to, adriamycin, alkeran, allopurinol, altretamine, amifostine, anastrozole, araC, arsenic trioxide, azathioprine, bexarotene, biCNU, bleomycin, busulfan intravenous, busulfan oral, capecitabine (Xeloda), carboplatin, carmustine, CCNU, celecoxib, chlorambucil, cisplatin, cladribine, cyclosporin A, cytarabine, cytosine arabinoside, daunorubicin, cytoxan, daunorubicin, dexamethasone, dexrazoxane, dodetaxel, doxorubicin, doxorubicin, DTIC, epirubicin, estramustine, etoposide phosphate, etoposide and VP-16, exemestane, FK506, fludarabine, fluorouracil, 5-FU, gemcitabine (Gemzar), gemtuzumab-ozogamicin, goserelin acetate, hydrea, hydroxyurea, idarubicin, ifosfamide, imatinib mesylate, interferon, irinotecan (Camptostar, CPT-111), letrozole, leucovorin, leustatin, leuprolide, levamisole, litretinoin, megastrol, melphalan, L-PAM, mesna, methotrexate, methoxsalen, mithramycin, mitomycin, mitoxantrone, nitrogen mustard, paclitaxel, pamidronate, Pegademase, pentostatin, porfimer sodium, prednisone, rituxan, streptozocin, STI-571, tamoxifen, taxotere, temozolamide, teniposide, VM-26, topotecan (Hycamtin), toremifene, tretinoin, ATRA, valrubicin, velban, vinblastine, vincristine, VP16, and vinorelbine. Other examples of oncology drugs that may be used according to the invention are ellipticin and ellipticin analogs or derivatives, epothilones, intracellular kinase inhibitors and camptothecins.

Additional Formulations

Emulsions

The compositions of the present invention may be prepared and formulated as emulsions. Emulsions are typically heterogeneous systems of one liquid dispersed in another in the form of droplets usually exceeding 0.1 μm in diameter (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising two immiscible liquid phases intimately mixed and dispersed with each other. In general, emulsions may be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions may contain additional components in addition to the dispersed phases, and the active drug which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosage Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, non-swelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Since emulsions often contain a number of ingredients such as carbohydrates, proteins, sterols and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of ease of formulation, as well as efficacy from an absorption and bioavailability standpoint (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins and high fat nutritive preparations are among the materials that have commonly been administered orally as o/w emulsions.

In one embodiment of the present invention, the compositions of dsRNAs and nucleic acids are formulated as microemulsions. A microemulsion may be defined as a system of water, oil and amphiphile which is a single optically isotropic and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 185-215). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (P0310), hexaglycerol pentaoleate (P0500), decaglycerol monocaprate (MCA750), decaglycerol monooleate (M0750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides or dsRNAs. Microemulsions have also been effective in the transdermal delivery of active components in both cosmetic and pharmaceutical applications. It is expected that the microemulsion compositions and formulations of the present invention will facilitate the increased systemic absorption of dsRNAs and nucleic acids from the gastrointestinal tract, as well as improve the local cellular uptake of dsRNAs and nucleic acids.

Microemulsions of the present invention may also contain additional components and additives such as sorbitan monostearate (Grill 3), Labrasol, and penetration enhancers to improve the properties of the formulation and to enhance the absorption of the dsRNAs and nucleic acids of the present invention. Penetration enhancers used in the microemulsions of the present invention may be classified as belonging to one of five broad categories--surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Penetration Enhancers

In one embodiment, the present invention employs various penetration enhancers to effect the efficient delivery of nucleic acids, particularly dsRNAs, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating non-surfactants (Lee et al., Critical Reviews in Therapeutic Drug Carrier Systems, 1991, p.92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of dsRNAs through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p.92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacycloheptan-2-one, acylcarnitines, acylcholines, C₁₋₁₀ alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p.92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds., McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of dsRNAs through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, non-chelating non-surfactant penetration enhancing compounds can be defined as compounds that demonstrate insignificant activity as chelating agents or as surfactants but that nonetheless enhance absorption of dsRNAs through the alimentary mucosa (Muranishi, Critical Reviews in Therapeutic Drug Carrier Systems, 1990, 7, 1-33). This class of penetration enhancers include, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin and phenylbutazone (Yamashita et al., J. Pharm. Pharmacol., 1987, 39, 621-626).

Agents that enhance uptake of dsRNAs at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of dsRNAs.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

dsRNAs of the present invention can be formulated in a pharmaceutically acceptable carrier or diluent. A “pharmaceutically acceptable carrier” (also referred to herein as an “excipient”) is a pharmaceutically acceptable solvent, suspending agent, or any other pharmacologically inert vehicle. Pharmaceutically acceptable carriers can be liquid or solid, and can be selected with the planned manner of administration in mind so as to provide for the desired bulk, consistency, and other pertinent transport and chemical properties. Typical pharmaceutically acceptable carriers include, by way of example and not limitation: water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The co-administration of a nucleic acid and a carrier compound, typically with an excess of the latter substance, can result in a substantial reduction of the amount of nucleic acid recovered in the liver, kidney or other extra-circulatory reservoirs, presumably due to competition between the carrier compound and the nucleic acid for a common receptor. For example, the recovery of a partially phosphorothioate dsRNA in hepatic tissue can be reduced when it is co-administered with polyinosinic acid, dextran sulfate, polycytidic acid or 4-acetamido-4′isothiocyano-stilbene-2,2′-disulfonic acid (Miyao et al., DsRNA Res. Dev., 1995, 5, 115-121; Takakura et al., DsRNA & Nucl. Acid Drug Dev., 1996, 6, 177-183.

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents and other suitable additives. Pharmaceutically acceptable organic or inorganic excipients suitable for non-parenteral administration which do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances which increase the viscosity of the suspension including, for example, sodium carboxymethylcellulose, sorbitol and/or dextran. The suspension may also contain stabilizers.

Combination Therapy

In one aspect, a composition of the invention can be used in combination therapy. The term “combination therapy” includes the administration of the subject compounds in further combination with other biologically active ingredients (such as, but not limited to, a second and different antineoplastic agent) and non-drug therapies (such as, but not limited to, surgery or radiation treatment). For instance, the compounds of the invention can be used in combination with other pharmaceutically active compounds, preferably compounds that are able to enhance the effect of the compounds of the invention. The compounds of the invention can be administered simultaneously (as a single preparation or separate preparation) or sequentially to the other drug therapy. In general, a combination therapy envisions administration of two or more drugs during a single cycle or course of therapy.

In one aspect of the invention, the subject compounds may be administered in combination with one or more separate agents that modulate protein kinases involved in various disease states. Examples of such kinases may include, but are not limited to: serine/threonine specific kinases, receptor tyrosine specific kinases and non-receptor tyrosine specific kinases. Serine/threonine kinases include mitogen activated protein kinases (MAPK), meiosis specific kinase (MEK), RAF and aurora kinase. Examples of receptor kinase families include epidermal growth factor receptor (EGFR) (e.g., HER2/neu, HER3, HER4, ErbB, ErbB2, ErbB3, ErbB4, Xmrk, DER, Let23); fibroblast growth factor (FGF) receptor (e.g. FGF-R1, GFF-R2/BEK/CEK3, FGF-R3/CEK2, FGF-R4/TKF, KGF-R); hepatocyte growth/scatter factor receptor (HGFR) (e.g., MET, RON, SEA, SEX); insulin receptor (e.g. IGFI-R); Eph (e.g. CEK5, CEK8, EBK, ECK, EEK, EHK-I, EHK-2, ELK, EPH, ERK, HEK, MDK2, MDK5, SEK); AxI (e.g. Mer/Nyk, Rse); RET; and platelet-derived growth factor receptor (PDGFR) (e.g. PDGFα-R, PDGβ-R, CSF1-R/FMS, SCF- R/C-KIT, VEGF-R/FLT, NEK/FLK1, FLT3/FLK2/STK-1). Non-receptor tyrosine kinase families include, but are not limited to, BCR-ABL (e.g. p43^{abl}, ARG); BTK (e.g. ITK/EMT, TEC); CSK, FAK, FPS, JAK, SRC, BMX, FER, CDK and SYK.

In another aspect of the invention, the subject compounds may be administered in combination with one or more agents that modulate non-kinase biological targets or processes. Such targets include histone deacetylases (HDAC), DNA methyltransferase (DNMT), heat shock proteins (e.g., HSP90), and proteosomes.

In one embodiment, subject compounds may be combined with antineoplastic agents (e.g. small molecules, monoclonal antibodies, antisense RNA, and fusion proteins) that inhibit one or more biological targets such as Zolinza, Tarceva, Iressa, Tykerb, Gleevec, Sutent, Sprycel, Nexavar, Sorafenib, CNF2024, RG108, BMS387032, Affmitak, Avastin, Herceptin, Erbitux, AG24322, PD325901 , ZD6474, PD 184322, Obatodax, ABT737 and AEE788. Such combinations may enhance therapeutic efficacy over efficacy achieved by any of the agents alone and may prevent or delay the appearance of resistant mutational variants.

In certain preferred embodiments, the compounds of the invention are administered in combination with a chemotherapeutic agent. Chemotherapeutic agents encompass a wide range of therapeutic treatments in the field of oncology. These agents are administered at various stages of the disease for the purposes of shrinking tumors, destroying remaining cancer cells left over after surgery, inducing remission, maintaining remission and/or alleviating symptoms relating to the cancer or its treatment. Examples of such agents include, but are not limited to, alkylating agents such as mustard gas derivatives (Mechlorethamine, cylophosphamide, chlorambucil, melphalan, ifosfamide), ethylenimines (thiotepa, hexamethylmelanine), Alkylsulfonates (Busulfan), Hydrazines and Triazines (Altretamine, Procarbazine, Dacarbazine and Temozolomide), Nitrosoureas (Carmustine, Lomustine and Streptozocin), Ifosfamide and metal salts (Carboplatin, Cisplatin, and Oxaliplatin); plant alkaloids such as Podophyllotoxins (Etoposide and Tenisopide), Taxanes (Paclitaxel and Docetaxel), Vinca alkaloids (Vincristine, Vinblastine, Vindesine and Vinorelbine), and Camptothecan analogs (Irinotecan and Topotecan); anti-tumor antibiotics such as Chromomycins (Dactinomycin and Plicamycin), Anthracyclines (Doxorubicin, Daunorubicin, Epirubicin, Mitoxantrone, Valrubicin and Idarubicin), and miscellaneous antibiotics such as Mitomycin, Actinomycin and Bleomycin; anti-metabolites such as folic acid antagonists (Methotrexate, Pemetrexed, Raltitrexed, Aminopterin), pyrimidine antagonists (5-Fluorouracil, Floxuridine, Cytarabine, Capecitabine, and Gemcitabine), purine antagonists (6-Mercaptopurine and 6-Thioguanine) and adenosine deaminase inhibitors (Cladribine, Fludarabine, Mercaptopurine, Clofarabine, Thioguanine, Nelarabine and Pentostatin); topoisomerase inhibitors such as topoisomerase I inhibitors (Ironotecan, topotecan) and topoisomerase II inhibitors (Amsacrine, etoposide, etoposide phosphate, teniposide); monoclonal antibodies (Alemtuzumab, Gemtuzumab ozogamicin, Rituximab, Trastuzumab, Ibritumomab Tioxetan, Cetuximab, Panitumumab, Tositumomab, Bevacizumab); and miscellaneous anti-neoplasties such as ribonucleotide reductase inhibitors (Hydroxyurea); adrenocortical steroid inhibitor (Mitotane); enzymes (Asparaginase and Pegaspargase); anti-microtubule agents (Estramustine); and retinoids (Bexarotene, Isotretinoin, Tretinoin (ATRA). In certain preferred embodiments, the compounds of the invention are administered in combination with a chemoprotective agent. Chemoprotective agents act to protect the body or minimize the side effects of chemotherapy. Examples of such agents include, but are not limited to, amfostine, mesna, and dexrazoxane.

In one aspect of the invention, the subject compounds are administered in combination with radiation therapy. Radiation is commonly delivered internally (implantation of radioactive material near cancer site) or externally from a machine that employs photon (x-ray or gamma-ray) or particle radiation. Where the combination therapy further comprises radiation treatment, the radiation treatment may be conducted at any suitable time so long as a beneficial effect from the co-action of the combination of the therapeutic agents and radiation treatment is achieved. For example, in appropriate cases, the beneficial effect is still achieved when the radiation treatment is temporally removed from the administration of the therapeutic agents, perhaps by days or even weeks.

It will be appreciated that compounds of the invention can be used in combination with an immunotherapeutic agent. One form of immunotherapy is the generation of an active systemic tumor-specific immune response of host origin by administering a vaccine composition at a site distant from the tumor. Various types of vaccines have been proposed, including isolated tumor-antigen vaccines and anti-idiotype vaccines. Another approach is to use tumor cells from the subject to be treated, or a derivative of such cells (reviewed by Schirmacher et al. (1995) J. Cancer Res. Clin. Oncol. 121 :487). In U.S. Pat. No. 5,484,596, Hanna Jr. et al. claim a method for treating a resectable carcinoma to prevent recurrence or metastases, comprising surgically removing the tumor, dispersing the cells with collagenase, irradiating the cells, and vaccinating the patient with at least three consecutive doses of about 10⁷ cells.

It will be appreciated that the compounds of the invention may advantageously be used in conjunction with one or more adjunctive therapeutic agents. Examples of suitable agents for adjunctive therapy include steroids, such as corticosteroids (amcinonide, betamethasone, betamethasone dipropionate, betamethasone valerate, budesonide, clobetasol, clobetasol acetate, clobetasol butyrate, clobetasol 17-propionate, cortisone, deflazacort, desoximetasone, diflucortolone valerate, dexamethasone, dexamethasone sodium phosphate, desonide, furoate, fluocinonide, fluocinolone acetonide, halcinonide, hydrocortisone, hydrocortisone butyrate, hydrocortisone sodium succinate, hydrocortisone valerate, methyl prednisolone, mometasone, prednicarbate, prednisolone, triamcinolone, triamcinolone acetonide, and halobetasol proprionate); a 5HTi agonist, such as a triptan (e.g. sumatriptan or naratriptan); an adenosine Al agonist; an EP ligand; an NMDA modulator, such as a glycine antagonist; a sodium channel blocker (e.g. lamotrigine); a substance P antagonist (e.g. an NKi antagonist); a cannabinoid; acetaminophen or phenacetin; a 5 -lipoxygenase inhibitor; a leukotriene receptor antagonist; a DMARD (e.g. methotrexate); gabapentin and related compounds; a tricyclic antidepressant (e.g. amitryptilline); a neurone stabilizing antiepileptic drug; a mono-aminergic uptake inhibitor (e.g. venlafaxine); a matrix metalloproteinase inhibitor; a nitric oxide synthase (NOS) inhibitor, such as an iNOS or an nNOS inhibitor; an inhibitor of the release, or action, of tumour necrosis factor a; an antibody therapy, such as a monoclonal antibody therapy; an antiviral agent, such as a nucleoside inhibitor (e.g. lamivudine) or an immune system modulator (e.g. interferon); an opioid analgesic; a local anaesthetic; a stimulant, including caffeine; an H2-antagonist (e.g. ranitidine); a proton pump inhibitor (e.g. omeprazole); an antacid (e.g. aluminium or magnesium hydroxide; an antiflatulent (e.g. simethicone); a decongestant (e.g. phenylephrine, phenylpropanolamine, pseudoephedrine, oxymetazoline, epinephrine, naphazoline, xylometazoline, propylhexedrine, or levo-desoxyephedrine); an antitussive (e.g. codeine, hydrocodone, carmiphen, carbetapentane, or dextramethorphan); a diuretic; or a sedating or non-sedating antihistamine.

The compounds of the invention can be co-administered with siRNA that target other genes. For example, a compound of the invention can be co-administered with an siRNA targeted to a c-Myc gene. In one example, AD-12115 can be co-administered with a c-Myc siRNA. Examples of c-Myc targeted siRNAs are disclosed in U.S. patent application Ser. No. 12/373,039 which is herein incorporated by reference.

Methods for Treating Diseases caused by Expression of the Eg5 and VEGF Genes

The invention relates in particular to the use of a composition containing at least two dsRNAs, one targeting an Eg5 gene, and one targeting a VEGF gene, for the treatment of a cancer, such as liver cancer, e.g., for inhibiting tumor growth and tumor metastasis. For example, a composition, such as pharmaceutical composition, may be used for the treatment of solid tumors, like intrahepatic tumors such as may occur in cancers of the liver. A composition containing a dsRNA targeting Eg5 and a dsRNA targeting VEGF may also be used to treat other tumors and cancers, such as breast cancer, lung cancer, head and neck cancer, brain cancer, abdominal cancer, colon cancer, colorectal cancer, esophagus cancer, gastrointestinal cancer, glioma, tongue cancer, neuroblastoma, osteosarcoma, ovarian cancer, pancreatic cancer, prostate cancer, retinoblastoma, Wilm's tumor, multiple myeloma and for the treatment of skin cancer, like melanoma, for the treatment of lymphomas and blood cancer. The invention further relates to the use of a composition containing an Eg5 dsRNA and a VEGF dsRNA for inhibiting accumulation of ascites fluid and pleural effusion in different types of cancer, e.g., liver cancer, breast cancer, lung cancer, head cancer, neck cancer, brain cancer, abdominal cancer, colon cancer, colorectal cancer, esophagus cancer, gastrointestinal cancer, glioma, tongue cancer, neuroblastoma, osteosarcoma, ovarian cancer, pancreatic cancer, prostate cancer, retinoblastoma, Wilm's tumor, multiple myeloma, skin cancer, melanoma, lymphomas and blood cancer. Owing to the inhibitory effects on Eg5 and VEGF expression, a composition according to the invention or a pharmaceutical composition prepared therefrom can enhance the quality of life.

In one embodiment, a patient having a tumor associated with AFP expression, or a tumor secreting AFP, e.g., a hepatoma or teratoma, is treated. In certain embodiments, the patient has a malignant teratoma, an endodermal sinus tumor (yolk sac carcinoma), a neuroblastoma, a hepatoblastoma, a heptocellular carcinoma, testicular cancer or ovarian cancer.

The invention furthermore relates to the use of a dsRNA or a pharmaceutical composition thereof, e.g., for treating cancer or for preventing tumor metastasis, in combination with other pharmaceuticals and/or other therapeutic methods, e.g., with known pharmaceuticals and/or known therapeutic methods, such as, for example, those which are currently employed for treating cancer and/or for preventing tumor metastasis. Preference is given to a combination with radiation therapy and chemotherapeutic agents, such as cisplatin, cyclophosphamide, 5-fluorouracil, adriamycin, daunorubicin or tamoxifen.

The invention can also be practiced by including with a specific RNAi agent, in combination with another anti-cancer chemotherapeutic agent, such as any conventional chemotherapeutic agent. The combination of a specific binding agent with such other agents can potentiate the chemotherapeutic protocol. Numerous chemotherapeutic protocols will present themselves in the mind of the skilled practitioner as being capable of incorporation into the method of the invention. Any chemotherapeutic agent can be used, including alkylating agents, antimetabolites, hormones and antagonists, radioisotopes, as well as natural products. For example, the compound of the invention can be administered with antibiotics such as doxorubicin and other anthracycline analogs, nitrogen mustards such as cyclophosphamide, pyrimidine analogs such as 5-fluorouracil, cisplatin, hydroxyurea, taxol and its natural and synthetic derivatives, and the like. As another example, in the case of mixed tumors, such as adenocarcinoma of the breast, where the tumors include gonadotropin-dependent and gonadotropin-independent cells, the compound can be administered in conjunction with leuprolide or goserelin (synthetic peptide analogs of LH-RH). Other antineoplastic protocols include the use of a tetracycline compound with another treatment modality, e.g., surgery, radiation, etc., also referred to herein as “adjunct antineoplastic modalities.” Thus, the method of the invention can be employed with such conventional regimens with the benefit of reducing side effects and enhancing efficacy.

Methods for Inhibiting Expression of the Eg5 Gene and the VEGF Gene

In yet another aspect, the invention provides a method for inhibiting the expression of the Eg5 gene and the VEGF gene in a mammal. The method includes administering a composition featured in the invention to the mammal such that expression of the target Eg5 gene and the target VEGF gene is silenced.

In one embodiment, a method for inhibiting Eg5 gene expression and VEGF gene expression includes administering a composition containing two different dsRNA molecules, one having a nucleotide sequence that is complementary to at least a part of an RNA transcript of the Eg5 gene and the other having a nucleotide sequence that is complementary to at least a part of an RNA transcript of the VEGF gene of the mammal to be treated. When the organism to be treated is a mammal such as a human, the composition may be administered by any means known in the art including, but not limited to oral or parenteral routes, including intravenous, intramuscular, subcutaneous, transdermal, airway (aerosol), nasal, rectal, and topical (including buccal and sublingual) administration. In preferred embodiments, the compositions are administered by intravenous infusion or injection.

Methods of Preparing Lipid Particles

The methods and compositions of the invention make use of certain cationic lipids, the synthesis, preparation and characterization of which is described below and in the accompanying Examples. In addition, the present invention provides methods of preparing lipid particles, including those associated with a therapeutic agent, e.g., a nucleic acid. In the methods described herein, a mixture of lipids is combined with a buffered aqueous solution of nucleic acid to produce an intermediate mixture containing nucleic acid encapsulated in lipid particles wherein the encapsulated nucleic acids are present in a nucleic acid/lipid ratio of about 3 wt % to about 25 wt %, preferably 5 to 15 wt %. The intermediate mixture may optionally be sized to obtain lipid-encapsulated nucleic acid particles wherein the lipid portions are unilamellar vesicles, preferably having a diameter of 30 to 150 nm, more preferably about 40 to 90 nm. The pH is then raised to neutralize at least a portion of the surface charges on the lipid-nucleic acid particles, thus providing an at least partially surface-neutralized lipid-encapsulated nucleic acid composition.

As described above, several of these cationic lipids are amino lipids that are charged at a pH below the pKₐ of the amino group and substantially neutral at a pH above the pKₐ. These cationic lipids are termed titratable cationic lipids and can be used in the formulations of the invention using a two-step process. First, lipid vesicles can be formed at the lower pH with titratable cationic lipids and other vesicle components in the presence of nucleic acids. In this manner, the vesicles will encapsulate and entrap the nucleic acids. Second, the surface charge of the newly formed vesicles can be neutralized by increasing the pH of the medium to a level above the pKₐ of the titratable cationic lipids present, i.e., to physiological pH or higher. Particularly advantageous aspects of this process include both the facile removal of any surface adsorbed nucleic acid and a resultant nucleic acid delivery vehicle which has a neutral surface. Liposomes or lipid particles having a neutral surface are expected to avoid rapid clearance from circulation and to avoid certain toxicities which are associated with cationic liposome preparations. Additional details concerning these uses of such titratable cationic lipids in the formulation of nucleic acid-lipid particles are provided in U.S. Pat. No. 6,287,591 and U.S. Pat. No. 6,858,225, incorporated herein by reference.

It is further noted that the vesicles formed in this manner provide formulations of uniform vesicle size with high content of nucleic acids. Additionally, the vesicles have a size range of from about 30 to about 150 nm, more preferably about 30 to about 90 nm.

Without intending to be bound by any particular theory, it is believed that the very high efficiency of nucleic acid encapsulation is a result of electrostatic interaction at low pH. At acidic pH (e.g. pH 4.0) the vesicle surface is charged and binds a portion of the nucleic acids through electrostatic interactions. When the external acidic buffer is exchanged for a more neutral buffer (e.g. pH 7.5) the surface of the lipid particle or liposome is neutralized, allowing any external nucleic acid to be removed. More detailed information on the formulation process is provided in various publications (e.g., U.S. Pat. No. 6,287,591 and U.S. Pat. No. 6,858,225).

In view of the above, the present invention provides methods of preparing lipid/nucleic acid formulations. In the methods described herein, a mixture of lipids is combined with a buffered aqueous solution of nucleic acid to produce an intermediate mixture containing nucleic acid encapsulated in lipid particles, e.g., wherein the encapsulated nucleic acids are present in a nucleic acid/lipid ratio of about 10 wt % to about 20 wt %. The intermediate mixture may optionally be sized to obtain lipid-encapsulated nucleic acid particles wherein the lipid portions are unilamellar vesicles, preferably having a diameter of 30 to 150 nm, more preferably about 40 to 90 nm. The pH is then raised to neutralize at least a portion of the surface charges on the lipid-nucleic acid particles, thus providing an at least partially surface-neutralized lipid-encapsulated nucleic acid composition.

In certain embodiments, the mixture of lipids includes at least two lipid components: a first amino lipid component of the present invention that is selected from among lipids which have a pKa such that the lipid is cationic at pH below the pKa and neutral at pH above the pKa, and a second lipid component that is selected from among lipids that prevent particle aggregation during lipid-nucleic acid particle formation. In particular embodiments, the amino lipid is a novel cationic lipid of the present invention.

In preparing the nucleic acid-lipid particles of the invention, the mixture of lipids is typically a solution of lipids in an organic solvent. This mixture of lipids can then be dried to form a thin film or lyophilized to form a powder before being hydrated with an aqueous buffer to form liposomes. Alternatively, in a preferred method, the lipid mixture can be solubilized in a water miscible alcohol, such as ethanol, and this ethanolic solution added to an aqueous buffer resulting in spontaneous liposome formation. In most embodiments, the alcohol is used in the form in which it is commercially available. For example, ethanol can be used as absolute ethanol (100%), or as 95% ethanol, the remainder being water. This method is described in more detail in U.S. Pat. No. 5,976,567).

In accordance with the invention, the lipid mixture is combined with a buffered aqueous solution that may contain the nucleic acids. The buffered aqueous solution of is typically a solution in which the buffer has a pH of less than the pKₐ of the protonatable lipid in the lipid mixture. Examples of suitable buffers include citrate, phosphate, acetate, and MES. A particularly preferred buffer is citrate buffer. Preferred buffers will be in the range of 1-1000 mM of the anion, depending on the chemistry of the nucleic acid being encapsulated, and optimization of buffer concentration may be significant to achieving high loading levels (see, e.g., U.S. Pat. No. 6,287,591 and U.S. Pat. No. 6,858,225). Alternatively, pure water acidified to pH 5-6 with chloride, sulfate or the like may be useful. In this case, it may be suitable to add 5% glucose, or another non-ionic solute which will balance the osmotic potential across the particle membrane when the particles are dialyzed to remove ethanol, increase the pH, or mixed with a pharmaceutically acceptable carrier such as normal saline. The amount of nucleic acid in buffer can vary, but will typically be from about 0.01 mg/mL to about 200 mg/mL, more preferably from about 0.5 mg/mL to about 50 mg/mL.

The mixture of lipids and the buffered aqueous solution of therapeutic nucleic acids is combined to provide an intermediate mixture. The intermediate mixture is typically a mixture of lipid particles having encapsulated nucleic acids. Additionally, the intermediate mixture may also contain some portion of nucleic acids which are attached to the surface of the lipid particles (liposomes or lipid vesicles) due to the ionic attraction of the negatively-charged nucleic acids and positively-charged lipids on the lipid particle surface (the amino lipids or other lipid making up the protonatable first lipid component are positively charged in a buffer having a pH of less than the pKₐ of the protonatable group on the lipid). In one group of preferred embodiments, the mixture of lipids is an alcohol solution of lipids and the volumes of each of the solutions is adjusted so that upon combination, the resulting alcohol content is from about 20% by volume to about 45% by volume. The method of combining the mixtures can include any of a variety of processes, often depending upon the scale of formulation produced. For example, when the total volume is about 10-20 mL or less, the solutions can be combined in a test tube and stirred together using a vortex mixer. Large-scale processes can be carried out in suitable production scale glassware.

Optionally, the lipid-encapsulated therapeutic agent (e.g., nucleic acid) complexes which are produced by combining the lipid mixture and the buffered aqueous solution of therapeutic agents (nucleic acids) can be sized to achieve a desired size range and relatively narrow distribution of lipid particle sizes. Preferably, the compositions provided herein will be sized to a mean diameter of from about 70 to about 200 nm, more preferably about 90 to about 130 nm. Several techniques are available for sizing liposomes to a desired size. One sizing method is described in U.S. Pat. No. 4,737,323, incorporated herein by reference. Sonicating a liposome suspension either by bath or probe sonication produces a progressive size reduction down to small unilamellar vesicles (SUVs) less than about 0.05 microns in size. Homogenization is another method which relies on shearing energy to fragment large liposomes into smaller ones. In a typical homogenization procedure, multilamellar vesicles are recirculated through a standard emulsion homogenizer until selected liposome sizes, typically between about 0.1 and 0.5 microns, are observed. In both methods, the particle size distribution can be monitored by conventional laser-beam particle size determination. For certain methods herein, extrusion is used to obtain a uniform vesicle size.

Extrusion of liposome compositions through a small-pore polycarbonate membrane or an asymmetric ceramic membrane results in a relatively well-defined size distribution. Typically, the suspension is cycled through the membrane one or more times until the desired liposome complex size distribution is achieved. The liposomes may be extruded through successively smaller-pore membranes, to achieve a gradual reduction in liposome size. In some instances, the lipid-nucleic acid compositions which are formed can be used without any sizing.

In particular embodiments, methods of the present invention further comprise a step of neutralizing at least some of the surface charges on the lipid portions of the lipid-nucleic acid compositions. By at least partially neutralizing the surface charges, unencapsulated nucleic acid is freed from the lipid particle surface and can be removed from the composition using conventional techniques. Preferably, unencapsulated and surface adsorbed nucleic acids are removed from the resulting compositions through exchange of buffer solutions. For example, replacement of a citrate buffer (pH about 4.0, used for forming the compositions) with a HEPES-buffered saline (HBS pH about 7.5) solution, results in the neutralization of liposome surface and nucleic acid release from the surface. The released nucleic acid can then be removed via chromatography using standard methods, and then switched into a buffer with a pH above the pKa of the lipid used.

Optionally the lipid vesicles (i.e., lipid particles) can be formed by hydration in an aqueous buffer and sized using any of the methods described above prior to addition of the nucleic acid. As described above, the aqueous buffer should be of a pH below the pKa of the amino lipid. A solution of the nucleic acids can then be added to these sized, preformed vesicles. To allow encapsulation of nucleic acids into such “pre-formed” vesicles the mixture should contain an alcohol, such as ethanol. In the case of ethanol, it should be present at a concentration of about 20% (w/w) to about 45% (w/w). In addition, it may be necessary to warm the mixture of pre-formed vesicles and nucleic acid in the aqueous buffer-ethanol mixture to a temperature of about 25° C. to about 50° C. depending on the composition of the lipid vesicles and the nature of the nucleic acid. It will be apparent to one of ordinary skill in the art that optimization of the encapsulation process to achieve a desired level of nucleic acid in the lipid vesicles will require manipulation of variable such as ethanol concentration and temperature. Examples of suitable conditions for nucleic acid encapsulation are provided in the Examples. Once the nucleic acids are encapsulated within the preformed vesicles, the external pH can be increased to at least partially neutralize the surface charge. Unencapsulated and surface adsorbed nucleic acids can then be removed as described above.

Method of Use

The lipid particles of the invention may be used to deliver a therapeutic agent to a cell, in vitro or in vivo. In particular embodiments, the therapeutic agent is a nucleic acid, which is delivered to a cell using a nucleic acid-lipid particles of the invention. While the following description of various methods of using the lipid particles and related pharmaceutical compositions of the invention are exemplified by description related to nucleic acid-lipid particles, it is understood that these methods and compositions may be readily adapted for the delivery of any therapeutic agent for the treatment of any disease or disorder that would benefit from such treatment.

In certain embodiments, the invention provides methods for introducing a nucleic acid into a cell. Preferred nucleic acids for introduction into cells are siRNA, immune-stimulating oligonucleotides, plasmids, antisense and ribozymes. These methods may be carried out by contacting the particles or compositions of the invention with the cells for a period of time sufficient for intracellular delivery to occur.

The compositions of the invention can be adsorbed to almost any cell type. Once adsorbed, the nucleic acid-lipid particles can either be endocytosed by a portion of the cells, exchange lipids with cell membranes, or fuse with the cells. Transfer or incorporation of the nucleic acid portion of the complex can take place via any one of these pathways. Without intending to be limited with respect to the scope of the invention, it is believed that in the case of particles taken up into the cell by endocytosis the particles then interact with the endosomal membrane, resulting in destabilization of the endosomal membrane, possibly by the formation of non-bilayer phases, resulting in introduction of the encapsulated nucleic acid into the cell cytoplasm. Similarly in the case of direct fusion of the particles with the cell plasma membrane, when fusion takes place, the liposome membrane is integrated into the cell membrane and the contents of the liposome combine with the intracellular fluid. Contact between the cells and the lipid-nucleic acid compositions, when carried out in vitro, will take place in a biologically compatible medium. The concentration of compositions can vary widely depending on the particular application, but is generally between about 1 μmol and about 10 mmol. In certain embodiments, treatment of the cells with the lipid-nucleic acid compositions will generally be carried out at physiological temperatures (about 37° C.) for periods of time from about 1 to 24 hours, preferably from about 2 to 8 hours. For in vitro applications, the delivery of nucleic acids can be to any cell grown in culture, whether of plant or animal origin, vertebrate or invertebrate, and of any tissue or type. In preferred embodiments, the cells will be animal cells, more preferably mammalian cells, and most preferably human cells.

In one group of embodiments, a lipid-nucleic acid particle suspension is added to 60-80% confluent plated cells having a cell density of from about 10³ to about 10⁵ cells/mL, more preferably about 2×10⁴ cells/mL. The concentration of the suspension added to the cells is preferably of from about 0.01 to 20 μg/mL, more preferably about 1 μg/mL.

Typical applications include using well known procedures to provide intracellular delivery of siRNA to knock down or silence specific cellular targets. Alternatively applications include delivery of DNA or mRNA sequences that code for therapeutically useful polypeptides. In this manner, therapy is provided for genetic diseases by supplying deficient or absent gene products (i.e., for Duchenne's dystrophy, see Kunkel, et al., Brit. Med. Bull. 45(3):630-643 (1989), and for cystic fibrosis, see Goodfellow, Nature 341:102-103 (1989)). Other uses for the compositions of the invention include introduction of antisense oligonucleotides in cells (see, Bennett, et al., Mol. Pharm. 41:1023-1033 (1992)).

Alternatively, the compositions of the invention can also be used for deliver of nucleic acids to cells in vivo, using methods which are known to those of skill in the art. With respect to application of the invention for delivery of DNA or mRNA sequences, Zhu, et al., Science 261:209-211 (1993), incorporated herein by reference, describes the intravenous delivery of cytomegalovirus (CMV)-chloramphenicol acetyltransferase (CAT) expression plasmid using DOTMA-DOPE complexes. Hyde, et al., Nature 362:250-256 (1993), incorporated herein by reference, describes the delivery of the cystic fibrosis transmembrane conductance regulator (CFTR) gene to epithelia of the airway and to alveoli in the lung of mice, using liposomes. Brigham, et al., Am. J. Med. Sci. 298:278-281 (1989), incorporated herein by reference, describes the in vivo transfection of lungs of mice with a functioning prokaryotic gene encoding the intracellular enzyme, chloramphenicol acetyltransferase (CAT). Thus, the compositions of the invention can be used in the treatment of infectious diseases.

For in vivo administration, the pharmaceutical compositions are preferably administered parenterally, i.e., intraarticularly, intravenously, intraperitoneally, subcutaneously, or intramuscularly. In particular embodiments, the pharmaceutical compositions are administered intravenously or intraperitoneally by a bolus injection. For one example, see Stadler, et al., U.S. Pat. No. 5,286,634, which is incorporated herein by reference. Intracellular nucleic acid delivery has also been discussed in Straubringer, et al., METHODS IN ENZYMOLOGY, Academic Press, New York. 101:512-527 (1983); Mannino, et al., Biotechniques 6:682-690 (1988); Nicolau, et al., Crit. Rev. Ther. Drug Carrier Syst. 6:239-271 (1989), and Behr, Acc. Chem. Res. 26:274-278 (1993). Still other methods of administering lipid-based therapeutics are described in, for example, Rahman et al., U.S. Pat. No. 3,993,754; Sears, U.S. Pat. No. 4,145,410; Papahadjopoulos et al., U.S. Pat. No. 4,235,871; Schneider, U.S. Pat. No. 4,224,179; Lenk et al., U.S. Pat. No. 4,522,803; and Fountain et al., U.S. Pat. No. 4,588,578.

In other methods, the pharmaceutical preparations may be contacted with the target tissue by direct application of the preparation to the tissue. The application may be made by topical, “open” or “closed” procedures. By “topical,” it is meant the direct application of the pharmaceutical preparation to a tissue exposed to the environment, such as the skin, oropharynx, external auditory canal, and the like. “Open” procedures are those procedures which include incising the skin of a patient and directly visualizing the underlying tissue to which the pharmaceutical preparations are applied. This is generally accomplished by a surgical procedure, such as a thoracotomy to access the lungs, abdominal laparotomy to access abdominal viscera, or other direct surgical approach to the target tissue. “Closed” procedures are invasive procedures in which the internal target tissues are not directly visualized, but accessed via inserting instruments through small wounds in the skin. For example, the preparations may be administered to the peritoneum by needle lavage. Likewise, the pharmaceutical preparations may be administered to the meninges or spinal cord by infusion during a lumbar puncture followed by appropriate positioning of the patient as commonly practiced for spinal anesthesia or metrazamide imaging of the spinal cord. Alternatively, the preparations may be administered through endoscopic devices.

The lipid-nucleic acid compositions can also be administered in an aerosol inhaled into the lungs (see, Brigham, et al., Am. J. Sci. 298(4):278-281 (1989)) or by direct injection at the site of disease (Culver, Human Gene Therapy, MaryAnn Liebert, Inc., Publishers, New York. pp. 70-71 (1994)).

The methods of the invention may be practiced in a variety of hosts. Preferred hosts include mammalian species, such as humans, non-human primates, dogs, cats, cattle, horses, sheep, and the like.

Dosages for the lipid-therapeutic agent particles of the invention will depend on the ratio of therapeutic agent to lipid and the administrating physician's opinion based on age, weight, and condition of the patient.

In one embodiment, the invention provides a method of modulating the expression of a target polynucleotide or polypeptide. These methods generally comprise contacting a cell with a lipid particle of the invention that is associated with a nucleic acid capable of modulating the expression of a target polynucleotide or polypeptide. As used herein, the term “modulating” refers to altering the expression of a target polynucleotide or polypeptide. In different embodiments, modulating can mean increasing or enhancing, or it can mean decreasing or reducing. Methods of measuring the level of expression of a target polynucleotide or polypeptide are known and available in the arts and include, e.g., methods employing reverse transcription-polymerase chain reaction (RT-PCR) and immunohistochemical techniques. In particular embodiments, the level of expression of a target polynucleotide or polypeptide is increased or reduced by at least 10%, 20%, 30%, 40%, 50%, or greater than 50% as compared to an appropriate control value. For example, if increased expression of a polypeptide desired, the nucleic acid may be an expression vector that includes a polynucleotide that encodes the desired polypeptide. On the other hand, if reduced expression of a polynucleotide or polypeptide is desired, then the nucleic acid may be, e.g., an antisense oligonucleotide, siRNA, or microRNA that comprises a polynucleotide sequence that specifically hybridizes to a polynucleotide that encodes the target polypeptide, thereby disrupting expression of the target polynucleotide or polypeptide. Alternatively, the nucleic acid may be a plasmid that expresses such an antisense oligonucleotide, siRNA, or microRNA.

In one particular embodiment, the invention provides a method of modulating the expression of a polypeptide by a cell, comprising providing to a cell a lipid particle that consists of or consists essentially of a cationic lipid of formula A, a neutral lipid, a sterol, a PEG of PEG-modified lipid, e.g., in a molar ratio of about 35-65% of cationic lipid of formula A, 3-12% of the neutral lipid, 15-45% of the sterol, and 0.5-10% of the PEG or PEG-modified lipid, wherein the lipid particle is associated with a nucleic acid capable of modulating the expression of the polypeptide. In particular embodiments, the molar lipid ratio is approximately 60/7.5/31/1.5 or 57.5/7.5/31.5/3.5 (mol % LIPID A/DSPC/Chol/PEG-DMG). In another group of embodiments, the neutral lipid in these compositions is replaced with DPPC (dipalmitoylphosphatidylcholine), POPC, DOPE or SM.

In particular embodiments, the therapeutic agent is selected from an siRNA, a microRNA, an antisense oligonucleotide, and a plasmid capable of expressing an siRNA, a microRNA, or an antisense oligonucleotide, and wherein the siRNA, microRNA, or antisense RNA comprises a polynucleotide that specifically binds to a polynucleotide that encodes the polypeptide, or a complement thereof, such that the expression of the polypeptide is reduced.

In other embodiments, the nucleic acid is a plasmid that encodes the polypeptide or a functional variant or fragment thereof, such that expression of the polypeptide or the functional variant or fragment thereof is increased.

In related embodiments, the invention provides a method of treating a disease or disorder characterized by overexpression of a polypeptide in a subject, comprising providing to the subject a pharmaceutical composition of the invention, wherein the therapeutic agent is selected from an siRNA, a microRNA, an antisense oligonucleotide, and a plasmid capable of expressing an siRNA, a microRNA, or an antisense oligonucleotide, and wherein the siRNA, microRNA, or antisense RNA comprises a polynucleotide that specifically binds to a polynucleotide that encodes the polypeptide, or a complement thereof.

In one embodiment, the pharmaceutical composition comprises a lipid particle that consists of or consists essentially of Lipid A, DSPC, Chol and PEG-DMG, PEG-C-DOMG or PEG-DMA, e.g., in a molar ratio of about 35-65% of cationic lipid of formula A, 3-12% of the neutral lipid, 15-45% of the sterol, and 0.5-10% of the PEG or PEG-modified lipid PEG-DMG, PEG-C-DOMG or PEG-DMA, wherein the lipid particle is associated with the therapeutic nucleic acid. In particular embodiments, the molar lipid ratio is approximately 60/7.5/31/1.5 or 57.5/7.5/31.5/3.5 (mol % LIPID A/DSPC/Chol/PEG-DMG). In another group of embodiments, the neutral lipid in these compositions is replaced with DPPC, POPC, DOPE or SM.

In another related embodiment, the invention includes a method of treating a disease or disorder characterized by underexpression of a polypeptide in a subject, comprising providing to the subject a pharmaceutical composition of the invention, wherein the therapeutic agent is a plasmid that encodes the polypeptide or a functional variant or fragment thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### Examples

### Example 1

### dsRNA Synthesis

Source of Reagents

Where the source of a reagent is not specifically given herein, such reagent may be obtained from any supplier of reagents for molecular biology at a quality/purity standard for application in molecular biology.

siRNA Synthesis

For screening of dsRNA, single-stranded RNAs were produced by solid phase synthesis on a scale of 1 μmole using an Expedite 8909 synthesizer (Applied Biosystems, Applera Deutschland GmbH, Darmstadt, Germany) and controlled pore glass (CPG, 500 Å, Proligo Biochemie GmbH, Hamburg, Germany) as solid support. RNA and RNA containing 2′-O-methyl nucleotides were generated by solid phase synthesis employing the corresponding phosphoramidites and 2′-O-methyl phosphoramidites, respectively (Proligo Biochemie GmbH, Hamburg, Germany). These building blocks were incorporated at selected sites within the sequence of the oligoribonucleotide chain using standard nucleoside phosphoramidite chemistry such as described in Current protocols in nucleic acid chemistry, Beaucage, S. L. et al. (Edrs.), John Wiley & Sons, Inc., New York, N.Y., USA. Phosphorothioate linkages were introduced by replacement of the iodine oxidizer solution with a solution of the Beaucage reagent (Chruachem Ltd, Glasgow, UK) in acetonitrile (1%). Further ancillary reagents were obtained from Mallinckrodt Baker (Griesheim, Germany).

Deprotection and purification of the crude oligoribonucleotides by anion exchange HPLC were carried out according to established procedures. Yields and concentrations were determined by UV absorption of a solution of the respective RNA at a wavelength of 260 nm using a spectral photometer (DU 640B, Beckman Coulter GmbH, Unterschleiβheim, Germany). Double stranded RNA was generated by mixing an equimolar solution of complementary strands in annealing buffer (20 mM sodium phosphate, pH 6.8; 100 mM sodium chloride), heated in a water bath at 85-90° C. for 3 minutes and cooled to room temperature over a period of 3-4 hours. The annealed RNA solution was stored at −20 ° C. until use.

dsRNA Targeting the Eg5 Gene

Initial Screening Set

siRNA design was carried out to identify siRNAs targeting Eg5 (also known as KIF11, HSKP, KNSL1 and TRIP5). Human mRNA sequences to Eg5, RefSeq ID number:NM_{—}004523, was used.

siRNA duplexes cross-reactive to human and mouse Eg5 were designed. Twenty-four duplexes were synthesized for screening. (Table 1a). A second screening set was defined with 266 siRNAs targeting human Eg5, as well as its rhesus monkey ortholog (Table 2a). An expanded screening set was selected with 328 siRNA targeting human Eg5, with no necessity to hit any Eg5 mRNA of other species (Table 3a).

The sequences for human and a partial rhesus Eg5 mRNAs were downloaded from NCBI Nucleotide database and the human sequence was further on used as reference sequence (Human EG5:NM_{—}004523.2, 4908 bp, and Rhesus EG5: XM_{—}001087644.1, 878 by (only 5′ part of human EG5).

For the Tables: Key: A,G,C,U-ribonucleotides: T-deoxythymidine: u,c-2′-O-methyl nucleotides: s-phosphorothioate linkage.

TABLE 1a
Sequences of Eg5/KSP dsRNA duplexes

| | | | | | | | |
|---|---|---|---|---|---|---|---|
| position in | | | | | | | |
| human | SEQ | | SEQ | | SEQ | | |
| Eg5/KSP | ID | sequence of 23mer target | ID | | ID | | duplex |
| sequence | NO: | site | NO: | sense sequence (5′-3′) | No: | antisense sequence (5′-3′) | name |

| | | | | | | | |
|---|---|---|---|---|---|---|---|
| 385-407 | 1244 | ACCGAAGUGUUGUUUGUC | 1 | cGAAGuGuuGuuuGuccA | 2 | UUGGAcAAAcAAcACUUCG | AL-DP- |
| | | CAAUU | | ATsT | | TsT | 6226 |
| 347-369 | 1245 | UAUGGUGUUUGGAGCAUC | 3 | uGGuGuuuGGAGcAucuA | 4 | GuAGAUGCUCcAAAcACcA | AL-DP- |
| | | UACUA | | cTsT | | TsT | 6227 |
| 1078-1100 | 1246 | AAUCUAAACUAACUAGAA | 5 | ucuAAAcuAAcuAGAAuc | 6 | GGAUUCuAGUuAGUUuAGA | AL-DP- |
| | | UCCUC | | cTsT | | TsT | 6228 |
| 1067-1089 | 1247 | UCCUUAUCGAGAAUCUAA | 7 | cuuAucGAGAAucuAAAc | 8 | AGUUuAGAUUCUCGAuAAG | AL-DP- |
| | | ACUAA | | uTsT | | TsT | 6229 |
| 374-396 | 1248 | GAUUGAUGUUUACCGAAG | 9 | uuGAuGuuuAccGAAGuG | 10 | AcACUUCGGuAAAcAUcAA | AL-DP- |
| | | UGUUG | | uTsT | | TsT | 6230 |
| 205-227 | 1249 | UGGUGAGAUGCAGACCAU | 11 | GuGAGAuGcAGAccAuuu | 12 | uAAAUGGUCUGcAUCUcAC | AL-DP- |
| | | UUAAU | | ATsT | | TsT | 6231 |
| 1176-1198 | 1250 | ACUCUGAGUACAUUGGAA | 13 | ucuGAGuAcAuuGGAAuA | 14 | AuAUUCcAAUGuACUcAGA | AL-DP- |
| | | UAUGC | | uTsT | | TsT | 6232 |
| 386-408 | 1251 | CCGAAGUGUUGUUUGUCC | 15 | GAAGuGuuGuuuGuccAA | 16 | AUUGGAcAAAcAAcACUUC | AL-DP- |
| | | AAUUC | | uTsT | | TsT | 6233 |
| 416-438 | 1252 | AGUUAUUAUGGGCUAUAA | 17 | uuAuuAuGGGcuAuAAuu | 18 | cAAUuAuAGCCcAuAAuAA | AL-DP- |
| | | UUGCA | | GTsT | | TsT | 6234 |
| 485-507 | 1253 | GGAAGGUGAAAGGUCACC | 19 | AAGGuGAAAGGucAccuA | 20 | UuAGGUGACCUUUcACCUU | AL-DP- |
| | | UAAUG | | ATsT | | TsT | 6235 |
| 476-498 | 1254 | UUUUACAAUGGAAGGUGA | 21 | uuAcAAuGGAAGGuGAAA | 22 | CUUUcACCUUCcAUUGuAA | AL-DP- |
| | | AAGGU | | GTsT | | TsT | 6236 |
| 486-508 | 1255 | GAAGGUGAAAGGUCACCU | 23 | AGGuGAAAGGucAccuAA | 24 | AUuAGGUGACCUUUcACCU | AL-DP- |
| | | AAUGA | | uTsT | | TsT | 6237 |
| 487-509 | 1256 | AAGGUGAAAGGUCACCUA | 25 | GGuGAAAGGucAccuAAu | 26 | cAUuAGGUGACCUUUcACC | AL-DP- |
| | | AUGAA | | GTsT | | TsT | 6238 |
| 1066-1088 | 1257 | UUCCUUAUCGAGAAUCUA | 27 | ccuuAucGAGAAucuAAA | 28 | GUUuAGAUUCUCGAuAAGG | AL-DP- |
| | | AACUA | | cTsT | | TsT | 6239 |
| 1256-1278 | 1258 | AGCUCUUAUUAAGGAGUA | 29 | cucuuAuuAAGGAGuAuA | 30 | GuAuACUCCUuAAuAAGAG | AL-DP- |
| | | UACGG | | cTsT | | TsT | 6240 |
| 2329-2351 | 1259 | CAGAGAGAUUCUGUGCUU | 31 | GAGAGAuucuGuGcuuuG | 32 | CcAAAGcAcAGAAUCUCUC | AL-DP- |
| | | UGGAG | | GTsT | | TsT | 6241 |
| 1077-1099 | 1260 | GAAUCUAAACUAACUAGA | 33 | AucuAAAcuAAcuAGAAu | 34 | GAUUCuAGUuAGUUuAGAU | AL-DP- |
| | | AUCCU | | cTsT | | TsT | 6242 |
| 1244-1266 | 1261 | ACUCACCAAAAAAGCUCU | 35 | ucAccAAAAAAGcucuuA | 36 | AuAAGAGCUUUUUUGGUGA | AL-DP- |
| | | UAUUA | | uTsT | | TsT | 6243 |
| 637-659 | 1262 | AAGAGCUUUUUGAUCUUC | 37 | GAGcuuuuuGAucuucuu | 38 | uAAGAAGAUcAAAAAGCUC | AL-DP- |
| | | UUAAU | | ATsT | | TsT | 6244 |
| 1117-1139 | 1263 | GGCGUACAAGAACAUCUA | 39 | cGuAcAAGAAcAucuAuA | 40 | UuAuAGAUGUUCUUGuACG | AL-DP- |
| | | UAAUU | | ATsT | | TsT | 6245 |
| 373-395 | 1264 | AGAUUGAUGUUUACCGAA | 41 | AuuGAuGuuuAccGAAGu | 42 | cACUUCGGuAAAcAUcAAU | AL-DP- |
| | | GUGUU | | GTsT | | TsT | 6246 |
| 1079-1101 | 1265 | AUCUAAACUAACUAGAAU | 43 | cuAAAcuAAcuAGAAucc | 44 | AGGAUUCuAGUuAGUUuAG | AL-DP- |
| | | CCUCC | | uTsT | | TsT | 6247 |
| 383-405 | 1266 | UUACCGAAGUGUUGUUUG | 45 | AccGAAGuGuuGuuuGuc | 46 | GGAcAAAcAAcACUUCGGU | AL-DP- |
| | | UCCAA | | cTsT | | TsT | 6248 |
| 200-222 | 1267 | GGUGGUGGUGAGAUGCAG | 47 | uGGuGGuGAGAuGcAGAc | 48 | GGUCUGcAUCUcACcACcA | AL-DP- |
| | | ACCAU | | cTsT | | TsT | 6249 |

TABLE 1b
Analysis of Eg5/KSP ds duplexes

| | single | |
|---|---|---|
| | dose | |
| | screen @ | |
| | 25 nM [% | SDs 2nd screen |
| duplex | residual | (among |
| name | mRNA] | quadruplicates) |
| AL-DP-6226 | 23% | 3% |
| AL-DP-6227 | 69% | 10% |
| AL-DP-6228 | 33% | 2% |
| AL-DP-6229 | 2% | 2% |
| AL-DP-6230 | 66% | 11% |
| AL-DP-6231 | 17% | 1% |
| AL-DP-6232 | 9% | 3% |
| AL-DP-6233 | 24% | 6% |
| AL-DP-6234 | 91% | 2% |
| AL-DP-6235 | 112% | 4% |
| AL-DP-6236 | 69% | 4% |
| AL-DP-6237 | 42% | 2% |
| AL-DP-6238 | 45% | 2% |
| AL-DP-6239 | 2% | 1% |
| AL-DP-6240 | 48% | 2% |
| AL-DP-6241 | 41% | 2% |
| AL-DP-6242 | 8% | 2% |
| AL-DP-6243 | 7% | 1% |
| AL-DP-6244 | 6% | 2% |
| AL-DP-6245 | 12% | 2% |
| AL-DP-6246 | 28% | 3% |
| AL-DP-6247 | 71% | 4% |
| AL-DP-6248 | 5% | 2% |
| AL-DP-6249 | 28% | 3% |

TABLE 2a
Sequences of Eg5/KSP dsRNA duplexes

| SEQ ID | sequence of 19-mer | SEQ ID | | SEQ ID | antisense sequence (5′- | duplex |
|---|---|---|---|---|---|---|
| NO: | target site | NO. | sense sequence (5′-3′) | NO. | 3′) | name |

| | | | | | | |
|---|---|---|---|---|---|---|
| 1268 | CAUACUCUAGUCGUUCCCA | 49 | cAuAcucuAGucGuucccATsT | 50 | UGGGAACGACuAGAGuAUGTsT | AD-12072 |
| 1269 | AGCGCCCAUUCAAUAGUAG | 51 | AGcGcccAuucAAuAGuAGTsT | 52 | CuACuAUUGAAUGGGCGCUTsT | AD-12073 |
| 1270 | GGAAAGCUAGCGCCCAUUC | 53 | GGAAAGcuAGcGcccAuucTsT | 54 | GAAUGGGCGCuAGCUUUCCTsT | AD-12074 |
| 1271 | GAAAGCUAGCGCCCAUUCA | 55 | GAAAGcuAGcGcccAuucATsT | 56 | UGAAUGGGCGCuAGCUUUCTsT | AD-12075 |
| 1272 | AGAAACUACGAUUGAUGGA | 57 | AGAAAcuAcGAuuGAuGGATsT | 58 | UCcAUcAAUCGuAGUUUCUTsT | AD-12076 |
| 1273 | UGUUCCUUAUCGAGAAUCU | 59 | uGuuccuuAucGAGAAucuTsT | 60 | AGAUUCUCGAuAAGGAAcATsT | AD-12077 |
| 1274 | CAGAUUACCUCUGCGAGCC | 61 | cAGAuuAccucuGcGAGccTsT | 62 | GGCUCGcAGAGGuAAUCUGTsT | AD-12078 |
| 1275 | GCGCCCAUUCAAUAGUAGA | 63 | GcGcccAuucAAuAGuAGATsT | 64 | UCuACuAUUGAAUGGGCGCTsT | AD-12079 |
| 1276 | UUGCACUAUCUUUGCGUAU | 65 | uuGcAcuAucuuuGcGuAuTsT | 66 | AuACGcAAAGAuAGUGcAATsT | AD-12080 |
| 1277 | CAGAGCGGAAAGCUAGCGC | 67 | cAGAGcGGAAAGcuAGcGcTsT | 68 | GCGCuAGCUUUCCGCUCUGTsT | AD-12081 |
| 1278 | AGACCUUAUUUGGUAAUCU | 69 | AGAccuuAuuuGGuAAucuTsT | 70 | AGAUuACcAAAuAAGGUCUTsT | AD-12082 |
| 1279 | AUUCUCUUGGAGGGCGUAC | 71 | AuucucuuGGAGGGcGuAcTsT | 72 | GuACGCCCUCcAAGAGAAUTsT | AD-12083 |
| 1280 | GGCUGGUAUAAUUCCACGU | 73 | GGcuGGuAuAAuuccAcGuTsT | 74 | ACGUGGAAUuAuACcAGCCTsT | AD-12084 |
| 1281 | GCGGAAAGCUAGCGCCCAU | 75 | GcGGAAAGcuAGcGcccAuTsT | 76 | AUGGGCGCuAGCUUUCCGCTsT | AD-12085 |
| 1282 | UGCACUAUCUUUGCGUAUG | 77 | uGcAcuAucuuuGcGuAuGTsT | 78 | cAuACGcAAAGAuAGUGcATsT | AD-12086 |
| 1283 | GUAUAAUUCCACGUACCCU | 79 | GuAuAAuuccAcGuAcccuTsT | 80 | AGGGuACGUGGAAUuAuACTsT | AD-12087 |
| 1284 | AGAAUCUAAACUAACUAGA | 81 | AGAAucuAAAcuAAcuAGATsT | 82 | UCuAGUuAGUUuAGAUUCUTsT | AD-12088 |
| 1285 | AGGAGCUGAAUAGGGUUAC | 83 | AGGAGcuGAAuAGGGuuAcTsT | 84 | GuAACCCuAUUcAGCUCCUTsT | AD-12089 |
| 1286 | GAAGUACAUAAGACCUUAU | 85 | GAAGuAcAuAAGAccuuAuTsT | 86 | AuAAGGUCUuAUGuACUUCTsT | AD-12090 |
| 1287 | GACAGUGGCCGAUAAGAUA | 87 | GAcAGuGGccGAuAAGAuATsT | 88 | uAUCUuAUCGGCcACUGUCTsT | AD-12091 |
| 1288 | AAACCACUUAGUAGUGUCC | 89 | AAAccAcuuAGuAGuGuccTsT | 90 | GGAcACuACuAAGUGGUUUTsT | AD-12092 |
| 1289 | UCCCUAGACUUCCCUAUUU | 91 | ucccuAGAcuucccuAuuuTsT | 92 | AAAuAGGGAAGUCuAGGGATsT | AD-12093 |
| 1290 | UAGACUUCCCUAUUUCGCU | 93 | uAGAcuucccuAuuucGcuTsT | 94 | AGCGAAAuAGGGAAGUCuATsT | AD-12094 |
| 1291 | GCGUCGCAGCCAAAUUCGU | 95 | GcGucGcAGccAAAuucGuTsT | 96 | ACGAAUUUGGCUGCGACGCTsT | AD-12095 |
| 1292 | AGCUAGCGCCCAUUCAAUA | 97 | AGcuAGcGcccAuucAAuATsT | 98 | uAUUGAAUGGGCGCuAGCUTsT | AD-12096 |
| 1293 | GAAACUACGAUUGAUGGAG | 99 | GAAAcuAcGAuuGAuGGAGTsT | 100 | CUCcAUcAAUCGuAGUUUCTsT | AD-12097 |
| 1294 | CCGAUAAGAUAGAAGAUCA | 101 | ccGAuAAGAuAGAAGAucATsT | 102 | UGAUCUUCuAUCUuAUCGGTsT | AD-12098 |
| 1295 | UAGCGCCCAUUCAAUAGUA | 103 | uAGcGcccAuucAAuAGuATsT | 104 | uACuAUUGAAUGGGCGCuATsT | AD-12099 |
| 1296 | UUUGCGUAUGGCCAAACUG | 105 | uuuGcGuAuGGccAAAcuGTsT | 106 | cAGUUUGGCcAuACGcAAATsT | AD-12100 |
| 1297 | CACGUACCCUUCAUCAAAU | 107 | cAcGuAcccuucAucAAAuTsT | 108 | AUUUGAUGAAGGGuACGUGTsT | AD-12101 |
| 1298 | UCUUUGCGUAUGGCCAAAC | 109 | ucuuuGcGuAuGGccAAAcTsT | 110 | GUUUGGCcAuACGcAAAGATsT | AD-12102 |
| 1299 | CCGAAGUGUUGUUUGUCCA | 111 | ccGAAGuGuuGuuuGuccATsT | 112 | UGGAcAAAcAAcACUUCGGTsT | AD-12103 |
| 1300 | AGAGCGGAAAGCUAGCGCC | 113 | AGAGcGGAAAGcuAGcGccTsT | 114 | GGCGCuAGCUUUCCGCUCUTsT | AD-12104 |
| 1301 | GCUAGCGCCCAUUCAAUAG | 115 | GcuAGcGcccAuucAAuAGTsT | 116 | CuAUUGAAUGGGCGCuAGCTsT | AD-12105 |
| 1302 | AAGUUAGUGUACGAACUGG | 117 | AAGuuAGuGuAcGAAcuGGTsT | 118 | CcAGUUCGuAcACuAACUUTsT | AD-12106 |
| 1303 | GUACGAACUGGAGGAUUGG | 119 | GuAcGAAcuGGAGGAuuGGTsT | 120 | CcAAUCCUCcAGUUCGuACTsT | AD-12107 |
| 1304 | ACGAACUGGAGGAUUGGCU | 121 | AcGAAcuGGAGGAuuGGcuTsT | 122 | AGCcAAUCCUCcAGUUCGUTsT | AD-12108 |
| 1305 | AGAUUGAUGUUUACCGAAG | 123 | AGAuuGAuGuuuAccGAAGTsT | 124 | CUUCGGuAAAcAUcAAUCUTsT | AD-12109 |
| 1306 | UAUGGGCUAUAAUUGCACU | 125 | uAuGGGcuAuAAuuGcAcuTsT | 126 | AGUGcAAUuAuAGCCcAuATsT | AD-12110 |
| 1307 | AUCUUUGCGUAUGGCCAAA | 127 | AucuuuGcGuAuGGccAAATsT | 128 | UUUGGCcAuACGcAAAGAUTsT | AD-12111 |
| 1308 | ACUCUAGUCGUUCCCACUC | 129 | AcucuAGucGuucccAcucTsT | 130 | GAGUGGGAACGACuAGAGUTsT | AD-12112 |
| 1309 | AACUACGAUUGAUGGAGAA | 131 | AAcuAcGAuuGAuGGAGAATsT | 132 | UUCUCcAUcAAUCGuAGUUTsT | AD-12113 |
| 1310 | GAUAAGAGAGCUCGGGAAG | 133 | GAuAAGAGAGcucGGGAAGTsT | 134 | CUUCCCGAGCUCUCUuAUCTsT | AD-12114 |
| 1311 | UCGAGAAUCUAAACUAACU | 135 | ucGAGAAucuAAAcuAAcuTsT | 136 | AGUuAGUUuAGAUUCUCGATsT | AD-12115 |
| 1312 | AACUAACUAGAAUCCUCCA | 137 | AAcuAAcuAGAAuccuccATsT | 138 | UGGAGGAUUCuAGUuAGUUTsT | AD-12116 |
| 1313 | GGAUCGUAAGAAGGCAGUU | 139 | GGAucGuAAGAAGGcAGuuTsT | 140 | AACUGCCUUCUuACGAUCCTsT | AD-12117 |
| 1314 | AUCGUAAGAAGGCAGUUGA | 141 | AucGuAAGAAGGcAGuuGATsT | 142 | UcAACUGCCUUCUuACGAUTsT | AD-12118 |
| 1315 | AGGCAGUUGACCAACACAA | 143 | AGGcAGuuGAccAAcAcAATsT | 144 | UUGUGUUGGUcAACUGCCUTsT | AD-12119 |
| 1316 | UGGCCGAUAAGAUAGAAGA | 145 | uGGccGAuAAGAuAGAAGATsT | 146 | UCUUCuAUCUuAUCGGCcATsT | AD-12120 |
| 1317 | UCUAAGGAUAUAGUCAACA | 147 | ucuAAGGAuAuAGucAAcATsT | 148 | UGUUGACuAuAUCCUuAGATsT | AD-12121 |
| 1318 | ACUAAGCUUAAUUGCUUUC | 149 | AcuAAGcuuAAuuGcuuucTsT | 150 | GAAAGcAAUuAAGCUuAGUTsT | AD-12122 |
| 1319 | GCCCAGAUCAACCUUUAAU | 151 | GcccAGAucAAccuuuAAuTsT | 152 | AUuAAAGGUUGAUCUGGGCTsT | AD-12123 |
| 1320 | UUAAUUUGGCAGAGCGGAA | 153 | uuAAuuuGGcAGAGcGGAATsT | 154 | UUCCGCUCUGCcAAAUuAATsT | AD-12124 |
| 1321 | UUAUCGAGAAUCUAAACUA | 155 | uuAucGAGAAucuAAAcuATsT | 156 | uAGUUuAGAUUCUCGAuAATsT | AD-12125 |
| 1322 | CUAGCGCCCAUUCAAUAGU | 157 | cuAGcGcccAuucAAuAGuTsT | 158 | ACuAUUGAAUGGGCGCuAGTsT | AD-12126 |
| 1323 | AAUAGUAGAAUGUGAUCCU | 159 | AAuAGuAGAAuGuGAuccuTsT | 160 | AGGAUcAcAUUCuACuAUUTsT | AD-12127 |
| 1324 | UACGAAAAGAAGUUAGUGU | 161 | uAcGAAAAGAAGuuAGuGuTsT | 162 | AcACuAACUUCUUUUCGuATsT | AD-12128 |
| 1325 | AGAAGUUAGUGUACGAACU | 163 | AGAAGuuAGuGuAcGAAcuTsT | 164 | AGUUCGuAcACuAACUUCUTsT | AD-12129 |
| 1326 | ACUAAACAGAUUGAUGUUU | 165 | AcuAAAcAGAuuGAuGuuuTsT | 166 | AAAcAUcAAUCUGUUuAGUTsT | AD-12130 |
| 1327 | CUUUGCGUAUGGCCAAACU | 167 | cuuuGcGuAuGGccAAAcuTsT | 168 | AGUUUGGCcAuACGcAAAGTsT | AD-12131 |
| 1328 | AAUGAAGAGUAUACCUGGG | 169 | AAuGAAGAGuAuAccuGGGTsT | 170 | CCcAGGuAuACUCUUcAUUTsT | AD-12132 |
| 1329 | AUAAUUCCACGUACCCUUC | 171 | AuAAuuccAcGuAcccuucTsT | 172 | GAAGGGuACGUGGAAUuAUTsT | AD-12133 |
| 1330 | ACGUACCCUUCAUCAAAUU | 173 | AcGuAcccuucAucAAAuuTsT | 174 | AAUUUGAUGAAGGGuACGUTsT | AD-12134 |
| 1331 | CGUACCCUUCAUCAAAUUU | 175 | cGuAcccuucAucAAAuuuTsT | 176 | AAAUUUGAUGAAGGGuACGTsT | AD-12135 |
| 1332 | GUACCCUUCAUCAAAUUUU | 177 | GuAcccuucAucAAAuuuuTsT | 178 | AAAAUUUGAUGAAGGGuACTsT | AD-12136 |
| 1333 | AACUUACUGAUAAUGGUAC | 179 | AAcuuAcuGAuAAuGGuAcTsT | 180 | GuACcAUuAUcAGuAAGUUTsT | AD-12137 |
| 1334 | UUCAGUCAAAGUGUCUCUG | 181 | uucAGucAAAGuGucucuGTsT | 182 | cAGAGAcACUUUGACUGAATsT | AD-12138 |
| 1335 | UUCUUAAUCCAUCAUCUGA | 183 | uucuuAAuccAucAucuGATsT | 184 | UcAGAUGAUGGAUuAAGAATsT | AD-12139 |
| 1336 | ACAGUACACAACAAGGAUG | 185 | AcAGuAcAcAAcAAGGAuGTsT | 186 | cAUCCUUGUUGUGuACUGUTsT | AD-12140 |
| 1337 | AAGAAACUACGAUUGAUGG | 187 | AAGAAAcuAcGAuuGAuGGTsT | 188 | CcAUcAAUCGuAGUUUCUUTsT | AD-12141 |
| 1338 | AAACUACGAUUGAUGGAGA | 189 | AAAcuAcGAuuGAuGGAGATsT | 190 | UCUCcAUcAAUCGuAGUUUTsT | AD-12142 |
| 1339 | UGGAGCUGUUGAUAAGAGA | 191 | uGGAGcuGuuGAuAAGAGATsT | 192 | UCUCUuAUcAAcAGCUCcATsT | AD-12143 |
| 1340 | CUAACUAGAAUCCUCCAGG | 193 | cuAAcuAGAAuccuccAGGTsT | 194 | CCUGGAGGAUUCuAGUuAGTsT | AD-12144 |
| 1341 | GAAUAUGCUCAUAGAGCAA | 195 | GAAuAuGcucAuAGAGcAATsT | 196 | UUGCUCuAUGAGcAuAUUCTsT | AD-12145 |
| 1342 | AUGCUCAUAGAGCAAAGAA | 197 | AuGcucAuAGAGcAAAGAATsT | 198 | UUCUUUGCUCuAUGAGcAUTsT | AD-12146 |
| 1343 | AAAAAUUGGUGCUGUUGAG | 199 | AAAAAuuGGuGcuGuuGAGTsT | 200 | CUcAAcAGcACcAAUUUUUTsT | AD-12147 |
| 1344 | GAGGAGCUGAAUAGGGUUA | 201 | GAGGAGcuGAAuAGGGuuATsT | 202 | uAACCCuAUUcAGCUCCUCTsT | AD-12148 |
| 1345 | GGAGCUGAAUAGGGUUACA | 203 | GGAGcuGAAuAGGGuuAcATsT | 204 | UGuAACCCuAUUcAGCUCCTsT | AD-12149 |
| 1346 | GAGCUGAAUAGGGUUACAG | 205 | GAGcuGAAuAGGGuuAcAGTsT | 206 | CUGuAACCCuAUUcAGCUCTsT | AD-12150 |
| 1347 | AGCUGAAUAGGGUUACAGA | 207 | AGcuGAAuAGGGuuAcAGATsT | 208 | UCUGuAACCCuAUUcAGCUTsT | AD-12151 |
| 1348 | GCUGAAUAGGGUUACAGAG | 209 | GcuGAAuAGGGuuAcAGAGTsT | 210 | CUCUGuAACCCuAUUcAGCTsT | AD-12152 |
| 1349 | CCAAACUGGAUCGUAAGAA | 211 | ccAAAcuGGAucGuAAGAATsT | 212 | UUCUuACGAUCcAGUUUGGTsT | AD-12153 |
| 1350 | GAUCGUAAGAAGGCAGUUG | 213 | GAucGuAAGAAGGcAGuuGTsT | 214 | cAACUGCCUUCUuACGAUCTsT | AD-12154 |
| 1351 | ACCUUAUUUGGUAAUCUGC | 215 | AccuuAuuuGGuAAucuGcTsT | 216 | GcAGAUuACcAAAuAAGGUTsT | AD-12155 |
| 1352 | UUAGAUACCAUUACUACAG | 217 | uuAGAuAccAuuAcuAcAGTsT | 218 | CUGuAGuAAUGGuAUCuAATsT | AD-12156 |
| 1353 | AUACCAUUACUACAGUAGC | 219 | AuAccAuuAcuAcAGuAGcTsT | 220 | GCuACUGuAGuAAUGGuAUTsT | AD-12157 |
| 1354 | UACUACAGUAGCACUUGGA | 221 | uAcuAcAGuAGcAcuuGGATsT | 222 | UCcAAGUGCuACUGuAGuATsT | AD-12158 |
| 1355 | AAAGUAAAACUGUACUACA | 223 | AAAGuAAAAcuGuAcuAcATsT | 224 | UGuAGuAcAGUUUuACUUUTsT | AD-12159 |
| 1356 | CUCAAGACUGAUCUUCUAA | 225 | cucAAGAcuGAucuucuAATsT | 226 | UuAGAAGAUcAGUCUUGAGTsT | AD-12160 |
| 1357 | UUGACAGUGGCCGAUAAGA | 227 | uuGAcAGuGGccGAuAAGATsT | 228 | UCUuAUCGGCcACUGUcAATsT | AD-12161 |
| 1358 | UGACAGUGGCCGAUAAGAU | 229 | uGAcAGuGGccGAuAAGAuTsT | 230 | AUCUuAUCGGCcACUGUcATsT | AD-12162 |
| 1359 | GCAAUGUGGAAACCUAACU | 231 | GcAAuGuGGAAAccuAAcuTsT | 232 | AGUuAGGUUUCcAcAUUGCTsT | AD-12163 |
| 1360 | CCACUUAGUAGUGUCCAGG | 233 | ccAcuuAGuAGuGuccAGGTsT | 234 | CCUGGAcACuACuAAGUGGTsT | AD-12164 |
| 1361 | AGAAGGUACAAAAUUGGUU | 235 | AGAAGGuAcAAAAuuGGuuTsT | 236 | AACcAAUUUUGuACCUUCUTsT | AD-12165 |
| 1362 | UGGUUUGACUAAGCUUAAU | 237 | uGGuuuGAcuAAGcuuAAuTsT | 238 | AUuAAGCUuAGUcAAACcATsT | AD-12166 |
| 1363 | GGUUUGACUAAGCUUAAUU | 239 | GGuuuGAcuAAGcuuAAuuTsT | 240 | AAUuAAGCUuAGUcAAACCTsT | AD-12167 |
| 1364 | UCUAAGUCAAGAGCCAUCU | 241 | ucuAAGucAAGAGccAucuTsT | 242 | AGAUGGCUCUUGACUuAGATsT | AD-12168 |
| 1365 | UCAUCCCUAUAGUUCACUU | 243 | ucAucccuAuAGuucAcuuTsT | 244 | AAGUGAACuAuAGGGAUGATsT | AD-12169 |
| 1366 | CAUCCCUAUAGUUCACUUU | 245 | cAucccuAuAGuucAcuuuTsT | 246 | AAAGUGAACuAuAGGGAUGTsT | AD-12170 |
| 1367 | CCCUAGACUUCCCUAUUUC | 247 | cccuAGAcuucccuAuuucTsT | 248 | GAAAuAGGGAAGUCuAGGGTsT | AD-12171 |
| 1368 | AGACUUCCCUAUUUCGCUU | 249 | AGAcuucccuAuuucGcuuTsT | 250 | AAGCGAAAuAGGGAAGUCUTsT | AD-12172 |
| 1369 | UCACCAAACCAUUUGUAGA | 251 | ucAccAAAccAuuuGuAGATsT | 252 | UCuAcAAAUGGUUUGGUGATsT | AD-12173 |
| 1370 | UCCUUUAAGAGGCCUAACU | 253 | uccuuuAAGAGGccuAAcuTsT | 254 | AGUuAGGCCUCUuAAAGGATsT | AD-12174 |
| 1371 | UUUAAGAGGCCUAACUCAU | 255 | uuuAAGAGGccuAAcucAuTsT | 256 | AUGAGUuAGGCCUCUuAAATsT | AD-12175 |
| 1372 | UUAAGAGGCCUAACUCAUU | 257 | uuAAGAGGccuAAcucAuuTsT | 258 | AAUGAGUuAGGCCUCUuAATsT | AD-12176 |
| 1373 | GGCCUAACUCAUUCACCCU | 259 | GGccuAAcucAuucAcccuTsT | 260 | AGGGUGAAUGAGUuAGGCCTsT | AD-12177 |
| 1374 | UGGUAUUUUUGAUCUGGCA | 261 | uGGuAuuuuuGAucuGGcATsT | 262 | UGCcAGAUcAAAAAuACcATsT | AD-12178 |
| 1375 | AGUUUAGUGUGUAAAGUUU | 263 | AGuuuAGuGuGuAAAGuuuTsT | 264 | AAACUUuAcAcACuAAACUTsT | AD-12179 |
| 1376 | GCCAAAUUCGUCUGCGAAG | 265 | GccAAAuucGucuGcGAAGTsT | 266 | CUUCGcAGACGAAUUUGGCTsT | AD-12180 |
| 1377 | AAUUCGUCUGCGAAGAAGA | 267 | AAuucGucuGcGAAGAAGATsT | 268 | UCUUCUUCGcAGACGAAUUTsT | AD-12181 |
| 1378 | UGAAAGGUCACCUAAUGAA | 269 | uGAAAGGucAccuAAuGAATsT | 270 | UUcAUuAGGUGACCUUUcATsT | AD-12182 |
| 1379 | CAGACCAUUUAAUUUGGCA | 271 | cAGAccAuuuAAuuuGGcATsT | 272 | UGCcAAAUuAAAUGGUCUGTsT | AD-12183 |
| 1380 | AGACCAUUUAAUUUGGCAG | 273 | AGAccAuuuAAuuuGGcAGTsT | 274 | CUGCcAAAUuAAAUGGUCUTsT | AD-12184 |
| 1381 | AGUUAUUAUGGGCUAUAAU | 275 | AGuuAuuAuGGGcuAuAAuTsT | 276 | AUuAuAGCCcAuAAuAACUTsT | AD-12185 |
| 1382 | GCUGGUAUAAUUCCACGUA | 277 | GcuGGuAuAAuuccAcGuATsT | 278 | uACGUGGAAUuAuACcAGCTsT | AD-12186 |
| 1383 | AUUUAAUUUGGCAGAGCGG | 279 | AuuuAAuuuGGcAGAGcGGTsT | 280 | CCGCUCUGCcAAAUuAAAUTsT | AD-12187 |
| 1384 | UUUAAUUUGGCAGAGCGGA | 281 | uuuAAuuuGGcAGAGcGGATsT | 282 | UCCGCUCUGCcAAAUuAAATsT | AD-12188 |
| 1385 | UUUGGCAGAGCGGAAAGCU | 283 | uuuGGcAGAGcGGAAAGcuTsT | 284 | AGCUUUCCGCUCUGCcAAATsT | AD-12189 |
| 1386 | UUUUACAAUGGAAGGUGAA | 285 | uuuuAcAAuGGAAGGuGAATsT | 286 | UUcACCUUCcAUUGuAAAATsT | AD-12190 |
| 1387 | AAUGGAAGGUGAAAGGUCA | 287 | AAuGGAAGGuGAAAGGucATsT | 288 | UGACCUUUcACCUUCcAUUTsT | AD-12191 |
| 1388 | UGAGAUGCAGACCAUUUAA | 289 | uGAGAuGcAGAccAuuuAATsT | 290 | UuAAAUGGUCUGcAUCUcATsT | AD-12192 |
| 1389 | UCGCAGCCAAAUUCGUCUG | 291 | ucGcAGccAAAuucGucuGTsT | 292 | cAGACGAAUUUGGCUGCGATsT | AD-12193 |
| 1390 | GGCUAUAAUUGCACUAUCU | 293 | GGcuAuAAuuGcAcuAucuTsT | 294 | AGAuAGUGcAAUuAuAGCCTsT | AD-12194 |
| 1391 | AUUGACAGUGGCCGAUAAG | 295 | AuuGAcAGuGGccGAuAAGTsT | 296 | CUuAUCGGCcACUGUcAAUTsT | AD-12195 |
| 1392 | CUAGACUUCCCUAUUUCGC | 297 | cuAGAcuucccuAuuucGcTsT | 298 | GCGAAAuAGGGAAGUCuAGTsT | AD-12196 |
| 1393 | ACUAUCUUUGCGUAUGGCC | 299 | AcuAucuuuGcGuAuGGccTsT | 300 | GGCcAuACGcAAAGAuAGUTsT | AD-12197 |
| 1394 | AUACUCUAGUCGUUCCCAC | 301 | AuAcucuAGucGuucccAcTsT | 302 | GUGGGAACGACuAGAGuAUTsT | AD-12198 |
| 1395 | AAAGAAACUACGAUUGAUG | 303 | AAAGAAAcuAcGAuuGAuGTsT | 304 | cAUcAAUCGuAGUUUCUUUTsT | AD-12199 |
| 1396 | GCCUUGAUUUUUUGGCGGG | 305 | GccuuGAuuuuuuGGcGGGTsT | 306 | CCCGCcAAAAAAUcAAGGCTsT | AD-12200 |
| 1397 | CGCCCAUUCAAUAGUAGAA | 307 | cGcccAuucAAuAGuAGAATsT | 308 | UUCuACuAUUGAAUGGGCGTsT | AD-12201 |
| 1398 | CCUUAUUUGGUAAUCUGCU | 309 | ccuuAuuuGGuAAucuGcuTsT | 310 | AGcAGAUuACcAAAuAAGGTsT | AD-12202 |
| 1399 | AGAGACAAUUCCGGAUGUG | 311 | AGAGAcAAuuccGGAuGuGTsT | 312 | cAcAUCCGGAAUUGUCUCUTsT | AD-12203 |
| 1400 | UGACUUUGAUAGCUAAAUU | 313 | uGAcuuuGAuAGcuAAAuuTsT | 314 | AAUUuAGCuAUcAAAGUcATsT | AD-12204 |
| 1401 | UGGCAGAGCGGAAAGCUAG | 315 | uGGcAGAGcGGAAAGcuAGTsT | 316 | CuAGCUUUCCGCUCUGCcATsT | AD-12205 |
| 1402 | GAGCGGAAAGCUAGCGCCC | 317 | GAGcGGAAAGcuAGcGcccTsT | 318 | GGGCGCuAGCUUUCCGCUCTsT | AD-12206 |
| 1403 | AAAGAAGUUAGUGUACGAA | 319 | AAAGAAGuuAGuGuAcGAATsT | 320 | UUCGuAcACuAACUUCUUUTsT | AD-12207 |
| 1404 | AUUGCACUAUCUUUGCGUA | 321 | AuuGcAcuAucuuuGcGuATsT | 322 | uACGcAAAGAuAGUGcAAUTsT | AD-12208 |
| 1405 | GGUAUAAUUCCACGUACCC | 323 | GGuAuAAuuccAcGuAcccTsT | 324 | GGGuACGUGGAAUuAuACCTsT | AD-12209 |
| 1406 | UACUCUAGUCGUUCCCACU | 325 | uAcucuAGucGuucccAcuTsT | 326 | AGUGGGAACGACuAGAGuATsT | AD-12210 |
| 1407 | UAUGAAAGAAACUACGAUU | 327 | uAuGAAAGAAAcuAcGAuuTsT | 328 | AAUCGuAGUUUCUUUcAuATsT | AD-12211 |
| 1408 | AUGCUAGAAGUACAUAAGA | 329 | AuGcuAGAAGuAcAuAAGATsT | 330 | UCUuAUGuACUUCuAGcAUTsT | AD-12212 |
| 1409 | AAGUACAUAAGACCUUAUU | 331 | AAGuAcAuAAGAccuuAuuTsT | 332 | AAuAAGGUCUuAUGuACUUTsT | AD-12213 |
| 1410 | ACAGCCUGAGCUGUUAAUG | 333 | AcAGccuGAGcuGuuAAuGTsT | 334 | cAUuAAcAGCUcAGGCUGUTsT | AD-12214 |
| 1411 | AAAGAAGAGACAAUUCCGG | 335 | AAAGAAGAGAcAAuuccGGTsT | 336 | CCGGAAUUGUCUCUUCUUUTsT | AD-12215 |
| 1412 | CACACUGGAGAGGUCUAAA | 337 | cAcAcuGGAGAGGucuAAATsT | 338 | UUuAGACCUCUCcAGUGUGTsT | AD-12216 |
| 1413 | CACUGGAGAGGUCUAAAGU | 339 | cAcuGGAGAGGucuAAAGuTsT | 340 | ACUUuAGACCUCUCcAGUGTsT | AD-12217 |
| 1414 | ACUGGAGAGGUCUAAAGUG | 341 | AcuGGAGAGGucuAAAGuGTsT | 342 | cACUUuAGACCUCUCcAGUTsT | AD-12218 |
| 1415 | CGUCGCAGCCAAAUUCGUC | 343 | cGucGcAGccAAAuucGucTsT | 344 | GACGAAUUUGGCUGCGACGTsT | AD-12219 |
| 1416 | GAAGGCAGUUGACCAACAC | 345 | GAAGGcAGuuGAccAAcAcTsT | 346 | GUGUUGGUcAACUGCCUUCTsT | AD-12220 |
| 1417 | CAUUCACCCUGACAGAGUU | 347 | cAuucAcccuGAcAGAGuuTsT | 348 | AACUCUGUcAGGGUGAAUGTsT | AD-12221 |
| 1418 | AAGAGGCCUAACUCAUUCA | 349 | AAGAGGccuAAcucAuucATsT | 350 | UGAAUGAGUuAGGCCUCUUTsT | AD-12222 |
| 1419 | GAGACAAUUCCGGAUGUGG | 351 | GAGAcAAuuccGGAuGuGGTsT | 352 | CcAcAUCCGGAAUUGUCUCTsT | AD-12223 |
| 1420 | UUCCGGAUGUGGAUGUAGA | 353 | uuccGGAuGuGGAuGuAGATsT | 354 | UCuAcAUCcAcAUCCGGAATsT | AD-12224 |
| 1421 | AAGCUAGCGCCCAUUCAAU | 355 | AAGcuAGcGcccAuucAAuTsT | 356 | AUUGAAUGGGCGCuAGCUUTsT | AD-12225 |
| 1422 | GAAGUUAGUGUACGAACUG | 357 | GAAGuuAGuGuAcGAAcuGTsT | 358 | cAGUUCGuAcACuAACUUCTsT | AD-12226 |
| 1423 | UAUAAUUCCACGUACCCUU | 359 | uAuAAuuccAcGuAcccuuTsT | 360 | AAGGGuACGUGGAAUuAuATsT | AD-12227 |
| 1424 | ACAGUGGCCGAUAAGAUAG | 361 | AcAGuGGccGAuAAGAuAGTsT | 362 | CuAUCUuAUCGGCcACUGUTsT | AD-12228 |
| 1425 | UCUGUCAUCCCUAUAGUUC | 363 | ucuGucAucccuAuAGuucTsT | 364 | GAACuAuAGGGAUGAcAGATsT | AD-12229 |
| 1426 | UUCUUGCUAUGACUUGUGU | 365 | uucuuGcuAuGAcuuGuGuTsT | 366 | AcAcAAGUcAuAGcAAGAATsT | AD-12230 |
| 1427 | GUAAGAAGGCAGUUGACCA | 367 | GuAAGAAGGcAGuuGAccATsT | 368 | UGGUcAACUGCCUUCUuACTsT | AD-12231 |
| 1428 | CAUUGACAGUGGCCGAUAA | 369 | cAuuGAcAGuGGccGAuAATsT | 370 | UuAUCGGCcACUGUcAAUGTsT | AD-12232 |
| 1429 | AGAAACCACUUAGUAGUGU | 371 | AGAAAccAcuuAGuAGuGuTsT | 372 | AcACuACuAAGUGGUUUCUTsT | AD-12233 |
| 1430 | GGAUUGUUCAUCAAUUGGC | 373 | GGAuuGuucAucAAuuGGcTsT | 374 | GCcAAUUGAUGAAcAAUCCTsT | AD-12234 |
| 1431 | UAAGAGGCCUAACUCAUUC | 375 | uAAGAGGccuAAcucAuucTsT | 376 | GAAUGAGUuAGGCCUCUuATsT | AD-12235 |
| 1432 | AGUUAGUGUACGAACUGGA | 377 | AGuuAGuGuAcGAAcuGGATsT | 378 | UCcAGUUCGuAcACuAACUTsT | AD-12236 |
| 1433 | AGUACAUAAGACCUUAUUU | 379 | AGuAcAuAAGAccuuAuuuTsT | 380 | AAAuAAGGUCUuAUGuACUTsT | AD-12237 |
| 1434 | UGAGCCUUGUGUAUAGAUU | 381 | uGAGccuuGuGuAuAGAuuTsT | 382 | AAUCuAuAcAcAAGGCUcATsT | AD-12238 |
| 1435 | CCUUUAAGAGGCCUAACUC | 383 | ccuuuAAGAGGccuAAcucTsT | 384 | GAGUuAGGCCUCUuAAAGGTsT | AD-12239 |
| 1436 | ACCACUUAGUAGUGUCCAG | 385 | AccAcuuAGuAGuGuccAGTsT | 386 | CUGGAcACuACuAAGUGGUTsT | AD-12240 |
| 1437 | GAAACUUCCAAUUAUGUCU | 387 | GAAAcuuccAAuuAuGucuTsT | 388 | AGAcAuAAUUGGAAGUUUCTsT | AD-12241 |
| 1438 | UGCAUACUCUAGUCGUUCC | 389 | uGcAuAcucuAGucGuuccTsT | 390 | GGAACGACuAGAGuAUGcATsT | AD-12242 |
| 1439 | AGAAGGCAGUUGACCAACA | 391 | AGAAGGcAGuuGAccAAcATsT | 392 | UGUUGGUcAACUGCCUUCUTsT | AD-12243 |
| 1440 | GUACAUAAGACCUUAUUUG | 393 | GuAcAuAAGAccuuAuuuGTsT | 394 | cAAAuAAGGUCUuAUGuACTsT | AD-12244 |
| 1441 | UAUAAUUGCACUAUCUUUG | 395 | uAuAAuuGcAcuAucuuuGTsT | 396 | cAAAGAuAGUGcAAUuAuATsT | AD-12245 |
| 1442 | UCUCUGUUACAAUACAUAU | 397 | ucucuGuuAcAAuAcAuAuTsT | 398 | AuAUGuAUUGuAAcAGAGATsT | AD-12246 |
| 1443 | UAUGCUCAUAGAGCAAAGA | 399 | uAuGcucAuAGAGcAAAGATsT | 400 | UCUUUGCUCuAUGAGcAuATsT | AD-12247 |
| 1444 | UGUUGUUUGUCCAAUUCUG | 401 | uGuuGuuuGuccAAuucuGTsT | 402 | cAGAAUUGGAcAAAcAAcATsT | AD-12248 |
| 1445 | ACUAACUAGAAUCCUCCAG | 403 | AcuAAcuAGAAuccuccAGTsT | 404 | CUGGAGGAUUCuAGUuAGUTsT | AD-12249 |
| 1446 | UGUGGUGUCUAUACUGAAA | 405 | uGuGGuGucuAuAcuGAAATsT | 406 | UUUcAGuAuAGAcACcAcATsT | AD-12250 |
| 1447 | UAUUAUGGGAGACCACCCA | 407 | uAuuAuGGGAGAccAcccATsT | 408 | UGGGUGGUCUCCcAuAAuATsT | AD-12251 |
| 1448 | AAGGAUGAAGUCUAUCAAA | 409 | AAGGAuGAAGucuAucAAATsT | 410 | UUUGAuAGACUUcAUCCUUTsT | AD-12252 |
| 1449 | UUGAUAAGAGAGCUCGGGA | 411 | uuGAuAAGAGAGcucGGGATsT | 412 | UCCCGAGCUCUCUuAUcAATsT | AD-12253 |
| 1450 | AUGUUCCUUAUCGAGAAUC | 413 | AuGuuccuuAucGAGAAucTsT | 414 | GAUUCUCGAuAAGGAAcAUTsT | AD-12254 |
| 1451 | GGAAUAUGCUCAUAGAGCA | 415 | GGAAuAuGcucAuAGAGcATsT | 416 | UGCUCuAUGAGcAuAUUCCTsT | AD-12255 |
| 1452 | CCAUUCCAAACUGGAUCGU | 417 | ccAuuccAAAcuGGAucGuTsT | 418 | ACGAUCcAGUUUGGAAUGGTsT | AD-12256 |
| 1453 | GGCAGUUGACCAACACAAU | 419 | GGcAGuuGAccAAcAcAAuTsT | 420 | AUUGUGUUGGUcAACUGCCTsT | AD-12257 |
| 1454 | CAUGCUAGAAGUACAUAAG | 421 | cAuGcuAGAAGuAcAuAAGTsT | 422 | CUuAUGuACUUCuAGcAUGTsT | AD-12258 |
| 1455 | CUAGAAGUACAUAAGACCU | 423 | cuAGAAGuAcAuAAGAccuTsT | 424 | AGGUCUuAUGuACUUCuAGTsT | AD-12259 |
| 1456 | UUGGAUCUCUCACAUCUAU | 425 | uuGGAucucucAcAucuAuTsT | 426 | AuAGAUGUGAGAGAUCcAATsT | AD-12260 |
| 1457 | AACUGUGGUGUCUAUACUG | 427 | AAcuGuGGuGucuAuAcuGTsT | 428 | cAGuAuAGAcACcAcAGUUTsT | AD-12261 |
| 1458 | UCAUUGACAGUGGCCGAUA | 429 | ucAuuGAcAGuGGccGAuATsT | 430 | uAUCGGCcACUGUcAAUGATsT | AD-12262 |
| 1459 | AUAAAGCAGACCCAUUCCC | 431 | AuAAAGcAGAcccAuucccTsT | 432 | GGGAAUGGGUCUGCUUuAUTsT | AD-12263 |
| 1460 | ACAGAAACCACUUAGUAGU | 433 | AcAGAAAccAcuuAGuAGuTsT | 434 | ACuACuAAGUGGUUUCUGUTsT | AD-12264 |
| 1461 | GAAACCACUUAGUAGUGUC | 435 | GAAAccAcuuAGuAGuGucTsT | 436 | GAcACuACuAAGUGGUUUCTsT | AD-12265 |
| 1462 | AAAUCUAAGGAUAUAGUCA | 437 | AAAucuAAGGAuAuAGucATsT | 438 | UGACuAuAUCCUuAGAUUUTsT | AD-12266 |
| 1463 | UUAUUUAUACCCAUCAACA | 439 | uuAuuuAuAcccAucAAcATsT | 440 | UGUUGAUGGGuAuAAAuAATsT | AD-12267 |
| 1464 | ACAGAGGCAUUAACACACU | 441 | AcAGAGGcAuuAAcAcAcuTsT | 442 | AGUGUGUuAAUGCCUCUGUTsT | AD-12268 |
| 1465 | ACACACUGGAGAGGUCUAA | 443 | AcAcAcuGGAGAGGucuAATsT | 444 | UuAGACCUCUCcAGUGUGUTsT | AD-12269 |
| 1466 | ACACUGGAGAGGUCUAAAG | 445 | AcAcuGGAGAGGucuAAAGTsT | 446 | CUUuAGACCUCUCcAGUGUTsT | AD-12270 |
| 1467 | CGAGCCCAGAUCAACCUUU | 447 | cGAGcccAGAucAAccuuuTsT | 448 | AAAGGUUGAUCUGGGCUCGTsT | AD-12271 |
| 1468 | UCCCUAUUUCGCUUUCUCC | 449 | ucccuAuuucGcuuucuccTsT | 450 | GGAGAAAGCGAAAuAGGGATsT | AD-12272 |
| 1469 | UCUAAAAUCACUGUCAACA | 451 | ucuAAAAucAcuGucAAcATsT | 452 | UGUUGAcAGUGAUUUuAGATsT | AD-12273 |
| 1470 | AGCCAAAUUCGUCUGCGAA | 453 | AGccAAAuucGucuGcGAATsT | 454 | UUCGcAGACGAAUUUGGCUTsT | AD-12274 |
| 1471 | CCCAUUCAAUAGUAGAAUG | 455 | cccAuucAAuAGuAGAAuGTsT | 456 | cAUUCuACuAUUGAAUGGGTsT | AD-12275 |
| 1472 | GAUGAAUGCAUACUCUAGU | 457 | GAuGAAuGcAuAcucuAGuTsT | 458 | ACuAGAGuAUGcAUUcAUCTsT | AD-12276 |
| 1473 | CUCAUGUUCCUUAUCGAGA | 459 | cucAuGuuccuuAucGAGATsT | 460 | UCUCGAuAAGGAAcAUGAGTsT | AD-12277 |
| 1474 | GAGAAUCUAAACUAACUAG | 461 | GAGAAucuAAAcuAAcuAGTsT | 462 | CuAGUuAGUUuAGAUUCUCTsT | AD-12278 |
| 1475 | UAGAAGUACAUAAGACCUU | 463 | uAGAAGuAcAuAAGAccuuTsT | 464 | AAGGUCUuAUGuACUUCuATsT | AD-12279 |
| 1476 | CAGCCUGAGCUGUUAAUGA | 465 | cAGccuGAGcuGuuAAuGATsT | 466 | UcAUuAAcAGCUcAGGCUGTsT | AD-12280 |
| 1477 | AAGAAGAGACAAUUCCGGA | 467 | AAGAAGAGAcAAuuccGGATsT | 468 | UCCGGAAUUGUCUCUUCUUTsT | AD-12281 |
| 1478 | UGCUGGUGUGGAUUGUUCA | 469 | uGcuGGuGuGGAuuGuucATsT | 470 | UGAAcAAUCcAcACcAGcATsT | AD-12282 |
| 1479 | AAAUUCGUCUGCGAAGAAG | 471 | AAAuucGucuGcGAAGAAGTsT | 472 | CUUCUUCGcAGACGAAUUUTsT | AD-12283 |
| 1480 | UUUCUGGAAGUUGAGAUGU | 473 | uuucuGGAAGuuGAGAuGuTsT | 474 | AcAUCUcAACUUCcAGAAATsT | AD-12284 |
| 1481 | UACUAAACAGAUUGAUGUU | 475 | uAcuAAAcAGAuuGAuGuuTsT | 476 | AAcAUcAAUCUGUUuAGuATsT | AD-12285 |
| 1482 | GAUUGAUGUUUACCGAAGU | 477 | GAuuGAuGuuuAccGAAGuTsT | 478 | ACUUCGGuAAAcAUcAAUCTsT | AD-12286 |
| 1483 | GCACUAUCUUUGCGUAUGG | 479 | GcAcuAucuuuGcGuAuGGTsT | 480 | CcAuACGcAAAGAuAGUGCTsT | AD-12287 |
| 1484 | UGGUAUAAUUCCACGUACC | 481 | uGGuAuAAuuccAcGuAccTsT | 482 | GGuACGUGGAAUuAuACcATsT | AD-12288 |
| 1485 | AGCAAGCUGCUUAACACAG | 483 | AGcAAGcuGcuuAAcAcAGTsT | 484 | CUGUGUuAAGcAGCUUGCUTsT | AD-12289 |
| 1486 | CAGAAACCACUUAGUAGUG | 485 | cAGAAAccAcuuAGuAGuGTsT | 486 | cACuACuAAGUGGUUUCUGTsT | AD-12290 |
| 1487 | AACUUAUUGGAGGUUGUAA | 487 | AAcuuAuuGGAGGuuGuAATsT | 488 | UuAcAACCUCcAAuAAGUUTsT | AD-12291 |
| 1488 | CUGGAGAGGUCUAAAGUGG | 489 | cuGGAGAGGucuAAAGuGGTsT | 490 | CcACUUuAGACCUCUCcAGTsT | AD-12292 |
| 1489 | AAAAAAGAUAUAAGGCAGU | 491 | AAAAAAGAuAuAAGGcAGuTsT | 492 | ACUGCCUuAuAUCUUUUUUTsT | AD-12293 |
| 1490 | GAAUUUUGAUAUCUACCCA | 493 | GAAuuuuGAuAucuAcccATsT | 494 | UGGGuAGAuAUcAAAAUUCTsT | AD-12294 |
| 1491 | GUAUUUUUGAUCUGGCAAC | 495 | GuAuuuuuGAucuGGcAAcTsT | 496 | GUUGCcAGAUcAAAAAuACTsT | AD-12295 |
| 1492 | AGGAUCCCUUGGCUGGUAU | 497 | AGGAucccuuGGcuGGuAuTsT | 498 | AuACcAGCcAAGGGAUCCUTsT | AD-12296 |
| 1493 | GGAUCCCUUGGCUGGUAUA | 499 | GGAucccuuGGcuGGuAuATsT | 500 | uAuACcAGCcAAGGGAUCCTsT | AD-12297 |
| 1494 | CAAUAGUAGAAUGUGAUCC | 501 | cAAuAGuAGAAuGuGAuccTsT | 502 | GGAUcAcAUUCuACuAUUGTsT | AD-12298 |
| 1495 | GCUAUAAUUGCACUAUCUU | 503 | GcuAuAAuuGcAcuAucuuTsT | 504 | AAGAuAGUGcAAUuAuAGCTsT | AD-12299 |
| 1496 | UACCCUUCAUCAAAUUUUU | 505 | uAcccuucAucAAAuuuuuTsT | 506 | AAAAAUUUGAUGAAGGGuATsT | AD-12300 |
| 1497 | AGAACAUAUUGAAUAAGCC | 507 | AGAAcAuAuuGAAuAAGccTsT | 508 | GGCUuAUUcAAuAUGUUCUTsT | AD-12301 |
| 1498 | AAAUUGGUGCUGUUGAGGA | 509 | AAAuuGGuGcuGuuGAGGATsT | 510 | UCCUcAAcAGcACcAAUUUTsT | AD-12302 |
| 1499 | UGAAUAGGGUUACAGAGUU | 511 | uGAAuAGGGuuAcAGAGuuTsT | 512 | AACUCUGuAACCCuAUUcATsT | AD-12303 |
| 1500 | AAGAACUUGAAACCACUCA | 513 | AAGAAcuuGAAAccAcucATsT | 514 | UGAGUGGUUUcAAGUUCUUTsT | AD-12304 |
| 1501 | AAUAAAGCAGACCCAUUCC | 515 | AAuAAAGcAGAcccAuuccTsT | 516 | GGAAUGGGUCUGCUUuAUUTsT | AD-12305 |
| 1502 | AUACCCAUCAACACUGGUA | 517 | AuAcccAucAAcAcuGGuATsT | 518 | uACcAGUGUUGAUGGGuAUTsT | AD-12306 |
| 1503 | UGGAUUGUUCAUCAAUUGG | 519 | uGGAuuGuucAucAAuuGGTsT | 520 | CcAAUUGAUGAAcAAUCcATsT | AD-12307 |
| 1504 | UGGAGAGGUCUAAAGUGGA | 521 | uGGAGAGGucuAAAGuGGATsT | 522 | UCcACUUuAGACCUCUCcATsT | AD-12308 |
| 1505 | GUCAUCCCUAUAGUUCACU | 523 | GucAucccuAuAGuucAcuTsT | 524 | AGUGAACuAuAGGGAUGACTsT | AD-12309 |
| 1506 | AUAAUGGCUAUAAUUUCUC | 525 | AuAAuGGcuAuAAuuucucTsT | 526 | GAGAAAUuAuAGCcAUuAUTsT | AD-12310 |
| 1507 | AUCCCUUGGCUGGUAUAAU | 527 | AucccuuGGcuGGuAuAAuTsT | 528 | AUuAuACcAGCcAAGGGAUTsT | AD-12311 |
| 1508 | GGGCUAUAAUUGCACUAUC | 529 | GGGcuAuAAuuGcAcuAucTsT | 530 | GAuAGUGcAAUuAuAGCCCTsT | AD-12312 |
| 1509 | GAUUCUCUUGGAGGGCGUA | 531 | GAuucucuuGGAGGGcGuATsT | 532 | uACGCCCUCcAAGAGAAUCTsT | AD-12313 |
| 1510 | GCAUCUCUCAAUCUUGAGG | 533 | GcAucucucAAucuuGAGGTsT | 534 | CCUcAAGAUUGAGAGAUGCTsT | AD-12314 |
| 1511 | CAGCAGAAAUCUAAGGAUA | 535 | cAGcAGAAAucuAAGGAuATsT | 536 | uAUCCUuAGAUUUCUGCUGTsT | AD-12315 |
| 1512 | GUCAAGAGCCAUCUGUAGA | 537 | GucAAGAGccAucuGuAGATsT | 538 | UCuAcAGAUGGCUCUUGACTsT | AD-12316 |
| 1513 | AAACAGAGGCAUUAACACA | 539 | AAAcAGAGGcAuuAAcAcATsT | 540 | UGUGUuAAUGCCUCUGUUUTsT | AD-12317 |
| 1514 | AGCCCAGAUCAACCUUUAA | 541 | AGcccAGAucAAccuuuAATsT | 542 | UuAAAGGUUGAUCUGGGCUTsT | AD-12318 |
| 1515 | UAUUUUUGAUCUGGCAACC | 543 | uAuuuuuGAucuGGcAAccTsT | 544 | GGUUGCcAGAUcAAAAAuATsT | AD-12319 |
| 1516 | UGUUUGGAGCAUCUACUAA | 545 | uGuuuGGAGcAucuAcuAATsT | 546 | UuAGuAGAUGCUCcAAAcATsT | AD-12320 |
| 1517 | GAAAUUACAGUACACAACA | 547 | GAAAuuAcAGuAcAcAAcATsT | 548 | UGUUGUGuACUGuAAUUUCTsT | AD-12321 |
| 1518 | ACUUGACCAGUGUAAAUCU | 549 | AcuuGAccAGuGuAAAucuTsT | 550 | AGAUUuAcACUGGUcAAGUTsT | AD-12322 |
| 1519 | ACCAGUGUAAAUCUGACCU | 551 | AccAGuGuAAAucuGAccuTsT | 552 | AGGUcAGAUUuAcACUGGUTsT | AD-12323 |
| 1520 | AGAACAAUCAUUAGCAGCA | 553 | AGAAcAAucAuuAGcAGcATsT | 554 | UGCUGCuAAUGAUUGUUCUTsT | AD-12324 |
| 1521 | CAAUGUGGAAACCUAACUG | 555 | cAAuGuGGAAAccuAAcuGTsT | 556 | cAGUuAGGUUUCcAcAUUGTsT | AD-12325 |
| 1522 | ACCAAGAAGGUACAAAAUU | 557 | AccAAGAAGGuAcAAAAuuTsT | 558 | AAUUUUGuACCUUCUUGGUTsT | AD-12326 |
| 1523 | GGUACAAAAUUGGUUGAAG | 559 | GGuAcAAAAuuGGuuGAAGTsT | 560 | CUUcAACcAAUUUUGuACCTsT | AD-12327 |
| 1524 | GGUGUGGAUUGUUCAUCAA | 561 | GGuGuGGAuuGuucAucAATsT | 562 | UUGAUGAAcAAUCcAcACCTsT | AD-12328 |
| 1525 | AGAGUUCACAAAAAGCCCA | 563 | AGAGuucAcAAAAAGcccATsT | 564 | UGGGCUUUUUGUGAACUCUTsT | AD-12329 |
| 1526 | UGAUAGCUAAAUUAAACCA | 565 | uGAuAGcuAAAuuAAAccATsT | 566 | UGGUUuAAUUuAGCuAUcATsT | AD-12330 |
| 1527 | AAUAAGCCUGAAGUGAAUC | 567 | AAuAAGccuGAAGuGAAucTsT | 568 | GAUUcACUUcAGGCUuAUUTsT | AD-12331 |
| 1528 | CAGUUGACCAACACAAUGC | 569 | cAGuuGAccAAcAcAAuGcTsT | 570 | GcAUUGUGUUGGUcAACUGTsT | AD-12332 |
| 1529 | UGGUGUGGAUUGUUCAUCA | 571 | uGGuGuGGAuuGuucAucATsT | 572 | UGAUGAAcAAUCcAcACcATsT | AD-12333 |
| 1530 | AUUCACCCUGACAGAGUUC | 573 | AuucAcccuGAcAGAGuucTsT | 574 | GAACUCUGUcAGGGUGAAUTsT | AD-12334 |
| 1531 | UAAGACCUUAUUUGGUAAU | 575 | uAAGAccuuAuuuGGuAAuTsT | 576 | AUuACcAAAuAAGGUCUuATsT | AD-12335 |
| 1532 | AAGCAAUGUGGAAACCUAA | 577 | AAGcAAuGuGGAAAccuAATsT | 578 | UuAGGUUUCcAcAUUGCUUTsT | AD-12336 |
| 1533 | UCUGAAACUGGAUAUCCCA | 579 | ucuGAAAcuGGAuAucccATsT | 580 | UGGGAuAUCcAGUUUcAGATsT | AD-12337 |

TABLE 2b
Analysis of Eg5/KSP dsRNA duplexes

| | 1st single | | 2nd single | | | |
|---|---|---|---|---|---|---|
| | dose | | dose | | 3rd | |
| | screen @ | | screen @ | | single | |
| Eg5/KSP | 50 nM [% | SDs 1st screen | 25 nM [% | SDs 2nd screen | dose | SDs 3rd screen |
| duplex | resudual | (among | resudual | (among | screen | (among |
| Name | mRNA] | quadruplicates) | mRNA] | quadruplicates) | @ 25 nM | quadruplicates) |
| AD-12072 | 65% | 2% | 82% | 5% | | |
| AD-12073 | 84% | 1% | 61% | 6% | | |
| AD-12074 | 51% | 3% | 36% | 9% | | |
| AD-12075 | 56% | 4% | 36% | 4% | | |
| AD-12076 | 21% | 4% | 13% | 3% | | |
| AD-12077 | 11% | 2% | 6% | 1% | | |
| AD-12078 | 22% | 3% | 9% | 2% | | |
| AD-12079 | 22% | 10% | 15% | 7% | | |
| AD-12080 | 68% | 4% | 52% | 13% | | |
| AD-12081 | 34% | 8% | 35% | 24% | | |
| AD-12082 | 20% | 2% | 92% | 5% | | |
| AD-12083 | 85% | 6% | 63% | 10% | | |
| AD-12084 | 18% | 6% | 17% | 4% | | |
| AD-12085 | 13% | 4% | 12% | 4% | | |
| AD-12086 | 26% | 5% | 17% | 3% | | |
| AD-12087 | 95% | 4% | 80% | 4% | | |
| AD-12088 | 29% | 6% | 29% | 2% | | |
| AD-12089 | 69% | 5% | 64% | 7% | | |
| AD-12090 | 46% | 15% | 34% | 5% | | |
| AD-12091 | 16% | 6% | 17% | 3% | | |
| AD-12092 | 82% | 26% | 63% | 5% | | |
| AD-12093 | 84% | 4% | 70% | 4% | | |
| AD-12094 | 46% | 3% | 34% | 1% | | |
| AD-12095 | 14% | 2% | 13% | 1% | | |
| AD-12096 | 26% | 11% | 17% | 1% | | |
| AD-12097 | 23% | 2% | 21% | 1% | | |
| AD-12098 | 41% | 14% | 17% | 3% | | |
| AD-12099 | 57% | 2% | 48% | 6% | | |
| AD-12100 | 101% | 11% | 98% | 8% | | |
| AD-12101 | 46% | 7% | 32% | 2% | | |
| AD-12102 | 96% | 17% | 88% | 18% | | |
| AD-12103 | 19% | 5% | 20% | 2% | | |
| AD-12104 | 40% | 8% | 24% | 2% | | |
| AD-12105 | 39% | 2% | 36% | 10% | | |
| AD-12106 | 87% | 6% | 79% | 19% | | |
| AD-12107 | 29% | 2% | 32% | 16% | | |
| AD-12108 | 38% | 4% | 39% | 8% | | |
| AD-12109 | 49% | 3% | 44% | 10% | | |
| AD-12110 | 85% | 5% | 80% | 14% | | |
| AD-12111 | 64% | 6% | 71% | 18% | | |
| AD-12112 | 48% | 4% | 41% | 5% | | |
| AD-12113 | 13% | 0% | 14% | 3% | | |
| AD-12114 | 32% | 6% | 16% | 4% | | |
| AD-12115 | 8% | 4% | 7% | 5% | | |
| AD-12116 | 74% | 5% | 61% | 7% | | |
| AD-12117 | 21% | 4% | 20% | 2% | | |
| AD-12118 | 44% | 4% | 42% | 6% | | |
| AD-12119 | 37% | 4% | 24% | 3% | | |
| AD-12120 | 22% | 2% | 15% | 4% | | |
| AD-12121 | 32% | 1% | 22% | 2% | | |
| AD-12122 | 36% | 16% | 19% | 5% | | |
| AD-12123 | 28% | 1% | 16% | | | |
| AD-12124 | 28% | 2% | 16% | | | |
| AD-12125 | 15% | 1% | 14% | | | |
| AD-12126 | 51% | 22% | 27% | | | |
| AD-12127 | 54% | 4% | 42% | 9% | | |
| AD-12128 | 29% | 1% | 20% | 2% | | |
| AD-12129 | 22% | 3% | 19% | 3% | | |
| AD-12130 | 53% | 6% | 42% | 7% | | |
| AD-12131 | 28% | 5% | 22% | 3% | | |
| AD-12132 | 88% | 2% | 90% | 18% | | |
| AD-12133 | 34% | 2% | 26% | 6% | | |
| AD-12134 | 18% | 3% | 14% | 2% | | |
| AD-12135 | 50% | 6% | 37% | 4% | | |
| AD-12136 | 42% | 19% | 22% | 2% | | |
| AD-12137 | 85% | 12% | 92% | 4% | | |
| AD-12138 | 47% | 6% | 49% | 1% | | |
| AD-12139 | 80% | 5% | 72% | 4% | | |
| AD-12140 | 97% | 22% | 67% | 9% | | |
| AD-12141 | 120% | 4% | 107% | 10% | | |
| AD-12142 | 55% | 8% | 33% | 4% | | |
| AD-12143 | 64% | 34% | 19% | 2% | | |
| AD-12144 | 58% | 29% | 17% | 2% | | |
| AD-12145 | 27% | 8% | 18% | 2% | | |
| AD-12146 | 19% | 20% | 15% | 1% | | |
| AD-12147 | 29% | 9% | 35% | 3% | | |
| AD-12148 | 30% | 3% | 56% | 5% | | |
| AD-12149 | 8% | 2% | 12% | 3% | | |
| AD-12150 | 31% | 2% | 31% | 7% | | |
| AD-12151 | 9% | 5% | 14% | 2% | | |
| AD-12152 | 3% | 3% | 23% | 3% | | |
| AD-12153 | 20% | 6% | 34% | 4% | | |
| AD-12154 | 24% | 7% | 44% | 3% | | |
| AD-12155 | 33% | 6% | 53% | 11% | | |
| AD-12156 | 35% | 5% | 40% | 5% | | |
| AD-12157 | 8% | 3% | 23% | 4% | | |
| AD-12158 | 13% | 2% | 22% | 5% | | |
| AD-12159 | 34% | 6% | 46% | 5% | | |
| AD-12160 | 19% | 3% | 31% | 4% | | |
| AD-12161 | 88% | 4% | 83% | 7% | | |
| AD-12162 | 26% | 7% | 32% | 7% | | |
| AD-12163 | 55% | 9% | 40% | 3% | | |
| AD-12164 | | | 21% | 3% | | |
| AD-12165 | 30% | 3% | 41% | 4% | | |
| AD-12166 | 9% | 10% | 22% | 9% | | |
| AD-12167 | 26% | 3% | 30% | 2% | | |
| AD-12168 | 54% | 4% | 59% | 20% | | |
| AD-12169 | 41% | 4% | 51% | 16% | | |
| AD-12170 | 43% | 4% | 52% | 20% | | |
| AD-12171 | 67% | 3% | 73% | 25% | | |
| AD-12172 | 53% | 15% | 37% | 2% | | |
| AD-12173 | 39% | 0% | 39% | 0% | | |
| AD-12174 | 41% | 5% | 27% | 0% | | |
| AD-12175 | 29% | 0% | 38% | 14% | | |
| AD-12176 | 43% | 2% | 56% | 25% | | |
| AD-12177 | 68% | 6% | 74% | 30% | | |
| AD-12178 | 41% | 4% | 41% | 6% | | |
| AD-12179 | 53% | 5% | 44% | 5% | | |
| AD-12180 | 16% | 2% | 13% | 4% | | |
| AD-12181 | 19% | 3% | 14% | 2% | | |
| AD-12182 | 16% | 4% | 18% | 8% | | |
| AD-12183 | 26% | 3% | 19% | 4% | | |
| AD-12184 | 54% | 2% | 77% | 8% | | |
| AD-12185 | 8% | 1% | 9% | 1% | | |
| AD-12186 | 36% | 3% | 41% | 6% | | |
| AD-12187 | 34% | 17% | 27% | 1% | | |
| AD-12188 | 30% | 3% | 27% | 4% | | |
| AD-12189 | 51% | 4% | 48% | 5% | | |
| AD-12190 | 33% | 2% | 26% | 4% | | |
| AD-12191 | 20% | 2% | 13% | 0% | | |
| AD-12192 | 21% | 1% | 23% | 10% | | |
| AD-12193 | 64% | 8% | 98% | 6% | | |
| AD-12194 | 8% | 2% | 15% | 4% | | |
| AD-12195 | 34% | 2% | 48% | 3% | | |
| AD-12196 | 34% | 2% | 51% | 3% | | |
| AD-12197 | 75% | 4% | 93% | 6% | | |
| AD-12198 | 55% | 5% | 48% | 2% | | |
| AD-12199 | 102% | 6% | 118% | 9% | | |
| AD-12200 | 75% | 6% | 60% | 12% | | |
| AD-12201 | 42% | 3% | 16% | 4% | | |
| AD-12202 | 29% | 4% | 9% | 3% | | |
| AD-12203 | 114% | 14% | 89% | 20% | | |
| AD-12204 | 64% | 7% | 26% | 5% | | |
| AD-12205 | 66% | 12% | 35% | 4% | | |
| AD-12206 | 46% | 3% | 32% | 12% | | |
| AD-12207 | 57% | 5% | 40% | 6% | | |
| AD-12208 | 30% | 8% | 10% | 5% | | |
| AD-12209 | 101% | 6% | 102% | 23% | | |
| AD-12210 | 38% | 11% | 27% | 14% | | |
| AD-12211 | 16% | 6% | 10% | 5% | | |
| AD-12212 | 59% | 8% | 65% | 5% | | |
| AD-12213 | 24% | 9% | 12% | 2% | | |
| AD-12214 | 67% | 14% | 70% | 12% | | |
| AD-12215 | 29% | 13% | 13% | 4% | | |
| AD-12216 | 36% | 4% | 13% | 1% | | |
| AD-12217 | 36% | 9% | 11% | 2% | | |
| AD-12218 | 35% | 5% | 17% | 3% | | |
| AD-12219 | 41% | 9% | 14% | 1% | | |
| AD-12220 | 37% | 5% | 23% | 3% | | |
| AD-12221 | 58% | 7% | 39% | 6% | | |
| AD-12222 | 74% | 9% | 53% | 3% | | |
| AD-12223 | 74% | 10% | 67% | 7% | | |
| AD-12224 | 24% | 2% | 11% | 2% | | |
| AD-12225 | 75% | 5% | 76% | 14% | | |
| AD-12226 | 45% | 8% | 40% | 3% | | |
| AD-12227 | 61% | 6% | 47% | 5% | | |
| AD-12228 | 28% | 3% | 25% | 5% | | |
| AD-12229 | 54% | 13% | 37% | 6% | | |
| AD-12230 | 70% | 17% | 65% | 4% | | |
| AD-12231 | 32% | 12% | 22% | 6% | | |
| AD-12232 | 30% | 3% | 17% | 2% | | |
| AD-12233 | 38% | 2% | 32% | 3% | | |
| AD-12234 | 90% | 5% | 95% | 7% | | |
| AD-12235 | 57% | 7% | 46% | 3% | | |
| AD-12236 | 34% | 8% | 16% | 2% | | |
| AD-12237 | 42% | 9% | 32% | 8% | | |
| AD-12238 | 42% | 6% | 34% | 6% | | |
| AD-12239 | 42% | 3% | 40% | 4% | | |
| AD-12240 | 47% | 6% | 36% | 5% | | |
| AD-12241 | 69% | 5% | 70% | 8% | | |
| AD-12242 | 61% | 2% | 47% | 3% | | |
| AD-12243 | 26% | 7% | 15% | 1% | | |
| AD-12244 | 25% | 6% | 15% | 1% | | |
| AD-12245 | 65% | 6% | 83% | 13% | | |
| AD-12246 | 29% | 7% | 31% | 6% | | |
| AD-12247 | 57% | 13% | 50% | 3% | | |
| AD-12248 | 36% | 8% | 20% | 3% | 15% | 7% |
| AD-12249 | 44% | 3% | 70% | 11% | 103% | 34% |
| AD-12250 | 47% | 5% | 18% | 5% | 17% | 4% |
| AD-12251 | 121% | 28% | 35% | 8% | 60% | 42% |
| AD-12252 | 94% | 19% | 8% | 3% | 5% | 3% |
| AD-12253 | 94% | 33% | 42% | 8% | 49% | 27% |
| AD-12254 | 101% | 58% | 70% | 5% | 80% | 32% |
| AD-12255 | 163% | 27% | 28% | 6% | 36% | 10% |
| AD-12256 | 112% | 62% | 18% | 3% | 9% | 4% |
| AD-12257 | 10% | 4% | 9% | 2% | 6% | 2% |
| AD-12258 | 27% | 9% | 18% | 3% | 20% | 6% |
| AD-12259 | 20% | 5% | 12% | 2% | 13% | 5% |
| AD-12260 | 22% | 7% | 81% | 7% | 65% | 13% |
| AD-12261 | 122% | 11% | 66% | 7% | 80% | 22% |
| AD-12262 | 97% | 30% | 33% | 6% | 44% | 18% |
| AD-12263 | 177% | 57% | 85% | 11% | 84% | 15% |
| AD-12264 | 37% | 6% | 10% | 1% | 10% | 4% |
| AD-12265 | 40% | 8% | 17% | 1% | 20% | 10% |
| AD-12266 | 33% | 9% | 9% | 1% | 8% | 4% |
| AD-12267 | 34% | 13% | 11% | 1% | 6% | 2% |
| AD-12268 | 34% | 6% | 11% | 1% | 9% | 2% |
| AD-12269 | 54% | 6% | 33% | 4% | 29% | 7% |
| AD-12270 | 52% | 5% | 29% | 4% | 27% | 6% |
| AD-12271 | 53% | 7% | 27% | 3% | 19% | 6% |
| AD-12272 | 85% | 15% | 57% | 7% | 51% | 16% |
| AD-12273 | 36% | 6% | 26% | 2% | 30% | 5% |
| AD-12274 | 75% | 21% | 40% | 2% | 50% | 19% |
| AD-12275 | 29% | 9% | 8% | 1% | 8% | 4% |
| AD-12276 | 45% | 19% | 15% | 2% | 16% | 12% |
| AD-12277 | 58% | 17% | 32% | 2% | 55% | 14% |
| AD-12278 | 120% | 35% | 96% | 10% | 124% | 38% |
| AD-12279 | 47% | 29% | 17% | 1% | 12% | 4% |
| AD-12280 | 2% | 0% | 3% | 1% | | |
| AD-12281 | 2% | 0% | 5% | 2% | | |
| AD-12282 | 3% | 0% | 25% | 5% | | |
| AD-12283 | 3% | 1% | 35% | 4% | | |
| AD-12284 | 5% | 2% | 49% | 8% | | |
| AD-12285 | 7% | 7% | 21% | 26% | | |
| AD-12286 | 28% | 34% | 12% | 7% | | |
| AD-12287 | 40% | 21% | 51% | 23% | | |
| AD-12288 | 26% | 7% | 155% | 146% | | |
| AD-12289 | 43% | 21% | 220% | 131% | | |
| AD-12290 | 2% | 1% | 81% | 23% | | |
| AD-12291 | 4% | 1% | 70% | 3% | | |
| AD-12292 | 2% | 1% | 6% | 2% | | |
| AD-12293 | 4% | 2% | 36% | 3% | | |
| AD-12294 | 10% | 6% | 38% | 3% | | |
| AD-12295 | 29% | 31% | 37% | 3% | | |
| AD-12296 | 82% | 4% | 89% | 2% | | |
| AD-12297 | 75% | 3% | 65% | 2% | | |
| AD-12298 | 73% | 4% | 60% | 3% | | |
| AD-12299 | 76% | 4% | 66% | 4% | | |
| AD-12300 | 36% | 4% | 15% | 1% | | |
| AD-12301 | 33% | 4% | 18% | 2% | | |
| AD-12302 | 66% | 5% | 65% | 3% | | |
| AD-12303 | 35% | 6% | 17% | 2% | | |
| AD-12304 | 70% | 8% | 70% | 6% | | |
| AD-12305 | 63% | 8% | 80% | 7% | | |
| AD-12306 | 23% | 6% | 20% | 3% | | |
| AD-12307 | 78% | 10% | 58% | 5% | | |
| AD-12308 | 27% | 8% | 15% | 2% | | |
| AD-12309 | 58% | 11% | 42% | 3% | | |
| AD-12310 | 106% | 23% | 80% | 2% | | |
| AD-12311 | 73% | 12% | 60% | 2% | | |
| AD-12312 | 39% | 3% | 36% | 3% | | |
| AD-12313 | 64% | 9% | 49% | 6% | | |
| AD-12314 | 28% | 7% | 14% | 6% | | |
| AD-12315 | 31% | 7% | 13% | 2% | | |
| AD-12316 | 42% | 5% | 14% | 2% | | |
| AD-12317 | 34% | 9% | 15% | 5% | | |
| AD-12318 | 46% | 4% | 28% | 4% | | |
| AD-12319 | 77% | 3% | 56% | 4% | | |
| AD-12320 | 55% | 7% | 41% | 3% | | |
| AD-12321 | 21% | 3% | 10% | 2% | | |
| AD-12322 | 27% | 8% | 30% | 12% | | |
| AD-12323 | 26% | 7% | 35% | 18% | | |
| AD-12324 | 27% | 8% | 27% | 14% | | |
| AD-12325 | 32% | 12% | 32% | 22% | | |
| AD-12326 | 42% | 22% | 45% | 41% | | |
| AD-12327 | 36% | 14% | 37% | 32% | | |
| AD-12328 | 45% | 2% | 31% | 3% | | |
| AD-12329 | 61% | 4% | 34% | 3% | | |
| AD-12330 | 63% | 5% | 38% | 4% | | |
| AD-12331 | 50% | 2% | 26% | 5% | | |
| AD-12332 | 80% | 4% | 51% | 7% | | |
| AD-12333 | 34% | 6% | 12% | 2% | | |
| AD-12334 | 27% | 2% | 18% | 3% | | |
| AD-12335 | 84% | 6% | 60% | 7% | | |
| AD-12336 | 45% | 4% | 36% | 4% | | |
| AD-12337 | 30% | 7% | 19% | 2% | | |

TABLE 3
Sequences and analysis of Eg5/KSP dsRNA duplexes

| | | | | | single | |
|---|---|---|---|---|---|---|
| | | | | | dose | SDs |
| | | | | | screen @ | 2nd |
| | | | | | 25 nM [% | screen |
| | SEQ ID | Antisense sequence (5′- | SEQ ID | duplex | residual | (among |
| Sense sequence (5′-3′) | NO. | 3′) | NO. | name | mRNA] | quadruplicates) |

| | | | | | | |
|---|---|---|---|---|---|---|
| ccAuuAcuAcAGuAGcAcuTsT | 582 | AGUGCuACUGuAGuAAUGGTsT | 583 | AD-14085 | 19% | 1% |
| AucuGGcAAccAuAuuucuTsT | 584 | AGAAAuAUGGUUGCcAGAUTsT | 585 | AD-14086 | 38% | 1% |
| GAuAGcuAAAuuAAAccAATsT | 586 | UUGGUUuAAUUuAGCuAUCTsT | 587 | AD-14087 | 75% | 10% |
| AGAuAccAuuAcuAcAGuATsT | 588 | uACUGuAGuAAUGGuAUCUTsT | 589 | AD-14088 | 22% | 8% |
| GAuuGuucAucAAuuGGcGTsT | 590 | CGCcAAUUGAUGAAcAAUCTsT | 591 | AD-14089 | 70% | 12% |
| GcuuucuccucGGcucAcuTsT | 592 | AGuGAGCCGAGGAGAAAGCTsT | 593 | AD-14090 | 79% | 11% |
| GGAGGAuuGGcuGAcAAGATsT | 594 | UCUUGUcAGCcAAUCCUCCTsT | 595 | AD-14091 | 29% | 3% |
| uAAuGAAGAGuAuAccuGGTsT | 596 | CcAGGuAuACUCUUcAUuATsT | 597 | AD-14092 | 23% | 2% |
| uuucAccAAAccAuuuGuATsT | 598 | uAcAAAUGGUUUGGUGAAATsT | 599 | AD-14093 | 60% | 2% |
| cuuAuuAAGGAGuAuAcGGTsT | 600 | CCGuAuACUCCUuAAuAAGTsT | 601 | AD-14094 | 11% | 3% |
| GAAAucAGAuGGAcGuAAGTsT | 602 | CUuACGUCcAUCUGAUUUCTsT | 603 | AD-14095 | 10% | 2% |
| cAGAuGucAGcAuAAGcGATsT | 604 | UCGCUuAUGCUGAcAUCUGTsT | 605 | AD-14096 | 27% | 2% |
| AucuAAcccuAGuuGuAucTsT | 606 | GAuAcAACuAGGGUuAGAUTsT | 607 | AD-14097 | 45% | 6% |
| AAGAGcuuGuuAAAAucGGTsT | 608 | CCGAUUUuAAcAAGCUCUUTsT | 609 | AD-14098 | 50% | 10% |
| uuAAGGAGuAuAcGGAGGATsT | 610 | UCCUCCGuAuACUCCUuAATsT | 611 | AD-14099 | 12% | 4% |
| uuGcAAuGuAAAuAcGuAuTsT | 612 | AuACGuAUUuAcAUUGcAATsT | 613 | AD-14100 | 49% | 7% |
| ucuAAcccuAGuuGuAuccTsT | 614 | GGAuAcAACuAGGGUuAGATsT | 615 | AD-14101 | 36% | 1% |
| cAuGuAucuuuuucucGAuTsT | 616 | AUCGAGAAAAAGAuAcAUGTsT | 617 | AD-14102 | 49% | 3% |
| GAuGucAGcAuAAGcGAuGTsT | 618 | cAUCGCUuAUGCUGAcAUCTsT | 619 | AD-14103 | 74% | 5% |
| ucccAAcAGGuAcGAcAccTsT | 620 | GGUGUCGuACCUGUUGGGATsT | 621 | AD-14104 | 27% | 3% |
| uGcucAcGAuGAGuuuAGuTsT | 622 | ACuAAACUcAUCGUGAGcATsT | 623 | AD-14105 | 34% | 4% |
| AGAGcuuGuuAAAAucGGATsT | 624 | UCCGAUUUuAAcAAGCUCUTsT | 625 | AD-14106 | 9% | 2% |
| GcGuAcAAGAAcAucuAuATsT | 626 | uAuAGAUGUUCUUGuACGCTsT | 627 | AD-14107 | 5% | 1% |
| GAGGuuGuAAGccAAuGuuTsT | 628 | AAcAUUGGCUuAcAACCUCTsT | 629 | AD-14108 | 15% | 1% |
| AAcAGGuAcGAcAccAcAGTsT | 630 | CUGUGGUGUCGuACCUGUUTsT | 631 | AD-14109 | 91% | 2% |
| AAcccuAGuuGuAucccucTsT | 632 | GAGGGAuAcAACuAGGGUUTsT | 633 | AD-14110 | 66% | 5% |
| GcAuAAGcGAuGGAuAAuATsT | 634 | uAUuAUCcAUCGCUuAUGCTsT | 635 | AD-14111 | 33% | 3% |
| AAGcGAuGGAuAAuAccuATsT | 636 | uAGGuAUuAUCcAUCGCUUTsT | 637 | AD-14112 | 51% | 3% |
| uGAuccuGuAcGAAAAGAATsT | 638 | UUCUUUUCGuAcAGGAUcATsT | 639 | AD-14113 | 22% | 3% |
| AAAAcAuuGGccGuucuGGTsT | 640 | CcAGAACGGCcAAUGUUUUTsT | 641 | AD-14114 | 117% | 8% |
| cuuGGAGGGcGuAcAAGAATsT | 642 | UUCUUGuACGCCCUCcAAGTsT | 643 | AD-14115 | 50% | 8% |
| GGcGuAcAAGAAcAucuAuTsT | 644 | AuAGAUGUUCUUGuACGCCTsT | 645 | AD-14116 | 14% | 3% |
| AcucuGAGuAcAuuGGAAuTsT | 646 | AUUCcAAUGuACUcAGAGUTsT | 647 | AD-14117 | 12% | 4% |
| uuAuuAAGGAGuAuAcGGATsT | 648 | UCCGuAuACUCCUuAAuAATsT | 649 | AD-14118 | 26% | 4% |
| uAAGGAGuAuAcGGAGGAGTsT | 650 | CUCCUCCGuAuACUCCUuATsT | 651 | AD-14119 | 24% | 5% |
| AAAucAAuAGucAAcuAAATsT | 652 | UUuAGUUGACuAUUGAUUUTsT | 653 | AD-14120 | 8% | 1% |
| AAucAAuAGucAAcuAAAGTsT | 654 | CUUuAGUUGACuAUUGAUUTsT | 655 | AD-14121 | 24% | 2% |
| uucucAGuAuAcuGuGuAATsT | 656 | UuAcAcAGuAuACUGAGAATsT | 657 | AD-14122 | 10% | 1% |
| uGuGAAAcAcucuGAuAAATsT | 658 | UUuAUcAGAGUGUUUCAcATsT | 659 | AD-14123 | 8% | 1% |
| AGAuGuGAAucucuGAAcATsT | 660 | UGUUcAGAGAUUcAcAUCUTsT | 661 | AD-14124 | 9% | 2% |
| AGGuuGuAAGccAAuGuuGTsT | 662 | cAAcAUUGGCUuAcAACCUTsT | 663 | AD-14125 | 114% | 6% |
| uGAGAAAucAGAuGGAcGuTsT | 664 | ACGUCcAUCUGAUUUCUcATsT | 665 | AD-14126 | 9% | 1% |
| AGAAAucAGAuGGAcGuAATsT | 666 | UuACGUCcAUCUGAUUUCUTsT | 667 | AD-14127 | 57% | 6% |
| AuAucccAAcAGGuAcGAcTsT | 668 | GUCGuACCUGUUGGGAuAUTsT | 669 | AD-14128 | 104% | 6% |
| cccAAcAGGuAcGAcAccATsT | 670 | UGGUGUCGuACCUGUUGGGTsT | 671 | AD-14129 | 21% | 2% |
| AGuAuAcuGAAGAAccucuTsT | 672 | AGAGGUUCUUcAGuAuACUTsT | 673 | AD-14130 | 57% | 6% |
| AuAuAuAucAGccGGGcGcTsT | 674 | GCGCCCGGCUGAuAuAuAUTsT | 675 | AD-14131 | 93% | 6% |
| AAucuAAcccuAGuuGuAuTsT | 676 | AuAcAACuAGGGUuAGAUUTsT | 677 | AD-14132 | 75% | 8% |
| cuAAcccuAGuuGuAucccTsT | 678 | GGGAuAcAACuAGGGUuAGTsT | 679 | AD-14133 | 66% | 4% |
| cuAGuuGuAucccuccuuuTsT | 680 | AAAGGAGGGAuAcAACuAGTsT | 681 | AD-14134 | 44% | 6% |
| AGAcAucuGAcuAAuGGcuTsT | 682 | AGCcAUuAGUcAGAUGUCUTsT | 683 | AD-14135 | 55% | 6% |
| GAAGcucAcAAuGAuuuAATsT | 684 | UuAAAUcAUUGUGAGCUUCTsT | 685 | AD-14136 | 29% | 3% |
| AcAuGuAucuuuuucucGATsT | 686 | UCGAGAAAAAGAuAcAUGUTsT | 687 | AD-14137 | 40% | 3% |
| ucGAuucAAAucuuAAcccTsT | 688 | GGGUuAAGAUUUGAAUCGATsT | 689 | AD-14138 | 39% | 5% |
| ucuuAAcccuuAGGAcucuTsT | 690 | AGAGUCCuAAGGGUuAAGATsT | 691 | AD-14139 | 71% | 11% |
| GcucAcGAuGAGuuuAGuGTsT | 692 | cACuAAACUcAUCGUGAGCTsT | 693 | AD-14140 | 43% | 15% |
| cAuAAGcGAuGGAuAAuAcTsT | 694 | GuAUuAUCcAUCGCUuAUGTsT | 695 | AD-14141 | 33% | 6% |
| AuAAGcGAuGGAuAAuAccTsT | 696 | GGuAUuAUCcAUCGCUuAUTsT | 697 | AD-14142 | 51% | 14% |
| ccuAAuAAAcuGcccucAGTsT | 698 | CUGAGGGcAGUUuAUuAGGTsT | 699 | AD-14143 | 42% | 1% |
| ucGGAAAGuuGAAcuuGGuTsT | 700 | ACcAAGUUcAACUUUCCGATsT | 701 | AD-14144 | 4% | 4% |
| GAAAAcAuuGGccGuucuGTsT | 702 | cAGAACGGCcAAUGUUUUCTsT | 703 | AD-14145 | 92% | 5% |
| AAGAcuGAucuucuAAGuuTsT | 704 | AACUuAGAAGAUcAGUCUUTsT | 705 | AD-14146 | 13% | 2% |
| GAGcuuGuuAAAAucGGAATsT | 706 | UUCCGAUUUuAAcAAGCUCTsT | 707 | AD-14147 | 8% | 1% |
| AcAuuGGccGuucuGGAGcTsT | 708 | GCUCcAGAACGGCcAAUGUTsT | 709 | AD-14148 | 80% | 7% |
| AAGAAcAucuAuAAuuGcATsT | 710 | UGcAAUuAuAGAUGUUCUUTsT | 711 | AD-14149 | 44% | 7% |
| AAAuGuGucuAcucAuGuuTsT | 712 | AAcAUGAGuAGAcAcAUUUTsT | 713 | AD-14150 | 32% | 29% |
| uGucuAcucAuGuuucucATsT | 714 | UGAGAAAcAUGAGuAGAcATsT | 715 | AD-14151 | 75% | 11% |
| GuAuAcuGuGuAAcAAucuTsT | 716 | AGAUUGUuAcAcAGuAuACTsT | 717 | AD-14152 | 8% | 5% |
| uAuAcuGuGuAAcAAucuATsT | 718 | uAGAUUGUuAcAcAGuAuATsT | 719 | AD-14153 | 17% | 11% |
| cuuAGuAGuGuccAGGAAATsT | 720 | UUUCCUGGAcACuACuAAGTsT | 721 | AD-14154 | 16% | 4% |
| ucAGAuGGAcGuAAGGcAGTsT | 722 | CUGCCUuACGUCcAUCUGATsT | 723 | AD-14155 | 11% | 1% |
| AGAuAAAuuGAuAGcAcAATsT | 724 | UUGUGCuAUcAAUUuAUCUTsT | 725 | AD-14156 | 10% | 1% |
| cAAcAGGuAcGAcAccAcATsT | 726 | UGUGGUGUCGuACCUGUUGTsT | 727 | AD-14157 | 29% | 3% |
| uGcAAuGuAAAuAcGuAuuTsT | 728 | AAuACGuAUUuAcAUUGcATsT | 729 | AD-14158 | 51% | 3% |
| AGucAGAAuuuuAucuAGATsT | 730 | UCuAGAuAAAAUUCUGACUTsT | 731 | AD-14159 | 53% | 5% |
| cuAGAAAucuuuuAAcAccTsT | 732 | GGUGUuAAAAGAUUUCuAGTsT | 733 | AD-14160 | 40% | 3% |
| AAuAAAucuAAcccuAGuuTsT | 734 | AACuAGGGUuAGAUUuAUUTsT | 735 | AD-14161 | 83% | 7% |
| AAuuuucuGcucAcGAuGATsT | 736 | UcAUCGUGAGcAGAAAAUUTsT | 737 | AD-14162 | 44% | 6% |
| GcccucAGuAAAuccAuGGTsT | 738 | CcAUGGAUUuACUGAGGGCTsT | 739 | AD-14163 | 57% | 3% |
| AcGuuuAAAAcGAGAucuuTsT | 740 | AAGAUCUCGUUUuAAACGUTsT | 741 | AD-14164 | 4% | 1% |
| AGGAGAuAGAAcGuuuAAATsT | 742 | UUuAAACGUUCuAUCUCCUTsT | 743 | AD-14165 | 11% | 1% |
| GAccGucAuGGcGucGcAGTsT | 744 | CUGCGACGCcAUGACGGUCTsT | 745 | AD-14166 | 90% | 5% |
| AccGucAuGGcGucGcAGcTsT | 746 | GCUGCGACGCcAUGACGGUTsT | 747 | AD-14167 | 49% | 1% |
| GAAcGuuuAAAAcGAGAucTsT | 748 | GAUCUCGUUUuAAACGUUCTsT | 749 | AD-14168 | 12% | 2% |
| uuGAGcuuAAcAuAGGuAATsT | 750 | UuACCuAUGUuAAGCUcAATsT | 751 | AD-14169 | 66% | 4% |
| AcuAAAuuGAucucGuAGATsT | 752 | UCuACGAGAUcAAUUuAGUTsT | 753 | AD-14170 | 52% | 6% |
| ucGuAGAAuuAucuuAAuATsT | 754 | uAUuAAGAuAAUUCuACGATsT | 755 | AD-14171 | 42% | 4% |
| GGAGAuAGAAcGuuuAAAATsT | 756 | UUUuAAACGUUCuAUCUCCTsT | 757 | AD-14172 | 3% | 1% |
| AcAAcuuAuuGGAGGuuGuTsT | 758 | AcAACCUCcAAuAAGUUGUTsT | 759 | AD-14173 | 29% | 2% |
| uGAGcuuAAcAuAGGuAAATsT | 760 | UUuACCuAUGUuAAGCUcATsT | 761 | AD-14174 | 69% | 2% |
| AucucGuAGAAuuAucuuATsT | 762 | uAAGAuAAUUCuACGAGAUTsT | 763 | AD-14175 | 53% | 3% |
| cuGcGuGcAGucGGuccucTsT | 764 | GAGGACCGACUGcACGcAGTsT | 765 | AD-14176 | 111% | 4% |
| cAcGcAGcGcccGAGAGuATsT | 766 | uACUCUCGGGCGCUGCGUGTsT | 767 | AD-14177 | 87% | 6% |
| AGuAccAGGGAGAcuccGGTsT | 768 | CCGGAGUCUCCCUGGuACUTsT | 769 | AD-14178 | 59% | 2% |
| AcGGAGGAGAuAGAAcGuuTsT | 770 | AACGUUCuAUCUCCUCCGUTsT | 771 | AD-14179 | 9% | 2% |
| AGAAcGuuuAAAAcGAGAuTsT | 772 | AUCUCGUUUuAAACGUUCUTsT | 773 | AD-14180 | 43% | 2% |
| AAcGuuuAAAAcGAGAucuTsT | 774 | AGAUCUCGUUUuAAACGUUTsT | 775 | AD-14181 | 70% | 10% |
| AGcuuGAGcuuAAcAuAGGTsT | 776 | CCuAUGUuAAGCUcAAGCUTsT | 777 | AD-14182 | 100% | 7% |
| AGcuuAAcAuAGGuAAAuATsT | 778 | uAUUuACCuAUGUuAAGCUTsT | 779 | AD-14183 | 60% | 5% |
| uAGAGcuAcAAAAccuAucTsT | 780 | GAuAGGUUUUGuAGCUCuATsT | 781 | AD-14184 | 129% | 6% |
| uAGuuGuAucccuccuuuATsT | 782 | uAAAGGAGGGAuAcAACuATsT | 783 | AD-14185 | 62% | 4% |
| AccAcccAGAcAucuGAcuTsT | 784 | AGUcAGAUGUCUGGGUGGUTsT | 785 | AD-14186 | 42% | 3% |
| AGAAAcuAAAuuGAucucGTsT | 786 | CGAGAUcAAUUuAGUUUCUTsT | 787 | AD-14187 | 123% | 12% |
| ucucGuAGAAuuAucuuAATsT | 788 | UuAAGAuAAUUCuACGAGATsT | 789 | AD-14188 | 38% | 2% |
| cAAcuuAuuGGAGGuuGuATsT | 790 | uAcAACCUCcAAuAAGUUGTsT | 791 | AD-14189 | 13% | 1% |
| uuGuAucccuccuuuAAGuTsT | 792 | ACUuAAAGGAGGGAuAcAATsT | 793 | AD-14190 | 59% | 3% |
| ucAcAAcuuAuuGGAGGuuTsT | 794 | AACCUCcAAuAAGUUGUGATsT | 795 | AD-14191 | 93% | 3% |
| AGAAcuGuAcucuucucAGTsT | 796 | CUGAGAAGAGuAcAGUUCUTsT | 797 | AD-14192 | 45% | 5% |
| GAGcuuAAcAuAGGuAAAuTsT | 798 | AUUuACCuAUGUuAAGCUCTsT | 799 | AD-14193 | 57% | 3% |
| cAccAAcAucuGuccuuAGTsT | 800 | CuAAGGAcAGAUGUUGGUGTsT | 801 | AD-14194 | 51% | 4% |
| AAAGcccAcuuuAGAGuAuTsT | 802 | AuACUCuAAAGUGGGCUUUTsT | 803 | AD-14195 | 77% | 5% |
| AAGcccAcuuuAGAGuAuATsT | 804 | uAuACUCuAAAGUGGGCUUTsT | 805 | AD-14196 | 42% | 6% |
| GAccuuAuuuGGuAAucuGTsT | 806 | cAGAUuACcAAAuAAGGUCTsT | 807 | AD-14197 | 15% | 2% |
| GAuuAAuGuAcucAAGAcuTsT | 808 | AGUCUUGAGuAcAUuAAUCTsT | 809 | AD-14198 | 12% | 2% |
| cuuuAAGAGGccuAAcucATsT | 810 | UGAGUuAGGCCUCUuAAAGTsT | 811 | AD-14199 | 18% | 2% |
| uuAAAccAAAcccuAuuGATsT | 812 | UcAAuAGGGUUUGGUUuAATsT | 813 | AD-14200 | 72% | 9% |
| ucuGuuGGAGAucuAuAAuTsT | 814 | AUuAuAGAUCUCcAAcAGATsT | 815 | AD-14201 | 9% | 3% |
| cuGAuGuuucuGAGAGAcuTsT | 816 | AGUCUCUcAGAAAcAUcAGTsT | 817 | AD-14202 | 25% | 3% |
| GcAuAcucuAGucGuucccTsT | 818 | GGGAACGACuAGAGuAUGCTsT | 819 | AD-14203 | 21% | 1% |
| GuuccuuAucGAGAAucuATsT | 820 | uAGAUUCUCGAuAAGGAACTsT | 821 | AD-14204 | 4% | 2% |
| GcAcuuGGAucucucAcAuTsT | 822 | AUGUGAGAGAUCcAAGUGCTsT | 823 | AD-14205 | 5% | 1% |
| AAAAAAGGAAcuAGAuGGcTsT | 824 | GCcAUCuAGUUCCUUUUUUTsT | 825 | AD-14206 | 79% | 6% |
| AGAGcAGAuuAccucuGcGTsT | 826 | CGcAGAGGuAAUCUGCUCUTsT | 827 | AD-14207 | 55% | 2% |
| AGcAGAuuAccucuGcGAGTsT | 828 | CUCGcAGAGGuAAUCUGCUTsT | 829 | AD-14208 | 100% | 4% |
| cccuGAcAGAGuucAcAAATsT | 830 | UUUGUGAACUCUGUcAGGGTsT | 831 | AD-14209 | 34% | 3% |
| GuuuAccGAAGuGuuGuuuTsT | 832 | AAAcAAcACUUCGGuAAACTsT | 833 | AD-14210 | 13% | 2% |
| uuAcAGuAcAcAAcAAGGATsT | 834 | UCCUUGUUGUGuACUGuAATsT | 835 | AD-14211 | 9% | 1% |
| AcuGGAucGuAAGAAGGcATsT | 836 | UGCCUUCUuACGAUCcAGUTsT | 837 | AD-14212 | 20% | 3% |
| GAGcAGAuuAccucuGcGATsT | 838 | UCGcAGAGGuAAUCUGCUCTsT | 839 | AD-14213 | 48% | 5% |
| AAAAGAAGuuAGuGuAcGATsT | 840 | UCGuAcACuAACUUCUUUUTsT | 841 | AD-14214 | 28% | 18% |
| GAccAuuuAAuuuGGcAGATsT | 842 | UCUGCcAAAUuAAAUGGUCTsT | 843 | AD-14215 | 132% | 0% |
| GAGAGGAGuGAuAAuuAAATsT | 844 | UUuAAUuAUcACUCCUCUCTsT | 845 | AD-14216 | 3% | 0% |
| cuGGAGGAuuGGcuGAcAATsT | 846 | UUGUcAGCcAAUCCUCcAGTsT | 847 | AD-14217 | 19% | 1% |
| cucuAGucGuucccAcucATsT | 848 | UGAGUGGGAACGACuAGAGTsT | 849 | AD-14218 | 67% | 8% |
| GAuAccAuuAcuAcAGuAGTsT | 850 | CuACUGuAGuAAUGGuAUCTsT | 851 | AD-14219 | 76% | 4% |
| uucGucuGcGAAGAAGAAATsT | 852 | UUUCUUCUUCGcAGACGAATsT | 853 | AD-14220 | 33% | 8% |
| GAAAAGAAGuuAGuGuAcGTsT | 854 | CGuAcACuAACUUCUUUUCTsT | 855 | AD-14221 | 25% | 2% |
| uGAuGuuuAccGAAGuGuuTsT | 856 | AAcACUUCGGuAAAcAUcATsT | 857 | AD-14222 | 7% | 2% |
| uGuuuGuccAAuucuGGAuTsT | 858 | AUCcAGAAUUGGAcAAAcATsT | 859 | AD-14223 | 19% | 2% |
| AuGAAGAGuAuAccuGGGATsT | 860 | UCCcAGGuAuACUCUUcAUTsT | 861 | AD-14224 | 13% | 1% |
| GcuAcucuGAuGAAuGcAuTsT | 862 | AUGcAUUcAUcAGAGuAGCTsT | 863 | AD-14225 | 15% | 2% |
| GcccuuGuAGAAAGAAcAcTsT | 864 | GUGUUCUUUCuAcAAGGGCTsT | 865 | AD-14226 | 11% | 0% |
| ucAuGuuccuuAucGAGAATsT | 866 | UUCUCGAuAAGGAAcAUGATsT | 867 | AD-14227 | 5% | 1% |
| GAAuAGGGuuAcAGAGuuGTsT | 868 | cAACUCUGuAACCCuAUUCTsT | 869 | AD-14228 | 34% | 3% |
| cAAAcuGGAucGuAAGAAGTsT | 870 | CUUCUuACGAUCcAGUUUGTsT | 871 | AD-14229 | 15% | 2% |
| cuuAuuuGGuAAucuGcuGTsT | 872 | cAGcAGAUuACcAAAuAAGTsT | 873 | AD-14230 | 20% | 1% |
| AGcAAuGuGGAAAccuAAcTsT | 874 | GUuAGGUUUCcAcAUUGCUTsT | 875 | AD-14231 | 18% | 1% |
| AcAAuAAAGcAGAcccAuuTsT | 876 | AAUGGGUCUGCUUuAUUGUTsT | 877 | AD-14232 | 21% | 1% |
| AAccAcuuAGuAGuGuccATsT | 878 | UGGAcACuACuAAGUGGUUTsT | 879 | AD-14233 | 106% | 12% |
| AGucAAGAGccAucuGuAGTsT | 880 | CuAcAGAUGGCUCUUGACUTsT | 881 | AD-14234 | 35% | 3% |
| cucccuAGAcuucccuAuuTsT | 882 | AAuAGGGAAGUCuAGGGAGTsT | 883 | AD-14235 | 48% | 4% |
| AuAGcuAAAuuAAAccAAATsT | 884 | UUUGGUUuAAUUuAGCuAUTsT | 885 | AD-14236 | 23% | 3% |
| uGGcuGGuAuAAuuccAcGTsT | 886 | CGUGGAAUuAuACcAGCcATsT | 887 | AD-14237 | 79% | 9% |
| uuAuuuGGuAAucuGcuGuTsT | 888 | AcAGcAGAUuACcAAAuAATsT | 889 | AD-14238 | 92% | 7% |
| AAcuAGAuGGcuuucucAGTsT | 890 | CUGAGAAAGCcAUCuAGUUTsT | 891 | AD-14239 | 20% | 2% |
| ucAuGGcGucGcAGccAAATsT | 892 | UUUGGCUGCGACGCcAUGATsT | 893 | AD-14240 | 71% | 6% |
| AcuGGAGGAuuGGcuGAcATsT | 894 | UGUcAGCcAAUCCUCcAGUTsT | 895 | AD-14241 | 14% | 1% |
| cuAuAAuuGcAcuAucuuuTsT | 896 | AAAGAuAGUGcAAUuAuAGTsT | 897 | AD-14242 | 11% | 2% |
| AAAGGucAccuAAuGAAGATsT | 898 | UCUUcAUuAGGUGACCUUUTsT | 899 | AD-14243 | 11% | 1% |
| AuGAAuGcAuAcucuAGucTsT | 900 | GACuAGAGuAUGcAUUcAUTsT | 901 | AD-14244 | 15% | 2% |
| AAcAuAuuGAAuAAGccuGTsT | 902 | cAGGCUuAUUcAAuAUGUUTsT | 903 | AD-14245 | 80% | 7% |
| AAGAAGGcAGuuGAccAAcTsT | 904 | GUUGGUcAACUGCCUUCUUTsT | 905 | AD-14246 | 57% | 5% |
| GAuAcuAAAAGAAcAAucATsT | 906 | UGAUUGUUCUUUuAGuAUCTsT | 907 | AD-14247 | 9% | 3% |
| AuAcuGAAAAucAAuAGucTsT | 908 | GACuAUUGAUUUUcAGuAUTsT | 909 | AD-14248 | 39% | 4% |
| AAAAAGGAAcuAGAuGGcuTsT | 910 | AGCcAUCuAGUUCCUUUUUTsT | 911 | AD-14249 | 64% | 2% |
| GAAcuAGAuGGcuuucucATsT | 912 | UGAGAAAGCcAUCuAGUUCTsT | 913 | AD-14250 | 18% | 2% |
| GAAAccuAAcuGAAGAccuTsT | 914 | AGGUCUUcAGUuAGGUUUCTsT | 915 | AD-14251 | 56% | 6% |
| uAcccAucAAcAcuGGuAATsT | 916 | UuACcAGUGUUGAUGGGuATsT | 917 | AD-14252 | 48% | 6% |
| AuuuuGAuAucuAcccAuuTsT | 918 | AAUGGGuAGAuAUcAAAAUTsT | 919 | AD-14253 | 39% | 5% |
| AucccuAuAGuucAcuuuGTsT | 920 | cAAAGUGAACuAuAGGGAUTsT | 921 | AD-14254 | 44% | 8% |
| AuGGGcuAuAAuuGcAcuATsT | 922 | uAGUGcAAUuAuAGCCcAUTsT | 923 | AD-14255 | 108% | 8% |
| AGAuuAccucuGcGAGcccTsT | 924 | GGGCUCGcAGAGGuAAUCUTsT | 925 | AD-14256 | 108% | 6% |
| uAAuuccAcGuAcccuucATsT | 926 | UGAAGGGuACGUGGAAUuATsT | 927 | AD-14257 | 23% | 2% |
| GucGuucccAcucAGuuuuTsT | 928 | AAAACuGAGuGGGAACGACTsT | 929 | AD-14258 | 21% | 3% |
| AAAucAAucccuGuuGAcuTsT | 930 | AGUcAAcAGGGAUUGAUUUTsT | 931 | AD-14259 | 19% | 2% |
| ucAuAGAGcAAAGAAcAuATsT | 932 | uAUGUUCUUUGCUCuAUGATsT | 933 | AD-14260 | 10% | 1% |
| uuAcuAcAGuAGcAcuuGGTsT | 934 | CcAAGUGCuACUGuAGuAATsT | 935 | AD-14261 | 76% | 3% |
| AuGuGGAAAccuAAcuGAATsT | 936 | UUcAGUuAGGUUUCcAcAUTsT | 937 | AD-14262 | 13% | 2% |
| uGuGGAAAccuAAcuGAAGTsT | 938 | CUUcAGUuAGGUUUCcAcATsT | 939 | AD-14263 | 14% | 2% |
| ucuuccuuAAAuGAAAGGGTsT | 940 | CCCUUUcAUUuAAGGAAGATsT | 941 | AD-14264 | 65% | 3% |
| uGAAGAAccucuAAGucAATsT | 942 | UUGACUuAGAGGUUCUUcATsT | 943 | AD-14265 | 13% | 1% |
| AGAGGucuAAAGuGGAAGATsT | 944 | UCUUCcACUUuAGACCUCUTsT | 945 | AD-14266 | 18% | 3% |
| AuAucuAcccAuuuuucuGTsT | 946 | cAGAAAAAUGGGuAGAuAUTsT | 947 | AD-14267 | 50% | 9% |
| uAAGccuGAAGuGAAucAGTsT | 948 | CUGAUUcACUUcAGGCUuATsT | 949 | AD-14268 | 13% | 3% |
| AGAuGcAGAccAuuuAAuuTsT | 950 | AAUuAAAUGGUCUGcAUCUTsT | 951 | AD-14269 | 19% | 4% |
| AGuGuuGuuuGuccAAuucTsT | 952 | GAAUUGGAcAAAcAAcACUTsT | 953 | AD-14270 | 11% | 2% |
| cuAuAAuGAAGAGcuuuuuTsT | 954 | AAAAAGCUCUUcAUuAuAGTsT | 955 | AD-14271 | 11% | 1% |
| AGAGGAGuGAuAAuuAAAGTsT | 956 | CUUuAAUuAUcACUCCUCUTsT | 957 | AD-14272 | 7% | 1% |
| uuucucuGuuAcAAuAcAuTsT | 958 | AUGuAUUGuAAcAGAGAAATsT | 959 | AD-14273 | 14% | 2% |
| AAcAucuAuAAuuGcAAcATsT | 960 | UGUUGcAAUuAuAGAUGUUTsT | 961 | AD-14274 | 73% | 4% |
| uGcuAGAAGuAcAuAAGAcTsT | 962 | GUCUuAUGuACUUCuAGcATsT | 963 | AD-14275 | 10% | 1% |
| AAuGuAcucAAGAcuGAucTsT | 964 | GAUcAGUCUUGAGuAcAUUTsT | 965 | AD-14276 | 89% | 2% |
| GuAcucAAGAcuGAucuucTsT | 966 | GAAGAUcAGUCUUGAGuACTsT | 967 | AD-14277 | 7% | 1% |
| cAcucuGAuAAAcucAAuGTsT | 968 | cAUUGAGUUuAUcAGAGUGTsT | 969 | AD-14278 | 12% | 1% |
| AAGAGcAGAuuAccucuGcTsT | 970 | GcAGAGGuAAUCUGCUCUUTsT | 971 | AD-14279 | 104% | 3% |
| ucuGcGAGcccAGAucAAcTsT | 972 | GUUGAUCUGGGCUCGcAGATsT | 973 | AD-14280 | 21% | 2% |
| AAcuuGAGccuuGuGuAuATsT | 974 | uAuAcAcAAGGCUcAAGUUTsT | 975 | AD-14281 | 43% | 3% |
| GAAuAuAuAuAucAGccGGTsT | 976 | CCGGCUGAuAuAuAuAUUCTsT | 977 | AD-14282 | 45% | 6% |
| uGucAucccuAuAGuucAcTsT | 978 | GUGAACuAuAGGGAUGAcATsT | 979 | AD-14283 | 35% | 5% |
| GAucuGGcAAccAuAuuucTsT | 980 | GAAAuAUGGUUGCcAGAUCTsT | 981 | AD-14284 | 58% | 3% |
| uGGcAAccAuAuuucuGGATsT | 982 | UCcAGAAAuAUGGUUGCcATsT | 983 | AD-14285 | 48% | 3% |
| GAuGuuuAccGAAGuGuuGTsT | 984 | cAAcACUUCGGuAAAcAUCTsT | 985 | AD-14286 | 49% | 3% |
| uuccuuAucGAGAAucuAATsT | 986 | UuAGAUUCUCGAuAAGGAATsT | 987 | AD-14287 | 6% | 1% |
| AGcuuAAuuGcuuucuGGATsT | 988 | UCcAGAAAGcAAUuAAGCUTsT | 989 | AD-14288 | 50% | 2% |
| uuGcuAuuAuGGGAGAccATsT | 990 | UGGUCUCCcAuAAuAGcAATsT | 991 | AD-14289 | 48% | 1% |
| GucAuGGcGucGcAGccAATsT | 992 | UUGGCUGCGACGCcAUGACTsT | 993 | AD-14290 | 112% | 7% |
| uAAuuGcAcuAucuuuGcGTsT | 994 | CGcAAAGAuAGUGcAAUuATsT | 995 | AD-14291 | 77% | 2% |
| cuAucuuuGcGuAuGGccATsT | 996 | UGGCcAuACGcAAAGAuAGTsT | 997 | AD-14292 | 80% | 6% |
| ucccuAuAGuucAcuuuGuTsT | 998 | AcAAAGUGAACuAuAGGGATsT | 999 | AD-14293 | 58% | 2% |
| ucAAccuuuAAuucAcuuGTsT | 1000 | cAAGUGAAUuAAAGGUUGATsT | 1001 | AD-14294 | 77% | 2% |
| GGcAAccAuAuuucuGGAATsT | 1002 | UUCcAGAAAuAUGGUUGCCTsT | 1003 | AD-14295 | 62% | 2% |
| AuGuAcucAAGAcuGAucuTsT | 1004 | AGAUcAGUCUUGAGuAcAUTsT | 1005 | AD-14296 | 59% | 4% |
| GcAGAccAuuuAAuuuGGcTsT | 1006 | GCcAAAUuAAAUGGUCUGCTsT | 1007 | AD-14297 | 37% | 1% |
| ucuGAGAGAcuAcAGAuGuTsT | 1008 | AcAUCUGuAGUCUCUcAGATsT | 1009 | AD-14298 | 21% | 1% |
| uGcucAuAGAGcAAAGAAcTsT | 1010 | GUUCUUUGCUCuAUGAGcATsT | 1011 | AD-14299 | 6% | 1% |
| AcAuAAGAccuuAuuuGGuTsT | 1012 | ACcAAAuAAGGUCUuAUGUTsT | 1013 | AD-14300 | 17% | 2% |
| uuuGuGcuGAuucuGAuGGTsT | 1014 | CcAUcAGAAUcAGcAcAAATsT | 1015 | AD-14301 | 97% | 6% |
| ccAucAAcAcuGGuAAGAATsT | 1016 | UUCUuACcAGUGUUGAUGGTsT | 1017 | AD-14302 | 13% | 1% |
| AGAcAAuuccGGAuGuGGATsT | 1018 | UCcAcAUCCGGAAUUGUCUTsT | 1019 | AD-14303 | 13% | 3% |
| GAAcuuGAGccuuGuGuAuTsT | 1020 | AuAcAcAAGGCUcAAGUUCTsT | 1021 | AD-14304 | 38% | 2% |
| uAAuuuGGcAGAGcGGAAATsT | 1022 | UUUCCGCUCUGCcAAAUuATsT | 1023 | AD-14305 | 14% | 2% |
| uGGAuGAAGuuAuuAuGGGTsT | 1024 | CCcAuAAuAACUUcAUCcATsT | 1025 | AD-14306 | 22% | 4% |
| AucuAcAuGAAcuAcAAGATsT | 1026 | UCUUGuAGUUcAUGuAGAUTsT | 1027 | AD-14307 | 26% | 6% |
| GGuAuuuuuGAucuGGcAATsT | 1028 | UUGCcAGAUcAAAAAuACCTsT | 1029 | AD-14308 | 62% | 8% |
| cuAAuGAAGAGuAuAccuGTsT | 1030 | cAGGuAuACUCUUcAUuAGTsT | 1031 | AD-14309 | 52% | 5% |
| uuuGAGAAAcuuAcuGAuATsT | 1032 | uAUcAGuAAGUUUCUcAAATsT | 1033 | AD-14310 | 32% | 3% |
| cGAuAAGAuAGAAGAucAATsT | 1034 | UUGAUCUUCuAUCUuAUCGTsT | 1035 | AD-14311 | 23% | 2% |
| cuGGcAAccAuAuuucuGGTsT | 1036 | CcAGAAAuAUGGUUGCcAGTsT | 1037 | AD-14312 | 49% | 6% |
| uAGAuAccAuuAcuAcAGuTsT | 1038 | ACUGuAGuAAUGGuAUCuATsT | 1039 | AD-14313 | 69% | 4% |
| GuAuuAAAuuGGGuuucAuTsT | 1040 | AUGAAACCcAAUUuAAuACTsT | 1041 | AD-14314 | 52% | 3% |
| AAGAccuuAuuuGGuAAucTsT | 1042 | GAUuACcAAAuAAGGUCUUTsT | 1043 | AD-14315 | 66% | 4% |
| GcuGuuGAuAAGAGAGcucTsT | 1044 | GAGCUCUCUuAUcAAcAGCTsT | 1045 | AD-14316 | 19% | 4% |
| uAcucAuGuuucucAGAuuTsT | 1046 | AAUCUGAGAAAcAUGAGuATsT | 1047 | AD-14317 | 16% | 5% |
| cAGAuGGAcGuAAGGcAGcTsT | 1048 | GCUGCCUuACGUCcAUCUGTsT | 1049 | AD-14318 | 52% | 11% |
| uAucccAAcAGGuAcGAcATsT | 1050 | UGUCGuACCUGUUGGGAuATsT | 1051 | AD-14319 | 28% | 11% |
| cAuuGcuAuuAuGGGAGAcTsT | 1052 | GUCUCCcAuAAuAGcAAUGTsT | 1053 | AD-14320 | 52% | 10% |
| cccucAGuAAAuccAuGGuTsT | 1054 | ACcAUGGAUUuACUGAGGGTsT | 1055 | AD-14321 | 53% | 6% |
| GGucAuuAcuGcccuuGuATsT | 1056 | uAcAAGGGcAGuAAUGACCTsT | 1057 | AD-14322 | 20% | 2% |
| AAccAcucAAAAAcAuuuGTsT | 1058 | cAAAUGUUUUUGAGUGGUUTsT | 1059 | AD-14323 | 116% | 6% |
| uuuGcAAGuuAAuGAAucuTsT | 1060 | AGAUUcAUuAACUUGcAAATsT | 1061 | AD-14324 | 14% | 2% |
| uuAuuuucAGuAGucAGAATsT | 1062 | UUCUGACuACUGAAAAuAATsT | 1063 | AD-14325 | 50% | 2% |
| uuuucucGAuucAAAucuuTsT | 1064 | AAGAUUuGAAUCGAGAAAATsT | 1065 | AD-14326 | 47% | 3% |
| GuAcGAAAAGAAGuuAGuGTsT | 1066 | cACuAACUUCUUUUCGuACTsT | 1067 | AD-14327 | 18% | 2% |
| uuuAAAAcGAGAucuuGcuTsT | 1068 | AGcAAGAUCUCGUUUuAAATsT | 1069 | AD-14328 | 19% | 1% |
| GAAuuGAuuAAuGuAcucATsT | 1070 | UGAGuAcAUuAAUcAAUUCTsT | 1071 | AD-14329 | 94% | 10% |
| GAuGGAcGuAAGGcAGcucTsT | 1072 | GAGCUGCCUuACGUCcAUCTsT | 1073 | AD-14330 | 60% | 4% |
| cAucuGAcuAAuGGcucuGTsT | 1074 | cAGAGCcAUuAGUcAGAUGTsT | 1075 | AD-14331 | 54% | 7% |
| GuGAuccuGuAcGAAAAGATsT | 1076 | UCUUUUCGuAcAGGAUcACTsT | 1077 | AD-14332 | 22% | 4% |
| AGcucuuAuuAAGGAGuAuTsT | 1078 | AuACUCCUuAAuAAGAGCUTsT | 1079 | AD-14333 | 70% | 10% |
| GcucuuAuuAAGGAGuAuATsT | 1080 | uAuACUCCUuAAuAAGAGCTsT | 1081 | AD-14334 | 18% | 3% |
| ucuuAuuAAGGAGuAuAcGTsT | 1082 | CGuAuACUCCUuAAuAAGATsT | 1083 | AD-14335 | 38% | 6% |
| uAuuAAGGAGuAuAcGGAGTsT | 1084 | CUCCGuAuACUCCUuAAuATsT | 1085 | AD-14336 | 16% | 3% |
| cuGcAGcccGuGAGAAAAATsT | 1086 | UUUUUCUcACGGGCUGcAGTsT | 1087 | AD-14337 | 65% | 4% |
| ucAAGAcuGAucuucuAAGTsT | 1088 | CUuAGAAGAUcAGUCUUGATsT | 1089 | AD-14338 | 18% | 0% |
| cuucuAAGuucAcuGGAAATsT | 1090 | UUUCcAGUGAACUuAGAAGTsT | 1091 | AD-14339 | 20% | 4% |
| uGcAAGuuAAuGAAucuuuTsT | 1092 | AAAGAUUcAUuAACUUGcATsT | 1093 | AD-14340 | 24% | 1% |
| AAucuAAGGAuAuAGucAATsT | 1094 | UUGACuAuAUCCUuAGAUUTsT | 1095 | AD-14341 | 27% | 3% |
| AucucuGAAcAcAAGAAcATsT | 1096 | UGUUCUUGUGUUcAGAGAUTsT | 1097 | AD-14342 | 13% | 1% |
| uucuGAAcAGuGGGuAucuTsT | 1098 | AGAuACCcACUGUUcAGAATsT | 1099 | AD-14343 | 19% | 1% |
| AGuuAuuuAuAcccAucAATsT | 1100 | UUGAUGGGuAuAAAuAACUTsT | 1101 | AD-14344 | 23% | 2% |
| AuGcuAAAcuGuucAGAAATsT | 1102 | UUUCUGAAcAGUUuAGcAUTsT | 1103 | AD-14345 | 21% | 4% |
| cuAcAGAGcAcuuGGuuAcTsT | 1104 | GuAACcAAGUGCUCUGuAGTsT | 1105 | AD-14346 | 18% | 2% |
| uAuAuAucAGccGGGcGcGTsT | 1106 | CGCGCCCGGCUGAuAuAuATsT | 1107 | AD-14347 | 67% | 2% |
| AuGuAAAuAcGuAuuucuATsT | 1108 | uAGAAAuACGuAUUuAcAUTsT | 1109 | AD-14348 | 39% | 3% |
| uuuuucucGAuucAAAucuTsT | 1110 | AGAUUuGAAUCGAGAAAAATsT | 1111 | AD-14349 | 83% | 6% |
| AAucuuAAcccuuAGGAcuTsT | 1112 | AGUCCuAAGGGUuAAGAUUTsT | 1113 | AD-14350 | 54% | 2% |
| ccuuAGGAcucuGGuAuuuTsT | 1114 | AAAuACcAGAGUCCuAAGGTsT | 1115 | AD-14351 | 57% | 8% |
| AAuAAAcuGcccucAGuAATsT | 1116 | UuACUGAGGGcAGUUuAUUTsT | 1117 | AD-14352 | 82% | 3% |
| GAuccuGuAcGAAAAGAAGTsT | 1118 | CUUCUUUUCGuAcAGGAUCTsT | 1119 | AD-14353 | 2% | 1% |
| AAuGuGAuccuGuAcGAAATsT | 1120 | UUUCGuAcAGGAUcAcAUUTsT | 1121 | AD-14354 | 18% | 11% |
| GuGAAAAcAuuGGccGuucTsT | 1122 | GAACGGCcAAUGUUUUcACTsT | 1123 | AD-14355 | 2% | 1% |
| cuuGAGGAAAcucuGAGuATsT | 1124 | uACUcAGAGUUUCCUcAAGTsT | 1125 | AD-14356 | 8% | 2% |
| cGuuuAAAAcGAGAucuuGTsT | 1126 | cAAGAUCUCGUUUuAAACGTsT | 1127 | AD-14357 | 6% | 3% |
| uuAAAAcGAGAucuuGcuGTsT | 1128 | cAGcAAGAUCUCGUUUuAATsT | 1129 | AD-14358 | 98% | 17% |
| AAAGAuGuAucuGGucuccTsT | 1130 | GGAGACcAGAuAcAUCUUUTsT | 1131 | AD-14359 | 10% | 1% |
| cAGAAAAuGuGucuAcucATsT | 1132 | UGAGuAGAcAcAUUUUCUGTsT | 1133 | AD-14360 | 6% | 4% |
| cAGGAAuuGAuuAAuGuAcTsT | 1134 | GuAcAUuAAUcAAUUCCUGTsT | 1135 | AD-14361 | 30% | 5% |
| AGucAAcuAAAGcAuAuuuTsT | 1136 | AAAuAUGCUUuAGUUGACUTsT | 1137 | AD-14362 | 28% | 2% |
| uGuGuAAcAAucuAcAuGATsT | 1138 | UcAUGuAGAUUGUuAcAcATsT | 1139 | AD-14363 | 60% | 6% |
| AuAccAuuuGuuccuuGGuTsT | 1140 | ACcAAGGAAcAAAUGGuAUTsT | 1141 | AD-14364 | 12% | 9% |
| GcAGAAAucuAAGGAuAuATsT | 1142 | uAuAUCCUuAGAUUUCUGCTsT | 1143 | AD-14365 | 5% | 2% |
| uGGcuucucAcAGGAAcucTsT | 1144 | GAGUUCCUGUGAGAAGCcATsT | 1145 | AD-14366 | 28% | 5% |
| GAGAuGuGAAucucuGAAcTsT | 1146 | GUUcAGAGAUUcAcAUCUCTsT | 1147 | AD-14367 | 42% | 4% |
| uGuAAGccAAuGuuGuGAGTsT | 1148 | CUcAcAAcAUUGGCUuAcATsT | 1149 | AD-14368 | 93% | 12% |
| AGccAAuGuuGuGAGGcuuTsT | 1150 | AAGCCUcAcAAcAUUGGCUTsT | 1151 | AD-14369 | 65% | 4% |
| uuGuGAGGcuucAAGuucATsT | 1152 | UGAACUUGAAGCCUcAcAATsT | 1153 | AD-14370 | 5% | 2% |
| AGGcAGcucAuGAGAAAcATsT | 1154 | UGUUUCUcAUGAGCUGCCUTsT | 1155 | AD-14371 | 54% | 5% |
| AuAAAuuGAuAGcAcAAAATsT | 1156 | UUUUGUGCuAUcAAUUuAUTsT | 1157 | AD-14372 | 4% | 1% |
| AcAAAAucuAGAAcuuAAuTsT | 1158 | AUuAAGUUCuAGAUUUUGUTsT | 1159 | AD-14373 | 5% | 1% |
| GAuAucccAAcAGGuAcGATsT | 1160 | UCGuACCUGUUGGGAuAUCTsT | 1161 | AD-14374 | 92% | 6% |
| AAGuuAuuuAuAcccAucATsT | 1162 | UGAUGGGuAuAAAuAACUUTsT | 1163 | AD-14375 | 76% | 4% |
| uGuAAAuAcGuAuuucuAGTsT | 1164 | CuAGAAAuACGuAUUuAcATsT | 1165 | AD-14376 | 70% | 5% |
| ucuAGuuuucAuAuAAAGuTsT | 1166 | ACUUuAuAUGAAAACuAGATsT | 1167 | AD-14377 | 48% | 4% |
| AuAAAGuAGuucuuuuAuATsT | 1168 | uAuAAAAGAACuACUUuAUTsT | 1169 | AD-14378 | 48% | 3% |
| ccAuuuGuAGAGcuAcAAATsT | 1170 | UUUGuAGCUCuAcAAAUGGTsT | 1171 | AD-14379 | 44% | 5% |
| uAuuuucAGuAGucAGAAuTsT | 1172 | AUUCUGACuACUGAAAAuATsT | 1173 | AD-14380 | 35% | 16% |
| AAAucuAAcccuAGuuGuATsT | 1174 | uAcAACuAGGGUuAGAUUUTsT | 1175 | AD-14381 | 44% | 5% |
| cuuuAGAGuAuAcAuuGcuTsT | 1176 | AGcAAUGuAuACUCuAAAGTsT | 1177 | AD-14382 | 28% | 1% |
| AucuGAcuAAuGGcucuGuTsT | 1178 | AcAGAGCcAUuAGUcAGAUTsT | 1179 | AD-14383 | 55% | 11% |
| cAcAAuGAuuuAAGGAcuGTsT | 1180 | cAGUCCUuAAAUcAUUGUGTsT | 1181 | AD-14384 | 48% | 9% |
| ucuuuuucucGAuucAAAuTsT | 1182 | AUUuGAAUCGAGAAAAAGATsT | 1183 | AD-14385 | 36% | 2% |
| cuuuuucucGAuucAAAucTsT | 1184 | GAUUuGAAUCGAGAAAAAGTsT | 1185 | AD-14386 | 41% | 7% |
| AuuuucuGcucAcGAuGAGTsT | 1186 | CUcAUCGUGAGcAGAAAAUTsT | 1187 | AD-14387 | 38% | 3% |
| uuucuGcucAcGAuGAGuuTsT | 1188 | AACUcAUCGUGAGcAGAAATsT | 1189 | AD-14388 | 50% | 4% |
| AGAGcuAcAAAAccuAuccTsT | 1190 | GGAuAGGUUUUGuAGCUCUTsT | 1191 | AD-14389 | 98% | 6% |
| GAGccAAAGGuAcAccAcuTsT | 1192 | AGUGGUGuACCUUUGGCUCTsT | 1193 | AD-14390 | 43% | 8% |
| GccAAAGGuAcAccAcuAcTsT | 1194 | GuAGUGGUGuACCUUUGGCTsT | 1195 | AD-14391 | 48% | 4% |
| GAAcuGuAcucuucucAGcTsT | 1196 | GCUGAGAAGAGuAcAGUUCTsT | 1197 | AD-14392 | 44% | 3% |
| AGGuAAAuAucAccAAcAuTsT | 1198 | AUGUUGGUGAuAUUuACCUTsT | 1199 | AD-14393 | 37% | 2% |
| AGcuAcAAAAccuAuccuuTsT | 1200 | AAGGAuAGGUUUUGuAGCUTsT | 1201 | AD-14394 | 114% | 7% |
| uGuGAAAGcAuuuAAuuccTsT | 1202 | GGAAUuAAAUGCUUUcAcATsT | 1203 | AD-14395 | 55% | 4% |
| GcccAcuuuAGAGuAuAcATsT | 1204 | UGuAuACUCuAAAGUGGGCTsT | 1205 | AD-14396 | 49% | 5% |
| uGuGccAcAcuccAAGAccTsT | 1206 | GGUCUUGGAGUGUGGcAcATsT | 1207 | AD-14397 | 71% | 6% |
| AAAcuAAAuuGAucucGuATsT | 1208 | uACGAGAUcAAUUuAGUUUTsT | 1209 | AD-14398 | 81% | 7% |
| uGAucucGuAGAAuuAucuTsT | 1210 | AGAuAAUUCuACGAGAUcATsT | 1211 | AD-14399 | 38% | 4% |
| GcGuGcAGucGGuccuccATsT | 1212 | UGGAGGACCGACUGcACGCTsT | 1213 | AD-14400 | 106% | 8% |
| AAAGuuuAGAGAcAucuGATsT | 1214 | UcAGAUGUCUCuAAACUUUTsT | 1215 | AD-14401 | 47% | 3% |
| cAGAAGGAAuAuGuAcAAATsT | 1216 | UUUGuAcAuAUUCCUUCUGTsT | 1217 | AD-14402 | 31% | 1% |
| cGcccGAGAGuAccAGGGATsT | 1218 | UCCCUGGuACUCUCGGGCGTsT | 1219 | AD-14403 | 105% | 4% |
| cGGAGGAGAuAGAAcGuuuTsT | 1220 | AAACGUUCuAUCUCCUCCGTsT | 1221 | AD-14404 | 3% | 1% |
| AGAuAGAAcGuuuAAAAcGTsT | 1222 | CGUUUuAAACGUUCuAUCUTsT | 1223 | AD-14405 | 15% | 1% |
| GGAAcAGGAAcuucAcAAcTsT | 1224 | GUuGuGAAGUUCCuGUUCCTsT | 1225 | AD-14406 | 44% | 5% |
| GuGAGccAAAGGuAcAccATsT | 1226 | UGGUGuACCUUUGGCUcACTsT | 1227 | AD-14407 | 41% | 4% |
| AuccucccuAGAcuucccuTsT | 1228 | AGGGAAGUCuAGGGAGGAUTsT | 1229 | AD-14408 | 104% | 3% |
| cAcAcuccAAGAccuGuGcTsT | 1230 | GcAcAGGUCUUGGAGUGUGTsT | 1231 | AD-14409 | 67% | 4% |
| AcAGAAGGAAuAuGuAcAATsT | 1232 | UUGuAcAuAUUCCUUCUGUTsT | 1233 | AD-14410 | 22% | 1% |
| uuAGAGAcAucuGAcuuuGTsT | 1234 | cAAAGUcAGAUGUCUCuAATsT | 1235 | AD-14411 | 29% | 3% |
| AAuuGAucucGuAGAAuuATsT | 1236 | uAAUUCuACGAGAUcAAUUTsT | 1237 | AD-14412 | 31% | 4% |

dsRNA Targeting the VEGF Gene

Four hundred target sequences were identified within exons 1-5 of the VEGF-A121 mRNA sequence. Reference transcript is: NM_{—}003376.

| (SEQ ID NO: 1539) | |
|---|---|
| 1 | augaacuuuc ugcugucuug ggugcauugg agccuugccu ugcugcucua ccuccaccau |
| 61 | gccaaguggu cccaggcugc acccauggca gaaggaggag ggcagaauca ucacgaagug |
| 121 | gugaaguuca uggaugucua ucagcgcagc uacugccauc caaucgagac ccugguggac |
| 181 | aucuuccagg aguacccuga ugagaucgag uacaucuuca agccauccug ugugccccug |
| 241 | augcgaugcg ggggcugcug caaugacgag ggccuggagu gugugcccac ugaggagucc |
| 301 | aacaucacca ugcagauuau gcggaucaaa ccucaccaag gccagcacau aggagagaug |
| 361 | agcuuccuac agcacaacaa augugaaugc agaccaaaga aagauagagc aagacaagaa |
| 421 | aaaugugaca agccgaggcg guga |

Table 4a includes the identified target sequences. Corresponding siRNAs targeting these sequences were subjected to a bioinformatics screen.

To ensure that the sequences were specific to VEGF sequence and not to sequences from any other genes, the target sequences were checked against the sequences in Genbank using the BLAST search engine provided by NCBI. The use of the BLAST algorithm is described in Altschul et al., J. Mol. Biol. 215:403, 1990; and Altschul and Gish, Meth. Enzymol. 266:460, 1996.

siRNAs were also prioritized for their ability to cross react with monkey, rat and human VEGF sequences.

Of these 400 potential target sequences 80 were selected for analysis by experimental screening in order to identify a small number of lead candidates. A total of 114 siRNA molecules were designed for these 80 target sequences 114 (Table 4b).

TABLE 4a
Target sequences in VEGF-121

| | position | TARGET SEQUENCE IN |
|---|---|---|
| SEQ ID | in VEGF- | VEGF121 mRNA |
| NO: | 121 ORF | 5′ to 3′ |
| 1540 | 1 | AUGAACUUUCUGCUGUCUUGGGU |
| 1541 | 2 | UGAACUUUCUGCUGUCUUGGGUG |
| 1542 | 3 | GAACUUUCUGCUGUCUUGGGUGC |
| 1543 | 4 | AACUUUCUGCUGUCUUGGGUGCA |
| 1544 | 5 | ACUUUCUGCUGUCUUGGGUGCAU |
| 1545 | 6 | CUUUCUGCUGUCUUGGGUGCAUU |
| 1546 | 7 | UUUCUGCUGUCUUGGGUGCAUUG |
| 1547 | 8 | UUCUGCUGUCUUGGGUGCAUUGG |
| 1548 | 9 | UCUGCUGUCUUGGGUGCAUUGGA |
| 1549 | 10 | CUGCUGUCUUGGGUGCAUUGGAG |
| 1550 | 11 | UGCUGUCUUGGGUGCAUUGGAGC |
| 1551 | 12 | GCUGUCUUGGGUGCAUUGGAGCC |
| 1552 | 13 | CUGUCUUGGGUGCAUUGGAGCCU |
| 1553 | 14 | UGUCUUGGGUGCAUUGGAGCCUU |
| 1554 | 15 | GUCUUGGGUGCAUUGGAGCCUUG |
| 1555 | 16 | UCUUGGGUGCAUUGGAGCCUUGC |
| 1556 | 17 | CUUGGGUGCAUUGGAGCCUUGCC |
| 1557 | 18 | UUGGGUGCAUUGGAGCCUUGCCU |
| 1558 | 19 | UGGGUGCAUUGGAGCCUUGCCUU |
| 1559 | 20 | GGGUGCAUUGGAGCCUUGCCUUG |
| 1560 | 21 | GGUGCAUUGGAGCCUUGCCUUGC |
| 1561 | 22 | GUGCAUUGGAGCCUUGCCUUGCU |
| 1562 | 23 | UGCAUUGGAGCCUUGCCUUGCUG |
| 1563 | 24 | GCAUUGGAGCCUUGCCUUGCUGC |
| 1564 | 25 | CAUUGGAGCCUUGCCUUGCUGCU |
| 1565 | 26 | AUUGGAGCCUUGCCUUGCUGCUC |
| 1566 | 27 | UUGGAGCCUUGCCUUGCUGCUCU |
| 1567 | 28 | UGGAGCCUUGCCUUGCUGCUCUA |
| 1568 | 29 | GGAGCCUUGCCUUGCUGCUCUAC |
| 1569 | 30 | GAGCCUUGCCUUGCUGCUCUACC |
| 1570 | 31 | AGCCUUGCCUUGCUGCUCUACCU |
| 1571 | 32 | GCCUUGCCUUGCUGCUCUACCUC |
| 1572 | 33 | CCUUGCCUUGCUGCUCUACCUCC |
| 1573 | 34 | CUUGCCUUGCUGCUCUACCUCCA |
| 1574 | 35 | UUGCCUUGCUGCUCUACCUCCAC |
| 1575 | 36 | UGCCUUGCUGCUCUACCUCCACC |
| 1576 | 37 | GCCUUGCUGCUCUACCUCCACCA |
| 1577 | 38 | CCUUGCUGCUCUACCUCCACCAU |
| 1578 | 39 | CUUGCUGCUCUACCUCCACCAUG |
| 1579 | 40 | UUGCUGCUCUACCUCCACCAUGC |
| 1580 | 41 | UGCUGCUCUACCUCCACCAUGCC |
| 1581 | 42 | GCUGCUCUACCUCCACCAUGCCA |
| 1582 | 43 | CUGCUCUACCUCCACCAUGCCAA |
| 1583 | 44 | UGCUCUACCUCCACCAUGCCAAG |
| 1584 | 45 | GCUCUACCUCCACCAUGCCAAGU |
| 1585 | 46 | CUCUACCUCCACCAUGCCAAGUG |
| 1586 | 47 | UCUACCUCCACCAUGCCAAGUGG |
| 1587 | 48 | CUACCUCCACCAUGCCAAGUGGU |
| 1588 | 49 | UACCUCCACCAUGCCAAGUGGUC |
| 1589 | 50 | ACCUCCACCAUGCCAAGUGGUCC |
| 1590 | 51 | CCUCCACCAUGCCAAGUGGUCCC |
| 1591 | 52 | CUCCACCAUGCCAAGUGGUCCCA |
| 1592 | 53 | UCCACCAUGCCAAGUGGUCCCAG |
| 1593 | 54 | CCACCAUGCCAAGUGGUCCCAGG |
| 1594 | 55 | CACCAUGCCAAGUGGUCCCAGGC |
| 1595 | 56 | ACCAUGCCAAGUGGUCCCAGGCU |
| 1596 | 57 | CCAUGCCAAGUGGUCCCAGGCUG |
| 1597 | 58 | CAUGCCAAGUGGUCCCAGGCUGC |
| 1598 | 59 | AUGCCAAGUGGUCCCAGGCUGCA |
| 1599 | 60 | UGCCAAGUGGUCCCAGGCUGCAC |
| 1600 | 61 | GCCAAGUGGUCCCAGGCUGCACC |
| 1601 | 62 | CCAAGUGGUCCCAGGCUGCACCC |
| 1602 | 63 | CAAGUGGUCCCAGGCUGCACCCA |
| 1603 | 64 | AAGUGGUCCCAGGCUGCACCCAU |
| 1604 | 65 | AGUGGUCCCAGGCUGCACCCAUG |
| 1605 | 66 | GUGGUCCCAGGCUGCACCCAUGG |
| 1606 | 67 | UGGUCCCAGGCUGCACCCAUGGC |
| 1607 | 68 | GGUCCCAGGCUGCACCCAUGGCA |
| 1608 | 69 | GUCCCAGGCUGCACCCAUGGCAG |
| 1609 | 70 | UCCCAGGCUGCACCCAUGGCAGA |
| 1610 | 71 | CCCAGGCUGCACCCAUGGCAGAA |
| 1611 | 72 | CCAGGCUGCACCCAUGGCAGAAG |
| 1612 | 73 | CAGGCUGCACCCAUGGCAGAAGG |
| 1613 | 74 | AGGCUGCACCCAUGGCAGAAGGA |
| 1614 | 75 | GGCUGCACCCAUGGCAGAAGGAG |
| 1615 | 76 | GCUGCACCCAUGGCAGAAGGAGG |
| 1616 | 77 | CUGCACCCAUGGCAGAAGGAGGA |
| 1617 | 78 | UGCACCCAUGGCAGAAGGAGGAG |
| 1618 | 79 | GCACCCAUGGCAGAAGGAGGAGG |
| 1619 | 80 | CACCCAUGGCAGAAGGAGGAGGG |
| 1620 | 81 | ACCCAUGGCAGAAGGAGGAGGGC |
| 1621 | 82 | CCCAUGGCAGAAGGAGGAGGGCA |
| 1622 | 83 | CCAUGGCAGAAGGAGGAGGGCAG |
| 1623 | 84 | CAUGGCAGAAGGAGGAGGGCAGA |
| 1624 | 85 | AUGGCAGAAGGAGGAGGGCAGAA |
| 1625 | 86 | UGGCAGAAGGAGGAGGGCAGAAU |
| 1626 | 87 | GGCAGAAGGAGGAGGGCAGAAUC |
| 1627 | 88 | GCAGAAGGAGGAGGGCAGAAUCA |
| 1628 | 89 | CAGAAGGAGGAGGGCAGAAUCAU |
| 1629 | 90 | AGAAGGAGGAGGGCAGAAUCAUC |
| 1630 | 91 | GAAGGAGGAGGGCAGAAUCAUCA |
| 1631 | 92 | AAGGAGGAGGGCAGAAUCAUCAC |
| 1632 | 93 | AGGAGGAGGGCAGAAUCAUCACG |
| 1633 | 94 | GGAGGAGGGCAGAAUCAUCACGA |
| 1634 | 95 | GAGGAGGGCAGAAUCAUCACGAA |
| 1635 | 96 | AGGAGGGCAGAAUCAUCACGAAG |
| 1636 | 97 | GGAGGGCAGAAUCAUCACGAAGU |
| 1637 | 98 | GAGGGCAGAAUCAUCACGAAGUG |
| 1638 | 99 | AGGGCAGAAUCAUCACGAAGUGG |
| 1639 | 100 | GGGCAGAAUCAUCACGAAGUGGU |
| 1640 | 101 | GGCAGAAUCAUCACGAAGUGGUG |
| 1641 | 102 | GCAGAAUCAUCACGAAGUGGUGA |
| 1642 | 103 | CAGAAUCAUCACGAAGUGGUGAA |
| 1643 | 104 | AGAAUCAUCACGAAGUGGUGAAG |
| 1644 | 105 | GAAUCAUCACGAAGUGGUGAAGU |
| 1645 | 106 | AAUCAUCACGAAGUGGUGAAGUU |
| 1646 | 107 | AUCAUCACGAAGUGGUGAAGUUC |
| 1647 | 108 | UCAUCACGAAGUGGUGAAGUUCA |
| 1648 | 109 | CAUCACGAAGUGGUGAAGUUCAU |
| 1649 | 110 | AUCACGAAGUGGUGAAGUUCAUG |
| 1650 | 111 | UCACGAAGUGGUGAAGUUCAUGG |
| 1651 | 112 | CACGAAGUGGUGAAGUUCAUGGA |
| 1652 | 113 | ACGAAGUGGUGAAGUUCAUGGAU |
| 1653 | 114 | CGAAGUGGUGAAGUUCAUGGAUG |
| 1654 | 115 | GAAGUGGUGAAGUUCAUGGAUGU |
| 1655 | 116 | AAGUGGUGAAGUUCAUGGAUGUC |
| 1656 | 117 | AGUGGUGAAGUUCAUGGAUGUCU |
| 1657 | 118 | GUGGUGAAGUUCAUGGAUGUCUA |
| 1658 | 119 | UGGUGAAGUUCAUGGAUGUCUAU |
| 1659 | 120 | GGUGAAGUUCAUGGAUGUCUAUC |
| 1660 | 121 | GUGAAGUUCAUGGAUGUCUAUCA |
| 1661 | 122 | UGAAGUUCAUGGAUGUCUAUCAG |
| 1662 | 123 | GAAGUUCAUGGAUGUCUAUCAGC |
| 1663 | 124 | AAGUUCAUGGAUGUCUAUCAGCG |
| 1664 | 125 | AGUUCAUGGAUGUCUAUCAGCGC |
| 1665 | 126 | GUUCAUGGAUGUCUAUCAGCGCA |
| 1666 | 127 | UUCAUGGAUGUCUAUCAGCGCAG |
| 1667 | 128 | UCAUGGAUGUCUAUCAGCGCAGC |
| 1668 | 129 | CAUGGAUGUCUAUCAGCGCAGCU |
| 1669 | 130 | AUGGAUGUCUAUCAGCGCAGCUA |
| 1670 | 131 | UGGAUGUCUAUCAGCGCAGCUAC |
| 1671 | 132 | GGAUGUCUAUCAGCGCAGCUACU |
| 1672 | 133 | GAUGUCUAUCAGCGCAGCUACUG |
| 1673 | 134 | AUGUCUAUCAGCGCAGCUACUGC |
| 1674 | 135 | UGUCUAUCAGCGCAGCUACUGCC |
| 1675 | 136 | GUCUAUCAGCGCAGCUACUGCCA |
| 1676 | 137 | UCUAUCAGCGCAGCUACUGCCAU |
| 1677 | 138 | CUAUCAGCGCAGCUACUGCCAUC |
| 1678 | 139 | UAUCAGCGCAGCUACUGCCAUCC |
| 1679 | 140 | AUCAGCGCAGCUACUGCCAUCCA |
| 1680 | 141 | UCAGCGCAGCUACUGCCAUCCAA |
| 1681 | 142 | CAGCGCAGCUACUGCCAUCCAAU |
| 1682 | 143 | AGCGCAGCUACUGCCAUCCAAUC |
| 1683 | 144 | GCGCAGCUACUGCCAUCCAAUCG |
| 1684 | 145 | CGCAGCUACUGCCAUCCAAUCGA |
| 1685 | 146 | GCAGCUACUGCCAUCCAAUCGAG |
| 1686 | 147 | CAGCUACUGCCAUCCAAUCGAGA |
| 1687 | 148 | AGCUACUGCCAUCCAAUCGAGAC |
| 1688 | 149 | GCUACUGCCAUCCAAUCGAGACC |
| 1689 | 150 | CUACUGCCAUCCAAUCGAGACCC |
| 1690 | 151 | UACUGCCAUCCAAUCGAGACCCU |
| 1691 | 152 | ACUGCCAUCCAAUCGAGACCCUG |
| 1692 | 153 | CUGCCAUCCAAUCGAGACCCUGG |
| 1693 | 154 | UGCCAUCCAAUCGAGACCCUGGU |
| 1694 | 155 | GCCAUCCAAUCGAGACCCUGGUG |
| 1695 | 156 | CCAUCCAAUCGAGACCCUGGUGG |
| 1696 | 157 | CAUCCAAUCGAGACCCUGGUGGA |
| 1697 | 158 | AUCCAAUCGAGACCCUGGUGGAC |
| 1698 | 159 | UCCAAUCGAGACCCUGGUGGACA |
| 1699 | 160 | CCAAUCGAGACCCUGGUGGACAU |
| 1700 | 161 | CAAUCGAGACCCUGGUGGACAUC |
| 1701 | 162 | AAUCGAGACCCUGGUGGACAUCU |
| 1702 | 163 | AUCGAGACCCUGGUGGACAUCUU |
| 1703 | 164 | UCGAGACCCUGGUGGACAUCUUC |
| 1704 | 165 | CGAGACCCUGGUGGACAUCUUCC |
| 1705 | 166 | GAGACCCUGGUGGACAUCUUCCA |
| 1706 | 167 | AGACCCUGGUGGACAUCUUCCAG |
| 1707 | 168 | GACCCUGGUGGACAUCUUCCAGG |
| 1708 | 169 | ACCCUGGUGGACAUCUUCCAGGA |
| 1709 | 170 | CCCUGGUGGACAUCUUCCAGGAG |
| 1710 | 171 | CCUGGUGGACAUCUUCCAGGAGU |
| 1711 | 172 | CUGGUGGACAUCUUCCAGGAGUA |
| 1712 | 173 | UGGUGGACAUCUUCCAGGAGUAC |
| 1713 | 174 | GGUGGACAUCUUCCAGGAGUACC |
| 1714 | 175 | GUGGACAUCUUCCAGGAGUACCC |
| 1715 | 176 | UGGACAUCUUCCAGGAGUACCCU |
| 1716 | 177 | GGACAUCUUCCAGGAGUACCCUG |
| 1717 | 178 | GACAUCUUCCAGGAGUACCCUGA |
| 1718 | 179 | ACAUCUUCCAGGAGUACCCUGAU |
| 1719 | 180 | CAUCUUCCAGGAGUACCCUGAUG |
| 1720 | 181 | AUCUUCCAGGAGUACCCUGAUGA |
| 1721 | 182 | UCUUCCAGGAGUACCCUGAUGAG |
| 1722 | 183 | CUUCCAGGAGUACCCUGAUGAGA |
| 1723 | 184 | UUCCAGGAGUACCCUGAUGAGAU |
| 1724 | 185 | UCCAGGAGUACCCUGAUGAGAUC |
| 1725 | 186 | CCAGGAGUACCCUGAUGAGAUCG |
| 1726 | 187 | CAGGAGUACCCUGAUGAGAUCGA |
| 1727 | 188 | AGGAGUACCCUGAUGAGAUCGAG |
| 1728 | 189 | GGAGUACCCUGAUGAGAUCGAGU |
| 1729 | 190 | GAGUACCCUGAUGAGAUCGAGUA |
| 1730 | 191 | AGUACCCUGAUGAGAUCGAGUAC |
| 1731 | 192 | GUACCCUGAUGAGAUCGAGUACA |
| 1732 | 193 | UACCCUGAUGAGAUCGAGUACAU |
| 1733 | 194 | ACCCUGAUGAGAUCGAGUACAUC |
| 1734 | 195 | CCCUGAUGAGAUCGAGUACAUCU |
| 1735 | 196 | CCUGAUGAGAUCGAGUACAUCUU |
| 1736 | 197 | CUGAUGAGAUCGAGUACAUCUUC |
| 1737 | 198 | UGAUGAGAUCGAGUACAUCUUCA |
| 1738 | 199 | GAUGAGAUCGAGUACAUCUUCAA |
| 1739 | 200 | AUGAGAUCGAGUACAUCUUCAAG |
| 1740 | 201 | UGAGAUCGAGUACAUCUUCAAGC |
| 1741 | 202 | GAGAUCGAGUACAUCUUCAAGCC |
| 1742 | 203 | AGAUCGAGUACAUCUUCAAGCCA |
| 1743 | 204 | GAUCGAGUACAUCUUCAAGCCAU |
| 1744 | 205 | AUCGAGUACAUCUUCAAGCCAUC |
| 1745 | 206 | UCGAGUACAUCUUCAAGCCAUCC |
| 1746 | 207 | CGAGUACAUCUUCAAGCCAUCCU |
| 1747 | 208 | GAGUACAUCUUCAAGCCAUCCUG |
| 1748 | 209 | AGUACAUCUUCAAGCCAUCCUGU |
| 1749 | 210 | GUACAUCUUCAAGCCAUCCUGUG |
| 1750 | 211 | UACAUCUUCAAGCCAUCCUGUGU |
| 1751 | 212 | ACAUCUUCAAGCCAUCCUGUGUG |
| 1752 | 213 | CAUCUUCAAGCCAUCCUGUGUGC |
| 1753 | 214 | AUCUUCAAGCCAUCCUGUGUGCC |
| 1754 | 215 | UCUUCAAGCCAUCCUGUGUGCCC |
| 1755 | 216 | CUUCAAGCCAUCCUGUGUGCCCC |
| 1756 | 217 | UUCAAGCCAUCCUGUGUGCCCCU |
| 1757 | 218 | UCAAGCCAUCCUGUGUGCCCCUG |
| 1758 | 219 | CAAGCCAUCCUGUGUGCCCCUGA |
| 1759 | 220 | AAGCCAUCCUGUGUGCCCCUGAU |
| 1760 | 221 | AGCCAUCCUGUGUGCCCCUGAUG |
| 1761 | 222 | GCCAUCCUGUGUGCCCCUGAUGC |
| 1762 | 223 | CCAUCCUGUGUGCCCCUGAUGCG |
| 1763 | 224 | CAUCCUGUGUGCCCCUGAUGCGA |
| 1764 | 225 | AUCCUGUGUGCCCCUGAUGCGAU |
| 1765 | 226 | UCCUGUGUGCCCCUGAUGCGAUG |
| 1766 | 227 | CCUGUGUGCCCCUGAUGCGAUGC |
| 1767 | 228 | CUGUGUGCCCCUGAUGCGAUGCG |
| 1768 | 229 | UGUGUGCCCCUGAUGCGAUGCGG |
| 1769 | 230 | GUGUGCCCCUGAUGCGAUGCGGG |
| 1770 | 231 | UGUGCCCCUGAUGCGAUGCGGGG |
| 1771 | 232 | GUGCCCCUGAUGCGAUGCGGGGG |
| 1772 | 233 | UGCCCCUGAUGCGAUGCGGGGGC |
| 1773 | 234 | GCCCCUGAUGCGAUGCGGGGGCU |
| 1774 | 235 | CCCCUGAUGCGAUGCGGGGGCUG |
| 1775 | 236 | CCCUGAUGCGAUGCGGGGGCUGC |
| 1776 | 237 | CCUGAUGCGAUGCGGGGGCUGCU |
| 1777 | 238 | CUGAUGCGAUGCGGGGGCUGCUG |
| 1778 | 239 | UGAUGCGAUGCGGGGGCUGCUGC |
| 1779 | 240 | GAUGCGAUGCGGGGGCUGCUGCA |
| 1780 | 241 | AUGCGAUGCGGGGGCUGCUGCAA |
| 1781 | 242 | UGCGAUGCGGGGGCUGCUGCAAU |
| 1782 | 243 | GCGAUGCGGGGGCUGCUGCAAUG |
| 1783 | 244 | CGAUGCGGGGGCUGCUGCAAUGA |
| 1784 | 245 | GAUGCGGGGGCUGCUGCAAUGAC |
| 1785 | 246 | AUGCGGGGGCUGCUGCAAUGACG |
| 1786 | 247 | UGCGGGGGCUGCUGCAAUGACGA |
| 1787 | 248 | GCGGGGGCUGCUGCAAUGACGAG |
| 1788 | 249 | CGGGGGCUGCUGCAAUGACGAGG |
| 1789 | 250 | GGGGGCUGCUGCAAUGACGAGGG |
| 1790 | 251 | GGGGCUGCUGCAAUGACGAGGGC |
| 1791 | 252 | GGGCUGCUGCAAUGACGAGGGCC |
| 1792 | 253 | GGCUGCUGCAAUGACGAGGGCCU |
| 1793 | 254 | GCUGCUGCAAUGACGAGGGCCUG |
| 1794 | 255 | CUGCUGCAAUGACGAGGGCCUGG |
| 1795 | 256 | UGCUGCAAUGACGAGGGCCUGGA |
| 1796 | 257 | GCUGCAAUGACGAGGGCCUGGAG |
| 1797 | 258 | CUGCAAUGACGAGGGCCUGGAGU |
| 1798 | 259 | UGCAAUGACGAGGGCCUGGAGUG |
| 1799 | 260 | GCAAUGACGAGGGCCUGGAGUGU |
| 1800 | 261 | CAAUGACGAGGGCCUGGAGUGUG |
| 1801 | 262 | AAUGACGAGGGCCUGGAGUGUGU |
| 1802 | 263 | AUGACGAGGGCCUGGAGUGUGUG |
| 1803 | 264 | UGACGAGGGCCUGGAGUGUGUGC |
| 1804 | 265 | GACGAGGGCCUGGAGUGUGUGCC |
| 1805 | 266 | ACGAGGGCCUGGAGUGUGUGCCC |
| 1806 | 267 | CGAGGGCCUGGAGUGUGUGCCCA |
| 1807 | 268 | GAGGGCCUGGAGUGUGUGCCCAC |
| 1808 | 269 | AGGGCCUGGAGUGUGUGCCCACU |
| 1809 | 270 | GGGCCUGGAGUGUGUGCCCACUG |
| 1810 | 271 | GGCCUGGAGUGUGUGCCCACUGA |
| 1811 | 272 | GCCUGGAGUGUGUGCCCACUGAG |
| 1812 | 273 | CCUGGAGUGUGUGCCCACUGAGG |
| 1813 | 274 | CUGGAGUGUGUGCCCACUGAGGA |
| 1814 | 275 | UGGAGUGUGUGCCCACUGAGGAG |
| 1815 | 276 | GGAGUGUGUGCCCACUGAGGAGU |
| 1816 | 277 | GAGUGUGUGCCCACUGAGGAGUC |
| 1817 | 278 | AGUGUGUGCCCACUGAGGAGUCC |
| 1818 | 279 | GUGUGUGCCCACUGAGGAGUCCA |
| 1819 | 280 | UGUGUGCCCACUGAGGAGUCCAA |
| 1820 | 281 | GUGUGCCCACUGAGGAGUCCAAC |
| 1821 | 282 | UGUGCCCACUGAGGAGUCCAACA |
| 1822 | 283 | GUGCCCACUGAGGAGUCCAACAU |
| 1823 | 284 | UGCCCACUGAGGAGUCCAACAUC |
| 1824 | 285 | GCCCACUGAGGAGUCCAACAUCA |
| 1825 | 286 | CCCACUGAGGAGUCCAACAUCAC |
| 1826 | 287 | CCACUGAGGAGUCCAACAUCACC |
| 1827 | 288 | CACUGAGGAGUCCAACAUCACCA |
| 1828 | 289 | ACUGAGGAGUCCAACAUCACCAU |
| 1829 | 290 | CUGAGGAGUCCAACAUCACCAUG |
| 1830 | 291 | UGAGGAGUCCAACAUCACCAUGC |
| 1831 | 292 | GAGGAGUCCAACAUCACCAUGCA |
| 1832 | 293 | AGGAGUCCAACAUCACCAUGCAG |
| 1833 | 294 | GGAGUCCAACAUCACCAUGCAGA |
| 1834 | 295 | GAGUCCAACAUCACCAUGCAGAU |
| 1835 | 296 | AGUCCAACAUCACCAUGCAGAUU |
| 1836 | 297 | GUCCAACAUCACCAUGCAGAUUA |
| 1837 | 298 | UCCAACAUCACCAUGCAGAUUAU |
| 1838 | 299 | CCAACAUCACCAUGCAGAUUAUG |
| 1839 | 300 | CAACAUCACCAUGCAGAUUAUGC |
| 1840 | 301 | AACAUCACCAUGCAGAUUAUGCG |
| 1841 | 302 | ACAUCACCAUGCAGAUUAUGCGG |
| 1842 | 303 | CAUCACCAUGCAGAUUAUGCGGA |
| 1843 | 304 | AUCACCAUGCAGAUUAUGCGGAU |
| 1844 | 305 | UCACCAUGCAGAUUAUGCGGAUC |
| 1845 | 306 | CACCAUGCAGAUUAUGCGGAUCA |
| 1846 | 307 | ACCAUGCAGAUUAUGCGGAUCAA |
| 1847 | 308 | CCAUGCAGAUUAUGCGGAUCAAA |
| 1848 | 309 | CAUGCAGAUUAUGCGGAUCAAAC |
| 1849 | 310 | AUGCAGAUUAUGCGGAUCAAACC |
| 1850 | 311 | UGCAGAUUAUGCGGAUCAAACCU |
| 1851 | 312 | GCAGAUUAUGCGGAUCAAACCUC |
| 1852 | 313 | CAGAUUAUGCGGAUCAAACCUCA |
| 1853 | 314 | AGAUUAUGCGGAUCAAACCUCAC |
| 1854 | 315 | GAUUAUGCGGAUCAAACCUCACC |
| 1855 | 316 | AUUAUGCGGAUCAAACCUCACCA |
| 1856 | 317 | UUAUGCGGAUCAAACCUCACCAA |
| 1857 | 318 | UAUGCGGAUCAAACCUCACCAAG |
| 1858 | 319 | AUGCGGAUCAAACCUCACCAAGG |
| 1859 | 320 | UGCGGAUCAAACCUCACCAAGGC |
| 1860 | 321 | GCGGAUCAAACCUCACCAAGGCC |
| 1861 | 322 | CGGAUCAAACCUCACCAAGGCCA |
| 1862 | 323 | GGAUCAAACCUCACCAAGGCCAG |
| 1863 | 324 | GAUCAAACCUCACCAAGGCCAGC |
| 1864 | 325 | AUCAAACCUCACCAAGGCCAGCA |
| 1865 | 326 | UCAAACCUCACCAAGGCCAGCAC |
| 1866 | 327 | CAAACCUCACCAAGGCCAGCACA |
| 1867 | 328 | AAACCUCACCAAGGCCAGCACAU |
| 1868 | 329 | AACCUCACCAAGGCCAGCACAUA |
| 1869 | 330 | ACCUCACCAAGGCCAGCACAUAG |
| 1870 | 331 | CCUCACCAAGGCCAGCACAUAGG |
| 1871 | 332 | CUCACCAAGGCCAGCACAUAGGA |
| 1872 | 333 | UCACCAAGGCCAGCACAUAGGAG |
| 1873 | 334 | CACCAAGGCCAGCACAUAGGAGA |
| 1874 | 335 | ACCAAGGCCAGCACAUAGGAGAG |
| 1875 | 336 | CCAAGGCCAGCACAUAGGAGAGA |
| 1876 | 337 | CAAGGCCAGCACAUAGGAGAGAU |
| 1877 | 338 | AAGGCCAGCACAUAGGAGAGAUG |
| 1878 | 339 | AGGCCAGCACAUAGGAGAGAUGA |
| 1879 | 340 | GGCCAGCACAUAGGAGAGAUGAG |
| 1880 | 341 | GCCAGCACAUAGGAGAGAUGAGC |
| 1881 | 342 | CCAGCACAUAGGAGAGAUGAGCU |
| 1882 | 343 | CAGCACAUAGGAGAGAUGAGCUU |
| 1883 | 344 | AGCACAUAGGAGAGAUGAGCUUC |
| 1884 | 345 | GCACAUAGGAGAGAUGAGCUUCC |
| 1885 | 346 | CACAUAGGAGAGAUGAGCUUCCU |
| 1886 | 347 | ACAUAGGAGAGAUGAGCUUCCUA |
| 1887 | 348 | CAUAGGAGAGAUGAGCUUCCUAC |
| 1888 | 349 | AUAGGAGAGAUGAGCUUCCUACA |
| 1889 | 350 | UAGGAGAGAUGAGCUUCCUACAG |
| 1890 | 351 | AGGAGAGAUGAGCUUCCUACAGC |
| 1891 | 352 | GGAGAGAUGAGCUUCCUACAGCA |
| 1892 | 353 | GAGAGAUGAGCUUCCUACAGCAC |
| 1893 | 354 | AGAGAUGAGCUUCCUACAGCACA |
| 1894 | 355 | GAGAUGAGCUUCCUACAGCACAA |
| 1895 | 356 | AGAUGAGCUUCCUACAGCACAAC |
| 1896 | 357 | GAUGAGCUUCCUACAGCACAACA |
| 1897 | 358 | AUGAGCUUCCUACAGCACAACAA |
| 1898 | 359 | UGAGCUUCCUACAGCACAACAAA |
| 1899 | 360 | GAGCUUCCUACAGCACAACAAAU |
| 1900 | 361 | AGCUUCCUACAGCACAACAAAUG |
| 1901 | 362 | GCUUCCUACAGCACAACAAAUGU |
| 1902 | 363 | CUUCCUACAGCACAACAAAUGUG |
| 1903 | 364 | UUCCUACAGCACAACAAAUGUGA |
| 1904 | 365 | UCCUACAGCACAACAAAUGUGAA |
| 1905 | 366 | CCUACAGCACAACAAAUGUGAAU |
| 1906 | 367 | CUACAGCACAACAAAUGUGAAUG |
| 1907 | 368 | UACAGCACAACAAAUGUGAAUGC |
| 1908 | 369 | ACAGCACAACAAAUGUGAAUGCA |
| 1909 | 370 | CAGCACAACAAAUGUGAAUGCAG |
| 1910 | 371 | AGCACAACAAAUGUGAAUGCAGA |
| 1911 | 372 | GCACAACAAAUGUGAAUGCAGAC |
| 1912 | 373 | CACAACAAAUGUGAAUGCAGACC |
| 1913 | 374 | ACAACAAAUGUGAAUGCAGACCA |
| 1914 | 375 | CAACAAAUGUGAAUGCAGACCAA |
| 1915 | 376 | AACAAAUGUGAAUGCAGACCAAA |
| 1916 | 377 | ACAAAUGUGAAUGCAGACCAAAG |
| 1917 | 378 | CAAAUGUGAAUGCAGACCAAAGA |
| 1918 | 379 | AAAUGUGAAUGCAGACCAAAGAA |
| 1919 | 380 | AAUGUGAAUGCAGACCAAAGAAA |
| 1920 | 381 | AUGUGAAUGCAGACCAAAGAAAG |
| 1921 | 382 | UGUGAAUGCAGACCAAAGAAAGA |
| 1922 | 383 | GUGAAUGCAGACCAAAGAAAGAU |
| 1923 | 384 | UGAAUGCAGACCAAAGAAAGAUA |
| 1924 | 385 | GAAUGCAGACCAAAGAAAGAUAG |
| 1925 | 386 | AAUGCAGACCAAAGAAAGAUAGA |
| 1926 | 387 | AUGCAGACCAAAGAAAGAUAGAG |
| 1927 | 388 | UGCAGACCAAAGAAAGAUAGAGC |
| 1928 | 389 | GCAGACCAAAGAAAGAUAGAGCA |
| 1929 | 390 | CAGACCAAAGAAAGAUAGAGCAA |
| 1930 | 391 | AGACCAAAGAAAGAUAGAGCAAG |
| 1931 | 392 | GACCAAAGAAAGAUAGAGCAAGA |
| 1932 | 393 | ACCAAAGAAAGAUAGAGCAAGAC |
| 1933 | 394 | CCAAAGAAAGAUAGAGCAAGACA |
| 1934 | 395 | CAAAGAAAGAUAGAGCAAGACAA |
| 1935 | 396 | AAAGAAAGAUAGAGCAAGACAAG |
| 1936 | 397 | AAGAAAGAUAGAGCAAGACAAGA |
| 1937 | 398 | AGAAAGAUAGAGCAAGACAAGAA |
| 1938 | 399 | GAAAGAUAGAGCAAGACAAGAAA |
| 1939 | 400 | AAAGAUAGAGCAAGACAAGAAAA |

TABLE 4b
VEGF targeted duplexes

| position | SEQ | | | | SEQ | |
|---|---|---|---|---|---|---|
| in | ID | Target sequence | | | ID | |
| ORF | NO: | (5′-3′) | Duplex ID | Strand | NO: | Strand Sequences |

| | | | | | | |
|---|---|---|---|---|---|---|
| 1 | 2184 | AUGAACUUUCUGCUGUCUUGGGU | AL-DP-4043 | S | 1940 | 5 GAACUUUCUGCUGUCUUGGGU 3 |
| | | | | AS | 1941 | 3 UACUUGAAAGACGACAGAACCCA 5 |
| 22 | 2185 | GUGCAUUGGAGCCUUGCCUUGCU | AL-DP-4077 | S | 1942 | 5 GCAUUGGAGCCUUGCCUUGCU 3 |
| | | | | AS | 1943 | 3 CACGUAACCUCGGAACGGAACGA 5 |
| 47 | 2186 | UCUACCUCCACCAUGCCAAGUGG | AL-DP-4021 | S | 1944 | 5 UACCUCCACCAUGCCAAGUTT 3 |
| | | | | AS | 1945 | 3 TTAUGGAGGUGGUACGGUUCA 5 |
| 48 | 2187 | CUACCUCCACCAUGCCAAGUGGU | AL-DP-4109 | S | 1946 | 5 ACCUCCACCAUGCCAAGUGTT 3 |
| | | | | AS | 1947 | 3 TTUGGAGGUGGUACGGUUCAC 5 |
| 50 | 2188 | ACCUCCACCAUGCCAAGUGGUCC | AL-DP-4006 | S | 1948 | 5 CUCCACCAUGCCAAGUGGUCC 3 |
| | | | | AS | 1949 | 3 UGGAGGUGGUACGGUUCACCAGG 5 |
| | | | AL-DP-4083 | S | 1950 | 5 CUCCACCAUGCCAAGUGGUTT 3 |
| | | | | AS | 1951 | 3 TTGAGGUGGUACGGUUCACCA 5 |
| 51 | 2189 | CCUCCACCAUGCCAAGUGGUCCC | AL-DP-4047 | S | 1952 | 5 UCCACCAUGCCAAGUGGUCCC 3 |
| | | | | AS | 1953 | 3 GGAGGUGGUACGGUUCACCAGGG 5 |
| | | | AL-DP-4017 | S | 1954 | 5 UCCACCAUGCCAAGUGGUCTT 3 |
| | | | | AS | 1955 | 3 TTAGGUGGUACGGUUCACCAG 5 |
| 52 | 2190 | CUCCACCAUGCCAAGUGGUCCCA | AL-DP-4048 | S | 1956 | 5 CCACCAUGCCAAGUGGUCCCA 3 |
| | | | | AS | 1957 | 3 GAGGUGGUACGGUUCACCAGGGU 5 |
| | | | AL-DP-4103 | S | 1958 | 5 CCACCAUGCCAAGUGGUCCTT 3 |
| | | | | AS | 1959 | 3 TTGGUGGUACGGUUCACCAGG 5 |
| 53 | 2191 | UCCACCAUGCCAAGUGGUCCCAG | AL-DP-4035 | S | 1960 | 5 CACCAUGCCAAGUGGUCCCAG 3 |
| | | | | AS | 1961 | 3 AGGUGGUACGGUUCACCAGGGUC 5 |
| | | | AL-DP-4018 | S | 1962 | 5 CACCAUGCCAAGUGGUCCCTT 3 |
| | | | | AS | 1963 | 3 TTGUGGUACGGUUCACCAGGG 5 |
| 54 | 2192 | CCACCAUGCCAAGUGGUCCCAGG | AL-DP-4036 | S | 1964 | 5 ACCAUGCCAAGUGGUCCCAGG 3 |
| | | | | AS | 1965 | 3 GGUGGUACGGUUCACCAGGGUCC 5 |
| | | | AL-DP-4084 | S | 1966 | 5 ACCAUGCCAAGUGGUCCCATT 3 |
| | | | | AS | 1967 | 3 TTUGGUACGGUUCACCAGGGU 5 |
| 55 | 2193 | CACCAUGCCAAGUGGUCCCAGGC | AL-DP-4093 | S | 1968 | 5 CCAUGCCAAGUGGUCCCAGGC 3 |
| | | | | AS | 1969 | 3 GUGGUACGGUUCACCAGGGUCCG 5 |
| | | | AL-DP-4085 | S | 1970 | 5 CCAUGCCAAGUGGUCCCAGTT 3 |
| | | | | AS | 1971 | 3 TTGGUACGGUUCACCAGGGUC 5 |
| 56 | 2194 | ACCAUGCCAAGUGGUCCCAGGCU | AL-DP-4037 | S | 1972 | 5 CAUGCCAAGUGGUCCCAGGCU 3 |
| | | | | AS | 1973 | 3 UGGUACGGUUCACCAGGGUCCGA 5 |
| | | | AL-DP-4054 | S | 1974 | 5 CAUGCCAAGUGGUCCCAGGTT 3 |
| | | | | AS | 1975 | 3 TTGUACGGUUCACCAGGGUCC 5 |
| 57 | 2195 | CCAUGCCAAGUGGUCCCAGGCUG | AL-DP-4038 | S | 1976 | 5 AUGCCAAGUGGUCCCAGGCUG 3 |
| | | | | AS | 1977 | 3 GGUACGGUUCACCAGGGUCCGAC 5 |
| | | | AL-DP-4086 | S | 1978 | 5 AUGCCAAGUGGUCCCAGGCTT 3 |
| | | | | AS | 1979 | 3 TTUACGGUUCACCAGGGUCCG 5 |
| 58 | 2196 | CAUGCCAAGUGGUCCCAGGCUGC | AL-DP-4049 | S | 1980 | 5 UGCCAAGUGGUCCCAGGCUGC 3 |
| | | | | AS | 1981 | 3 GUACGGUUCACCAGGGUCCGACG 5 |
| | | | AL-DP-4087 | S | 1982 | 5 UGCCAAGUGGUCCCAGGCUTT 3 |
| | | | | AS | 1983 | 3 TTACGGUUCACCAGGGUCCGA 5 |
| 59 | 2197 | AUGCCAAGUGGUCCCAGGCUGCA | AL-DP-4001 | S | 1984 | 5 GCCAAGUGGUCCCAGGCUGCA 3 |
| | | | | AS | 1985 | 3 UACGGUUCACCAGGGUCCGACGU 5 |
| | | | AL-DP-4052 | A | 1986 | 5 GCCAAGUGGUCCCAGGCUGTT 3 |
| | | | | AS | 1987 | 3 TTCGGUUCACCAGGGUCCGAC 5 |
| 60 | 2198 | UGCCAAGUGGUCCCAGGCUGCAC | AL-DP-4007 | S | 1988 | 5 CCAAGUGGUCCCAGGCUGCAC 3 |
| | | | | AS | 1989 | 3 ACGGUUCACCAGGGUCCGACGUG 5 |
| | | | AL-DP-4088 | S | 1990 | 5 CCAAGUGGUCCCAGGCUGCTT 3 |
| | | | | AS | 1991 | 3 TTGGUUCACCAGGGUCCGACG 5 |
| 61 | 2199 | GCCAAGUGGUCCCAGGCUGCACC | AL-DP-4070 | S | 1992 | 5 CAAGUGGUCCCAGGCUGCACC 3 |
| | | | | AS | 1993 | 3 CGGUUCACCAGGGUCCGACGUGG 5 |
| | | | AL-DP-4055 | S | 1994 | 5 CAAGUGGUCCCAGGCUGCATT 3 |
| | | | | AS | 1995 | 3 TTGUUCACCAGGGUCCGACGU 5 |
| 62 | 2200 | CCAAGUGGUCCCAGGCUGCACCC | AL-DP-4071 | S | 1996 | 5 AAGUGGUCCCAGGCUGCACCC 3 |
| | | | | AS | 1997 | 3 GGUUCACCAGGGUCCGACGUGGG 5 |
| | | | AL-DP-4056 | S | 1998 | 5 AAGUGGUCCCAGGCUGCACTT 3 |
| | | | | AS | 1999 | 3 TTUUCACCAGGGUCCGACGUG 5 |
| 63 | 2201 | CAAGUGGUCCCAGGCUGCACCCA | AL-DP-4072 | S | 2000 | 5 AGUGGUCCCAGGCUGCACCCA 3 |
| | | | | AS | 2001 | 3 GUUCACCAGGGUCCGACGUGGGU 5 |
| | | | AL-DP-4057 | S | 2002 | 5 AGUGGUCCCAGGCUGCACCTT 3 |
| | | | | AS | 2003 | 3 TTUCACCAGGGUCCGACGUGG 5 |
| 64 | 2202 | AAGUGGUCCCAGGCUGCACCCAU | AL-DP-4066 | S | 2004 | 5 GUGGUCCCAGGCUGCACCCTT 3 |
| | | | | AS | 2005 | 3 TTCACCAGGGUCCGACGUGGG 5 |
| 99 | 2203 | AGGGCAGAAUCAUCACGAAGUGG | AL-DP-4022 | S | 2006 | 5 GGCAGAAUCAUCACGAAGUTT 3 |
| | | | | AS | 2007 | 3 TTCCGUCUUAGUAGUGCUUCA 5 |
| 100 | 2204 | GGGCAGAAUCAUCACGAAGUGGU | AL-DP-4023 | S | 2008 | 5 GCAGAAUCAUCACGAAGUGTT 3 |
| | | | | AS | 2009 | 3 TTCGUCUUAGUAGUGCUUCAC 5 |
| 101 | 2205 | GGCAGAAUCAUCACGAAGUGGUG | AL-DP-4024 | S | 2010 | 5 CAGAAUCAUCACGAAGUGGTT 3 |
| | | | | AS | 2011 | 3 TTGUCUUAGUAGUGCUUCACC 5 |
| 102 | 2206 | GCAGAAUCAUCACGAAGUGGUGA | AL-DP-4076 | S | 2012 | 5 AGAAUCAUCACGAAGUGGUGA 3 |
| | | | | AS | 2013 | 3 CGUCUUAGUAGUGCUUCACCACU 5 |
| | | | AL-DP-4019 | S | 2014 | 5 AGAAUCAUCACGAAGUGGUTT 3 |
| | | | | AS | 2015 | 3 TTUCUUAGUAGUGCUUCACCA 5 |
| 103 | 2207 | CAGAAUCAUCACGAAGUGGUGAA | AL-DP-4025 | S | 2016 | 5 GAAUCAUCACGAAGUGGUGTT 3 |
| | | | | AS | 2017 | 3 TTCUUAGUAGUGCUUCACCAC 5 |
| 104 | 2208 | AGAAUCAUCACGAAGUGGUGAAG | AL-DP-4110 | S | 2018 | 5 AAUCAUCACGAAGUGGUGATT 3 |
| | | | | AS | 2019 | 3 TTUUAGUAGUGCUUCACCACU 5 |
| 105 | 2209 | GAAUCAUCACGAAGUGGUGAAGU | AL-DP-4068 | S | 2020 | 5 AUCAUCACGAAGUGGUGAATT 3 |
| | | | | AS | 2021 | 3 TTUAGUAGUGCUUCACCACUU 5 |
| 113 | 2210 | ACGAAGUGGUGAAGUUCAUGGAU | AL-DP-4078 | S | 2022 | 5 GAAGUGGUGAAGUUCAUGGAU 3 |
| | | | | AS | 2023 | 3 UGCUUCACCACUUCAAGUACCUA 5 |
| 121 | 2211 | GUGAAGUUCAUGGAUGUCUAUCA | AL-DP-4080 | S | 2024 | 5 GAAGUUCAUGGAUGUCUAUCA 3 |
| | | | | AS | 2025 | 3 CACUUCAAGUACCUACAGAUAGU 5 |
| 129 | 2212 | CAUGGAUGUCUAUCAGCGCAGCU | AL-DP-4111 | S | 2026 | 5 UGGAUGUCUAUCAGCGCAGTT 3 |
| | | | | AS | 2027 | 3 TTACCUACAGAUAGUCGCGUC 5 |
| 130 | 2213 | AUGGAUGUCUAUCAGCGCAGCUA | AL-DP-4041 | S | 2028 | 5 GGAUGUCUAUCAGCGCAGCUA 3 |
| | | | | AS | 2029 | 3 UACCUACAGAUAGUCGCGUCGAU 5 |
| | | | AL-DP-4062 | S | 2030 | 5 GGAUGUCUAUCAGCGCAGCTT 3 |
| | | | | AS | 2031 | 3 TTCCUACAGAUAGUCGCGUCG 5 |
| 131 | 2214 | UGGAUGUCUAUCAGCGCAGCUAC | AL-DP-4069 | S | 2032 | 5 GAUGUCUAUCAGCGCAGCUTT 3 |
| | | | | AS | 2033 | 3 TTCUACAGAUAGUCGCGUCGA 5 |
| 132 | 2215 | GGAUGUCUAUCAGCGCAGCUACU | AL-DP-4112 | S | 2034 | 5 AUGUCUAUCAGCGCAGCUATT 3 |
| | | | | AS | 2035 | 3 TTUACAGAUAGUCGCGUCGAU 5 |
| 133 | 2216 | GAUGUCUAUCAGCGCAGCUACUG | AL-DP-4026 | S | 2036 | 5 UGUCUAUCAGCGCAGCUACTT 3 |
| | | | | AS | 2037 | 3 TTACAGAUAGUCGCGUCGAUG 5 |
| 134 | 2217 | AUGUCUAUCAGCGCAGCUACUGC | AL-DP-4095 | S | 2038 | 5 GUCUAUCAGCGCAGCUACUGC 3 |
| | | | | AS | 2039 | 3 UACAGAUAGUCGCGUCGAUGACG 5 |
| | | | AL-DP-4020 | S | 2040 | 5 GUCUAUCAGCGCAGCUACUTT 3 |
| | | | | AS | 2041 | 3 TTCAGAUAGUCGCGUCGAUGA 5 |
| 135 | 2218 | UGUCUAUCAGCGCAGCUACUGCC | AL-DP-4027 | S | 2042 | 5 UCUAUCAGCGCAGCUACUGTT 3 |
| | | | | AS | 2043 | 3 TTAGAUAGUCGCGUCGAUGAC 5 |
| 144 | 2219 | GCGCAGCUACUGCCAUCCAAUCG | AL-DP-4081 | S | 2044 | 5 GCAGCUACUGCCAUCCAAUCG 3 |
| | | | | AS | 2045 | 3 CGCGUCGAUGACGGUAGGUUAGC 5 |
| 146 | 2220 | GCAGCUACUGCCAUCCAAUCGAG | AL-DP-4098 | S | 2046 | 5 AGCUACUGCCAUCCAAUCGAG 3 |
| | | | | AS | 2047 | 3 CGUCGAUGACGGUAGGUUAGCUC 5 |
| 149 | 2221 | GCUACUGCCAUCCAAUCGAGACC | AL-DP-4028 | S | 2048 | 5 UACUGCCAUCCAAUCGAGATT 3 |
| | | | | AS | 2049 | 3 TTAUGACGGUAGGUUAGCUCU 5 |
| 150 | 2222 | CUACUGCCAUCCAAUCGAGACCC | AL-DP-4029 | S | 2050 | 5 ACUGCCAUCCAAUCGAGACTT 3 |
| | | | | AS | 2051 | 3 TTUGACGGUAGGUUAGCUCUG 5 |
| 151 | 2223 | UACUGCCAUCCAAUCGAGACCCU | AL-DP-4030 | S | 2052 | 5 CUGCCAUCCAAUCGAGACCTT 3 |
| | | | | AS | 2053 | 3 TTGACGGUAGGUUAGCUCUGG 5 |
| 152 | 2224 | ACUGCCAUCCAAUCGAGACCCUG | AL-DP-4031 | S | 2054 | 5 UGCCAUCCAAUCGAGACCCTT 3 |
| | | | | AS | 2055 | 3 TTACGGUAGGUUAGCUCUGGG 5 |
| 166 | 2225 | GAGACCCUGGUGGACAUCUUCCA | AL-DP-4008 | S | 2056 | 5 GACCCUGGUGGACAUCUUCCA 3 |
| | | | | AS | 2057 | 3 CUCUGGGACCACCUGUAGAAGGU 5 |
| | | | AL-DP-4058 | S | 2058 | 5 GACCCUGGUGGACAUCUUCTT 3 |
| | | | | AS | 2059 | 3 TTCUGGGACCACCUGUAGAAG 5 |
| 167 | 2226 | AGACCCUGGUGGACAUCUUCCAG | AL-DP-4009 | S | 2060 | 5 ACCCUGGUGGACAUCUUCCAG 3 |
| | | | | AS | 2061 | 3 UCUGGGACCACCUGUAGAAGGUC 5 |
| | | | AL-DP-4059 | S | 2062 | 5 ACCCUGGUGGACAUCUUCCTT 3 |
| | | | | AS | 2063 | 3 TTUGGGACCACCUGUAGAAGG 5 |
| 168 | 2227 | GACCCUGGUGGACAUCUUCCAGG | AL-DP-4010 | S | 2064 | 5 CCCUGGUGGACAUCUUCCAGG 3 |
| | | | | AS | 2065 | 3 CUGGGACCACCUGUAGAAGGUCC 5 |
| | | | AL-DP-4060 | S | 2066 | 5 CCCUGGUGGACAUCUUCCATT 3 |
| | | | | AS | 2067 | 3 TTGGGACCACCUGUAGAAGGU 5 |
| 169 | 2228 | ACCCUGGUGGACAUCUUCCAGGA | AL-DP-4073 | S | 2068 | 5 CCUGGUGGACAUCUUCCAGGA 3 |
| | | | | AS | 2069 | 3 UGGGACCACCUGUAGAAGGUCCU 5 |
| | | | AL-DP-4104 | S | 2070 | 5 CCUGGUGGACAUCUUCCAGTT 3 |
| | | | | AS | 2071 | 3 TTGGACCACCUGUAGAAGGUC 5 |
| 170 | 2229 | CCCUGGUGGACAUCUUCCAGGAG | AL-DP-4011 | S | 2072 | 5 CUGGUGGACAUCUUCCAGGAG 3 |
| | | | | AS | 2073 | 3 GGGACCACCUGUAGAAGGUCCUC 5 |
| | | | AL-DP-4089 | S | 2074 | 5 CUGGUGGACAUCUUCCAGGTT 3 |
| | | | | AS | 2075 | 3 TTGACCACCUGUAGAAGGUCC 5 |
| 171 | 2230 | CCUGGUGGACAUCUUCCAGGAGU | AL-DP-4074 | S | 2076 | 5 UGGUGGACAUCUUCCAGGAGU 3 |
| | | | | AS | 2077 | 3 GGACCACCUGUAGAAGGUCCUCA 5 |
| | | | AL-DP-4090 | S | 2078 | 5 UGGUGGACAUCUUCCAGGATT 3 |
| | | | | AS | 2079 | 3 TTACCACCUGUAGAAGGUCCU 5 |
| 172 | 2231 | CUGGUGGACAUCUUCCAGGAGUA | AL-DP-4039 | S | 2080 | 5 GGUGGACAUCUUCCAGGAGUA 3 |
| | | | | AS | 2081 | 3 GACCACCUGUAGAAGGUCCUCAU 5 |
| | | | AL-DP-4091 | S | 2082 | 5 GGUGGACAUCUUCCAGGAGTT 3 |
| | | | | AS | 2083 | 3 TTCCACCUGUAGAAGGUCCUC 5 |
| 175 | 2232 | GUGGACAUCUUCCAGGAGUACCC | AL-DP-4003 | S | 2084 | 5 GGACAUCUUCCAGGAGUACCC 3 |
| | | | | AS | 2085 | 3 CCUGUAGAAGGUCCUCAUGGG 5 |
| | | | AL-DP-4116 | S | 2086 | 5 GGACAUCUUCCAGGAGUACCC 3 |
| | | | | AS | 2087 | 3 CCUGUAGAAGGUCCUCAUGGG 5 |
| | | | AL-DP-4015 | S | 2088 | 5 GGACAUCUUCCAGGAGUACTT 3 |
| | | | | AS | 2089 | 3 TTCCUGUAGAAGGUCCUCAUG 5 |
| | | | AL-DP-4120 | S | 2090 | 5 GGACAUCUUCCAGGAGUAC 3 |
| | | | | AS | 2091 | 3 CCUGUAGAAGGUCCUCAUG 5 |
| 179 | 2233 | ACAUCUUCCAGGAGUACCCUGAU | AL-DP-4099 | S | 2092 | 5 AUCUUCCAGGAGUACCCUGAU 3 |
| | | | | AS | 2093 | 3 UGUAGAAGGUCCUCAUGGGACUA 5 |
| 191 | 2234 | AGUACCCUGAUGAGAUCGAGUAC | AL-DP-4032 | S | 2094 | 5 UACCCUGAUGAGAUCGAGUTT 3 |
| | | | | AS | 2095 | 3 TTAUGGGACUACUCUAGCUCA 5 |
| 192 | 2235 | GUACCCUGAUGAGAUCGAGUACA | AL-DP-4042 | S | 2096 | 5 ACCCUGAUGAGAUCGAGUACA 3 |
| | | | | AS | 2097 | 3 CAUGGGACUACUCUAGCUCAUGU 5 |
| | | | AL-DP-4063 | S | 2098 | 5 ACCCUGAUGAGAUCGAGUATT 3 |
| | | | | AS | 2099 | 3 TTUGGGACUACUCUAGCUCAU 5 |
| 209 | 2236 | AGUACAUCUUCAAGCCAUCCUGU | AL-DP-4064 | S | 2100 | 5 UACAUCUUCAAGCCAUCCUTT 3 |
| | | | | AS | 2101 | 3 TTAUGUAGAAGUUCGGUAGGA 5 |
| 260 | 2237 | GCAAUGACGAGGGCCUGGAGUGU | AL-DP-4044 | S | 2102 | 5 AAUGACGAGGGCCUGGAGUGU 3 |
| | | | | AS | 2103 | 3 CGUUACUGCUCCCGGACCUCACA 5 |
| 263 | 2238 | AUGACGAGGGCCUGGAGUGUGUG | AL-DP-4045 | S | 2104 | 5 GACGAGGGCCUGGAGUGUGUG 3 |
| | | | | AS | 2105 | 3 UACUGCUCCCGGACCUCACACAC 5 |
| 279 | 2239 | GUGUGUGCCCACUGAGGAGUCCA | AL-DP-4046 | S | 2106 | 5 GUGUGCCCACUGAGGAGUCCA 3 |
| | | | | AS | 2107 | 3 CACACACGGGUGACUCCUCAGGU 5 |
| 281 | 2240 | GUGUGCCCACUGAGGAGUCCAAC | AL-DP-4096 | S | 2108 | 5 GUGCCCACUGAGGAGUCCAAC 3 |
| | | | | AS | 2109 | 3 CACACGGGUGACUCCUCAGGUUG 5 |
| 283 | 2241 | GUGCCCACUGAGGAGUCCAACAU | AL-DP-4040 | S | 2110 | 5 GCCCACUGAGGAGUCCAACAU 3 |
| | | | | AS | 2111 | 3 CACGGGUGACUCCUCAGGUUGUA 5 |
| 289 | 2242 | ACUGAGGAGUCCAACAUCACCAU | AL-DP-4065 | S | 2112 | 5 UGAGGAGUCCAACAUCACCTT 3 |
| | | | | AS | 2113 | 3 TTACUCCUCAGGUUGUAGUGG 5 |
| 302 | 2243 | ACAUCACCAUGCAGAUUAUGCGG | AL-DP-4100 | S | 2114 | 5 AUCACCAUGCAGAUUAUGCGG 3 |
| | | | | AS | 2115 | 3 UGUAGUGGUACGUCUAAUACGCC 5 |
| 305 | 2244 | UCACCAUGCAGAUUAUGCGGAUC | AL-DP-4033 | S | 2116 | 5 ACCAUGCAGAUUAUGCGGATT 3 |
| | | | | AS | 2117 | 3 TTUGGUACGUCUAAUACGCCU 5 |
| 310 | 2245 | AUGCAGAUUAUGCGGAUCAAACC | AL-DP-4101 | S | 2118 | 5 GCAGAUUAUGCGGAUCAAACC 3 |
| | | | | AS | 2119 | 3 UACGUCUAAUACGCCUAGUUUGG 5 |
| 312 | 2246 | GCAGAUUAUGCGGAUCAAACCUC | AL-DP-4102 | S | 2120 | 5 AGAUUAUGCGGAUCAAACCUC 3 |
| | | | | AS | 2121 | 3 CGUCUAAUACGCCUAGUUUGGAG 5 |
| 315 | 2247 | GAUUAUGCGGAUCAAACCUCACC | AL-DP-4034 | S | 2122 | 5 UUAUGCGGAUCAAACCUCATT 3 |
| | | | | AS | 2123 | 3 TTAAUACGCCUAGUUUGGAGU 5 |
| 316 | 2248 | AUUAUGCGGAUCAAACCUCACCA | AL-DP-4113 | S | 2124 | 5 UAUGCGGAUCAAACCUCACTT 3 |
| | | | | AS | 2125 | 3 TTAUACGCCUAGUUUGGAGUG 5 |
| 317 | 2249 | UUAUGCGGAUCAAACCUCACCAA | AL-DP-4114 | S | 2126 | 5 AUGCGGAUCAAACCUCACCTT 3 |
| | | | | AS | 2127 | 3 TTUACGCCUAGUUUGGAGUGG 5 |
| 319 | 2250 | AUGCGGAUCAAACCUCACCAAGG | AL-DP-4002 | S | 2128 | 5 GCGGAUCAAACCUCACCAAGG 3 |
| | | | | AS | 2129 | 3 UACGCCUAGUUUGGAGUGGUUCC 5 |
| | | | AL-DP-4115 | S | 2130 | 5 GCGGAUCAAACCUCACCAA 3 |
| | | | | AS | 2131 | 3 CGCCUAGUUUGGAGUGGUU 5 |
| | | | AL-DP-4014 | S | 2132 | 5 GCGGAUCAAACCUCACCAATT 3 |
| | | | | AS | 2133 | 3 TTCGCCUAGUUUGGAGUGGUU 5 |
| | | | AL-DP-4119 | S | 2134 | 5 GCGGAUCAAACCUCACCAA 3 |
| | | | | AS | 2135 | 3 CGCCUAGUUUGGAGUGGUU 5 |
| 321 | 2251 | GCGGAUCAAACCUCACCAAGGCC | AL-DP-4013 | S | 2136 | 5 GGAUCAAACCUCACCAAGGCC 3 |
| | | | | AS | 2137 | 3 CGCCUAGUUUGGAGUGGUUCCGG 5 |
| 341 | 2252 | GCCAGCACAUAGGAGAGAUGAGC | AL-DP-4075 | S | 2138 | 5 CAGCACAUAGGAGAGAUGAGC 3 |
| | | | | AS | 2139 | 3 CGGUCGUGUAUCCUCUCUACUCG 5 |
| | | | AL-DP-4105 | S | 2140 | 5 CAGCACAUAGGAGAGAUGATT 3 |
| | | | | AS | 2141 | 3 TTGUCGUGUAUCCUCUCUACU 5 |
| 342 | 2253 | CCAGCACAUAGGAGAGAUGAGCU | AL-DP-4050 | S | 2142 | 5 AGCACAUAGGAGAGAUGAGCU 3 |
| | | | | AS | 2143 | 3 GGUCGUGUAUCCUCUCUACUCGA 5 |
| | | | AL-DP-4106 | S | 2144 | 5 AGCACAUAGGAGAGAUGAGTT 3 |
| | | | | AS | 2145 | 3 TTUCGUGUAUCCUCUCUACUC 5 |
| 343 | 2254 | CAGCACAUAGGAGAGAUGAGCUU | AL-DP-4094 | S | 2146 | 5 GCACAUAGGAGAGAUGAGCUU 3 |
| | | | | AS | 2147 | 3 GUCGUGUAUCCUCUCUACUCGAA 5 |
| | | | AL-DP-4118 | S | 2148 | 5 GCACAUAGGAGAGAUGAGCUU 3 |
| | | | | AS | 2149 | 3 CGUGUAUCCUCUCUACUCGAA 5 |
| | | | AL-DP-4107 | S | 2150 | 5 GCACAUAGGAGAGAUGAGCTT 3 |
| | | | | AS | 2151 | 3 TTCGUGUAUCCUCUCUACUCG 5 |
| | | | AL-DP-4122 | S | 2152 | 5 GCACAUAGGAGAGAUGAGC 3 |
| | | | | AS | 2153 | 3 CGUGUAUCCUCUCUACUCG 5 |
| 344 | 2255 | AGCACAUAGGAGAGAUGAGCUUC | AL-DP-4012 | S | 2154 | 5 CACAUAGGAGAGAUGAGCUUC 3 |
| | | | | AS | 2155 | 3 UCGUGUAUCCUCUCUACUCGAAG 5 |
| | | | AL-DP-4108 | S | 2156 | 5 CACAUAGGAGAGAUGAGCUTT 3 |
| | | | | AS | 2157 | 3 TTGUGUAUCCUCUCUACUCGA 5 |
| 346 | 2256 | CACAUAGGAGAGAUGAGCUUCCU | AL-DP-4051 | S | 2158 | 5 CAUAGGAGAGAUGAGCUUCCU 3 |
| | | | | AS | 2159 | 3 GUGUAUCCUCUCUACUCGAAGGA 5 |
| | | | AL-DP-4061 | S | 2160 | 5 CAUAGGAGAGAUGAGCUUCTT 3 |
| | | | | AS | 2161 | 3 TTGUAUCCUCUCUACUCGAAG 5 |
| 349 | 2257 | AUAGGAGAGAUGAGCUUCCUACA | AL-DP-4082 | S | 2162 | 5 AGGAGAGAUGAGCUUCCUACA 3 |
| | | | | AS | 2163 | 3 UAUCCUCUCUACUCGAAGGAUGU 5 |
| 369 | 2258 | ACAGCACAACAAAUGUGAAUGCA | AL-DP-4079 | S | 2164 | 5 AGCACAACAAAUGUGAAUGCA 3 |
| | | | | AS | 2165 | 3 UGUCGUGUUGUUUACACUUACGU 5 |
| 372 | 2259 | GCACAACAAAUGUGAAUGCAGAC | AL-DP-4097 | S | 2166 | 5 ACAACAAAUGUGAAUGCAGAC 3 |
| | | | | AS | 2167 | 3 CGUGUUGUUUACACUUACGUCUG 5 |
| 379 | 2260 | AAAUGUGAAUGCAGACCAAAGAA | AL-DP-4067 | S | 2168 | 5 AUGUGAAUGCAGACCAAAGTT 3 |
| | | | | AS | 2169 | 3 TTUACACUUACGUCUGGUUUC 5 |
| 380 | 2261 | AAUGUGAAUGCAGACCAAAGAAA | AL-DP-4092 | S | 2170 | 5 UGUGAAUGCAGACCAAAGATT 3 |
| | | | | AS | 2171 | 3 TTACACUUACGUCUGGUUUCU 5 |
| 381 | 2262 | AUGUGAAUGCAGACCAAAGAAAG | AL-DP-4004 | S | 2172 | 5 GUGAAUGCAGACCAAAGAAAG 3 |
| | | | | AS | 2173 | 3 UACACUUACGUCUGGUUUCUUUC 5 |
| | | | AL-DP-4117 | S | 2174 | 5 GUGAAUGCAGACCAAAGAAAG 3 |
| | | | | AS | 2175 | 3 CACUUACGUCUGGUUUCUUUC 5 |
| | | | AL-DP-4016 | S | 2176 | 5 GUGAAUGCAGACCAAAGAATT 3 |
| | | | | AS | 2177 | 3 TTCACUUACGUCUGGUUUCUU 5 |
| | | | AL-DP-4121 | S | 2178 | 5 GUGAAUGCAGACCAAAGAA 3 |
| | | | | AS | 2179 | 3 CACUUACGUCUGGUUUCUU 5 |
| 383 | 2263 | GUGAAUGCAGACCAAAGAAAGAU | AL-DP-4005 | S | 2180 | 5 GAAUGCAGACCAAAGAAAGAU 3 |
| | | | | AS | 2181 | 3 CACUUACGUCUGGUUUCUUUCUA 5 |
| | | | AL-DP-4053 | S | 2182 | 5 GAAUGCAGACCAAAGAAAGTT 3 |
| | | | | AS | 2183 | 3 TTCUUACGUCUGGUUUCUUUC 5 |

Strand:
S = sense,
AS = Antisense

### Example 2

### Eg5 siRNA in vitro Screening via Cell Proliferation

As silencing of Eg5 has been shown to cause mitotic arrest (Weil, D, et at [2002] Biotechniques 33: 1244-8), a cell viability assay was used for siRNA activity screening. HeLa cells (14000 per well [Screens 1 and 3] or 10000 per well [Screen2])) were seeded in 96-well plates and simultaneously transfected with Lipofectamine 2000 (Invitrogen) at a final siRNA concentration in the well of 30 nM and at final concentrations of 50 nM (1^{st} screen) and 25 nM (2^{nd} screen). A subset of duplexes was tested at 25 nM in a third screen (Table 5).

Seventy-two hours post-transfection, cell proliferation was assayed the addition of WST-1 reagent (Roche) to the culture medium, and subsequent absorbance measurement at 450 nm. The absorbance value for control (non-transfected) cells was considered 100 percent, and absorbances for the siRNA transfected wells were compared to the control value. Assays were performed in sextuplicate for each of three screens. A subset of the siRNAs was further tested at a range of siRNA concentrations. Assays were performed in HeLa cells (14000 per well;

method same as above, Table 5).

TABLE 5
Effects of Eg5 targeted duplexes on cell viability at 25 nM.

| | Relative absorbance at 450 nm | | | | | |
|---|---|---|---|---|---|---|
| | Screen I | | Screen II | | Screen III | |
| Duplex | mean | sd | Mean | sd | mean | Sd |
| AL-DP-6226 | 20 | 10 | 28 | 11 | 43 | 9 |
| AL-DP-6227 | 66 | 27 | 96 | 41 | 108 | 33 |
| AL-DP-6228 | 56 | 28 | 76 | 22 | 78 | 18 |
| AL-DP-6229 | 17 | 3 | 31 | 9 | 48 | 13 |
| AL-DP-6230 | 48 | 8 | 75 | 11 | 73 | 7 |
| AL-DP-6231 | 8 | 1 | 21 | 4 | 41 | 10 |
| AL-DP-6232 | 16 | 2 | 37 | 7 | 52 | 14 |
| AL-DP-6233 | 31 | 9 | 37 | 6 | 49 | 12 |
| AL-DP-6234 | 103 | 40 | 141 | 29 | 164 | 45 |
| AL-DP-6235 | 107 | 34 | 140 | 27 | 195 | 75 |
| AL-DP-6236 | 48 | 12 | 54 | 12 | 56 | 12 |
| AL-DP-6237 | 73 | 14 | 108 | 18 | 154 | 37 |
| AL-DP-6238 | 64 | 9 | 103 | 10 | 105 | 24 |
| AL-DP-6239 | 9 | 1 | 20 | 4 | 31 | 11 |
| AL-DP-6240 | 99 | 7 | 139 | 16 | 194 | 43 |
| AL-DP-6241 | 43 | 9 | 54 | 12 | 66 | 19 |
| AL-DP-6242 | 6 | 1 | 15 | 7 | 36 | 8 |
| AL-DP-6243 | 7 | 2 | 19 | 5 | 33 | 13 |
| AL-DP-6244 | 7 | 2 | 19 | 3 | 37 | 13 |
| AL-DP-6245 | 25 | 4 | 45 | 10 | 58 | 9 |
| AL-DP-6246 | 34 | 8 | 65 | 10 | 66 | 13 |
| AL-DP-6247 | 53 | 6 | 78 | 14 | 105 | 20 |
| AL-DP-6248 | 7 | 0 | 22 | 7 | 39 | 12 |
| AL-DP-6249 | 36 | 8 | 48 | 13 | 61 | 7 |

The nine siRNA duplexes that showed the greatest growth inhibition in Table 5 were re-tested at a range of siRNA concentrations in HeLa cells. The siRNA concentrations tested were 100 nM, 33.3 nM, 11.1 nM, 3.70 nM, 1.23 nM, 0.41 nM, 0.14 nM and 0.046 nM. Assays were performed in sextuplicate, and the concentration of each siRNA resulting in fifty percent inhibition of cell proliferation (IC₅₀) was calculated. This dose-response analysis was performed between two and four times for each duplex. Mean IC₅₀ values (nM) are given in Table 6.

TABLE 6
IC50 of siRNA: cell proliferation in HeLa cells

| Duplex | Mean IC₅₀ |
|---|---|
| AL-DP-6226 | 15.5 |
| AL-DP-6229 | 3.4 |
| AL-DP-6231 | 4.2 |
| AL-DP-6232 | 17.5 |
| AL-DP-6239 | 4.4 |
| AL-DP-6242 | 5.2 |
| AL-DP-6243 | 2.6 |
| AL-DP-6244 | 8.3 |
| AL-DP-6248 | 1.9 |

### Example 3

### Eg5 siRNA in vitro Screening via mRNA Inhibition

Directly before transfection, HeLa S3 (ATCC-Number: CCL-2.2, LCG Promochem GmbH, Wesel, Germany) cells were seeded at 1.5×10⁴ cells/well on 96-well plates (Greiner Bio-One GmbH, Frickenhausen, Germany) in 75 μl of growth medium (Ham's F12, 10% fetal calf serum, 100 u penicillin/100 μg/ml streptomycin, all from Bookroom AG, Berlin, Germany). Transfections were performed in quadruplicates. For each well 0.5 μl Lipofectamine2000 (Invitrogen GmbH, Karlsruhe, Germany) were mixed with 12 μl Opti-MEM (Invitrogen) and incubated for 15 min at room temperature. For the siRNA concentration being 50 nM in the 100 μl transfection volume, 1 μl of a 5 μM siRNA were mixed with 11.5 μl Opti-MEM per well, combined with the Lipofectamine2000-Opti-MEM mixture and again incubated for 15 minutes at room temperature. siRNA-Lipofectamine2000-complexes were applied completely (25 μl each per well) to the cells and cells were incubated for 24 h at 37° C. and 5% CO₂ in a humidified incubator (Heroes GmbH, Hanau). The single dose screen was done once at 50 nM and at 25 nM, respectively.

Cells were harvested by applying 50 μl of lysis mixture (content of the QuantiGene bDNA-kit from Genospectra, Fremont, USA) to each well containing 100 μl of growth medium and were lysed at 53° C. for 30 min. Afterwards, 50 μl of the lists were incubated with probe sets specific to human Eg5 and human GAPDH and proceeded according to the manufacturer's protocol for QuantiGene. In the end chemoluminescence was measured in a Victor2-Light (Perkin Elmer, Wiesbaden, Germany) as RLUs (relative light units) and values obtained with the hEg5 probe set were normalized to the respective GAPDH values for each well. Values obtained with siRNAs directed against Eg5 were related to the value obtained with an unspecific siRNA (directed against HCV) which was set to 100% (Tables 1b, 2b and 3b).

Effective siRNAs from the screen were further characterized by dose response curves. Transfections of dose response curves were performed at the following concentrations: 100 nM, 16.7 nM, 2.8 nM, 0.46 nM, 77 picoM, 12.8 picoM, 2.1 picoM, 0.35 picoM, 59.5 fM, 9.9 fM and mock (no siRNA) and diluted with Opti-MEM to a final concentration of 12.5 μl according to the above protocol. Data analysis was performed by using the Microsoft Excel add-in software XL-fit 4.2 (IDBS, Guildford, Surrey, UK) and applying the dose response model number 205 (Tables 1b, 2b and 3b).

The lead siRNA AD12115 was additionally analyzed by applying the WST-proliferation assay from Roche (as previously described).

A subset of 34 duplexes from Table 2 that showed greatest activity was assayed by transfection in HeLa cells at final concentrations ranging from 100 nM to 10 fM. Transfections were performed in quadruplicate. Two dose-response assays were performed for each duplex. The concentration giving 20% (IC20), 50% (IC50) and 80% (IC80) reduction of KSP mRNA was calculated for each duplex (Table 7).

TABLE 7
Dose response mRNA inhibition of Eg5/KSP duplexes in HeLa cells
Concentrations given in pM

| | IC20s | | IC50s | | IC80s | |
|---|---|---|---|---|---|---|
| | 1^{st} | 2^{nd} | 1st | 2nd | 1st | 2nd |
| Duplex name | screen | screen | screen | screen | screen | screen |
| AD12077 | 1.19 | 0.80 | 6.14 | 10.16 | 38.63 | 76.16 |
| AD12078 | 25.43 | 25.43 | 156.18 | 156.18 | ND | ND |
| AD12085 | 9.08 | 1.24 | 40.57 | 8.52 | 257.68 | 81.26 |
| AD12095 | 1.03 | 0.97 | 9.84 | 4.94 | 90.31 | 60.47 |
| AD12113 | 4.00 | 5.94 | 17.18 | 28.14 | 490.83 | 441.30 |
| AD12115 | 0.60 | 0.41 | 3.79 | 3.39 | 23.45 | 23.45 |
| AD12125 | 31.21 | 22.02 | 184.28 | 166.15 | 896.85 | 1008.11 |
| AD12134 | 2.59 | 5.51 | 17.87 | 22.00 | 116.36 | 107.03 |
| AD12149 | 0.72 | 0.50 | 4.51 | 3.91 | 30.29 | 40.89 |
| AD12151 | 0.53 | 6.84 | 4.27 | 10.72 | 22.88 | 43.01 |
| AD12152 | 155.45 | 7.56 | 867.36 | 66.69 | 13165.27 | ND |
| AD12157 | 0.30 | 26.23 | 14.60 | 92.08 | 14399.22 | 693.31 |
| AD12166 | 0.20 | 0.93 | 3.71 | 3.86 | 46.28 | 20.59 |
| AD12180 | 28.85 | 28.85 | 101.06 | 101.06 | 847.21 | 847.21 |
| AD12185 | 2.60 | 0.42 | 15.55 | 13.91 | 109.80 | 120.63 |
| AD12194 | 2.08 | 1.11 | 5.37 | 5.09 | 53.03 | 30.92 |
| AD12211 | 5.27 | 4.52 | 11.73 | 18.93 | 26.74 | 191.07 |
| AD12257 | 4.56 | 5.20 | 21.68 | 22.75 | 124.69 | 135.82 |
| AD12280 | 2.37 | 4.53 | 6.89 | 20.23 | 64.80 | 104.82 |
| AD12281 | 8.81 | 8.65 | 19.68 | 42.89 | 119.01 | 356.08 |
| AD12282 | 7.71 | 456.42 | 20.09 | 558.00 | ND | ND |
| AD12285 | ND | 1.28 | 57.30 | 7.31 | 261.79 | 42.53 |
| AD12292 | 40.23 | 12.00 | 929.11 | 109.10 | ND | ND |
| AD12252 | 0.02 | 18.63 | 6.35 | 68.24 | 138.09 | 404.91 |
| AD12275 | 25.76 | 25.04 | 123.89 | 133.10 | 1054.54 | 776.25 |
| AD12266 | 4.85 | 7.80 | 10.00 | 32.94 | 41.67 | 162.65 |
| AD12267 | 1.39 | 1.21 | 12.00 | 4.67 | 283.03 | 51.12 |
| AD12264 | 0.92 | 2.07 | 8.56 | 15.12 | 56.36 | 196.78 |
| AD12268 | 2.29 | 3.67 | 22.16 | 25.64 | 258.27 | 150.84 |
| AD12279 | 1.11 | 28.54 | 23.19 | 96.87 | 327.28 | 607.27 |
| AD12256 | 7.20 | 33.52 | 46.49 | 138.04 | 775.54 | 1076.76 |
| AD12259 | 2.16 | 8.31 | 8.96 | 40.12 | 50.05 | 219.42 |
| AD12276 | 19.49 | 6.14 | 89.60 | 59.60 | 672.51 | 736.72 |
| AD12321 | 4.67 | 4.91 | 24.88 | 19.43 | 139.50 | 89.49 |

(ND—not determined)

### Example 4

### Silencing of Liver Eg5/KSP in Juvenile Rats Following Single-bolus Administration of LNP01 Formulated siRNA

From birth until approximately 23 days of age, Eg5/KSP expression can be detected in the growing rat liver. Target silencing with a formulated Eg5/KSP siRNA was evaluated in juvenile rats using duplex AD-6248.

KSP Duplex Tested

| | | | |
|---|---|---|---|
| Duplex ID | Target | Sense | Antisense |

| | | | |
|---|---|---|---|
| AD6248 | KSP | AccGAAGuGuuGuuuGuccTsT | GGAcAAAcAAcACUUCGGUTsT |
| | | (SEQ ID NO: 1238) | (SEQ ID NO: 1239) |

Methods

Dosing of animals. Male, juvenile Sprague-Dawley rats (19 days old) were administered single doses of lipidoid (“LNP01”) formulated siRNA via tail vein injection. Groups of ten animals received doses of 10 milligrams per kilogram (mg/kg) bodyweight of either AD6248 or an unspecific siRNA. Dose level refers to the amount of siRNA duplex administered in the formulation. A third group received phosphate-buffered saline. Animals were sacrificed two days after siRNA administration. Livers were dissected, flash frozen in liquid Nitrogen and pulverized into powders.

mRNA measurements. Levels of Eg5/KSP mRNA were measured in livers from all treatment groups. Samples of each liver powder (approximately ten milligrams) were homogenized in tissue lysis buffer containing proteinase K. Levels of Eg5/KSP and GAPDH mRNA were measured in triplicate for each sample using the Quantigene branched DNA assay (GenoSpectra). Mean values for Eg5/KSP were normalized to mean GAPDH values for each sample. Group means were determined and normalized to the PBS group for each experiment.

Statistical analysis. Significance was determined by ANOVA followed by the Tukey post-hoc test.

Results

Data Summary

Mean values (±standard deviation) for Eg5/KSP mRNA are given. Statistical significance (p value) versus the PBS group is shown (ns, not significant [p>0.05]).

TABLE 8
Experiment 1

| | | KSP/GAPDH | p value |
|---|---|---|---|
| PBS | | 1.0 ± 0.47 | |
| AD6248 | 10 mg/kg | 0.47 ± 0.12 | <0.001 |
| unspec | 10 mg/kg | 1.0 ± 0.26 | ns |

A statistically significant reduction in liver Eg5/KSP mRNA was obtained following treatment with formulated AD6248 at a dose of 10 mg/kg.

### Example 5

### Silencing of Rat Liver VEGF Following Intravenous Infusion of LNP01 Formulated VSP

A “lipidoid” formulation comprising an equimolar mixture of two siRNAs was administered to rats. As used herein, VSP refers to a composition having two siRNAs, one directed to Eg5/KSP and one directed to VEGF. For this experiment the duplex AD3133 directed towards VEGF and AD12115 directed towards Eg5/KSP were used. Since Eg5/KSP expression is nearly undetectable in the adult rat liver, only VEGF levels were measured following siRNA treatment.

siRNA Duplexes Administered (VSP)

| Duplex | | | |
|---|---|---|---|
| ID | Target | Sense | Antisense |
| AD12115 | Eg5/KSP | ucGAGAAucuAAAcuAAcuTsT | AGUuAGUUuAGAUUCUCGATsT |
| | | (SEQ ID NO: 1240) | (SEQ ID NO: 1241) |
| AD3133 | VEGF | GcAcAuAGGAGAGAuGAGCUsU | AAGCUcAUCUCUCCuAuGuGCusG |
| | | (SEQ ID NO: 1242) | (SEQ ID NO: 1243) |

Key: A,G,C,U-ribonucleotides; c,u-2′-O-Me ribonucleotides; s-phosphorothioate.

Unmodified versions of each strand and the targets for each siRNA are as follows

| | | |
|---|---|---|
| Eg5/KSP | unmod sense | 5′ UCGAGAAUCUAAACUAACUTT 3′ SEQ ID NO: 1534 |
| | unmod antisense | 3′ TTAGUCCUUAGAUUUGAUUGA 5′ SEQ ID NO: 1535 |
| | target | 5′ UCGAGAAUCUAAACUAACU 3′ SEQ ID NO: 1311 |
| VEGF | unmod sense | 5′ GCACAUAGGAGAGAUGAGCUU 3′ SEQ ID NO: 1536 |
| | unmod antisense | 3′ GUCGUGUAUCCUCUCUACUCGAA 5′ SEQ ID NO: 1537 |
| | target | 5′ GCACAUAGGAGAGAUGAGCUU 3′ SEQ ID NO: 1538 |

### Methods

Dosing of animals. Adult, female Sprague-Dawley rats were administered lipidoid (“LNP01”) formulated siRNA by a two-hour infusion into the femoral vein. Groups of four animals received doses of 5, 10 and 15 milligrams per kilogram (mg/kg) bodyweight of formulated siRNA. Dose level refers to the total amount of siRNA duplex administered in the formulation. A fourth group received phosphate-buffered saline. Animals were sacrificed 72 hours after the end of the siRNA infusion. Livers were dissected, flash frozen in liquid Nitrogen and pulverized into powders.

Formulation Procedure

The lipidoid ND98.4HCl (MW 1487) (Formula 1, above), Cholesterol (Sigma-Aldrich), and PEG-Ceramide C16 (Avanti Polar Lipids) were used to prepare lipid-siRNA nanoparticles. Stock solutions of each in ethanol were prepared: ND98, 133 mg/mL; Cholesterol, 25 mg/mL, PEG-Ceramide C16, 100 mg/mL. ND98, Cholesterol, and PEG-Ceramide C16 stock solutions were then combined in a 42:48:10 molar ratio. Combined lipid solution was mixed rapidly with aqueous siRNA (in sodium acetate pH 5) such that the final ethanol concentration was 35-45% and the final sodium acetate concentration was 100-300 mM. Lipid-siRNA nanoparticles formed spontaneously upon mixing. Depending on the desired particle size distribution, the resultant nanoparticle mixture was in some cases extruded through a polycarbonate membrane (100 nm cut-off) using a thermobarrel extruder (Lipex Extruder, Northern Lipids, Inc). In other cases, the extrusion step was omitted. Ethanol removal and simultaneous buffer exchange was accomplished by either dialysis or tangential flow filtration. Buffer was exchanged to phosphate buffered saline (PBS) pH 7.2.

Characterization of Formulations

Formulations prepared by either the standard or extrusion-free method are characterized in a similar manner. Formulations are first characterized by visual inspection. They should be whitish translucent solutions free from aggregates or sediment. Particle size and particle size distribution of lipid-nanoparticles are measured by dynamic light scattering using a Malvern Zetasizer Nano ZS (Malvern, USA). Particles should be 20-300 nm, and ideally, 40-100 nm in size. The particle size distribution should be unimodal. The total siRNA concentration in the formulation, as well as the entrapped fraction, is estimated using a dye exclusion assay. A sample of the formulated siRNA is incubated with the RNA-binding dye Ribogreen (Molecular Probes) in the presence or absence of a formulation disrupting surfactant, 0.5% Triton-X100. The total siRNA in the formulation is determined by the signal from the sample containing the surfactant, relative to a standard curve. The entrapped fraction is determined by subtracting the “free” siRNA content (as measured by the signal in the absence of surfactant) from the total siRNA content. Percent entrapped siRNA is typically >85%. For SNALP formulation, the particle size is at least 30 nm, at least 40 nm, at least 50 nm, at least 60 nm, at least 70 nm, at least 80 nm, at least 90 nm, at least 100 nm, at least 110 nm, and at least 120 nm. The preferred range is about at least 50 nm to about at least 110 nm, preferably about at least 60 nm to about at least 100 nm, most preferably about at least 80 nm to about at least 90 nm. In one example, each of the particle size comprises at least about 1:1 ratio of Eg5 dsRNA to VEGF dsRNA.

mRNA measurements. Samples of each liver powder (approximately ten milligrams) were homogenized in tissue lysis buffer containing proteinase K. Levels of VEGF and GAPDH mRNA were measured in triplicate for each sample using the Quantigene branched DNA assay (GenoSpectra). Mean values for VEGF were normalized to mean GAPDH values for each sample. Group means were determined and normalized to the PBS group for each experiment.

Protein measurements. Samples of each liver powder (approximately 60 milligrams) were homogenized in 1 ml RIPA buffer. Total protein concentrations were determined using the Micro BCA protein assay kit (Pierce). Samples of total protein from each animal were used to determine VEGF protein levels using a VEGF ELISA assay (R&D systems). Group means were determined and normalized to the PBS group for each experiment.

Statistical analysis. Significance was determined by ANOVA followed by the Tukey post-hoc test

Results

Data Summary

Mean values (±standard deviation) for mRNA (VEGF/GAPDH) and protein (rel. VEGF) are shown for each treatment group. Statistical significance (p value) versus the PBS group for each experiment is shown.

TABLE 9

| | VEGF/GAPDH | p value | rel VEGF | p value |
|---|---|---|---|---|
| PBS | 1.0 ± 0.17 | | 1.0 ± 0.17 | |
| 5 mg/kg | 0.74 ± 0.12 | <0.05 | 0.23 ± 0.03 | <0.001 |
| 10 mg/kg | 0.65 ± 0.12 | <0.005 | 0.22 ± 0.03 | <0.001 |
| 15 mg/kg | 0.49 ± 0.17 | <0.001 | 0.20 ± 0.04 | <0.001 |

Statistically significant reductions in liver VEGF mRNA and protein were measured at all three siRNA dose levels.

### Example 6

### Assessment of VSP SNALP in Mouse Models of Human Hepatic tumors

These studies utilized a VSP siRNA cocktail containing dsRNAs targeting KSP/Eg5 and dsRNAs targeting VEGF. As used herein, VSP refers to a composition having two siRNAs, one directed to Eg5/KSP and one directed to VEGF. For this experiment the duplexes AD3133 (directed towards VEGF) and AD12115 (directed towards Eg5/KSP) were used. The siRNA cocktail was formulated in SNALP as described below.

The maximum study size utilized 20-25 mice. To test the efficacy of the siRNA SNALP cocktail to treat liver cancer, 1×10̂6 tumor cells were injected directly into the left lateral lobe of test mice. The incisions were closed by sutures, and the mice allowed to recover for 2-5 hours. The mice were fully recovered within 48-72 hours. The SNALP siRNA treatment was initiated 8-11 days after tumor seeding.

The SNALP formulations utilized were (i) VSP (KSP+VEGF siRNA cocktail (1:1 molar ratio)); (ii) KSP (KSP+Luc siRNA cocktail); and (iii) VEGF (VEGF+Luc siRNA cocktail). All formulations contained equal amounts (mg) of each active siRNA. All mice received a total siRNA/lipid dose, and each cocktail was formulated into 1:57 cDMA SNALP (1.4% PEG-cDMA; 57.1% DLinDMA; 7.1% DPPC; and 34.3% cholesterol), 6:1 lipid:drug using original citrate buffer conditions.

Human Hep3B Study A: Anti-Tumor Activity of VSP-SNALP

Human Hepatoma Hep3B tumors were established in scid/beige mice by intrahepatic seeding. Group A (n=6) animals were administered PBS; Group B (n=6) animals were administered VSP SNALP; Group C (n=5) animals were administered KSP/Luc SNALP; Group D (n=5) animals were administered VEGF/Luc SNALP.

SNALP treatment was initiated eight days after tumor seeding. The SNALP was dosed at 3 mg/kg total siRNA, twice weekly (Monday and Thursday), for a total of six doses (cumulative 18 mg/kg siRNA). The final dose was administered at day 25, and the terminal endpoint was at day 27.

Tumor burden was assayed by (a) body weight; (b) liver weight; (c) visual inspection+photography at day 27; (d) human-specific mRNA analysis; and (e) blood alpha-fetoprotein levels measured at day 27.

Table 10 below illustrates the results of visual scoring of tumor burden measured in the seeded (left lateral) liver lobe. Score: “−”=no visible tumor; “+”=evidence of tumor tissue at injection site; “++”=Discrete tumor nodule protruding from liver lobe; “+++”=large tumor protruding on both sides of liver lobe; “++++”=large tumor, multiple nodules throughout liver lobe.

TABLE 10

| | Mouse | Tumor Burden |
|---|---|---|
| Group A: PBS, day 27 | 1 | ++++ |
| | 2 | ++++ |
| | 3 | ++ |
| | 4 | +++ |
| | 5 | ++++ |
| | 6 | ++++ |
| Group B: VSP | 1 | + |
| (VEGF + KSP/Eg5, d. 27 | 2 | − |
| | 3 | − |
| | 4 | − |
| | 5 | ++ |
| | 6 | − |
| Group C: KSP | 1 | + |
| (Luc + KSP), d. 27 | 2 | ++ |
| | 3 | − |
| | 4 | + |
| | 5 | ++ |
| Group D: VEGF | 1 | ++++ |
| (Luc + VEGF), d. 27 | 2 | − |
| | 3 | ++++ |
| | 4 | +++ |
| | 5 | ++++ |

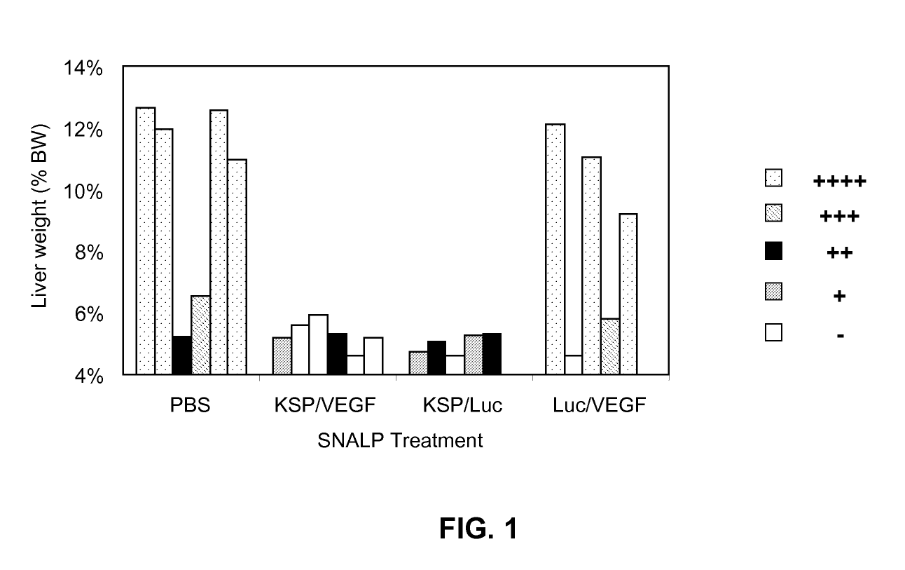

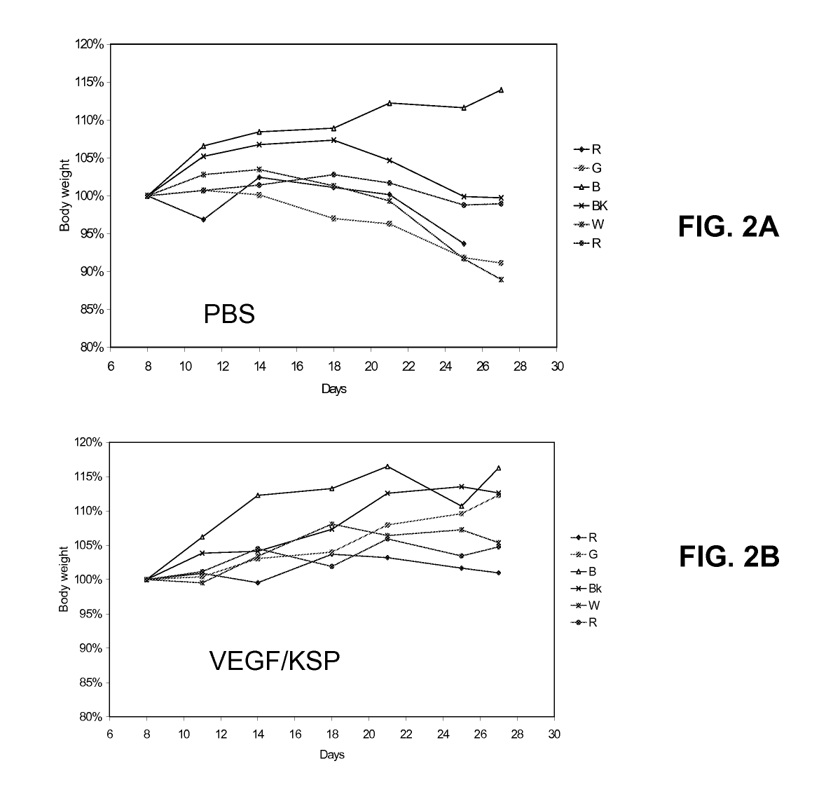

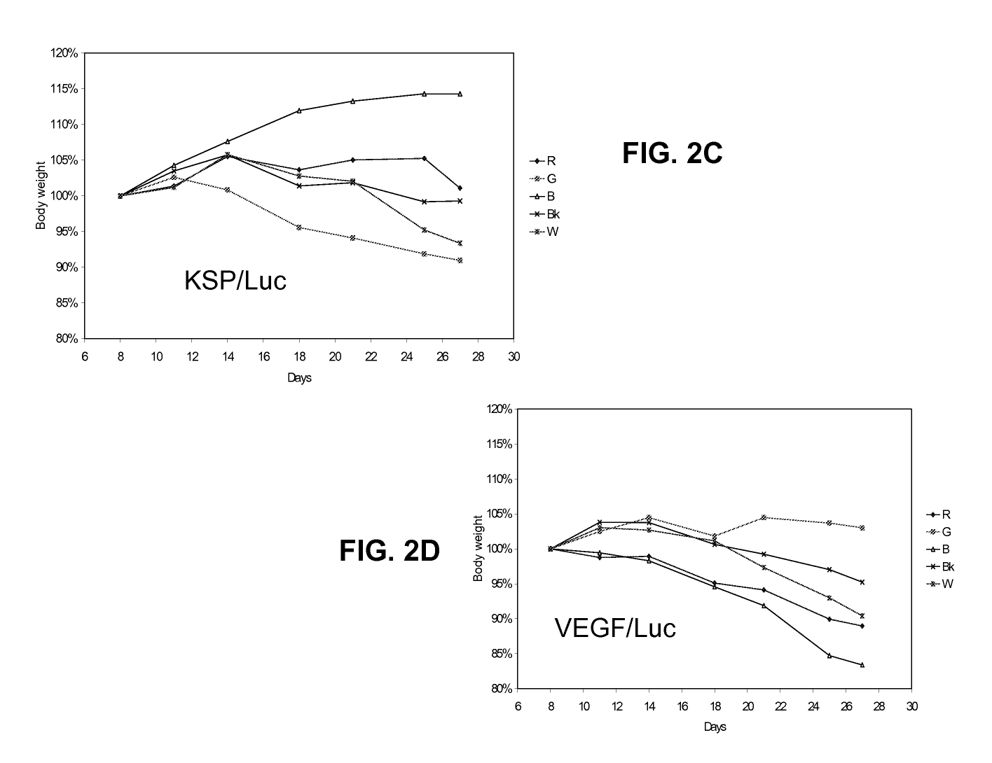

Liver weights, as percentage of body weight, are shown in FIG. 1. FIG. 2A, FIG. 2B, FIG. 2C and FIG. 2D show the effects of PBS, VSP, KSP and VEGF on body weight on Human Hepatoma Hep3B tumors in mice.

From this study, the following conclusions were made. (1) VSP SNALP demonstrated potent anti-tumor effects in Hep3B 1H model; (2) the anti-tumor activity of the VSP cocktail appeared largely associated with the KSP component; (3) anti-KSP activity was confirmed by single dose histological analysis; and (4) VEGF siRNA showed no measurable effect on inhibition of tumor growth in this model.

Human Hep3B Study B: Prolonged Survival with VSP Treatment

In a second Hep3B study, human hepatoma Hep3B tumors were established by intrahepatic seeding into scid/beige mice. These mice were deficient for lymphocytes and natural killer (NK) cells, which is the minimal scope for immune-mediated anti-tumor effects. Group A (n=6) mice were untreated; Group B (n=6) mice were administered luciferase (luc) 1955 SNALP (Lot No. AP10-02); and Group C (n=7) mice were administered VSP SNALP (Lot No. AP10-01). SNALP was 1:57 cDMA SNALP, and 6:1 lipid:drug.

SNALP treatment was initiated eight days after tumor seeding. SNALP was dosed at 3 mg/kg siRNA, twice weekly (Mondays and Thursdays), for a total of six doses (cumulative 18 mg/kg siRNA). The final dose was delivered at day 25, and the terminal endpoint of the study was at day 27.

Tumor burden was assayed by (1) body weight; (2) visual inspection+photography at day 27; (3) human-specific mRNA analysis; and (4) blood alpha-fetoprotein measured at day 27.

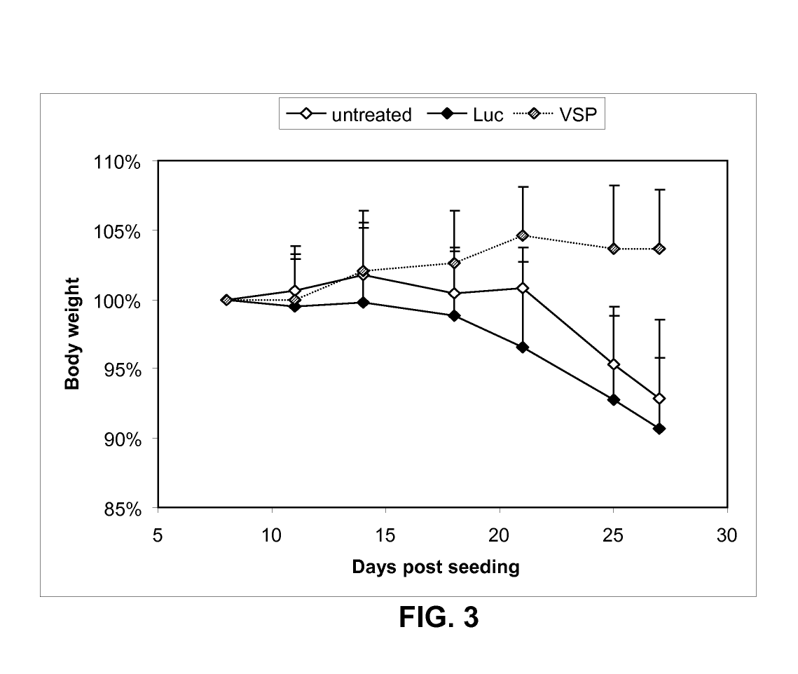

FIG. 3 shows body weights were measured at each day of dosing (days 8, 11, 14, 18, 21, and 25) and on the day of sacrifice.

TABLE 11

| | | Tumor Burden |
|---|---|---|
| | | by macroscopic |
| | Mouse | observation |
| Group A: untreated, | A1R | ++ |
| day 27 | A1G | ++++ |
| | A1W | − |
| | A2R | ++++ |
| | A2G | +++ |
| | A2W | ++++ |
| Group B: | B1R | ++++ |
| 1955 Luc SNALP, day 27 | B1G | ++++ |
| | B1W | +++ |
| | B2R | ++ |
| | B2G | +++ |
| | B2W | ++++ |
| Group C: | C1R | − |
| VSP SNALP, day 27 | C1G | − |
| | C1B | − |
| | C1W | + |
| | C2R | + |
| | C2G | + |
| | C2W | − |

Score:
“−” = no visible tumor;
“+” = evidence of tumor tissue at injection site;
“++” = Discrete tumor nodule protruding from liver lobe;
“+++” = large tumor protruding on both sides of liver lobe;
“++++” = large tumor, multiple nodules throughout liver lobe.

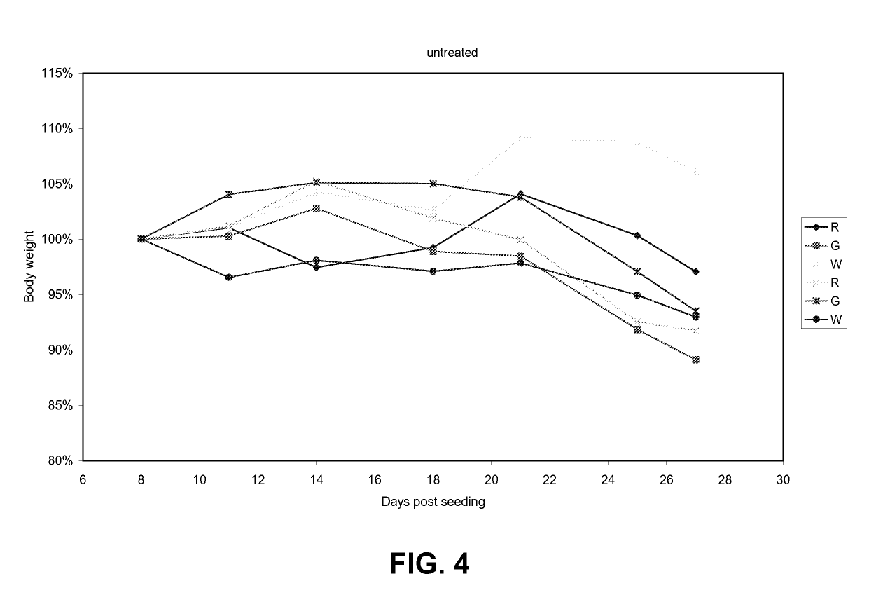

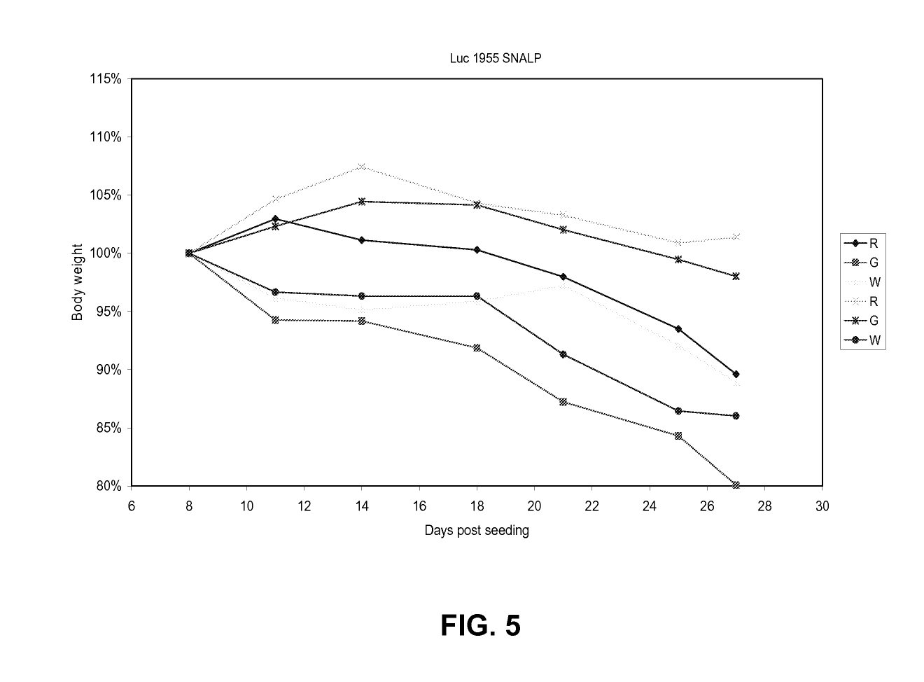

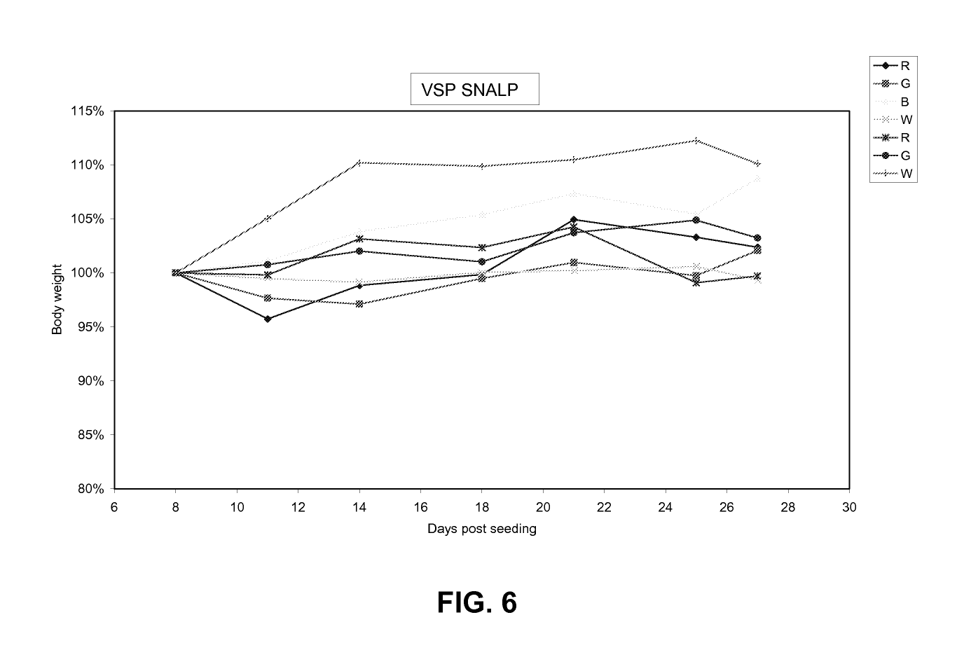

The correlation between body weights and tumor burden are shown in FIGS. 4, 5 and 6. FIG. 4 shows percentage body weight over 27 days in untreated mice. FIG. 5 shows percentage body weight over 27 days in 1955 Luc SNALP treated mice. FIG. 6 shows percentage body weight over 27 days in VSP SNALP treated mice.

A single dose of VSP SNALP (2 mg/kg) to Hep3B mice also resulted in the formation of mitotic spindles in liver tissue samples examined by histological staining

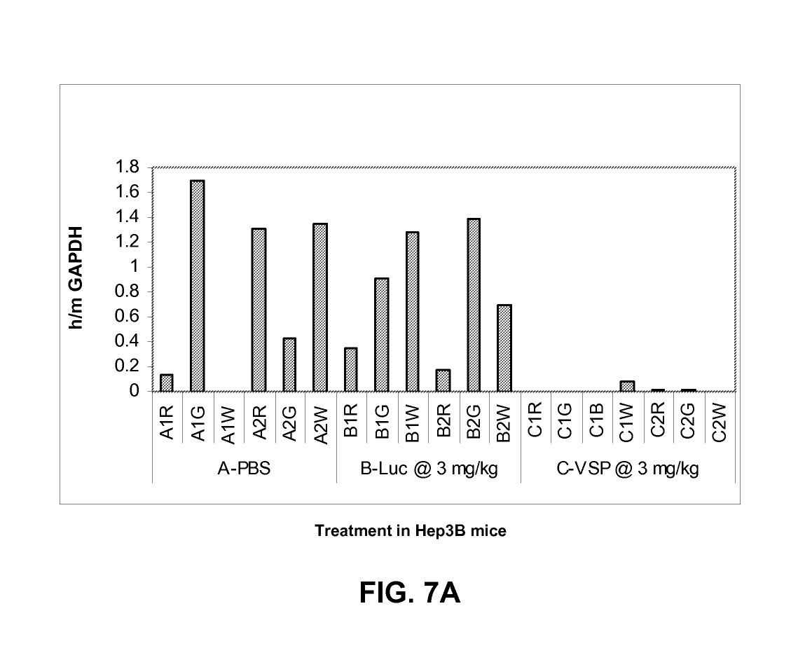

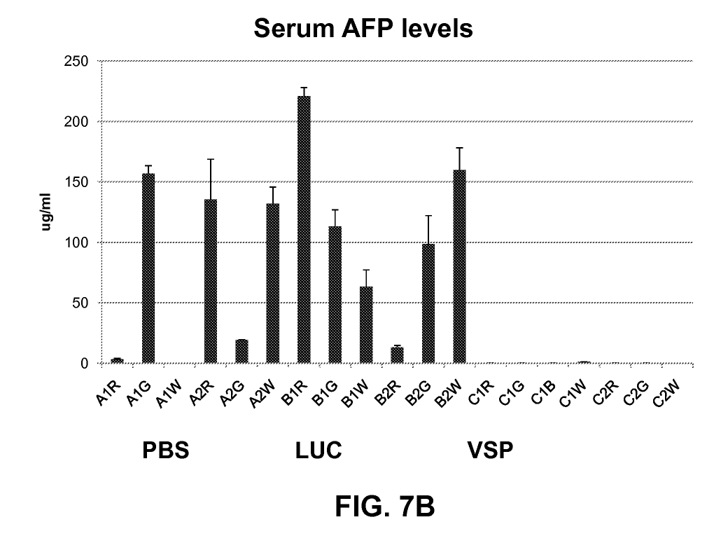

Tumor burden was quantified by quantitative RT-PCR (pRT-PCR) (Taqman). Human GAPDH was normalized to mouse GAPDH via species-specific Taqman assays. FIG. 7A shows tumor scores as shown by macroscopic observation in the table above correlated with GADPH levels.

Serum ELISA was performed to measure alpha-fetoprotein (AFP) secreted by the tumor. As described below, if levels of AFP go down after treatment, the tumor is not growing. FIG. 7B shows that the treatment with VSP lowered AFP levels in some animals compared to treatment with controls.

Human HepB3 Study C:

In a third study, human HCC cells (HepB3) were injected directly into the liver of SCID/beige mice, and treatment was initiated 20 days later. Group A animals were administered PBS; Group B animals were administered 4 mg/kg Luc-1955 SNALP; Group C animals were administered 4 mg/kg SNALP-VSP; Group D animals were administered 2 mg/kg SNALP-VSP; and Group E animals were administered 1 mg/kg SNALP-VSP. Treatment was with a single intravenous (iv) dose, and mice were sacrificed 24 hr. later.

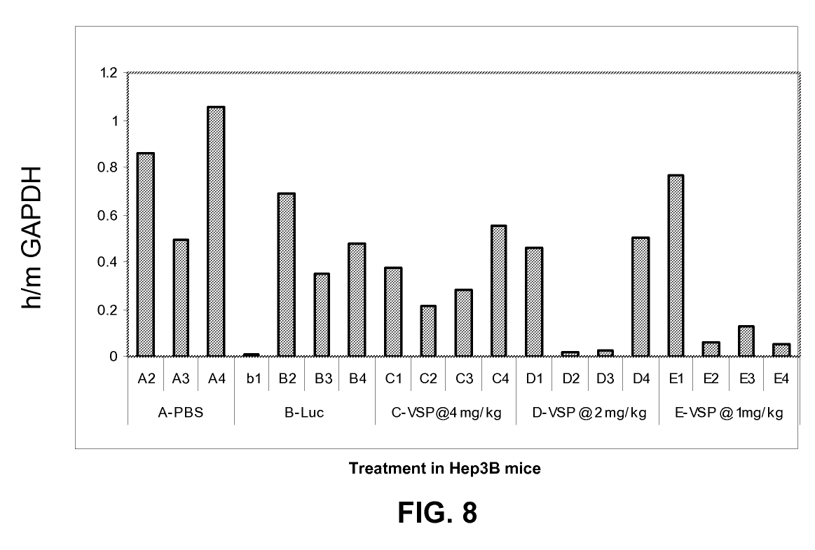

Tumor burden and target silencing was assayed by qRT-PCR (Taqman). Tumor score was also measured visually as described above, and the results are shown in the following table. hGAPDH levels, as shown in FIG. 8, correlates with macroscopic tumor score as shown in the table below.

TABLE 12

| | | Tumor Burden by macroscopic |
|---|---|---|
| | Mouse | observation |
| Group A: PBS | A2 | +++ |
| | A3 | +++ |
| | A4 | +++ |
| Group B: 4 mg/kg Luc- | B1 | + |
| 1955 SNALP | B2 | +++ |
| | B3 | +++ |
| | B4 | +++ |
| Group C: 4 mg/kg | C1 | ++ |
| SNALP-VSP | C2 | ++ |
| | C3 | ++ |
| | C4 | +++ |
| Group D: 2 mg/kg | D1 | ++ |
| SNALP-VSP | D2 | + |
| | D3 | + |
| | D4 | ++ |
| Group E: 1 mg/kg | E1 | +++ |
| SNALP-VSP | E2 | + |
| | E3 | ++ |
| | E4 | + |

Score: “+”=variable tumor take/some small tumors; “++”=Discrete tumor nodule protruding from liver lobe; “+++”=large tumor protruding on both sides of liver lobe

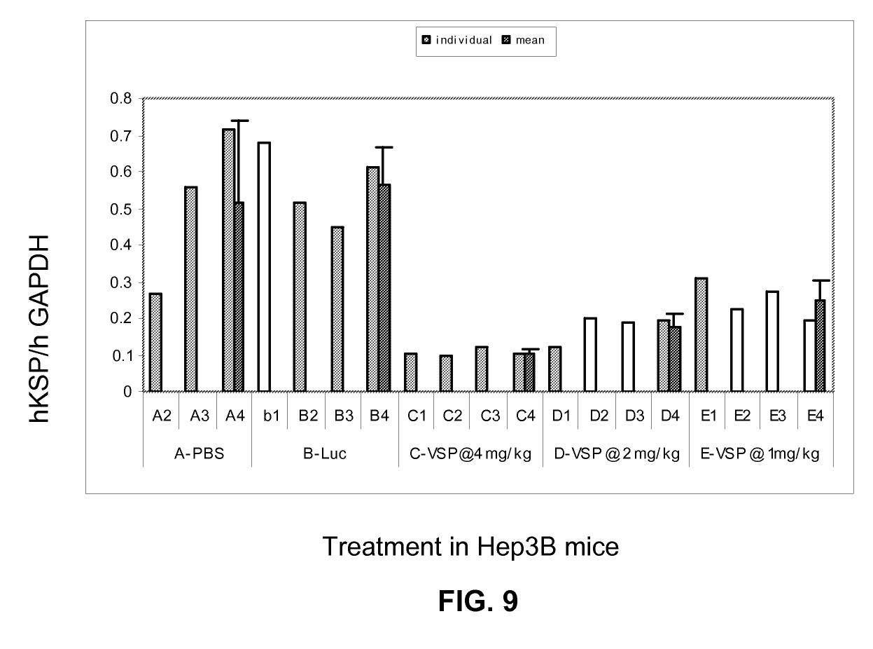

Human (tumor-derived) KSP silencing was assayed by Taqman analysis and the results are shown in FIG. 9. hKSP expression was normalized to hGAPDH. About 80% tumor KSP silencing was observed at 4 mg/kg SNALP-VSP, and efficacy was evident at 1 mg/kg. The clear bars in FIG. 9 represent the results from small (low GAPDH) tumors.

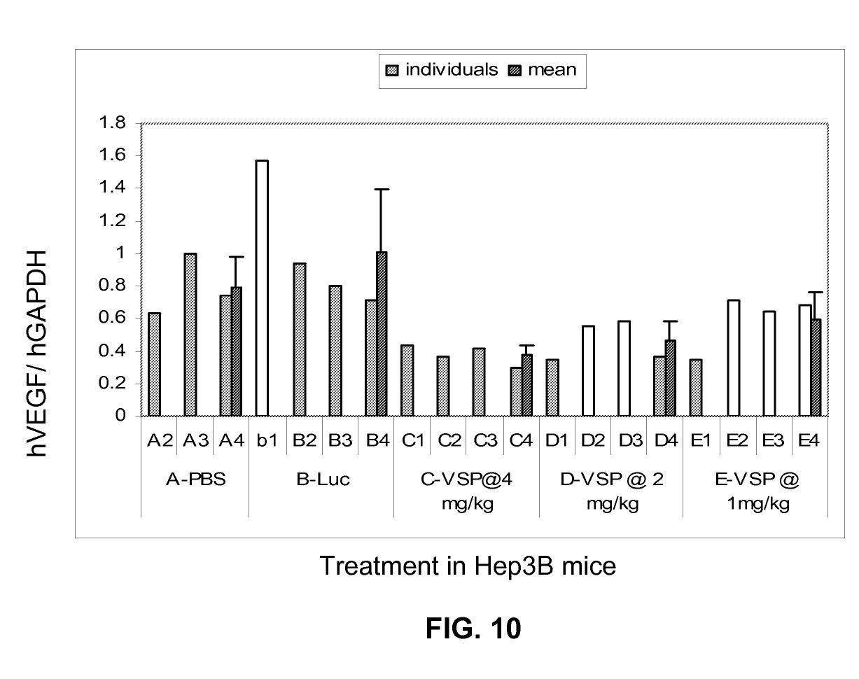

Human (tumor-derived) VEGF silencing was assayed by Taqman analysis and the results are shown in FIG. 10. hVEGF expression was normalized to hGAPDH. About 60% tumor VEGF silencing was observed at 4 mg/kg SNALP-VSP, and efficacy was evident at 1 mg/kg. The clear bars in FIG. 10 represent the results from small (low GAPDH) tumors.

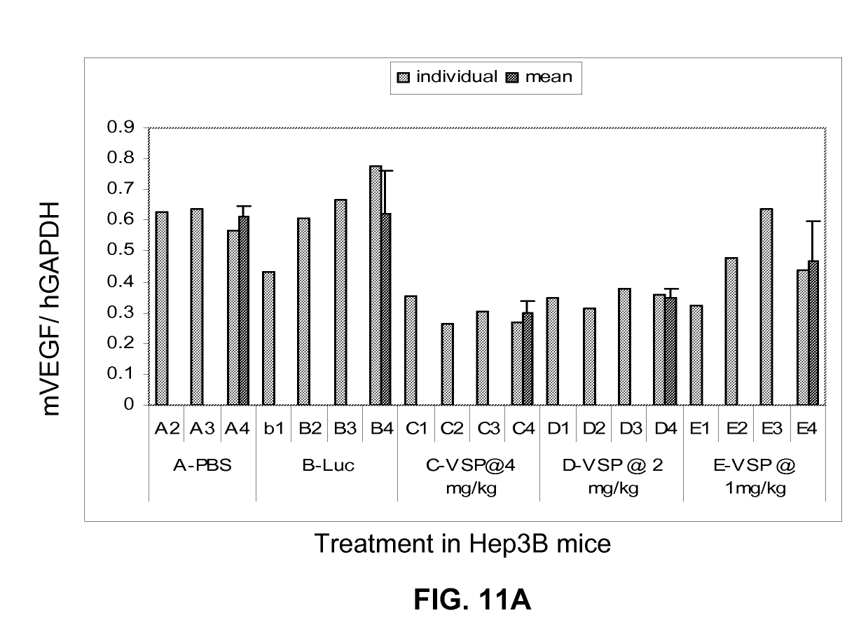

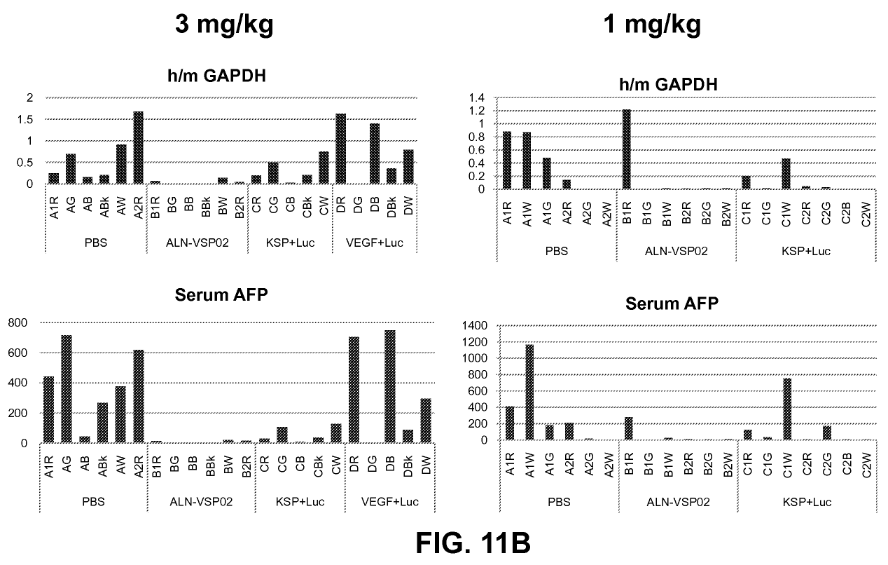

Mouse (liver-derived) VEGF silencing was assayed by Taqman analysis and the results are shown in FIG. 11A. mVEGF expression was normalized to hGAPDH. About 50% liver VEGF silencing was observed at 4 mg/kg SNALP-VSP, and efficacy was evident at 1 mg/kg.

Human HepB3 Study D: Contribution of Each dsRNA to Tumor Growth

In a fourth study, human HCC cells (HepB3) were injected directly into the liver of SCID/beige mice, and treatment was initiated 8 days later. Treatment was with intravenous (iv) bolus injections, twice weekly, for a total of six does. The final dose was administered at day 25, and the terminal endpoint was at day 27.

Tumor burden was assayed by gross histology, human-specific mRNA analysis (hGAPDH qPCR), and blood alpha-fetoprotein levels (serum AFP via ELISA).

In Study 1, Group A was treated with PBS, Group B was treated with SNALP-KSP+Luc (3 mg/kg), Group C was treated with SNALP-VEGF+Luc (3 mg/kg), and Group D was treated with SNALP-VSP (3 mg/kg).

In Study 2, Group A was treated with PBS; Group B was treated with SNALP-KSP+Luc (1 mg/kg), Group C was treated with ALN-VSP02 (1 mg/kg).

Both GAPDH mRNA levels and serum AFP levels were shown to decrease after treatment with SNALP-VSP (as shown in FIG. 11B).

Histology Studies:

Human hepatoma Hep3B tumors were established by intrahepatic seeding in mice. SNALP treatment was initiated 20 days after tumor seeding. Tumor-bearing mice (three per group) were treated with a single intravenous (IV) dose of (i) VSP SNALP or (ii) control (Luc) SNALP at 2 mg/kg total siRNA.

Liver/tumor samples were collected for conventional H&E histology 24 hours after single SNALP administration.

Large macroscopic tumor nodules (5-10 mm) were evident at necroscopy.

Effect of SNALP-VSP in Hep3B Mice:

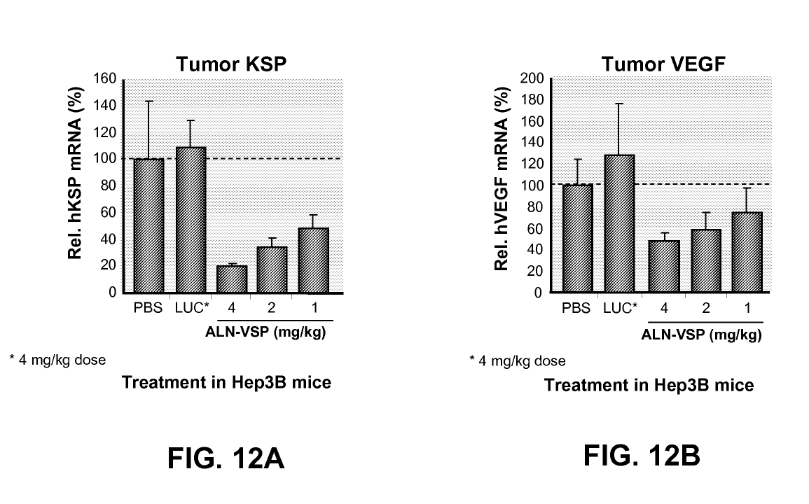

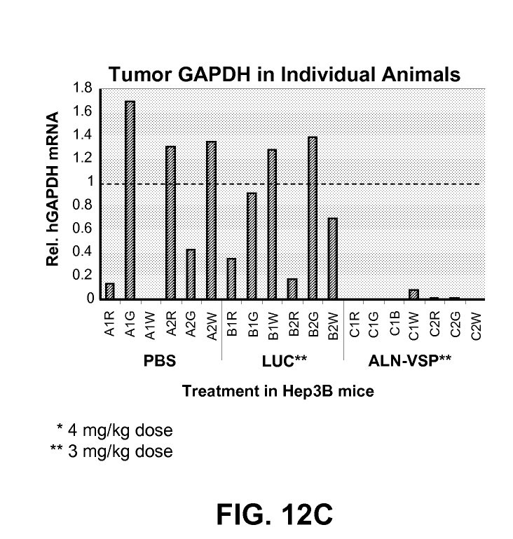

SNALP-VSP (a cocktail of KSP dsRNA and VEGF dsRNA) treatment reduced tumor burden and expression of tumor-derived KSP and VEGF. GAPDH mRNA levels, a measure of tumor burden, were also observed to decline following administration of SNALP-VSP dsRNA (shown in FIG. 12A, FIG. 12B and FIG. 12C). A decrease in tumor burden by visual macroscopic observation was also evident following administration of SNALP-VSP.

A single IV bolus injection of SNALP-VSP also resulted in mitotic spindle formation that was clearly detected in liver tissue samples from Hep3B mice. This observation indicated cell cycle arrest.

### Example 7

### Survival of SNALP-VSP Animals Versus SNALP-Luc Treated Animals

To test the effect of siRNA SNALP on survival rates of cancer subjects, tumors were established by intrahepatic seeding in mice and the mice were treated with SNALP-siRNA. These studies utilized a VSP siRNA cocktail containing dsRNAs targeting KSP/Eg5 and VEGF. Control was dsRNA targeting Luc. The siRNA cocktail was formulated in SNALPs.

Tumor cells (Human Hepatoma Hep3B, 1×10̂6) were injected directly into the left lateral lobe of scid/beige mice. These mice were deficient for lymphocytes and natural killer (NK) cells, which is the minimal scope for immune-mediated anti-tumor effects. The incisions were closed by sutures, and the mice allowed to recover for 2-5 hours. The mice were fully recovered within 48-72 hours.

All mice received a total siRNA/lipid intravenous (iv) dose, and each cocktail was formulated into 1:57 cDMA SNALP (1.4% PEG-cDMA; 57.1% DLinDMA; 7.1% DPPC; and 34.3% cholesterol), 6:1 lipid:drug using original citrate buffer conditions.

siRNA-SNALP treatment was initiated on the day indicated below (18 or 26 days) after tumor seeding. siRNA-SNALP were administered twice a week for three weeks after 18 or 26 days at a dose of 4 mg/kg. Survival was monitored and animals were euthanized based on humane surrogate endpoints (e.g., animal body weight, abdominal distension/discoloration, and overall health).

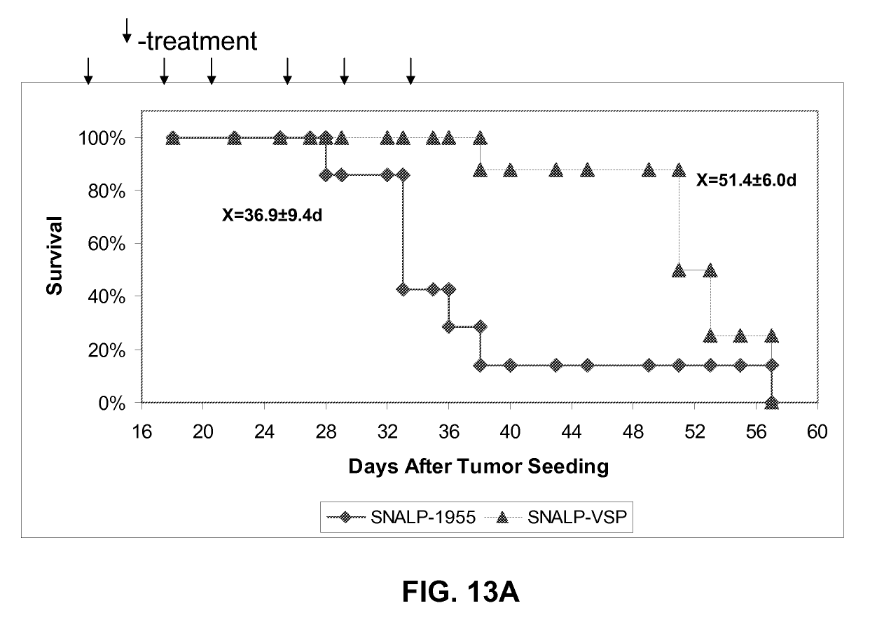

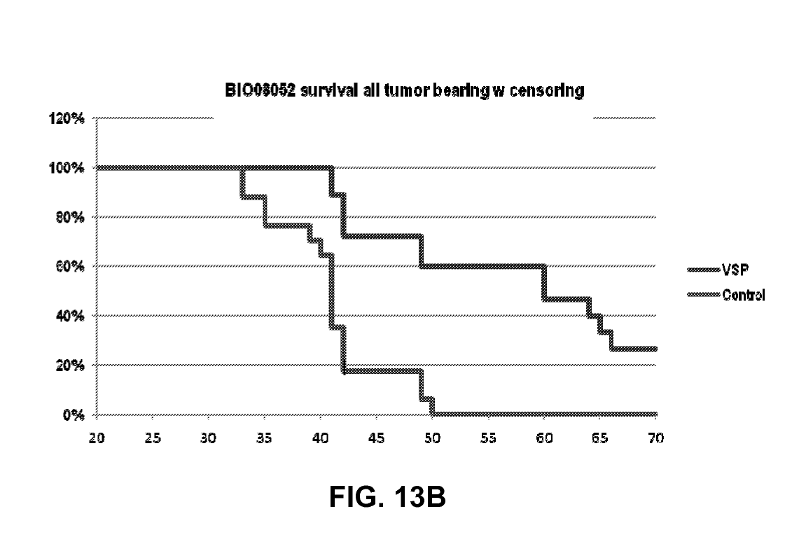

The survival data for treatment initiated 18 days after tumor seeing is summarized in Table 13, Table 14, and FIG. 13A.

TABLE 13
Kaplan-Meier (survival) data (% Surviving)

| | SNALP- | SNALP- |
|---|---|---|
| Day | Luc | VSP |
| 18 | 100% | 100% |
| 22 | 100% | 100% |
| 25 | 100% | 100% |
| 27 | 100% | 100% |
| 28 | 100% | 100% |
| 28 | 86% | 100% |
| 29 | 86% | 100% |
| 32 | 86% | 100% |
| 33 | 86% | 100% |
| 33 | 43% | 100% |
| 35 | 43% | 100% |
| 36 | 43% | 100% |
| 36 | 29% | 100% |
| 38 | 29% | 100% |
| 38 | 14% | 100% |
| 38 | 14% | 88% |
| 40 | 14% | 88% |
| 43 | 14% | 88% |
| 45 | 14% | 88% |
| 49 | 14% | 88% |
| 51 | 14% | 88% |
| 51 | 14% | 50% |
| 53 | 14% | 50% |
| 53 | 14% | 25% |
| 55 | 14% | 25% |
| 57 | 14% | 25% |
| 57 | 0% | 0% |

TABLE 14
Survival in days, for each animal.

| | Treatment | | |
|---|---|---|---|
| Animal | group | Survival | |
| 1 | SNALP-Luc | 28 | days |
| 2 | SNALP-Luc | 33 | days |
| 3 | SNALP-Luc | 33 | days |
| 4 | SNALP-Luc | 33 | days |
| 5 | SNALP-Luc | 36 | days |
| 6 | SNALP-Luc | 38 | days |
| 7 | SNALP-Luc | 57 | days |
| 8 | SNALP-VSP | 38 | days |
| 9 | SNALP-VSP | 51 | days |
| 10 | SNALP-VSP | 51 | days |
| 11 | SNALP-VSP | 51 | days |
| 12 | SNALP-VSP | 53 | days |
| 13 | SNALP-VSP | 53 | days |
| 14 | SNALP-VSP | 57 | days |
| 15 | SNALP-VSP | 57 | days |

FIG. 13A shows the mean survival of SNALP-VSP animals and SNALP-Luc treated animals versus days after tumor seeding. The mean survival of SNALP-VSP animals was extended by approximately 15 days versus SNALP-Luc treated animals.

TABLE 15
Serum alpha fetoprotein (AFP) concentration, for each animal, at a
time pre-treatment and at end of treatment (concentration in μg/ml)

| | | | End of |
|---|---|---|---|
| | | pre-Rx | Rx |
| 1 | SNALP-Luc | 30.858 | 454.454 |
| 2 | SNALP-Luc | 10.088 | 202.082 |
| 3 | SNALP-Luc | 23.736 | 648.952 |
| 4 | SNALP-Luc | 1.696 | 13.308 |
| 5 | SNALP-Luc | 4.778 | 338.688 |
| 6 | SNALP-Luc | 15.004 | 826.972 |
| 7 | SNALP-Luc | 11.036 | 245.01 |
| 8 | SNALP-VSP | 37.514 | 182.35 |
| 9 | SNALP-VSP | 91.516 | 248.06 |
| 10 | SNALP-VSP | 25.448 | 243.13 |
| 11 | SNALP-VSP | 24.862 | 45.514 |
| 12 | SNALP-VSP | 57.774 | 149.352 |
| 13 | SNALP-VSP | 12.446 | 78.724 |
| 14 | SNALP-VSP | 2.912 | 9.61 |
| 15 | SNALP-VSP | 4.516 | 11.524 |

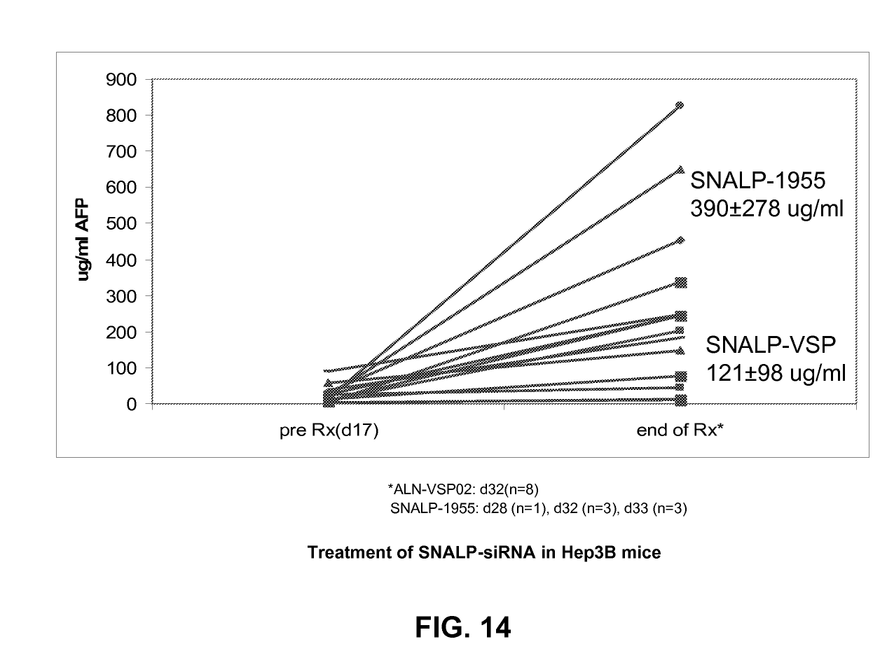

Tumor burden was monitored using serum AFP levels during the course of the experiment. Alpha-fetoprotein (AFP) is a major plasma protein produced by the yolk sac and the liver during fetal life. The protein is thought to be the fetal counterpart of serum albumin, and human AFP and albumin gene are present in tandem in the same transcriptional orientation on chromosome 4. AFP is found in monomeric as well as dimeric and trimeric forms, and binds copper, nickel, fatty acids and bilirubin. AFP levels decrease gradually after birth, reaching adult levels by 8-12 months. Normal adult AFP levels are low, but detectable. AFP has no known function in normal adults and AFP expression in adults is often associated with a subset of tumors such as hepatoma and teratoma. AFP is a tumor marker used to monitor testicular cancer, ovarian cancer, and malignant teratoma. Principle tumors that secrete AFP include endodermal sinus tumor (yolk sac carcinoma), neuroblastoma, hepatoblastoma, and heptocellular carcinoma. In patients with AFP-secreting tumors, serum levels of AFP often correlate with tumor size. Serum levels are useful in assessing response to treatment. Typically, if levels of AFP go down after treatment, the tumor is not growing. A temporary increase in AFP immediately following chemotherapy may indicate not that the tumor is growing but rather that it is shrinking (and releasing AFP as the tumor cells die). Resection is usually associated with a fall in serum levels. As shown in FIG. 14, tumor burden in SNALP-VSP treated animals was significantly reduced.

The experiment was repeated with SNALP-siRNA treatment at 26, 29, 32 35, 39, and 42 days after implantation. The data is shown in FIG. 13B. The mean survival of SNALP-VSP animals was extended by approximately 15 days versus SNALP-Luc treated animals by approximately 19 days, or 38%.

### Example 8

### Induction of Mono-Asters in Established Tumors

Inhibition of KSP in dividing cells leads to the formation of mono asters that are readily observable in histological sections. To determine whether mono aster formation occurred in SNALP-VSP treated tumors, tumor bearing animals (three weeks after Hep3B cell implantation) were administered 2 mg/kg SNALP-VSP via tail vein injection. Control animals received 2 mg/kg SNALP-Luc. Each cocktail was formulated into 1:57 cDMA SNALP (1.4% PEG-cDMA; 57.1% DLinDMA; 7.1% DPPC; and 34.3% cholesterol), 6:1 lipid:drug using original citrate buffer conditions.

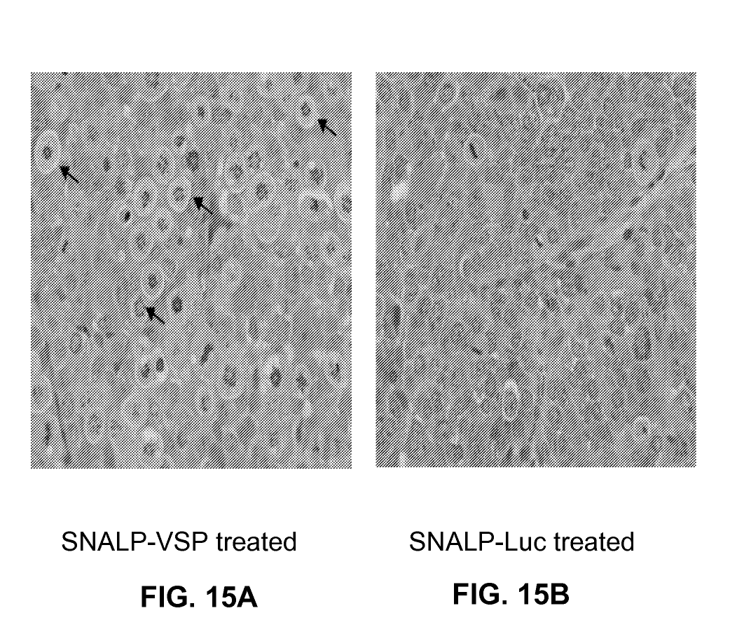

Twenty four hours later, animals were sacrificed, and tumor bearing liver lobes were processed for histological analysis. Representative images of H&E stained tissue sections are shown in FIG. 15. Extensive mono aster formation was evident in SNALP-VSP treated (A), but not SNALP-Luc treated (B), tumors. In the latter, normal mitotic figures were evident. The generation of mono asters is a characteristic feature of KSP inhibition and provides further evidence that SNALP-VSP has significant activity in established liver tumors.

### Example 9

### Manufacturing Process and Product Specification of ALN-VSP02 (SNALP-VSP)

ALN-VSP02 product contains 2 mg/mL of drug substance ALN-VSPDS01 formulated in a sterile lipid particle formulation (referred to as SNALP) for IV administration via infusion. Drug substance ALN-VSPDS01 consists of two siRNAs (ALN-12115 targeting KSP and ALN-3133 targeting VEGF) in an equimolar ratio. The drug product is packaged in 10 mL glass vials with a fill volume of 5 mL.

The drug substance can be formulated in other nucleic acid-lipid particle formulations as described herein, e.g., with cationic lipids XTC, ALNY-100, and MC3.

The following terminology is used herein:

| Drug Substance | siRNA Duplexes | Single Strand Intermediates |
|---|---|---|
| ALN-VSPDS01 | ALN-12115* | Sense: A-19562 |
| | | Antisense: A-19563 |
| | ALN-3133** | Sense: A-3981 |
| | | Antisense: A-3982 |

*Alternate names = AD-12115, AD12115;
**Alternate names = AD-3133, ADS3133

9.1 Preparation of Drug Substance ALN-VSPDS01

The two siRNA components of drug substance ALN-VSPDS01, ALN-12115 and ALN-3133, are chemically synthesized using commercially available synthesizers and raw materials. The manufacturing process consists of synthesizing the two single strand oligonucleotides of each duplex (A 19562 sense and A 19563 antisense of ALN 12115 and A 3981 sense and A 3982 antisense of ALN 3133) by conventional solid phase oligonucleotide synthesis using phosphoramidite chemistry and 5′ O dimethoxytriphenylmethyl (DMT) protecting group with the 2′ hydroxyl protected with tert butyldimethylsilyl (TBDMS) or the 2′ hydroxyl replaced with a 2′ methoxy group (2′ OMe). Assembly of an oligonucleotide chain by the phosphoramidite method on a solid support such as controlled pore glass or polystyrene. The cycle consists of 5′ deprotection, coupling, oxidation, and capping. Each coupling reaction is carried out by activation of the appropriately protected ribo, 2′ OMe, or deoxyribonucleoside amidite using 5 (ethylthio) 1H tetrazole reagent followed by the coupling of the free 5′ hydroxyl group of a support immobilized protected nucleoside or oligonucleotide. After the appropriate number of cycles, the final 5′ protecting group is removed by acid treatment. The crude oligonucleotide is cleaved from the solid support by aqueous methylamine treatment with concomitant removal of the cyanoethyl protecting group as well as nucleobase protecting groups. The 2′ O TBDMS group is then cleaved using a hydrogen fluoride containing reagent to yield the crude oligoribonucleotide, which is purified using strong anion exchange high performance liquid chromatography (HPLC) followed by desalting using ultrafiltration. The purified single strands are analyzed to confirm the correct molecular weight, the molecular sequence, impurity profile and oligonucleotide content, prior to annealing into the duplexes. The annealed duplex intermediates ALN 12115 and ALN 3133 are either lyophilized and stored at 20° C. or mixed in 1:1 molar ratio and the solution is lyophilized to yield drug substance ALN VSPDS01. If the duplex intermediates were stored as dry powder, they are re-dissolved in water before mixing. The equimolar ratio is achieved by monitoring the mixing process by an HPLC method.

Example specifications are shown in Table 16a.

TABLE 16a
Example specifications for ALN-VSPDS01

| Test | Method | Acceptance Criteria |
|---|---|---|
| Appearance | Visual | White to off-white powder |
| Identity, ALN-VSPDS01 | Duplex AX-HPLC | Duplex retention times are consistent |
| ALN-3133 | | with those of reference standards |
| ALN-12115 | | |
| Identity, ALN-VSPDS01 | MS | Molecular weight of single strands are |
| | | within the following ranges: |
| | | A-3981: 6869-6873 Da |
| | | A-3982: 7305-7309 Da |
| | | A-19562: 6762-6766 Da |
| | | A-19563: 6675-6679 Da |
| Sodium counter ion (% w/w on | Flame AAS or ICP-OES | Report data |
| anhydrous basis) | | |
| ALN-VSPDS01 assay | Denaturing AX-HPLC | 90-110% |
| Purity of ALN-VSPDS01 | SEC | ≧90.0 area % |
| Single strand purity, | Denaturing AX-HPLC | Report data |
| ALN-VSPDS01 | | Report area % for total impurities |
| siRNA molar ratio | Duplex AX-HPLC | 1.0 ± 0.1 |
| Moisture content | Karl Fischer titration | ≦15% |
| Residual solvents | HS-Capillary GC | |
| Acetonitrile | | ≦410 ppm |
| Ethanol | | ≦5000 ppm |
| Isopropanol | | ≦5000 ppm |
| pH of 1% solution | USP <791> | Report data |
| Heavy metals | ICP-MS | Report data |
| As, Cd, Cu, Cr, Fe, Ni, Pb, Sn | | |
| Bacterial endotoxins | USP <85> | ≦0.5 EU/mg |
| Bioburden | Modified USP <61> | <100 CFU/g |

The results of up to 12 month stability testing for ALN-VSPDS01 drug substance are shown in Tables 16b. The assay methods were chosen to assess physical property (appearance, pH, moisture), purity (by SEC and denaturing anion exchange chromatography) and potency (by denaturing anion exchange chromatography [AX-HPLC]).

TABLE 16b
Stability of drug substance

| Lot No.: A05M07001N | | | Study Storage Conditions: −20° C. (Storage Condition) | | | | |
|---|---|---|---|---|---|---|---|
| | | Acceptance | Results | | | | |
| Test | Method | Criteria | Initial | 1 Month | 3 Months | 6 Months | 12 Months |
| Appearance | Visual | White to off- | Pass | Pass | Pass | Pass | Pass |
| | | white powder | | | | | |
| pH | USP <791> | Report data | 6.7 | 6.4 | 6.6 | 6.4 | 6.8 |
| Moisture | Karl Fischer | ≦15% | 3.6* | 6.7 | 6.2 | 5.6 | 5.0 |
| content | titration | | | | | | |
| (% w/w) | | | | | | | |
| Purity (area | SEC | ≧90.0 area % | 95 | 95 | 94 | 92 | 95 |
| %) | | | | | | | |
| A-3981 | Denaturing AX- | Report data | 24 | 23 | 23 | 23 | 23 |
| (sense) | HPLC | | | | | | |
| (area %) | | | | | | | |
| A-3982 | Denaturing AX- | Report data | 23 | 23 | 23 | 23 | 24 |
| (antisense) | HPLC | | | | | | |
| (area %) | | | | | | | |
| A-19562 | Denaturing AX- | Report data | 22 | 21 | 21 | 21 | 21 |
| (sense) | HPLC | | | | | | |
| (area %) | | | | | | | |
| A-19563 | Denaturing AX- | Report data | 23 | 22 | 22 | 22 | 22 |
| (antisense) | HPLC | | | | | | |
| (area %) | | | | | | | |

9.2 Preparation of Drug Product ALN-VSP02

ALN VSP02, is a sterile formulation of the two siRNAs (in a 1:1 molar ratio) with lipid excipients in isotonic buffer. The lipid excipients associate with the two siRNAs, protect them from degradation in the circulatory system, and aid in their delivery to the target tissue. The specific lipid excipients and the quantitative proportion of each (shown in Table 17) have been selected through an iterative series of experiments comparing the physicochemical properties, stability, pharmacodynamics, pharmacokinetics, toxicity and product manufacturability of numerous different formulations. The excipient DLinDMA is a titratable aminolipid that is positively charged at low pH, such as that found in the endosome of mammalian cells, but relatively uncharged at the more neutral pH of whole blood. This feature facilitates the efficient encapsulation of the negatively charged siRNAs at low pH, preventing formation of empty particles, yet allows for adjustment (reduction) of the particle charge by replacing the formulation buffer with a more neutral storage buffer prior to use. Cholesterol and the neutral lipid DPPC are incorporated in order to provide physicochemical stability to the particles. The polyethyleneglycol lipid conjugate PEG2000 C DMA aids drug product stability, and provides optimum circulation time for the proposed use. ALN VSP02 lipid particles have a mean diameter of approximately 80-90 nm with low polydispersity values. At neutral pH, the particles are essentially uncharged, with Zeta Potential values of less than 6 mV. There is no evidence of empty (non loaded) particles based on the manufacturing process.

TABLE 17
Quantitative Composition of ALN-VSP02

| | Proportion |
|---|---|
| Component, grade | (mg/mL) |
| ALN-VSPDS01, cGMP | 2.0* |
| DLinDMA | 7.3 |
| (1,2-Dilinoleyloxy-N,N-dimethyl-3- | |
| aminopropane), cGMP | |
| DPPC (R-1,2-Dipalmitoyl-sn-glycero- | 1.1 |
| 3-phosphocholine), cGMP | |
| Cholesterol, Synthetic, cGMP | 2.8 |
| PEG2000-C-DMA | 0.8 |
| (3-N-[(ω-Methoxy poly(ethylene | |
| glycol) 2000) carbamoyl]-1,2- | |
| dimyristyloxy-propylamine), cGMP | |
| Phosphate Buffered Saline, cGMP | q.s. |

*The 1:1 molar ratio of the two siRNAs in the drug product is maintained throughout the size distribution of the drug product particles.

Solutions of lipid (in ethanol) and ALN VSPDS01 drug substance (in aqueous buffer) are mixed and diluted to form a colloidal dispersion of siRNA lipid particles with an average particle size of approximately 80-90 nm. This dispersion is then filtered through 0.45/0.2 μm filters, concentrated, and diafiltered by Tangential Flow Filtration. After in process testing and concentration adjustment to 2.0 mg/mL, the product is sterile filtered, aseptically filled into glass vials, stoppered, capped and placed at 5±3° C. The ethanol and all aqueous buffer components are USP grade; all water used is USP Sterile Water For Injection grade.ALN-VSP02.

A similar method is used to formulate ALN-VSPDS01 in other lipid formulations, e.g., those with cationic lipids XTC, ALNY-100, and MC3.

### Example 10

### In Vitro Efficacy of ALN-VSP02 in Human Cancer Cell Lines

The efficacy of ALN-VSP02 treatment in human cancer cell lines was determined via measurement of KSP mRNA, VEGF mRNA, and cell viability after treatment. IC50 (nM) values determined for KSP and VEGF in each cell line.

TABLE 19
cell lines

| Cell line tested | ATCC cat number |
|---|---|
| HELA | ATCC Cat N: CCL-2 |
| KB | ATCC Cat N: CCL-17 |
| HEP3B | ATCC Cat N: HB-8064 |
| SKOV-3 | ATCC Cat N: HTB-77 |
| HCT-116 | ATCC Cat N: CCL-247 |
| HT-29 | ATCC Cat N: HTB-38 |
| PC-3 | ATCC Cat N: CRL-1435 |
| A549 | ATCC Cat N: CCL-185 |
| MDA-MB-231 | ATCC Cat N: HTB-26 |

Cells were plated in 96 well plates in complete media at day 1 to reach a density of 70% on day 2. On day 2 media was replaced with Opti-MEM reduced serum media (Invitrogen Cat N: 11058-021) and cells were transfected with either ALN-VSP02 or control SNALP-Luc with concentration range starting at 1.8 μM down to 10 pM. After 6 hours the media was changed to complete media. Three replicate plates for each cell line for each experiment was done.

ALN-VSP02 was formulated as described in Table 17.

Cells were harvested 24 hours after transfection. KSP levels were measured using bDNA; VEGF mRNA levels were measured using human TaqMan assay.

Viability was measured using Cell Titer Blue reagent (Promega Cat N: G8080) at 48 and/or 72h following manufacturer's recommendations.

As shown in Table 20, nM concentrations of VSP02 are effective in reducing expression of both KSP and VEGF in multiple human cell lines. Viability of treated cells was not

TABLE 20
Results

| | IC50 (nM) | IC50 (nM) |
|---|---|---|
| Cell line | KSP | VEGF |
| HeLa | 8.79 | 672 |
| SKOV-3 | 142 | 1347 |
| HCT116 | 31.6 | 27.5 |
| Hep3B | 1.3 | 14.5 |
| HT-29 | 262 | ND |
| PC3 | 127 | ND |
| KB | 50.6 | ND |
| A549 | 201 | ND |
| MB231 | 187 | ND |

### Example 11

### Anti-Tumor Efficacy of VSP SNALP vs. Sorafenib in Established Hep3B Intrahepatic Tumors

The anti-tumor effects of multi-dosing VSP SNALP verses Sorafenib in scid/beige mice bearing established Hep3B intrahepatic tumors was studied. Sorafenib is a small molecule inhibitor of protein kinases approved for treatment of hepatic cellular carcinoma (HCC).

Tumors were established by intrahepatic seeding in scid/beige mice as described herein. Treatment was initiated 11 days post-seeding. Mice were treated with Sorafenib and a control siRNA-SNALP, Sorafenib and VSP siRNA-SNALP, or VSP siRNA-SNALP only. Control mice were treated with buffers only (DMSO for Sorafenib and PBS for siRNA-SNALP). Sorafenib was administered intraparenterally from Mon to Fri for three weeks, at 15 mg/kg according to body weight for a total of 15 injections. Sorafenib was administered a minimum of 1 hour after SNALP injections. The siRNA-SNALPS were administered intravenously via the lateral tail vein according at 3 mg/kg based on the most recently recorded body weight (10 ml/kg) for 3 weeks (total of 6 doses) on days 1, 4, 7, 10, 14, and 17.

Each siRNA-SNALP was formulated into 1:57 cDMA SNALP (1.4% PEG-cDMA; 57.1% DLinDMA; 7.1% DPPC; and 34.3% cholesterol), 6:1 lipid:drug using original citrate buffer conditions.

Mice were euthanized based on an assessment of tumor burden including progressive weight loss and clinical signs including condition, abdominal distension/discoloration and mobility.

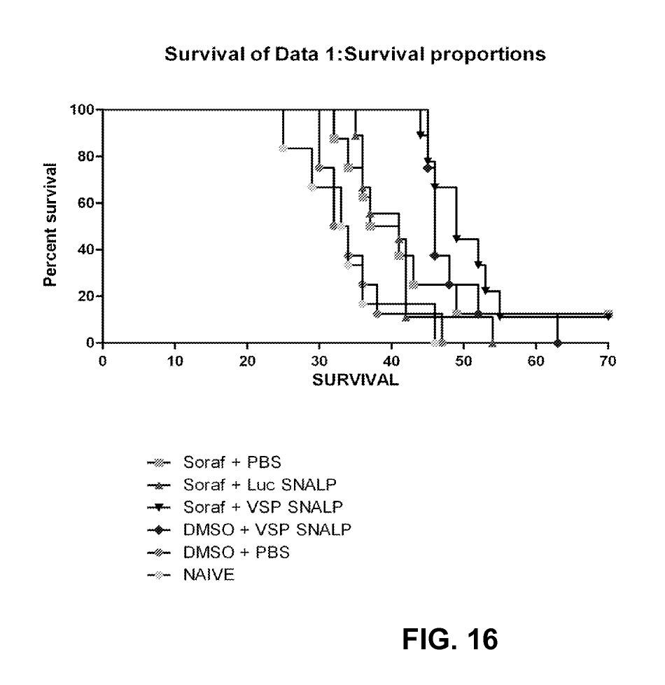

The percent survival data are shown in FIG. 16. Co-administration of VSP siRNA-SNALP with Sorafenib increased survival proportion compared to administration of Sorafenib or VSP siRNA-SNALP alone. VSP siRNA-SNALP increased survival proportion compared to Sorafenib.

### Example 12

### In vitro Efficacy of VSP Using Variants of AD-12115 and AD-3133

Two sets of duplexes targeted to Eg5/KSP and VEGF were designed and synthesized. Each set included duplexes tiling 10 nucleotides in each direction of the target sites for either AD-12115 and AD-3133.

Sequences of the target, sense strand, and antisense strand for each duplex are shown in the Table below.

Each duplex is assayed for inhibition of expression using the assays described herein. The duplexes are administered alone and/or in combination, e.g., an Eg5/KSP dsRNA in combination with a VEGF dsRNA. In some embodiments, the dsRNA are administered in a nucleic-acid lipid particle, e.g., SNALP, formulation as described herein.

TABLE 21
Sequences of dsRNA targeted to VEGF and Eg5/KSP (tiling)

| | | | SEQ | Sense Strand | SEQ |
|---|---|---|---|---|---|
| | target | target sequence | ID | Antisense strand | ID |
| Duplex ID | gene | 5′ to 3′ | NO: | 5′ to 3′ | NO: |
| AD-20447.1 | VEGFA | ACCAAGGCCAGCACAUAGG | 2264 | AccAAGGccAGcAcAuAGGTsT | 2304 |
| | | | | CCuAUGUGCUGGCCUUGGUTsT | 2305 |
| AD-20448.1 | VEGFA | CCAAGGCCAGCACAUAGGA | 2265 | ccAAGGccAGcAcAuAGGATsT | 2306 |
| | | | | UCCuAUGUGCUGGCCUUGGTsT | 2307 |
| AD-20449.1 | VEGFA | CCAAGGCCAGCACAUAGGA | 2266 | ccAAGGccAGcAcAuAGGATsT | 2308 |
| | | | | CUCCuAUGUGCUGGCCUUGTsT | 2309 |
| AD-20450.1 | VEGFA | AAGGCCAGCACAUAGGAGA | 2267 | AAGGccAGcAcAuAGGAGATsT | 2310 |
| | | | | UCUCCuAUGUGCUGGCCUUTsT | 2311 |
| AD-20451.1 | VEGFA | AGGCCAGCACAUAGGAGAG | 2268 | AGGccAGcAcAuAGGAGAGTsT | 2312 |
| | | | | CUCUCCuAUGUGCUGGCCUTsT | 2313 |
| AD-20452.1 | VEGFA | GGCCAGCACAUAGGAGAGA | 2269 | GGccAGcAcAuAGGAGAGATsT | 2314 |
| | | | | UCUCUCCuAUGUGCUGGCCTsT | 2315 |
| AD-20453.1 | VEGFA | GCCAGCACAUAGGAGAGAU | 2270 | GccAGcAcAuAGGAGAGAuTsT | 2316 |
| | | | | AUCUCUCCuAUGUGCUGGCTsT | 2317 |
| AD-20454.1 | VEGFA | CCAGCACAUAGGAGAGAUG | 2271 | ccAGcAcAuAGGAGAGAuGTsT | 2318 |
| | | | | cAUCUCUCCuAUGUGCUGGTsT | 2319 |
| AD-20455.1 | VEGFA | CAGCACAUAGGAGAGAUGA | 2272 | cAGcAcAuAGGAGAGAuGATsT | 2320 |
| | | | | UcAUCUCUCCuAUGUGCUGTsT | 2321 |
| AD-20456.1 | VEGFA | AGCACAUAGGAGAGAUGAG | 2273 | AGcAcAuAGGAGAGAuGAGTsT | 2322 |
| | | | | CUcAUCUCUCCuAUGUGCUTsT | 2323 |
| AD-20457.1 | VEGFA | CACAUAGGAGAGAUGAGCU | 2274 | cAcAuAGGAGAGAuGAGcuTsT | 2324 |
| | | | | AGCUcAUCUCUCCuAUGUGTsT | 2325 |
| AD-20458.1 | VEGFA | ACAUAGGAGAGAUGAGCUU | 2275 | AcAuAGGAGAGAuGAGcuuTsT | 2326 |
| | | | | AAGCUcAUCUCUCCuAUGUTsT | 2327 |
| AD-20459.1 | VEGFA | CAUAGGAGAGAUGAGCUUC | 2276 | cAuAGGAGAGAuGAGcuucTsT | 2328 |
| | | | | GAAGCUcAUCUCUCCuAUGTsT | 2329 |
| AD-20460.1 | VEGFA | AUAGGAGAGAUGAGCUUCC | 2277 | AuAGGAGAGAuGAGcuuccTsT | 2330 |
| | | | | GGAAGCUcAUCUCUCCuAUTsT | 2331 |
| AD-20461.1 | VEGFA | UAGGAGAGAUGAGCUUCCU | 2278 | uAGGAGAGAuGAGcuuccuTsT | 2332 |
| | | | | AGGAAGCUcAUCUCUCCuATsT | 2333 |
| AD-20462.1 | VEGFA | AGGAGAGAUGAGCUUCCUA | 2279 | AGGAGAGAuGAGcuuccuATsT | 2334 |
| | | | | uAGGAAGCUcAUCUCUCCUTsT | 2335 |
| AD-20463.1 | VEGFA | GGAGAGAUGAGCUUCCUAC | 2280 | GGAGAGAuGAGcuuccuAcTsT | 2336 |
| | | | | GuAGGAAGCUcAUCUCUCCTsT | 2337 |
| AD-20464.1 | VEGFA | GAGAGAUGAGCUUCCUACA | 2281 | GAGAGAuGAGcuuccuAcATsT | 2338 |
| | | | | UGuAGGAAGCUcAUCUCUCTsT | 2339 |
| AD-20465.1 | VEGFA | AGAGAUGAGCUUCCUACAG | 2282 | AGAGAuGAGcuuccuAcAGTsT | 2340 |
| | | | | CUGuAGGAAGCUcAUCUCUTsT | 2341 |
| AD-20466.1 | VEGFA | GAGAUGAGCUUCCUACAGC | 2283 | GAGAuGAGcuuccuAcAGcTsT | 2342 |
| | | | | GCUGuAGGAAGCUcAUCUCTsT | 2343 |
| AD-20467.1 | KSP | AUGUUCCUUAUCGAGAAUC | 2284 | AuGuuccuuAucGAGAAucTsT | 2344 |
| | | | | GAUUCUCGAuAAGGAAcAUTsT | 2345 |
| AD-20468.1 | KSP | UGUUCCUUAUCGAGAAUCU | 2285 | uGuuccuuAucGAGAAucuTsT | 2346 |
| | | | | AGAUUCUCGAuAAGGAAcATsT | 2347 |
| AD-20469.1 | KSP | GUUCCUUAUCGAGAAUCUA | 2286 | GuuccuuAucGAGAAucuATsT | 2348 |
| | | | | uAGAUUCUCGAuAAGGAACTsT | 2349 |
| AD-20470.1 | KSP | UUCCUUAUCGAGAAUCUAA | 2287 | uuccuuAucGAGAAucuAATsT | 2350 |
| | | | | UuAGAUUCUCGAuAAGGAATsT | 2351 |
| AD-20471.1 | KSP | UCCUUAUCGAGAAUCUAAA | 2288 | uccuuAucGAGAAucuAAATsT | 2352 |
| | | | | UUuAGAUUCUCGAuAAGGATsT | 2353 |
| AD-20472.1 | KSP | CCUUAUCGAGAAUCUAAAC | 2289 | ccuuAucGAGAAucuAAAcTsT | 2354 |
| | | | | GUUuAGAUUCUCGAuAAGGTsT | 2355 |
| AD-20473.1 | KSP | CUUAUCGAGAAUCUAAACU | 2290 | cuuAucGAGAAucuAAAcuTsT | 2356 |
| | | | | AGUUuAGAUUCUCGAuAAGTsT | 2357 |
| AD-20474.1 | KSP | UUAUCGAGAAUCUAAACUA | 2291 | uuAucGAGAAucuAAAcuATsT | 2358 |
| | | | | uAGUUuAGAUUCUCGAuAATsT | 2359 |
| AD-20475.1 | KSP | UAUCGAGAAUCUAAACUAA | 2292 | uAucGAGAAucuAAAcuAATsT | 2360 |
| | | | | UuAGUUuAGAUUCUCGAuATsT | 2361 |
| AD-20476.1 | KSP | AUCGAGAAUCUAAACUAAC | 2293 | AucGAGAAucuAAAcuAAcTsT | 2362 |
| | | | | GUuAGUUuAGAUUCUCGAUTsT | 2363 |
| AD-20477.1 | KSP | CGAGAAUCUAAACUAACUA | 2294 | cGAGAAucuAAAcuAAcuATsT | 2364 |
| | | | | | 2365 |
| AD-20478.1 | KSP | GAGAAUCUAAACUAACUAG | 2295 | GAGAAucuAAAcuAAcuAGTsT | 2366 |
| | | | | CuAGUuAGUUuAGAUUCUCTsT | 2367 |
| AD-20479.1 | KSP | AGAAUCUAAACUAACUAGA | 2296 | AGAAucuAAAcuAAcuAGATsT | 2368 |
| | | | | UCuAGUuAGUUuAGAUUCUTsT | 2369 |
| AD-20480.1 | KSP | GAAUCUAAACUAACUAGAA | 2297 | GAAucuAAAcuAAcuAGAATsT | 2370 |
| | | | | UUCuAGUuAGUUuAGAUUCTsT | 2371 |
| AD-20481.1 | KSP | AAUCUAAACUAACUAGAAU | 2298 | AAucuAAAcuAAcuAGAAuTsT | 2372 |
| | | | | AUUCuAGUuAGUUuAGAUUTsT | 2373 |
| AD-20482.1 | KSP | AUCUAAACUAACUAGAAUC | 2299 | AucuAAAcuAAcuAGAAucTsT | 2374 |
| | | | | GAUUCuAGUuAGUUuAGAUTsT | 2375 |
| AD-20483.1 | KSP | UCUAAACUAACUAGAAUCC | 2300 | ucuAAAcuAAcuAGAAuccTsT | 2376 |
| | | | | GGAUUCuAGUuAGUUuAGATsT | 2377 |
| AD-20484.1 | KSP | CUAAACUAACUAGAAUCCU | 2301 | cuAAAcuAAcuAGAAuccuTsT | 2378 |
| | | | | AGGAUUCuAGUuAGUUuAGTsT | 2379 |
| AD-20485.1 | KSP | UAAACUAACUAGAAUCCUC | 2302 | uAAAcuAAcuAGAAuccucTsT | 2380 |
| | | | | GAGGAUUCuAGUuAGUUuATsT | 2381 |
| AD-20486.1 | KSP | AAACUAACUAGAAUCCUCC | 2303 | AAAcuAAcuAGAAuccuccTsT | 2382 |
| | | | | GGAGGAUUCuAGUuAGUUUTsT | 2383 |

### Example 13

### VEGF Targeted dsRNA with a Single Blunt End

A set of dsRNA duplexes targeted to VEGF were designed and synthesized. The set included duplexes tiling 10 nucleotides in each direction of the target sites for AD-3133. Each duplex includes a 2 base overhang at the end corresponding to the 3′ end of the antisense strand and no overhang, e.g., a blunt end, at the end corresponding to the 5′ end of the antisense strand.

The sequences of each strand of these duplexes are shown in the following table.

Each duplex is assayed for inhibition of expression using the assays described herein. The VEGF duplexes are administered alone and/or in combination with an Eg5/KSP dsRNA (e.g., AD-12115). In some embodiments, the dsRNA are administered in a nucleic-acid lipid particle, e.g., SNALP, formulation as described herein.

TABLE 22
Target sequences of blunt ended dsRNA targeted
to VEGF

| | SEQ ID | VEGF target sequence | position on |
|---|---|---|---|
| duplex ID | NO: | 5′ to 3′ | VEGF gene |
| AD-20447.1 | 2384 | ACCAAGGCCAGCACAUAGG | 1365 |
| AD-20448.1 | 2385 | CCAAGGCCAGCACAUAGGA | 1366 |
| AD-20449.1 | 2386 | CAAGGCCAGCACAUAGGAG | 1367 |
| AD-20450.1 | 2387 | AAGGCCAGCACAUAGGAGA | 1368 |
| AD-20451.1 | 2388 | AGGCCAGCACAUAGGAGAG | 1369 |
| AD-20452.1 | 2389 | GGCCAGCACAUAGGAGAGA | 1370 |
| AD-20453.1 | 2390 | GCCAGCACAUAGGAGAGAU | 1371 |
| AD-20454.1 | 2391 | CCAGCACAUAGGAGAGAUG | 1372 |
| AD-20455.1 | 2392 | CAGCACAUAGGAGAGAUGA | 1373 |
| AD-20456.1 | 2393 | AGCACAUAGGAGAGAUGAG | 1374 |
| AD-20457.1 | 2394 | CACAUAGGAGAGAUGAGCU | 1376 |
| AD-20458.1 | 2395 | ACAUAGGAGAGAUGAGCUU | 1377 |
| AD-20459.1 | 2396 | CAUAGGAGAGAUGAGCUUC | 1378 |
| AD-20460.1 | 2397 | AUAGGAGAGAUGAGCUUCC | 1379 |
| AD-20461.1 | 2398 | UAGGAGAGAUGAGCUUCCU | 1380 |
| AD-20462.1 | 2399 | AGGAGAGAUGAGCUUCCUA | 1381 |
| AD-20463.1 | 2400 | GGAGAGAUGAGCUUCCUAC | 1382 |
| AD-20464.1 | 2401 | GAGAGAUGAGCUUCCUACA | 1383 |
| AD-20465.1 | 2402 | AGAGAUGAGCUUCCUACAG | 1384 |
| AD-20466.1 | 2403 | GAGAUGAGCUUCCUACAGC | 1385 |

TABLE 23
Strand sequences of blunt ended dsRNA targeted to VEGF

| | | SEQ | | SEQ |
|---|---|---|---|---|
| | Sense strand | ID | Antisense strand | ID |
| duplex ID | (5′ to 3′) | NO: | (5′ to 3′) | NO: |
| AD-20447.1 | ACCAAGGCCAGCACAUAGGAG | 2404 | CUCCUAUGUGCUGGCCUUGGUGA | 2424 |
| AD-20448.1 | CCAAGGCCAGCACAUAGGAGA | 2405 | UCUCCUAUGUGCUGGCCUUGGUG | 2425 |
| AD-20449.1 | CAAGGCCAGCACAUAGGAGAG | 2406 | CUCUCCUAUGUGCUGGCCUUGGU | 2426 |
| AD-20450.1 | AAGGCCAGCACAUAGGAGAGA | 2407 | UCUCUCCUAUGUGCUGGCCUUGG | 2427 |
| AD-20451.1 | AGGCCAGCACAUAGGAGAGAU | 2408 | AUCUCUCCUAUGUGCUGGCCUUG | 2428 |
| AD-20452.1 | GGCCAGCACAUAGGAGAGAUG | 2409 | CAUCUCUCCUAUGUGCUGGCCUU | 2429 |
| AD-20453.1 | GCCAGCACAUAGGAGAGAUGA | 2410 | UCAUCUCUCCUAUGUGCUGGCCU | 2430 |
| AD-20454.1 | CCAGCACAUAGGAGAGAUGAG | 2411 | CUCAUCUCUCCUAUGUGCUGGCC | 2431 |
| AD-20455.1 | CAGCACAUAGGAGAGAUGAGC | 2412 | GCUCAUCUCUCCUAUGUGCUGGC | 2432 |
| AD-20456.1 | AGCACAUAGGAGAGAUGAGCU | 2413 | AGCUCAUCUCUCCUAUGUGCUGG | 2433 |
| AD-20457.1 | CACAUAGGAGAGAUGAGCUUC | 2414 | GAAGCUCAUCUCUCCUAUGUGCU | 2434 |
| AD-20458.1 | ACAUAGGAGAGAUGAGCUUCC | 2415 | GGAAGCUCAUCUCUCCUAUGUGC | 2435 |
| AD-20459.1 | CAUAGGAGAGAUGAGCUUCCU | 2416 | AGGAAGCUCAUCUCUCCUAUGUG | 2436 |
| AD-20460.1 | AUAGGAGAGAUGAGCUUCCUA | 2417 | UAGGAAGCUCAUCUCUCCUAUGU | 2437 |
| AD-20461.1 | UAGGAGAGAUGAGCUUCCUAC | 2418 | GUAGGAAGCUCAUCUCUCCUAUG | 2438 |
| AD-20462.1 | AGGAGAGAUGAGCUUCCUACA | 2419 | UGUAGGAAGCUCAUCUCUCCUAU | 2439 |
| AD-20463.1 | GGAGAGAUGAGCUUCCUACAG | 2420 | CUGUAGGAAGCUCAUCUCUCCUA | 2440 |
| AD-20464.1 | GAGAGAUGAGCUUCCUACAGC | 2421 | GCUGUAGGAAGCUCAUCUCUCCU | 2441 |
| AD-20465.1 | AGAGAUGAGCUUCCUACAGCA | 2422 | UGCUGUAGGAAGCUCAUCUCUCC | 2442 |
| AD-20466.1 | GAGAUGAGCUUCCUACAGCAC | 2423 | GUGCUGUAGGAAGCUCAUCUCUC | 2443 |

### Example 14

### dsRNA Oligonucleotide Synthesis

Synthesis

All oligonucleotides are synthesized on an AKTAoligopilot synthesizer. Commercially available controlled pore glass solid support (dT-CPG, 500 \acute{Å}, Prime Synthesis) and RNA phosphoramidites with standard protecting groups, 5′-O-dimethoxytrityl N6-benzoyl-2′-t-butyldimethylsilyl-adenosine-3′-O—N,N′-diisopropyl-2-cyanoethylphosphoramidite, 5′-O-dimethoxytrityl-N4-acetyl-2′-t-butyldimethylsilyl-cytidine-3′-O—N,N′-diisopropy1-2-cyanoethylphosphoramidite, 5′-O-dimethoxytrityl-N2-isobutryl-2′-t-butyldimethylsilyl-guanosine-3′-O—N,N′-diisopropyl-2-cyanoethylphosphoramidite, and 5′-O-dimethoxytrityl-2′-t-butyldimethylsilyl-uridine-3′-O—N,N′-diisopropyl-2-cyanoethylphosphoramidite (Pierce Nucleic Acids Technologies) were used for the oligonucleotide synthesis. The 2′-F phosphoramidites, 5′-O-dimethoxytrityl-N4-acetyl-2′-fluro-cytidine-3′-O—N,N′-diisopropyl-2-cyanoethyl-phosphoramidite and 5′-O-dimethoxytrityl-2′-fluro-uridine-3′-O—N,N′-diisopropyl-2-cyanoethyl-phosphoramidite are purchased from (Promega). All phosphoramidites are used at a concentration of 0.2M in acetonitrile (CH₃CN) except for guanosine which is used at 0.2M concentration in 10% THF/ANC (v/v). Coupling/recycling time of 16 minutes is used. The activator is 5-ethyl thiotetrazole (0.75M, American International Chemicals); for the PO-oxidatioin iodine/water/pyridine is used and for the PS-oxidation PADS (2%) in 2,6-lutidine/ACN (1:1 v/v) is used.

3′-ligand conjugated strands are synthesized using solid support containing the corresponding ligand. For example, the introduction of cholesterol unit in the sequence is performed from a hydroxyprolinol-cholesterol phosphoramidite. Cholesterol is tethered to trans-4-hydroxyprolinol via a 6-aminohexanoate linkage to obtain a hydroxyprolinol-cholesterol moiety. 5′-end Cy-3 and Cy-5.5 (fluorophore) labeled siRNAs are synthesized from the corresponding Quasar-570 (Cy-3) phosphoramidite are purchased from Biosearch Technologies. Conjugation of ligands to 5′-end and or internal position is achieved by using appropriately protected ligand-phosphoramidite building block. An extended 15 min coupling of 0.1 M solution of phosphoramidite in anhydrous CH3CN in the presence of 5-(ethylthio)-1H-tetrazole activator to a solid-support-bound oligonucleotide. Oxidation of the internucleotide phosphite to the phosphate is carried out using standard iodine-water as reported (1) or by treatment with tert-butyl hydroperoxide/acetonitrile/water (10:87:3) with 10 min oxidation wait time conjugated oligonucleotide. Phosphorothioate is introduced by the oxidation of phosphite to phosphorothioate by using a sulfur transfer reagent such as DDTT (purchased from AM Chemicals), PADS and or Beaucage reagent. The cholesterol phosphoramidite is synthesized in house and used at a concentration of 0.1 M in dichloromethane. Coupling time for the cholesterol phosphoramidite is 16 minutes.

Deprotection I (Nucleobase Deprotection)

After completion of synthesis, the support is transferred to a 100 mL glass bottle (VWR). The oligonucleotide is cleaved from the support with simultaneous deprotection of base and phosphate groups with 80 mL of a mixture of ethanolic ammonia [ammonia: ethanol (3:1)] for 6.5 h at 55° C. The bottle is cooled briefly on ice and then the ethanolic ammonia mixture is filtered into a new 250-mL bottle. The CPG is washed with 2×40 mL portions of ethanol/water (1:1 v/v). The volume of the mixture is then reduced to ˜30 mL by roto-vap. The mixture is then frozen on dry ice and dried under vacuum on a speed vac.

Deprotection II (Removal of 2′-TBDMS Group)

The dried residue is resuspended in 26 mL of triethylamine, triethylamine trihydrofluoride (TEA.3HF) or pyridine-HF and DMSO (3:4:6) and heated at 60° C. for 90 minutes to remove the tert-butyldimethylsilyl (TBDMS) groups at the 2′ position. The reaction is then quenched with 50 mL of 20 mM sodium acetate and the pH is adjusted to 6.5. Oligonucleotide is stored in a freezer until purification.

Analysis

The oligonucleotides are analyzed by high-performance liquid chromatography (HPLC) prior to purification and selection of buffer and column depends on nature of the sequence and or conjugated ligand.

HPLC Purification

The ligand-conjugated oligonucleotides are purified by reverse-phase preparative HPLC. The unconjugated oligonucleotides are purified by anion-exchange HPLC on a TSK gel column packed in house. The buffers are 20 mM sodium phosphate (pH 8.5) in 10% CH₃CN (buffer A) and 20 mM sodium phosphate (pH 8.5) in 10% CH₃CN, 1M NaBr (buffer B). Fractions containing full-length oligonucleotides are pooled, desalted, and lyophilized. Approximately 0.15 OD of desalted oligonucleotides are diluted in water to 150 μL and then pipetted into special vials for CGE and LC/MS analysis. Compounds are then analyzed by LC-ESMS and CGE.

siRNA Preparation

For the preparation of siRNA, equimolar amounts of sense and antisense strand are heated in 1× PBS at 95° C. for 5 min and slowly cooled to room temperature. Integrity of the duplex is confirmed by HPLC analysis. AD-3133 and AD-AD-12115, described herein are synthesized.

### Example 15

### Synthesis of Collimated Lipids

The PEG-lipids, such as mPEG2000-1,2-Di-O-alkyl-sn3-carbomoylglyceride (PEG-DMG) were synthesized using the following procedures:

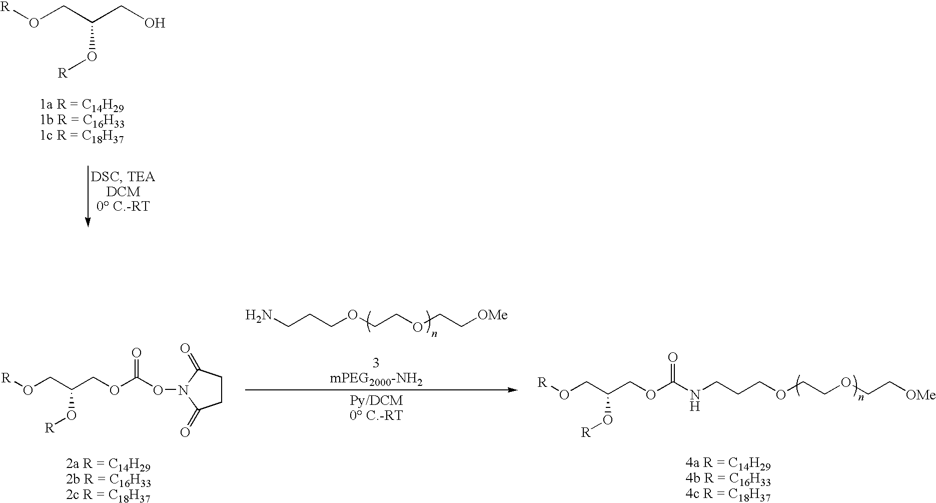

mPEG2000-1,2-Di-O-alkyl-sn3-carbomoylglyceride

Preparation of compound 4a: 1,2-Di-O-tetradecyl-sn-glyceride 1a (30 g, 61.80 mmol) and N,N′-succinimidylcarboante (DSC, 23.76 g, 1.5 eq) were taken in dichloromethane (DCM, 500 mL) and stirred over an ice water mixture. Triethylamine (25.30 mL, 3 eq) was added to stirring solution and subsequently the reaction mixture was allowed to stir overnight at ambient temperature. Progress of the reaction was monitored by TLC. The reaction mixture was diluted with DCM (400 mL) and the organic layer was washed with water (2×500 mL), aqueous NaHCO₃ solution (500 mL) followed by standard work-up. Residue obtained was dried at ambient temperature under high vacuum overnight. After drying the crude carbonate 2a thus obtained was dissolved in dichloromethane (500 mL) and stirred over an ice bath. To the stirring solution mPEG₂₀₀₀-NH₂ (3, 103.00 g, 47.20 mmol, purchased from NOF Corporation, Japan) and anhydrous pyridine (80 mL, excess) were added under argon. In some embodiments, the methoxy-(PEG)x-amine has an x=from 45-49, preferably 47-49, and more preferably 49. The reaction mixture was then allowed stir at ambient temperature overnight. Solvents and volatiles were removed under vacuum and the residue was dissolved in DCM (200 mL) and charged on a column of silica gel packed in ethyl acetate. The column was initially eluted with ethyl acetate and subsequently with gradient of 5-10% methanol in dichloromethane to afford the desired PEG-Lipid 4a as a white solid (105.30 g, 83%). ¹H NMR (CDCl₃, 400 MHz) δ=5.20-5.12(m, 1H), 4.18-4.01(m, 2H), 3.80-3.70(m, 2H), 3.70-3.20(m, —O—CH₂—CH₂—O—, PEG-CH₂), 2.10-2.01(m, 2H), 1.70-1.60 (m, 2H), 1.56-1.45(m, 4H), 1.31-1.15(m, 48H), 0.84(t, J=6.5 Hz, 6H). MS range found: 2660-2836.

Preparation of 4b: 1,2-Di-O-hexadecyl-sn-glyceride 1b (1.00 g, 1.848 mmol) and DSC (0.710 g, 1.5 eq) were taken together in dichloromethane (20 mL) and cooled down to 0° C. in an ice water mixture. Triethylamine (1.00 mL, 3 eq) was added to that and stirred overnight. The reaction was followed by TLC, diluted with DCM, washed with water (2 times), NaHCO₃ solution and dried over sodium sulfate. Solvents were removed under reduced pressure and the residue 2b under high vacuum overnight. This compound was directly used for the next reaction without further purification. MPEG₂₀₀₀-NH₂ 3 (1.50 g, 0.687 mmol, purchased from NOF Corporation, Japan) and compound from previous step 2b (0.702 g, 1.5 eq) were dissolved in dichloromethane (20 mL) under argon. The reaction was cooled to 0° C. Pyridine (1 mL, excess) was added to that and stirred overnight. The reaction was monitored by TLC. Solvents and volatiles were removed under vacuum and the residue was purified by chromatography (first Ethyl acetate then 5-10% MeOH/DCM as a gradient elution) to get the required compound 4b as white solid (1.46 g, 76%). ¹H NMR (CDCl₃, 400 MHz) δ=5.17(t, J=5.5 Hz, 1H), 4.13(dd, J=4.00 Hz, 11.00 Hz, 1H), 4.05(dd, J=5.00Hz, 11.00 Hz, 1H), 3.82-3.75(m, 2H), 3.70-3.20(m, —O—CH₂—CH₂—O—, PEG-CH₂), 2.05-1.90(m, 2H), 1.80-1.70 (m, 2H), 1.61-1.45(m, 6H), 1.35-1.17(m, 56H), 0.85(t, J=6.5 Hz, 6H). MS range found: 2716-2892.

Preparation of 4c: 1,2-Di-O-octadecyl-sn-glyceride 1c (4.00 g, 6.70 mmol) and DSC (2.58 g, 1.5 eq) were taken together in dichloromethane (60 mL) and cooled down to 0° C. in an ice water mixture. Triethylamine (2.75 mL, 3 eq) was added to that and stirred overnight. The reaction was followed by TLC, diluted with DCM, washed with water (2 times), NaHCO₃ solution and dried over sodium sulfate. Solvents were removed under reduced pressure and the residue under high vacuum overnight. This compound was directly used for the next reaction with further purification. MPEG₂₀₀₀-NH₂ 3 (1.50 g, 0.687 mmol, purchased from NOF Corporation, Japan) and compound from previous step 2c (0.760 g, 1.5 eq) were dissolved in dichloromethane (20 mL) under argon. The reaction was cooled to 0° C. Pyridine (1 mL, excess) was added to that and stirred overnight. The reaction was monitored by TLC. Solvents and volatiles were removed under vacuum and the residue was purified by chromatography (first Ethyl acetate then 5-10% MeOH/DCM as a gradient elution) to get the required compound 4c as white solid (0.92 g, 48%). ¹H NMR (CDCl₃, 400 MHz) δ=5.22-5.15(m, 1H), 4.16(dd, J=4.00Hz, 11.00 Hz, 1H), 4.06 (dd, J=5.00 Hz, 11.00 Hz, 1H), 3.81-3.75(m, 2H), 3.70-3.20(m, —O—CH₂—CH₂—O—, PEG-CH₂), 1.80-1.70 (m, 2H), 1.60-1.48(m, 4H), 1.31-1.15(m, 64H), 0.85(t, J=6.5 Hz, 6H). MS range found: 2774-2948.

### Example 16

### General Protocol for the Extrusion Method

Lipids (e.g., Lipid A, DSPC, cholesterol, DMG-PEG) are solubilized and mixed in ethanol according to the desired molar ratio. Liposomes are formed by an ethanol injection method where mixed lipids are added to sodium acetate buffer at pH 5.2. This results in the spontaneous formation of liposomes in 35% ethanol. The liposomes are extruded through a 0.08 μm polycarbonate membrane at least 2 times. A stock siRNA solution is prepared in sodium acetate and 35% ethanol and is added to the liposome to load. The siRNA-liposome solution is incubated at 37° C. for 30 min and, subsequently, diluted. Ethanol is removed and exchanged to PBS buffer by dialysis or tangential flow filtration.

### Example 17

### General Protocol for the In-Line Mixing Method

Individual and separate stock solutions are prepared—one containing lipid and the other siRNA. Lipid stock containing, e.g., lipid A, DSPC, cholesterol and PEG lipid is prepared by solubilized in 90% ethanol. The remaining 10% is low pH citrate buffer. The concentration of the lipid stock is 4 mg/mL. The pH of this citrate buffer can range between pH 3-5, depending on the type of fusogenic lipid employed. The siRNA is also solubilized in citrate buffer at a concentration of 4 mg/mL. For small scale, 5 mL of each stock solution is prepared.

Stock solutions are completely clear and lipids must be completely solubilized before combining with siRNA. Therefore stock solutions may be heated to completely solubilize the lipids. The siRNAs used in the process may be unmodified oligonucleotides or modified and may be conjugated with lipophilic moieties such as cholesterol.

The individual stocks are combined by pumping each solution to a T-junction. A dual-head Watson-Marlow pump is used to simultaneously control the start and stop of the two streams. A 1.6 mm polypropylene tubing is further downsized to a 0.8 mm tubing in order to increase the linear flow rate. The polypropylene line (ID=0.8 mm) are attached to either side of a T-junction. The polypropylene T has a linear edge of 1.6 mm for a resultant volume of 4.1 mm³. Each of the large ends (1.6 mm) of polypropylene line is placed into test tubes containing either solubilized lipid stock or solubilized siRNA. After the T-junction a single tubing is placed where the combined stream will emit. The tubing is then extending into a container with 2× volume of PBS. The PBS is rapidly stirring. The flow rate for the pump is at a setting of 300 rpm or 110 mL/min. Ethanol is removed and exchanged for PBS by dialysis. The lipid formulations are then concentrated using centrifugation or diafiltration to an appropriate working concentration.

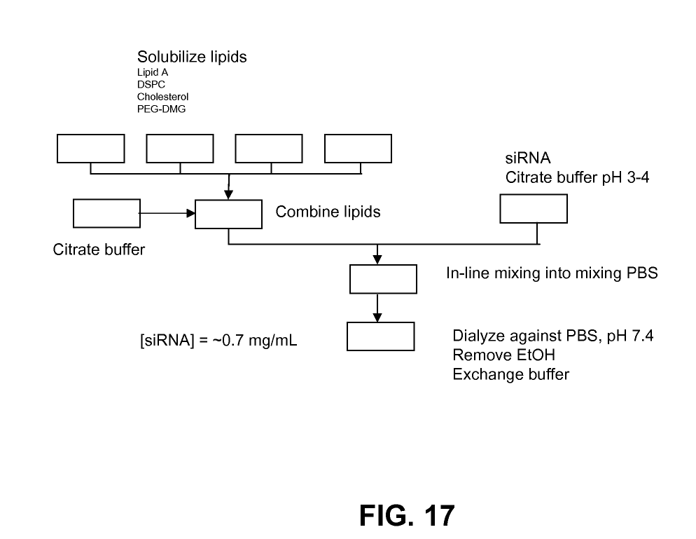

FIG. 17 shows a schematic of the in-line mixing method.

### Example 18

### siRNA Silencing by LNP-08 Formulated VSP in Intrahepatic Hep3B Tumors in Mice

Silencing of VSP (VEGF and KSP) was performed in orthotopic (intrahepatic) Hep3B tumors following intravenous administration of siRNAs formulated in XTC containing nucleic acid-lipid particles, e.g., LNP-08.

Tumors were established by implantation of 1×10⁶ Hep3B cells into the right flank of 8 week-old female Fox scid/beige mice. The cells were engineered to stably express firefly Luciferase. Tumor burden was monitored weekly by in vivo biophotonic imaging using the IVIS system (Caliper, Inc.). Approximately 4 weeks after tumor implantation, cohorts of tumor-bearing animals received intravenous (tail vein) injections of test article as follows:

| Group | Test article | Dose (siRNA) | n |
|---|---|---|---|
| 1 | LNP08-1955 | 4 mg/kg | 5 |
| 2 | LNP08-VSP | 4 mg/kg | 5 |

LNP08-1955 is siRNA AD-1955 (targeting firefly Luciferase) formulated in lipid nanoparticles comprising XTC (60 mol %), DSPC (7.5 mol %), Cholesterol (31 mol %) and PEG-cDMG (1.5 mol %) at an N:P ratio of approximately 3.0.

LNP08-VSP is siRNAs AD-12115 (targeting KSP) and AD-3133 (targeting VEGF) in a 1:1 molar ratio formulated in lipid nanoparticles comprising XTC (60 mol %), DSPC (7.5 mol %), Cholesterol (31 mol %) and PEG-cDMG (1.5 mol %) at an N:P ratio of approximately 3.0.

One day following treatment, animals were sacrificed and tumor-bearing liver lobes collected for analysis. Total RNA was extracted followed by cDNA synthesis by random priming. Levels of human KSP and human VEGF, normalized to human GAPDH, were measured using human-specific custom Taqman® assays (Applied Biosystems, Inc.). Group averages were calculated and normalized to the LNP08-1955 treatment group.

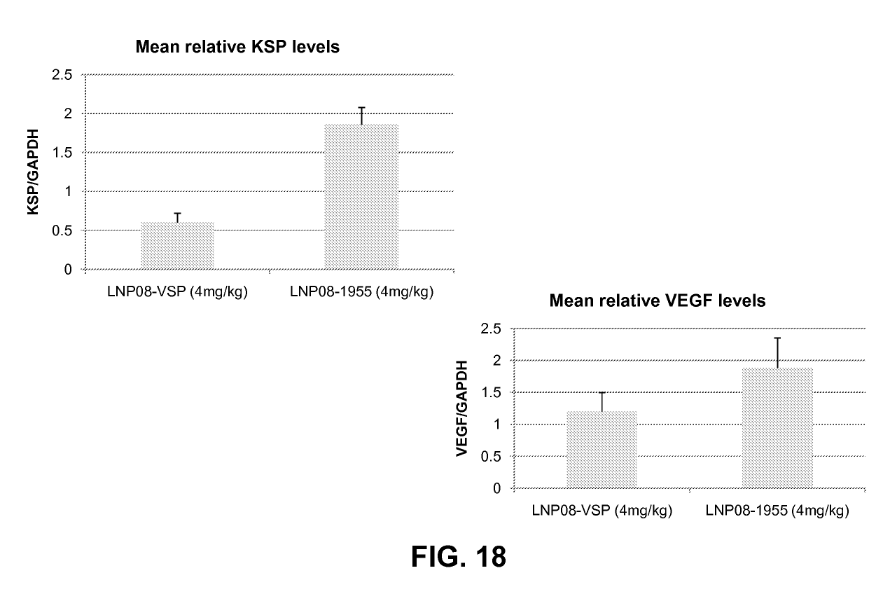

As shown in FIG. 18, treatment with LNP08-VSP (Group 2) resulted in a greater than 60%, e.g., 68% reduction in tumor KSP mRNA (p<0.001) and at least 40% reduction in VEGF mRNA (p<0.05) relative to the LNP08-1955 treatment (Group 1).

### Example 19

### Evaluation of LNP-011 and LNP-012 Lipid Formulations in the Mouse Hep3b Tumor Model

The effects of various VSP formulations on KSP and VEGF expression in intrahepatic Hep3B tumors in mice were compared. Thirty five female Fox Scid beige mice were injected with 1×10̂6 Hep3B-Luc cells suspeneded in 0.025 cc PBS via direct intrahepatic surgery. Tumor growth was monitered via Luc readings by Xenogen.

Mice received a single bolus dose (4 mg/kg) of one of the following: SNALP-1955 (luciferase control); ALN-VSP02; SNALP-T-VSP (with C-18 PEG)-VSP; LNP-11-VSP, and LNP-12 VSP. Animal were euthanized at 24 hours post does, and the TaqMan protocol was used for detection of tumor specific KSP and VEGF knockdown.

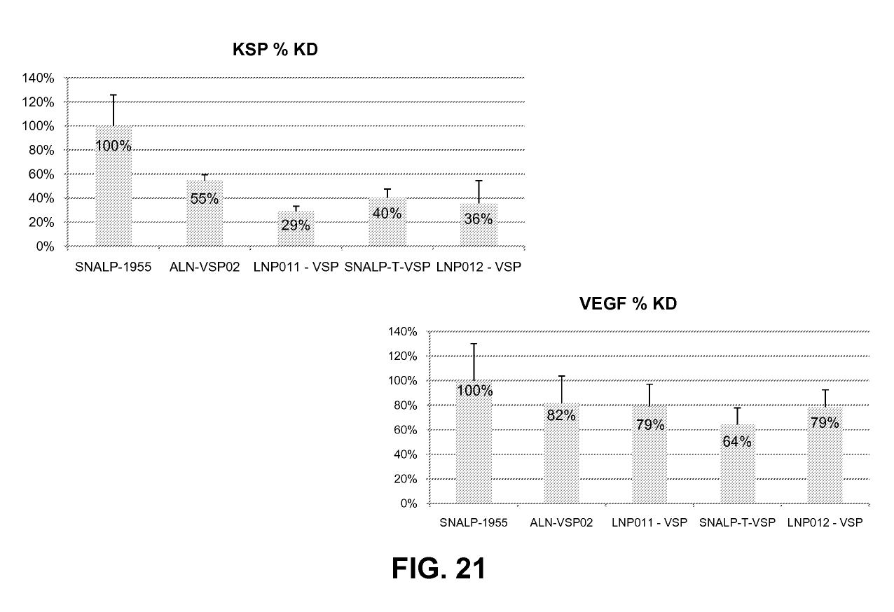

The results are shown in FIG. 21. SNAPL-T-VSP; LNP-11-VSP, and LNP-12 VSP demonstrated increased knockdown of KSP expression compared to ALN-VSP02.

### Example 20

### Evaluation of LNP-08 +/−C18 Lipid Formulations in the Mouse Hep3b Tumor Model

The effects of the following VSP formulations were tested in a HEP3B tumor model. Tumor-bearing (intrahepatic) mice were injected with one of the following formulations, prepared and administered as a single bolus IV dose according to protocols described above:

| Group | Test article | Dose (siRNA) | | n |
|---|---|---|---|---|
| 1 | ALN-VSP02 | 4 | mg/kg | 6 |
| 2 | LNPOS-Luc | 4 | mg/kg | 4 |
| 3 | LNP08-VSP | 4 | mg/kg | 7 |
| 4 | LNP08-VSP | 1 | mg/kg | 7 |
| 5 | LNP08-VSP | 0.25 | mg/kg | 7 |
| 6 | LNP08-C18-VSP | 4 | mg/kg | 7 |
| 7 | LNP08-C18-VSP | 1 | mg/kg | 7 |
| 8 | LNP08-C18-VSP | 0.25 | mg/kg | 7 |

Formulation of ALN-VSP02 was as described in Example 9.

LNP08-Luc is siRNA AD-1955 (targeting firefly Luciferase) formulated in lipid nanoparticles comprising XTC (60 mol %), DSPC (7.5 mol %), Cholesterol (31 mol %) and PEG-cDMG (1.5 mol %) at an N:P ratio of approximately 3.0.

LNP08-VSP is siRNA AD-12115 (targeting KSP) and AD-3133 (targeting VEGF) in a 1:1 molar ratio formulated in lipid nanoparticles comprising XTC (60 mol %), DSPC (7.5 mol %), Cholesterol (31 mol %) and PEG-cDMG (1.5 mol %) at an N:P ratio of approximately 3.0.

LNP08-C18-VSP is siRNA AD-12115 (targeting KSP) and AD-3133 (targeting VEGF) in a 1:1 molar ratio formulated in lipid nanoparticles comprising XTC (60 mol %), DSPC (7.5 mol %), Cholesterol (31 mol %) and PEG-cDSG (1.5 mol %) at an N:P ratio of approximately 3.0.

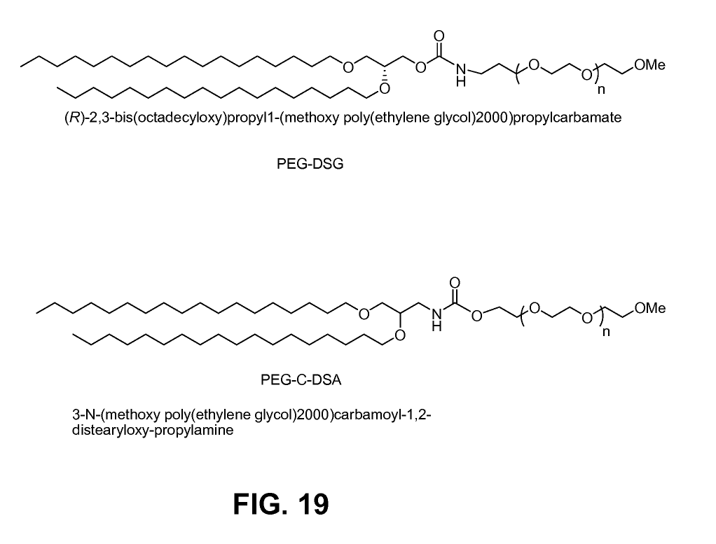

FIG. 19 illustrates the chemical structures of PEG-DSG and PEG-C-DSA. PEG-DSG is polyethylene glycol distyryl glycerol, in which PEG is either C18-PEG or PEG-C18 and the PEG has an average molecular weight of 2000 Da.

Twenty-four hours following treatment, animals were sacrificed and tumors collected for analysis. Total RNA was extracted from tumors, followed by cDNA synthesis by random priming. Levels of human KSP and human VEGF, normalized to human GAPDH, were measured using human-specific custom Taqman® assays (Applied Biosystems, Inc.).

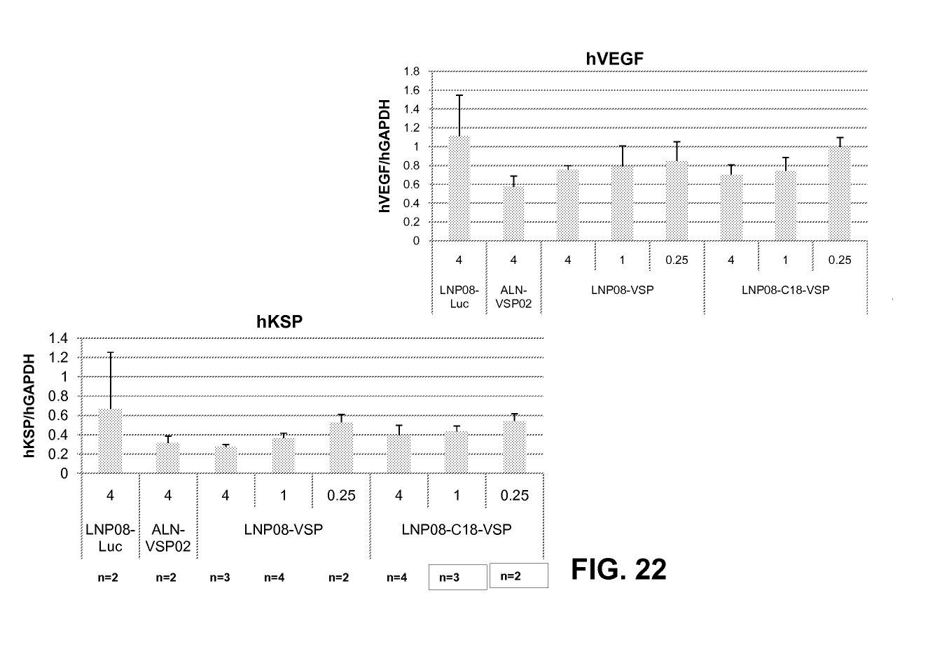

The results are shown the graphs in FIG. 22 and show KSP and VEGF silencing comparable to silencing by ALN-VSP02.

### Example 21

### Role of ApoE in the Cellular Uptake of Liposomes in HeLa Cells

LNP formulated dsRNAs are prepared with the addition of recombinant human ApoE. The resulting LNP-ApoE formulated dsRNA are tested in HeLa cells for the effect on uptake of the dsRNA by the cells. Compositions and methods utilizing ApoE in conjunction with ionizable lipids is described in International patent application No., PCT/US10/22614, which is herein incorporated by reference in its entirety.

Experimental Protocol:

HeLa cells are seeded in 96 well plates (Grenier) at 6000 cells per well overnight. Three different liposome formulations of Alexa-fluor 647 labeled GFP siRNA: 1) LNP01, 2) SNALP, 3) LNP05 are diluted in one of 3 media conditions to a 50 nM final concentration. Media conditions examined are OptiMem, DMEM with 10% FBS or DMEM with 10% FBS plus 10 ug/mL of human recombinant ApoE (Fitzgerald Industries). The indicated liposomes either in media or in media-precomplexed with ApoE for 10 minutes are added to cells for either 4, 6, or 24 hours. Three replicated are performed for each experimental condition. After addition to HeLa cells in plates for indicated time points cells are fixed in 4% paraformaldehyde for 15 minutes then nuclei and cytoplasm stained with DAPI and Syto dye. Images are acquired using an Opera spinning disc automated confocal system from Perkin Elmer. Quantitation of Alexa Fluor 647 siRNA uptake is performed using Acapella software. Four different parameters are quantified: 1) Cell number, 2) the number of siRNA positive spots per field, 3) the number of siRNA positive spots per cell and 4) the integrated spot signal or the average number of siRNA spots per cell times the average spot intensity. The average spot signal therefore is a rough estimate of the total amount of siRNA content per cell.

In addition, the 4 different LNP-ApoE formulated dsRNA are tested (SNALP (DLinDMa), XTC, MC3, ALNY-100) in the following cell lines and the effect on uptake of the dsRNA by the cells is determined:

A375 (melanoma), B16F10 (melanoma), BT-474 (breast), GTL-16 (gastric carcinoma), Hct116 (colon), Hep3b (Hepatic), HepG2 (liver), HeLa (cervical), HUH 7 (liver), MCF7 (breast), Mel-285 (uveal melanoma), NCI-H1975 (lung), OMM-1.3 (uveal melanoma), PC3 (prostate), SKOV-3 (ovarian), U87 (glioblastoma).

### Example 22

### K_{d} of KSP siRNA in the Presence of ApoE

The effect of ApoE on the Kd (affinity) of LNP-08 formulated siRNA targeting KSP was evaluated in multiple cell lines. Both LNP08 and LNP08 with C18PEG formulated siRNA were used. The KSP targeted siRNA duplex was AL-DP-6248.

| position in | | | | | |
|---|---|---|---|---|---|
| human | SEQ | | SEQ | | |
| Eg5/KSP | ID | | ID | antisense sequence | duplex |
| sequence | NO: | sense sequence (5′-3′) | No: | (5′-3′) | name |
| 383-405 | 45 | AccGAAGuGuuGuuuGuccTsT | 46 | GGAcAAAcAAcACUUCGGUTsT | AL-DP- |
| | | | | | 6248 |

The following cell lines were used.

| Cell Line | Cell Type | Species |
|---|---|---|
| HeLa | Cervical Adenocarcinoma | Human |
| HCT116 | Colorectal carcinoma | Human |
| A375 | Melanoma | Human |
| MCF7 | Breast adenocarcinoma | Human |
| B16F10 | Melanoma | Mouse |
| Hep3b | Hepatic | Human |
| HUH 7 | Hepatic | Human |
| HepG2 | Hepatic | Human |
| Skov 3 | Ovarian | Human |
| U87 | Glioblastoma | Human |
| PC3 | Prostate | Human |

On day 1, cells were plated in 96 well plates at 20,000 cells/well. On day 2, formulated siRNA were incubated with serum-containing media +/−ApoE at 37° C. for 15-30 minutes. Media was removed from cells and pre-warmed complexes were layered on the cells at 100 uL/well at an siRNA concentration of 20 nM. ApoE concentration was titrated at 1.0, 3.0, 9.0, and 20.0 μg/ml. Cells were incubated with formulated duplexes for 24 hours. At day 3, cells lysed and prepared for bDNA analysis and kD calculations.

The presence of Apo E improved kD in a number of cell lines including HCT-116, HeLa, A375, and B16F10 (data not shown).

### Example 23

### IC₅₀ of KSP siRNA in the Presence of ApoE

The effect of ApoE on the IC₅₀ (efficacy) of LNP-08 formulated siRNA targeting KSP was evaluated in multiple cell lines. Both LNP08 and LNP08 with Cl8PEG formulated siRNA were used. The KSP targeted siRNA duplex was AL-DP-6248.

At day 0, cells were plated at 15,000-20,000 per well in 96 well plates. At day 1, serum-containing media, formulated duplex, and +/−3 ug/ml ApoE were incubated at 37° C. for 15-30 minutes. Serial dilutions of siRNA were used in the 0.01 nM to 1.0 μM range. Media was removed from cells and pre-warmed complexes were layered on cells at 100 uL/well. Cells were incubated with siRNA for 24 hours. At day 2, cells were lysed and prepared for bDNA analysis as described herein. KSP mRNA levels were determined using a Quantigene 1.0 to determine KSP levels in comparison to GAPDH. Negative control was luciferase targeted siRNA, AD-1955.

The results are shown in the table below. LNP-08 formulated siRNA was active in all cell lines. In some cell lines the addition of ApoE improved efficacy of siRNA treatment as demonstrated by a lower IC₅₀.

| | | | IC₅₀ | | | |
|---|---|---|---|---|---|---|
| | | | | LNP08 C18 + | | LNP08 + |
| Cell Line | Cell Type | Species | LNP08 C18 | 3 ug/mL ApoE | LNP08 | 3 ug/mL ApoE |
| HeLa | Cervical | Human | 7.02 | 3.51 | 2.75 | 2.02 |
| | Adenocarcinoma | | | | | |
| HCT116 | Colorectal | Human | 4.71 | 3.89 | 0.4 | 0.44 |
| | carcinoma | | | | | |
| A375 | Melanoma | Human | >500 | 24.82 | 7.08 | 0.94 |
| MCF7 | Breast | Human | >500 | >500 | 19.98 | 10.26 |
| | adenocarcinoma | | | | | |
| B16F10 | Melanoma | Mouse | 13.92 | >500 | 18.52 | 2.37 |
| Hep3b | Hepatic | Human | 60.47*/NA | 22.13*/>600 | 1.4 | 8.98 |
| HUH 7 | Hepatic | Human | NA | >600 | 14.26 | 1.8 |
| HepG2 | Hepatic | Human | 433 nM | 67.3 (1 ug/ml)/ | 1.27 | 0.38 |
| | | | | 0.45 (3 ug/ml) | | |
| Skov 3 | Ovarian | Human | NA | NA | 3.95 | 7.26 |
| U87 | Glioblastoma | Human | NA | NA | 464.74 | 283.68 |
| PC3 | Prostate | Human | NA | >600 | 96.62 | 59 |

### Example 24

### Inhibition of Eg5/KSP and VEGF Expression in Humans

A human subject is treated with a pharmaceutical composition, e.g., a nucleic acid-lipid particle having both a dsRNA targeted to a Eg5/KSP gene and a dsRNA targeted to a VEGF gene to inhibit expression of the Eg5/KSP and VEGF genes in a nucleic acid-lipid particle. The nucleic acid-lipid particle comprises, e.g., XTC, MC3, or ALNY-100.

A subject in need of treatment is selected or identified. The subject can be in need of cancer treatment, e.g., liver cancer.

At time zero, a suitable first dose of the composition is subcutaneously administered to the subject. The composition is formulated as described herein. After a period of time, the subject's condition is evaluated, e.g., by measurement of tumor growth, measuring serum AFP levels, and the like. This measurement can be accompanied by a measurement of Eg5/KSP and/or VEGF expression in said subject, and/or the products of the successful siRNA-targeting of Eg5/KSP and/or VEGF mRNA. Other relevant criteria can also be measured. The number and strength of doses are adjusted according to the subject's needs.

After treatment, the subject's condition is compared to the condition existing prior to the treatment, or relative to the condition of a similarly afflicted but untreated subject.

Those skilled in the art are familiar with methods and compositions in addition to those specifically set out in the present disclosure which will allow them to practice this invention to the full scope of the claims hereinafter appended. Attorney Docket No: 26421-16564

We claim:

1. A composition comprising a nucleic acid lipid particle comprising a first double-stranded ribonucleic acid (dsRNA) for inhibiting the expression of a human kinesin family member 11 (Eg5/KSP) gene in a cell and a second dsRNA for inhibiting expression of a human VEGF in a cell, wherein:
the nucleic acid lipid particle comprises a lipid formulation comprising 45-65 mol % of a cationic lipid, 5 mol % to about 10 mol %, of a non-cationic lipid, 25-40 mol % of a sterol, and 0.5-5 mol % of a PEG or PEG-modified lipid,
the first dsRNA consists of a first sense strand and a first antisense strand, and the first sense strand comprises a first sequence and the first antisense strand comprises a second sequence complementary to at least 15 contiguous nucleotides of SEQ ID NO:1311 (5′-UCGAGAAUCUAAACUAACU-3′),
wherein the first sequence is complementary to the second sequence and wherein the first dsRNA is between 15 and 30 base pairs in length; and
the second dsRNA consists of a second sense strand and a second antisense strand, the second sense strand comprising a third sequence and the second antisense strand comprising a fourth sequence complementary to at least 15 contiguous nucleotides of SEQ ID NO:1538 (5′-GCACAUAGGAGAGAUGAGCUU-3′),
wherein the third sequence is complementary to the fourth sequence and wherein the second dsRNA is between 15 and 30 base pairs in length.

2. The composition of claim 1, wherein the cationic lipid comprises formula A wherein formula A is 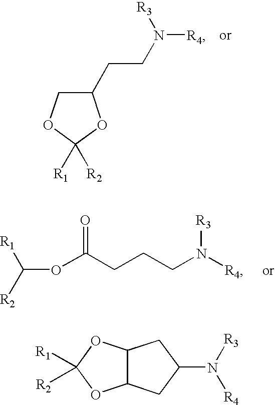
where R1 and R2 are independently alkyl, alkenyl or alkynyl, each can be optionally substituted, and R3 and R4 are independently lower alkyl or R3 and R4 can be taken together to form an optionally substituted heterocyclic ring.

3. The composition of claim 2, wherein the cationic lipid comprises XTC (2,2-Dilinoleyl-4-dimethylaminoethyl-[1,3]-dioxolane).

4. The composition of claim 2, wherein the cationic lipid comprises XTC, the non-cationic lipid comprises DSPC, the sterol comprises cholesterol and the PEG lipid comprises PEG-DMG.

5. The composition of claim 2, wherein the cationic lipid comprises XTC and the formulation is selected from the group consisting of:

| | |
|---|---|
| LNP05 | XTC/DSPC/Cholesterol/PEG-DMG |
| | 57.5/7.5/31.5/3.5 |
| | lipid:siRNA ~6:1 |
| LNP06 | XTC/DSPC/Cholesterol/PEG-DMG |
| | 57.5/7.5/31.5/3.5 |
| | lipid:siRNA ~11:1 |
| LNP07 | XTC/DSPC/Cholesterol/PEG-DMG |
| | 60/7.5/31/1.5, |
| | lipid:siRNA ~6:1 |
| LNP08 | XTC/DSPC/Cholesterol/PEG-DMG |
| | 60/7.5/31/1.5, |
| | lipid:siRNA ~11:1 |
| LNP09 | XTC/DSPC/Cholesterol/PEG-DMG |
| | 50/10/38.5/1.5 |
| | lipid:siRNA ~10:1 |
| LNP13 | XTC/DSPC/Cholesterol/PEG-DMG |
| | 50/10/38.5/1.5 |
| | lipid:siRNA ~33:1 |
| LNP22 | XTC/DSPC/Cholesterol/PEG-DSG |
| | 50/10/38.5/1.5 |
| | lipid:siRNA ~10 |

6. The composition of claim 1, wherein the cationic lipid comprises ALNY-100 ((3aR,5s,6aS)-N,N-dimethyl-2,2-di((9Z,12Z)-octadeca-9,12-dienyl)tetrahydro-3aH-cyclopenta[d][1,3]dioxol-5-amine)).

7. The composition of claim 6, wherein the cationic lipid comprises ALNY-100 and the formulation consists of:

| | |
|---|---|
| LNP10 | ALNY-100/DSPC/Cholesterol/PEG-DMG |
| | 50/10/38.5/1.5 |
| | lipid:siRNA ~10:1 |

8. The composition of claim 1, wherein the cationic lipid comprises MC3 (((6Z,9Z,28Z,31Z)-heptatriaconta-6,9,28,31-tetraen-19-yl 4-(dimethylamino)butanoate).

9. The composition of claim 8, wherein the cationic lipid comprises MC3 and the lipid formulation is selected from the group consisting of:

| | |
|---|---|
| LNP11 | MC3/DSPC/Cholesterol/PEG-DMG |
| | 50/10/38.5/1.5 |
| | lipid:siRNA ~10:1 |
| LNP14 | MC3/DSPC/Cholesterol/PEG-DMG |
| | 40/15/40/5 |
| | lipid:siRNA ~11 |
| LNP15 | MC3/DSPC/Cholesterol/PEG-DSG/GalNAc-PEG-DSG |
| | 50/10/35/4.5/0.5 |
| | lipid:siRNA ~11 |
| LNP16 | MC3/DSPC/Cholesterol/PEG-DMG |
| | 50/10/38.5/1.5 |
| | lipid:siRNA ~7 |
| LNP17 | MC3/DSPC/Cholesterol/PEG-DSG |
| | 50/10/38.5/1.5 |
| | lipid:siRNA ~10 |
| LNP18 | MC3/DSPC/Cholesterol/PEG-DMG |
| | 50/10/38.5/1.5 |
| | lipid:siRNA ~12 |
| LNP19 | MC3/DSPC/Cholesterol/PEG-DMG |
| | 50/10/35/5 |
| | lipid:siRNA ~8 |
| LNP20 | MC3/DSPC/Cholesterol/PEG-DPG |
| | 50/10/38.5/1.5 |
| | lipid:siRNA ~10 |

10. The composition of claim 1, wherein the first dsRNA consists of a sense strand consisting of SEQ ID NO:1534 (5′-UCGAGAAUCUAAACUAACUTT-3′) and an antisense strand consisting of SEQ ID NO:1535 (5′-AGUUAGUUUAGAUUCCUGATT-3′) and the second dsRNA consists of a sense strand consisting of SEQ ID NO:1536 (5′-GCACAUAGGAGAGAUGAGCUU-3′), and an antisense strand consisting of SEQ ID NO:1537 (5′-AAGCUCAUCUCUCCUAUGUGCUG-3′).

11. The composition of claim 10, wherein each strand is modified as follows to include a 2′-O-methyl ribonucleotide as indicated by a lower case letter “c” or “u” and a phosphorothioate as indicated by a lower case letter “s”:
the first dsRNA consists of a sense strand consisting of SEQ ID NO:1240 (5′-ucGAGAAucuAAAcuAAcuTsT-3′)
and an antisense strand consisting of SEQ ID NO:1241 (5′-AGUuAGUUuAGAUUCUCGATsT);
the second dsRNA consists of a sense strand consisting of SEQ ID NO:1242 (5′-GcAcAuAGGAGAGAuGAGCUsU-3′)
and an antisense strand consisting of SEQ ID NO:1243 (5′-AAGCUcAUCUCUCCuAuGuGCusG-3′).

12. The composition of claim 1, wherein the first and second dsRNA comprises at least one modified nucleotide.

13. The composition of claim 12, wherein the modified nucleotide is chosen from the group of: a 2′-O-methyl modified nucleotide, a nucleotide comprising a 5′-phosphorothioate group, and a terminal nucleotide linked to a cholesteryl derivative or dodecanoic acid bisdecylamide group.

14. The composition of claim 12, wherein the modified nucleotide is chosen from the group of: a 2′-deoxy-2′-fluoro modified nucleotide, a 2′-deoxy-modified nucleotide, a locked nucleotide, an abasic nucleotide, 2′-amino-modified nucleotide, 2′-alkyl-modified nucleotide, morpholino nucleotide, a phosphoramidate, and a non-natural base comprising nucleotide.

15. The composition of claim 1, wherein the first and second dsRNA each comprise at least one 2′-O-methyl modified ribonucleotide and at least one nucleotide comprising a 5′-phosphorothioate group.

16. The composition of claim 1, wherein each strand of each dsRNA is 19-23 bases in length.

17. The composition of claim 1, wherein each strand of each dsRNA is 21-23 bases in length.

18. The composition of claim 1, wherein each strand of the first dsRNA is 21 bases in length and the sense strand of the second dsRNA is 21 bases in length and the antisense strand of the second dsRNA is 23 bases in length.

19. The composition of claim 1, wherein the first and second dsRNA are present in an equimolar ratio.

20. The composition of claim 1, further comprising Sorafenib.

21. The composition of claim 1, further comprising a lipoprotein.

22. The composition of claim 1, further comprising apolipoprotein E (ApoE).

23. The composition of claim 1, wherein the composition, upon contact with a cell expressing Eg5, inhibits expression of Eg5 by at least 40%.

24. The composition of claim 1, wherein the composition, upon contact with a cell expressing VEGF, inhibits expression of VEGF by at least 40%.

25. The composition of claim 1 wherein administration of the composition to a cell decreases expression of Eg5 and VEGF in the cell.

26. The composition of claim 25, wherein the composition is administered in a nM concentration.

27. The composition of claim 1, wherein administration of the composition to a cell increases monoaster formation in the cell.

28. The composition of claim 1, wherein administration of the composition to a mammal results in at least one effect selected from the group consisting of prevention of tumor growth, reduction in tumor growth, or prolonged survival in the mammal.

29. The composition of claim 28, wherein the effect is measured using at least one assay selected from the group consisting of determination of body weight, determination of organ weight, visual inspection, mRNA analysis, serum AFP analysis and survival monitoring.

30. A method for inhibiting the expression of Eg5/KSP and VEGF in a cell comprising administering the composition of claim 1 to the cell.

31. A method for preventing tumor growth, reducing tumor growth, or prolonging survival in a mammal in need of treatment for cancer comprising administering the composition of claim 1 to the mammal.

32. The method of claim 31, wherein the mammal has liver cancer.

33. The method of claim 31, wherein the mammal is a human with liver cancer.

34. The method of claim 31, wherein a dose containing between 0.25 mg/kg and 4 mg/kg dsRNA is administered to the mammal.

35. The method of claim 31, wherein the dsRNA is administered to a human at about 0.01, 0.1, 0.5, 1.0, 2.5, or 5.0 mg/kg.

36. A method for reducing tumor growth in a mammal in need of treatment for cancer comprising administering the composition of claim 1 to the mammal, the method reducing tumor growth by at least 20%.

37. The method of claim 36, wherein the method reduces KSP expression by at least 60%.
